(19) United States
(12) Patent Application Publication
(10) Pub. No.: US 2010/0009927 A1
(43) Pub. Date: Jan. 14, 2010

(54) Anti-Inflammatory and Anti-Allergy Extracts from Nettle

(75) Inventors: Randall S. Alberte, Estero, FL (US); William P. Roschek, JR., Naples, FL (US); Dan Li, Singapore (SG)

Correspondence Address: FOLEY HOAG, LLP;PATENT GROUP, WORLD TRADE CENTER WEST, 155 SEAPORT BLVD, BOSTON, MA, 02110, US

(73) Assignee: HerbalScience Group LLC, Naples, FL (US)

(21) Appl. No.: 12/502,543

(22) Filed: Jul. 14, 2009

Related U.S. Application Data
(60) Provisional application No. 61/080,515, filed on Jul. 14, 2008.

(51) Int. Cl. A61K 31/70; C07D 239/20; A61K 31/505; C07C 59/185; A61K 31/19; C07C 229/06; A61K 31/195; C07C 229/28; A61K 31/192; C07D 311/02; A61K 31/352; C07F 9/02; A61K 31/685; C07D 221/28; A61K 31/439; C07H 15/00; A61P 29/00; A61P 37/08; A61P 19/02; A61P 17/00; A61P 11/06

(52) U.S. Cl. 514/35; 544/317; 514/274; 562/577; 514/557; 562/570; 514/561; 562/444; 514/567; 549/290; 514/457; 562/569; 514/563; 554/80; 514/78; 546/74; 514/289; 536/4.1

(57) ABSTRACT

The present invention relates in part to nettle extracts that are useful for treating or preventing seasonal allergies, allergic rhinitis, and other inflammatory conditions.

### RELATED APPLICATIONS

This application claims the benefit of priority to U.S. Provisional Application Ser. No. 61/080,515, filed on Jul. 14, 2008, which is herein incorporated by reference in its entirety.

### BACKGROUND OF THE INVENTION

Arthritis is an inflammation of the joints that can be chronic and is realized as joint swelling, immobility and pain. The disease, whether osteoarthritis, rheumatoid arthritis or gout, results from a dysregulation of pro-inflammatory cytokines (e.g., interleukins) and pro-inflammatory enzymes like COX that generate prostaglandins (R. Rottapel, 2001. Putting the brakes on arthritis: can suppressors of cytokine signaling (SOCS) suppress rheumatoid arthritis?, J. Clin. Invest. 108:1745-1747). Fundamental to this pro-inflammatory process is the activation of nuclear transcription factor κB (NF-κB). As a consequence, compounds that suppress the expression of tumor necrosis factor alpha (TNF-α) and COX and their products, or NF-κB directly have significant potential for arthritis treatments. Current estimates suggest that by 2030 about 25% of the US population will be doctor diagnosed with arthritis in some form, dramatically increasing the market for arthritis treatments (J. M. Hootman and C. G. Helmick, 2006. Projections of US prevalence of arthritis and associated activity limitations, Arthritis Rheum. 54:226-229).

The majorities of current drugs for arthritis are non-steroidal anti-inflammatory agents (NSAIDs), and range from OTC products like ibuprofen to prescription drugs like celecoxib (Celebrex). Most are non-selective COX-1 and COX-2 inhibitors (aspirin, ibuprofen, and naproxen), while others, like celecoxib, though not COX-2 specific, are highly selective for COX2 (Y. F. Chen, P. Jobanputra, P. Barton, S. Bryan, A. Fry-Smith, G. Harris and R. S. Taylor, 2008. Cyclooxygenase-2 selective non-steroidal anti-inflammatory drugs (etodolac, meloxicam, celecoxib, rofecoxib, etoricoxib, valdecoxib and lumiracoxib) for osteoarthritis and rheumatoid arthritis: a systematic review and economic evaluation, Health Technol. Assess. 12:1-278, iii). COX-1 inhibitors, those drugs with high COX-1 to COX-2 selectivity, have significant side-effects due to the key anti-inflammatory role of COX-1 in prostaglandin production critical for protection of the gastric mucosa (C. Hawkey, L. Laine, T. Simon, A. Beaulieu, J. Maldonado-Cocco, E. Acevedo, A. Shahane, H. Quan, J. Bolognese and E. Mortensen, 2000. Comparison of the effect of rofecoxib (a cyclooxygenase 2 inhibitor), ibuprofen, and placebo on the gastroduodenal mucosa of patients with osteoarthritis: a randomized, double-blind, placebo-controlled trial. The Rofecoxib Osteoarthritis Endoscopy Multinational Study Group, Arthritis Rheum. 43:370-377). More recently, it has been recognized that inhibition of COX enzymes shunts arachidonic acid, the key substrate for inflammatory pathways, into leukotrienes primarily by up-regulation of 5-LOX (S. K. Kulkarni and V. P. Singh, 2008. Positioning dual inhibitors in the treatment of pain and inflammatory disorders, Inflammopharmacology. 16:1-15; J. Martel-Pelletier, D. Lajeunesse, P. Reboul and J. P. Pelletier, 2003. Therapeutic role of dual inhibitors of 5-LOX and COX, selective and non-selective non-steroidal anti-inflammatory drugs, Ann. Rheum. Dis. 62:501-509; J. L. Masferrer, B. S. Zweifel, K. Seibert and P. Needleman, 1990. Selective regulation of cellular cyclooxygenase by dexamethasone and endotoxin in mice, J. Clin. Invest. 86:1375-1379; P. McPeak, R. Cheruvanky, C. R. S. V. and M. M., 2005. Methods for treating joint inflammation, pain, and loss of mobility. U.S. Pat. No. 6,902,739; Issued 7 Jul. 2005.). Therefore, significant effort has been directed towards the development of drugs or drug combinations that target both COX and 5-LOX. Licofelone is currently one of the most promising (S. K. Kulkarni and V. P. Singh, 2008. Positioning dual inhibitors in the treatment of pain and inflammatory disorders, Inflammopharmacology. 16:1-15; J. M. Alvaro-Gracia, 2004. Licofelone—clinical update on a novel LOX/COX inhibitor for the treatment of osteoarthritis, Rheumatol. 43 Suppl 1:121-i25) and it has a favorable cardiovascular profile (G. Shoba, D. Joy, T. Joseph, M. Majeed, R. Rajendran and P. S. Srinivas, 1998. Influence of piperine on the pharmacokinetics of curcumin in animals and human volunteers, Planta Med. 64:353-356).

The inflammatory cascades involved in the symptoms of osteoarthritis (OA) and rheumatoid arthritis (RA) have been the subjects of intense scientific scrutiny (W. F. Kean and W. W. Buchanan, 2005. The use of NSAIDs in rheumatic disorders 2005: a global perspective, Inflammopharmacology. 13:343-370; S. G. Trivedi, J. Newson, R. Rajakariar, T. S. Jacques, R. Hannon, Y. Kanaoka, N. Eguchi, P. Colville-Nash and D. W. Gilroy, 2006. Essential role for hematopoietic prostaglandin D2 synthase in the control of delayed type hypersensitivity, Proc. Natl. Acad. Sci. USA. 103:5179-5184). Central to these pathways is arachidonic acid, which serves as the substrate for the COX-1 and COX-2 (cyclooxygenase) enzymes as well as the family of lipoxygenases (S. K. Kulkarni and V. P. Singh, 2008. Positioning dual inhibitors in the treatment of pain and inflammatory disorders, Inflammopharmacology. 16:1-15; J. L. Masferrer, B. S. Zweifel, K. Seibert and P. Needleman, 1990. Selective regulation of cellular cyclooxygenase by dexamethasone and endotoxin in mice, J. Clin. Invest. 86:1375-1379; W. F. Kean and W. W. Buchanan, 2005. The use of NSAIDs in rheumatic disorders 2005: a global perspective, Inflammopharmacology. 13:343-370; J. N. Sharma and L. A. Mohammed, 2006. The role of leukotrienes in the pathophysiology of inflammatory disorders: is there a case for revisiting leukotrienes as therapeutic targets?, Inflammopharmacology. 14: 10-16). COX was identified as a target for OA in the early 1990's (J. L. Masferrer, B. S. Zweifel, K. Seibert and P. Needleman, 1990. Selective regulation of cellular cyclooxygenase by dexamethasone and endotoxin in mice, J. Clin. Invest. 86:1375-1379; D. A. Kubuju, B. S. Fletcher, B. C. Barnum, R. W. Lim and H. R. Herschman, 1991. TIS10, a phorbol ester tumor prompter-inducible mRNA from Swiss 3T3 cells, encodes a novel prostaglandin synthase/cyclooxygenase homologue, J. Biol. Chem. 266:12866-12872; W. L. Xie, J. G. Chipman, D. L. Robertson, R. L. Erikson and D. L. Simmons, 1991. Expression of a mitogen-responsive gene encoding prostaglandin synthase is regulated by mRNA splicing, Proc. Natl. Acad. Sci. USA. 88:2692-2696). Investigators discovered a new gene product (COX) that was induced in vitro while others found that COX activity could be induced by cytokines such as interleukin-1 (IL-1) and inhibited by corticosteroids. Steroids inhibited the IL-1-induced COX activity but not basal COX activity. These observations led to the hypothesis that there were two COX isozymes, one of which was constitutively expressed and responsible for basal prostaglandin generation, while the other was induced by inflammatory stimuli such as IL-1 and suppressed by glucocorticoids. The COX-1 enzyme is constitutively expressed and is found in nearly all tissues and cells, while the inducible COX-2 enzyme is the major factor in dramatically enhanced production of prostaglandins from arachidonic acid and their release at sites of inflammation.

COX-1 and COX-2 serve identical functions in catalyzing the conversion of arachidonic acid to prostanoids. The specific prostanoid(s) generated in any given cell is not determined by whether that specific cell expresses COX-1 or COX-2, but by which distal enzymes in the prostanoid synthetic pathways are expressed. Stimulated human synovial cells synthesize small amounts of prostaglandin E₂ (PGE₂) and prostacyclin, but not thromboxane (TxB₂), prostaglandin D (PGD), or prostaglandin F₂ₐ (PGF₂ₐ). Following exposure to IL-1, synovial cells make considerably more PGE₂ and prostacyclin, but they still do not synthesize PGD, TxB₂ or PGF₂ₐ (J. M. Bathon, F. H. Chilton, W. C. Hubbard, M. C. Towns, N. J. Solan and D. Proud, 1996. Mechanisms of prostanoid synthesis in human synovial cells: cytokine-peptide synergism, Inflammation. 20:537-554). The IL1-induced increase in PGE₂ and prostacyclin is mediated exclusively through COX-2 (L. J. Crofford, R. L. Wilder, A. P. Ristimaki, H. Sano, E. F. Remmers, H. R. Epps and T. Hla, 1994. Cyclooxygenase-1 and -2 expression in rheumatoid synovial tissues. Effects of interleukin-1 beta, phorbol ester, and corticosteroids, J. Clin. Invest. 93:1095-1101).

COX-1 is expressed in nearly all cells, indicating that at least low levels of prostanoids are important in serving critical physiological (homeostatic) functions in humans. COX1-mediated production of prostaglandins in the stomach serves to protect the mucosa against the ulcerogenic effects of acid and other insults, and COX1 mediated production of thromboxane in platelets promotes normal clotting. COX-2 levels, in contrast, are dramatically up-regulated in inflamed tissues (K. Yamagata, K. I. Andreasson, W. E. Kaufmann, C. A. Barnes and P. F. Worley, 1993. Expression of a mitogen-inducible cyclooxygenase in brain neurons: regulation by synaptic activity and glucocorticoids, Neuron. 11:371-386; C. D. Breder, D. Dewitt and R. P. Kraig, 1995. Characterization of inducible cyclooxygenase in rat brain, J. Comp. Neurol. 355:296-315). For example, COX-2 expression and concomitant PGE₂ production are greatly enhanced in rheumatoid synovium compared to the less inflamed osteoarthritic synovium, and in animal models of inflammatory arthritis (L. J. Crofford, R. L. Wilder, A. P. Ristimaki, H. Sano, E. F. Remmers, H. R. Epps and T. Hla, 1994. Cyclooxygenase-1 and -2 expression in rheumatoid synovial tissues. Effects of interleukin-1 beta, phorbol ester, and corticosteroids, J. Clin. Invest. 93:1095-1101; G. D. Anderson, S. D. Hauser, K. L. McGarity, M. E. Bremer, P. C. Isakson and S. A. Gregory, 1996. Selective inhibition of cyclooxygenase (COX)-2 reverses inflammation and expression of COX-2 and interleukin 6 in rat adjuvant arthritis, J. Clin. Invest. 97:2672-2679). This is clearly the result of excessive production of IL-1, tumor necrosis factor, and growth factors in the rheumatoid joint. Therefore, COX2 selective inhibitors are highly desirable for both OA and RA, and are key to down-regulating the downstream production of pro-inflammatory prostaglandins and leukotrienes.

The generation of pro-inflammatory prostanoids is a hallmark of cyclooxygenase activity (W. F. Kean and W. W. Buchanan, 2005. The use of NSAIDs in rheumatic disorders 2005: a global perspective, Inflammopharmacology. 13:343-370). There are at least 4 major pathways to the production of prostaglandins, depending on the tissue. In OA and RA, the production of prostaglandin H₂ (PGH₂) by COX-2 is converted to the pro-inflammatory prostanoid, PGE₂ by PGE₂ synthase (J. E. Jeffrey and R. M. Aspden, 2007. Cyclooxygenase inhibition lowers prostaglandin E2 release from articular cartilage and reduces apoptosis but not proteoglycan degradation following an impact load in vitro, Arthrit. Res. Ther. 9:R129; F. Kojima, H. Naraba, S. Miyamoto, M. Beppu, H. Aoki and S. Kawai, 2004. Membrane-associated prostaglandin E synthase-1 is upregulated by proinflammatory cytokines in chondrocytes from patients with osteoarthritis, Arthrit. Res. Ther. 6:R355-R365; K. D. Rainsford, 2004. Cytokines and eicosanoids in arthritis, The Eicosanoids.). However, hematopoietic prostaglandin D₂ (HPGD₂) synthase, which plays a well established role in the inflammatory cascade associated with allergic rhinitis (S. T. Holgate and D. Broide, 2003. New targets for allergic rhinitis—a disease of civilization, Nat. Rev. Drug Discov. 2:902-914; R. L. Thurmond, E. W. Gelfand and P. J. Dunford, 2008. The role of histamine H1 and H4 receptors in allergic inflammation: the search for new antihistamines, Nat. Rev. Drug Discov. 7:41-53), has recently been shown to play an essential role in the control of hypersensitivity and persistent inflammation (S. G. Trivedi, J. Newson, R. Rajakariar, T. S. Jacques, R. Hannon, Y. Kanaoka, N. Eguchi, P. Colville-Nash and D. W. Gilroy, 2006. Essential role for hematopoietic prostaglandin D2 synthase in the control of delayed type hypersensitivity, Proc. Natl. Acad. Sci. USA. 103:5179-5184). The anti-inflammatory role of HPDG₂ outside of allergy is still somewhat unclear, but its implication is key to persistent inflammation.

Several inflammatory processes play a critical role in brain aging and are associated with increased up regulation of COX-2. COX-2 is up-regulated in the central nervous system during aging and is associated with different aging-related brain pathologies (H. Y. Chung, M. Cesari, S. Anton, E. Marzetti, S. Giovannini, A. Y. Seo, C. Carter, B. P. Yu and C. Leeuwenburgh, 2008. Molecular inflammation: Underpinnings of aging and age-related diseases, Ageing Res. Rev.; H. Y. Chung, B. Sung, K. J. Jung, Y. Zou and B. P. Yu, 2006. The molecular inflammatory process in aging, Antioxid. Redox Signal. 8:572-581; D. Wu and S. N. Meydani, 2004. Mechanism of age-associated up-regulation in macrophage PGE2 synthesis, Brain, Behav., Immun. 18:487-494). COX-2 inhibitors have been shown to be a potential therapy for neuronal inflammation. In particular, COX-2 inhibition has been shown to significantly reverse the aging-induced retention deficit in mice (M. Bishnoi, C. S. Patil, A. Kumar and S. K. Kulkarni, 2005. Protective effects of nimesulide (COX Inhibitor), AKBA (5-LOX Inhibitor), and their combination in aging-associated abnormalities in mice, Methods Find. Exp. Clin. Pharmacol. 27:465-470). COX and LOX inhibitors, and their combination, also have been shown to reverse the aging-induced motor dysfunction in aged animals. On the basis of these observations, present findings indicate that COX inhibitors, especially in conjunction with LOX inhibitors (e.g. dual COX/LOX inhibitors), may provide a new therapeutic ovation for the treatment of aging-related brain disorders such as Alzheimer's disease and different motor dysfunctions with adequate gastrointestinal tolerability (D. Paris, T. Town, T. Parker, J. Humphrey and M. Mullan, 2000. A beta vasoactivity: an inflammatory reaction, Ann. N.Y. Acad. Sci. 903:97-109). Thus, both COX-1 and COX-2 activities increase with age contributing to neurodegeneration and inhibition of these enzymes reduces this process.

Alzheimer's disease (AD) is the most common form of dementia and is a mounting public health problem among the elderly. Pharmacoepidemiological data, analytical data from human tissue and body fluids, and mechanistic data mostly from murine models all have implicated oxidation products of two fatty acids, arachidonic acid (AA) and docosahexaenoic acid (DHA), in the pathogenesis of neurodegeneration (J. J. Hoozemans, J. M. Rozemuller, E. S. van Haastert, R. Veerhuis and P. Eikelenboom, 2008. Cyclooxygenase-1 and -2 in the different stages of Alzheimer's disease pathology, Curr. Pharm. Des. 14: 1419-1427). Reduction of COX1/COX2 activity reduces neurotoxicity and neurodegeneration (J. J. Hoozemans, J. M. Rozemuller, E. S. van Haastert, R. Veerhuis and P. Eikelenboom, 2008. Cyclooxygenase-1 and -2 in the different stages of Alzheimer's disease pathology, Curr. Pharm. Des. 14:1419-1427), as these reactions mediate AA oxidation and are key to the pathogenesis of dementias.

Prostaglandins (PG) as a whole have a broad range and impact in health (A. Pahl, S. J. E. and B. B. David, 2008. Prostaglandin-D Synthase, xPharm: The Comprehensive Pharmacology Reference. 1-5). These lipid compounds play numerous roles, including as mediators of nociception, inflammation, and sleep regulation, as well as attractants for TH2 cells, smooth muscle contraction, and bronchial constriction (C. Chen and N. G. Bazan, 2005. Lipid signaling: Sleep, synaptic plasticity, and neuroprotection, Prostaglandins Other Lipid Mediat. 77:65-76; H. Hirai, K. Tanaka, O. Yoshie, K. Ogawa, K. Kenmotsu, Y. Takamori, M. Ichimasa, K. Sugamura, M. Nakamura, S. Takano and K. Nagata, 2001. Prostaglandin D2 selectively induces chemotaxis in T helper type 2 cells, eosinophils, and basophils via seven-transmembrane receptor CRTH2, J. Exp. Med. 193:255-261; T. Matsuoka, M. Hirata, H. Tanaka, Y. Takahashi, T. Murata, K. Kabashima, Y. Sugimoto, T. Kobayashi, F. Ushikubi, Y. Aze, N. Eguchi, Y. Urade, N. Yoshida, K. Kimura, A. Mizoguchi, Y. Honda, H. Nagai and S. Narumiya, 2000. Prostaglandin D2 as a mediator of allergic asthma, Science. 287:2013-2017; T. R. Scott, A. R. Messersmith, W. J. McCrary, J. L. Herlong and S. C. Burgess, 2005. Hematopoietic prostaglandin D2 synthase in the chicken Harderian gland, Vet. Immunol. Immunopathol. 108:295-306; Y. Urade and O. Hayaishi, 1999. Prostaglandin D2 and sleep regulation, Biochim. Biophys. Acta. 1436:606-615; Y. Urade, O. Hayaishi, H. Matsumura and K. Watanabe, 1996. Molecular mechanism of sleep regulation by prostaglandin D2, J. Lipid Mediators Cell Signalling. 14:71-82). The primary ways in which PGs perform this function is as a ligand for specific PG receptors or serving as a precursor to another biologically significant molecule. One example of a PG of importance is Prostaglandin D2 (PGD). This PG has been found to be a major regulator of sleep and nociception in the central nervous system as well as regulating inflammation and allergies throughout the body due to secretion by mast cells and basophils (Y. Urade and N. Eguchi, 2002. Lipocalin-type and hematopoietic prostaglandin D synthases as a novel example of functional convergence, Prostaglandins Other Lipid Mediat. 68-69:375-382; Y. Urade and O. Hayaishi, 2000. Prostaglandin D synthase: structure and function, Vitam. Horm. 58:89-120). PGD also has a net effect throughout the body due to it being a precursor to other biologically relevant molecules, such as the J series of PGs (PGJ), which are important for signaling, especially as a ligand for PPAR gamma (B. Lohrke, T. Viergutz, S. K. Shahi, R. Pohland, K. Wollenhaupt, T. Goldammer, H. Walzel and W. Kanitz, 1998. Detection and functional characterisation of the transcription factor peroxisome proliferator-activated receptor gamma in lutein cells, J. Endocrinol. 159:429-439).

PGD is synthesized by the Prostaglandin D2 Synthases (PGDS). There are two types of PGDS enzymes, the hematopoietic PGDS (H-PGDS) and the Lipocalin PGDS (L-PGDS) (Y. Urade and N. Eguchi, 2002. Lipocalin-type and hematopoietic prostaglandin D synthases as a novel example of functional convergence, Prostaglandins Other Lipid Mediat. 68-69:375-382). L-PGDS is localized primarily to the central nervous system and male genitals (R. L. Gerena, D. Irikura, Y. Urade, N. Eguchi, D. A. Chapman and G. J. Killian, 1998. Identification of a fertility-associated protein in bull seminal plasma as lipocalin-type prostaglandin D synthase, Biol. Reprod. 58:826-833; S. Fouchecourt, F. Dacheux and J. L. Dacheux, 1999. Glutathione-independent prostaglandin D2 synthase in ram and stallion epididymal fluids: origin and regulation, Biol. Reprod. 60:558-566; K. Ikai, M. Ujihara, K. Fujii and Y. Urade, 1989. Inhibitory effect of tranilast on prostaglandin D synthetase, Biochem. Pharmacol. 38:2673-2676), whereas the H-PGDS is localized in cells such as mast cells, antigen-presenting cells and Th2 cells, as well as in peripheral tissues (K. Tanaka, K. Ogawa, K. Sugamura, M. Nakamura, S. Takano and K. Nagata, 2000. Cutting edge: differential production of prostaglandin D2 by human helper T cell subsets, J. Immunol. 164:2277-2280; M. Ujihara, Y. Urade, N. Eguchi, H. Hayashi, K. Ikai and O. Hayaishi, 1988. Prostaglandin D2 formation and characterization of its synthetases in various tissues of adult rats, Arch. Biochem. Biophys. 260:521-531; Y. Urade, M. Ujihara, Y. Horiguchi, M. Igarashi, A. Nagata, K. Ikai and O. Hayaishi, 1990. Mast cells contain spleen-type prostaglandin D synthetase, J. Biol. Chem. 265:371-375; Y. Urade, M. Ujihara, Y. Horiguchi, K. Ikai and O. Hayaishi, 1989. The major source of endogenous prostaglandin D2 production is likely antigen-presenting cells. Localization of glutathione-requiring prostaglandin D synthetase in histiocytes, dendritic, and Kupffer cells in various rat tissues, J. Immunol. 143:2982-2989).

H-PGDS localized expression is very important for its function in immunity response, allergic reactions, and inflammation. This enzyme, a member of the sigma glutathione dependant transferases, is expressed in peripheral tissues and in cells related to immune response, allergy, and asthma (Y. Urade and O. Hayaishi, 1999. Prostaglandin D2 and sleep regulation, Biochim. Biophys. Acta. 1436:606-615; K. Tanaka, K. Ogawa, K. Sugamura, M. Nakamura, S. Takano and K. Nagata, 2000. Cutting edge: differential production of prostaglandin D2 by human helper T cell subsets, J. Immunol. 164:2277-2280; Y. Urade, M. Ujihara, Y. Horiguchi, M. Igarashi, A. Nagata, K. Ikai and O. Hayaishi, 1990. Mast cells contain spleen-type prostaglandin D synthetase, J. Biol. Chem. 265:371-375; Y. Urade, M. Ujihara, Y. Horiguchi, K. Ikai and O. Hayaishi, 1989. The major source of endogenous prostaglandin D2 production is likely antigen-presenting cells. Localization of glutathione-requiring prostaglandin D synthetase in histiocytes, dendritic, and Kupffer cells in various rat tissues, J. Immunol. 143:2982-2989; M. Ujihara, Y. Horiguchi, K. Ikai and Y. Urade, 1988. Characterization and distribution of prostaglandin D synthetase in rat skin, J. Invest. Dermatol. 90:448-451). PGD production at these sites is important due to the receptors, namely D type prostaglandin (DP) and Chemo-attractant Receptor-homologous molecule expressed TH2 cells (CRTH2), and also because PGD serves as a precursor to PGJs (H. Giles and P. Leff, 1988. The biology and pharmacology of PGD2, Prostaglandins. 35:277-300; K. Kabashima and S. Narumiya, 2003. The DP receptor, allergic inflammation and asthma, Prostag. Leukotr. Ess. Fatty Acids. 69:187-194; Y. Kanaoka and Y. Urade, 2003. Hematopoietic prostaglandin D synthase, Prostag. Leukotr. Ess. Fatty Acids. 69:163-167; T. Satoh, R. Moroi, K. Aritake, Y. Urade, Y. Kanai, K. Sumi, H. Yokozeki, H. Hirai, K. Nagata, T. Hara, M. Utsuyama, K. Hirokawa, K. Sugamura, K. Nishioka and M. Nakamura, 2006. Prostaglandin D2 plays an essential role in chronic allergic inflammation of the skin via CRTH2 receptor, J. Immunol. 177:2621-2629; S. G. Trivedi, J. Newson, R. Rajakariar, T. S. Jacques, R. Hannon, Y. Kanaoka, N. Eguchi, P. Colville-Nash and D. W. Gilroy, 2006. Essential role for hematopoietic prostaglandin D2 synthase in the control of delayed type hypersensitivity, Proc. Natl. Acad. Sci. USA. 103:5179-5184). The receptors for DP have been shown in DP null mice studies to be less likely to react to irritants (T. Matsuoka, M. Hirata, H. Tanaka, Y. Takahashi, T. Murata, K. Kabashima, Y. Sugimoto, T. Kobayashi, F. Ushikubi, Y. Aze, N. Eguchi, Y. Urade, N. Yoshida, K. Kimura, A. Mizoguchi, Y. Honda, H. Nagai and S, Narumiya, 2000. Prostaglandin D2 as a mediator of allergic asthma, Science. 287:2013-2017). Also, studies have shown in transgenic mice with enhanced expression of PGDS in lung tissue displayed enhanced symptoms of the allergic response than wild type mice along with increased expression of DP receptors upon allergen exposure (Y. Fujitani, Y. Kanaoka, K. Aritake, N. Uodome, K. Okazaki-Hatake and Y. Urade, 2002. Pronounced eosinophilic lung inflammation and Th2 cytokine release in human lipocalin-type prostaglandin D synthase transgenic mice, J. Immunol. 168:443-449). Another means that H-PGDS has to mediate the inflammation, allergies, and asthma is through PGD serving as a precursor to PGJs that either serves as PPAR gamma agonists or as immune system modulators (S. G. Trivedi, J. Newson, R. Rajakariar, T. S. Jacques, R. Hannon, Y. Kanaoka, N. Eguchi, P. Colville-Nash and D. W. Gilroy, 2006. Essential role for hematopoietic prostaglandin D2 synthase in the control of delayed type hypersensitivity, Proc. Natl. Acad. Sci. USA. 103:5179-5184). Due to the extensive role that PGDS plays in the allergic response and immune system, an interest has developed into inhibitors of H-PGDS as a potential treatment for allergies, inflammation and asthma. This has lead to the finding of HQL-79, a H-PGDS selective inhibitor (K. Aritake, Y. Kado, T. Inoue, M. Miyano and Y. Urade, 2006. Structural and functional characterization of HQL-79, an orally active, selective inhibitor for human hematopoietic prostaglandin D synthase, J. Biol. Chem. M506431200). Clearly, there is a potential role for H-PGDS inhibitors for numerous conditions characterized by inflammation, allergies, and pulmonary disease.

Tryptase is a tetrameric serine protease with a molecular size of 134 kD. The four monomers weigh 32-34 kD and each possess one catalytic site. Its presence is restricted almost exclusively to mast cells, where tryptase exists contained in secretory granules complexed with cytokines, histamine and heparin proteoglycans (J. A. Cairns and A. F. Wells, 1997. Mast Cell Tryptase Stimulates the Synthesis of Type I Collagen in Human Lung Fibroblasts, J. Clin. Invest. 99:1313-1321). Some of the cytokines include interleukins 1, 4 and 6, tumor necrosis factor, transforming growth factor, and basic fibroblast growth factor with roles in controlling processes of inflammation and fibrosis (J. A. Cairns and A. F. Wells, 1997. Mast Cell Tryptase Stimulates the Synthesis of Type I Collagen in Human Lung Fibroblasts, J. Clin. Invest. 99:1313-1321).

Fibrosis is a prominent feature of chronically inflamed tissue. This pathology is characterized by progressive and extreme accumulation of extracellular matrix collagen as a result of increased proliferation of fibroblasts. Fibroblasts are the key mesenchymal cells in the synthesis of interstitial collagen. A characteristic of lung tissue from patients with fibrotic lung disease is an elevated number of mast cells, many of which are degranulated and located in close proximity to proliferating fibroblasts (J. A. Cairns and A. F. Wells, 1997. Mast Cell Tryptase Stimulates the Synthesis of Type I Collagen in Human Lung Fibroblasts, J. Clin. Invest. 99:1313-1321). Elevated concentrations of tryptase and other mast cell products are also present in bronchoalveolar fluid gathered from patients with fibrotic lung disease (J. A. Cairns and A. F. Wells, 1997. Mast Cell Tryptase Stimulates the Synthesis of Type I Collagen in Human Lung Fibroblasts, J. Clin. Invest. 99:1313-1321).

Due to the relationship between mast cell degranulation from tryptase, leading to a positive feed-back by even higher levels of tryptase, and allergic responses, medications have been developed that prevent mast cell degranulation. These medications are known as mast cell stabilizers. Currently, there are several mast cell stabilizers on the market serving as treatments for asthma, allergic rhinitis (hay fever), allergic conjunctivitis, allergic sinusitis, and mastocytosis (S. Lal, P. D. Dorow, K. K. Venho and S. S. Chatterjee, 1993. Nedocromil sodium is more effective than cromolyn sodium for the treatment of chronic reversible obstructive airway disease, Chest. 104:438-447; E. O. Meltzer, 2006. Allergic rhinitis: managing the pediatric spectrum, Allergy Asthma Proc. 27:2-8; M. L. Hayden and C. R. Womack, 2007. Caring for patients with allergic rhinitis, J. Am. Acad. Nurse Pract. 19:290-298; G. G. Shapiro and P. Konig, 1985. Cromolyn sodium: a review, Pharmacotherapy. 5:156-170; B. A. Berman, 1983. Cromolyn: past, present, and future, Pediatr. Clin. North Am. 30:915-930; N. A. Soter, K. F. Austen and S. I. Wasserman, 1979. Oral disodium cromoglycate in the treatment of systemic mastocytosis, New Engl. J. Med. 301:465-469; T. J. Ferkovic, T. R. Lanese and B. D. Long, 1982. Use of oral cromolyn sodium in systemic mastocytosis, Clin. Pharm. 1:377-379). Due to the effectiveness of mast cell stabilizers, tryptase inhibitors are likely to be also very effective therapeutics for allergic responses and inflammation. The anti-inflammatory action in the lungs would also decrease bronchoconstriction and have anti-tussive potential.

Histamine receptors in the body are associated with numerous physiological functions including mast cell chemotaxis, allergic responses throughout the body, antibody synthesis, t-cell proliferation, vasoconstriction, bronchodilation, nausea, as well as many other neurotransmitter activities throughout the CNS (S. J. Hill, C. R. Ganellin, H. Timmerman, J. C. Schwartz, N. P. Shankley, J. M. Young, W. Schunack, R. Levi and H. L. Haas, 1997. International Union of Pharmacology. XIII. Classification of histamine receptors, Pharmacol. Rev. 49:253-278; L. M. DuBuske, 1997. Clinical comparison of histamine HI-receptor antagonist drugs, J. Allergy Clin. Immunol. 98:307-318). As a result, antihistamines (histamine antagonists and/or negative/reverse agonists) are highly important for the treatment of the allergic response and bronco-constriction, as well as having immune enhancement potential.

Nausea and motion sickness have been associated with elevated levels of histamine. H1 antagonists, particularly dimenhydrinate (Dramamine®) and scopolamine, have been shown to be efficacious in the treatment of these symptoms (nausea and motion sickness) (S. E. Weinstein and R. M. Stern, 1997. Comparison of marezine and dramamine in preventing symptoms of motion sickness, Aviat. Space Environ. Med. 68:890-894; A. B. Spinks, J. Wasiak, E. V. Villanueva and V. Bernath, 2007. Scopolamine (hyoscine) for preventing and treating motion sickness, Cochrane Database Syst. Rev. CD002851). As such, antihistamines would be of great importance for the treatment of nausea and motion sickness. Many antihistamines also have sedative effects (A. N. Nicholson, P. A. Pascoe, C. Turner, C. R. Ganellin, P. M. Greengrass, A. F. Casy and A. D. Mercer, 1991. Sedation and histamine H1-receptor antagonism: studies in man with the enantiomers of chlorpheniramine and dimethindene, Br. J. Pharmacol. 104:270-276; P. B. Reiner and A. Kamondi, 1994. Mechanisms of antihistamine-induced sedation in the human brain: H1 receptor activation reduces a background leakage potassium current, Neurosci. 59:579-588).

More than 20 million people, over 40% of who are children, suffer from seasonal allergies in the United States (A. W. Law, S. D. Reed, J. S. Sundy and K. A. Schulman, 2003. Direct costs of allergic rhinitis in the United States: Estimates from the 1996 medical expenditure panel survey, Journal of Allergy and Clinical Immunology. 111:296-300; S. T. Holgate and D. Broide, 2003. New targets for allergic rhinitis—a disease of civilization, Nature Reviews Drug Discovery. 2:902-914). In the last few years, several investigators have shown that there is a strong genetic and environmental component to the allergic inflammatory response (S. T. Holgate and D. Broide, 2003. New targets for allergic rhinitis—a disease of civilization, Nature Reviews Drug Discovery. 2:902-914; M. Schatz, 2007. A survey of the burden of allergic rhinitis in the USA, Allergy. 62:9-16; Y. Schoefer, T. Schafer, C. Meisinger, H. E. Wichmann and J. Heinrich, 2008. Predictivity of allergic sensitization (RAST) for the onset of allergic diseases in adults, Allergy. 63:81-86; J. Bousquet, H. A. Boushey, W. W. Busse, G. W. Canonica, S. R. Durham, C. G. Irvin, J. P. Karpel, P. van Cauwenberge, R. Chen, D. G. Jezzoni and A. G. Harris, 2004. Characteristics of patients with seasonal allergic rhinitis and concomitant asthma, Clinical & Experimental Allergy. 34:897-903). Although there are numerous over-the-counter (OTC), prescription, and herbal-based medications on the market for allergies, many of these products suffer from undesirable side-effects like headache, dry mouth and/or drowsiness. Despite this, allergy and sinus treatment drugs for air borne pollen and/or particulate allergens are among the safest drugs in the world, with an extremely low number of adverse effects from use (J. Bousquet, T. Bieber, W. Fokkens, M. L. Kowalski, M. Humbert, B. Niggemann, H. U. Simon, P. Burney, P. van Cauwenberge, T. Zuberbier, C. A. Akdis and P. Demoly, 2008. Important questions in allergy: novel research areas, Allergy. 63:143-147; I. Hore, C. Georgalas and G. Scadding, 2005. Oral antihistamines for the symptom of nasal obstruction in persistent allergic rhinitis—a systematic review of randomized controlled trials, Clinical & Experimental Allergy. 35:207-212; M. Kawai, T. Hirano, S. Higa, J. Arimitsu, M. Maruta, Y. Kuwahara, T. Ohkawara, K. Hagihara, T. Yamadori, Y. Shima, A. Ogata, I. Kawase and T. Tanaka, 2007. Flavonoids and related compounds as anti-allergic substances, Allergology International 56:113-123; B. J. Lipworth, 2001. Emerging role of antileukotriene therapy in allergic rhinitis, Clinical & Experimental Allergy. 31:1813-1821; G. K. Scadding, 1999. Clinical assessment of antihistamines in rhinitis, Clinical & Experimental Allergy. 29:77-81).

Nettle (Urtica dioica L.) is a temperate species, which is cultivated commercially, but is a common and aggressive weed in moist soils throughout the US and Europe. Urtica dioica belongs to the family Urticaceae. The Latin root of Urtica is uro, meaning “I burn”, indicative of the small stings caused by the little hairs on the leaves of this plant that burn when contact is made with the skin. The leaves have a high density of glandular hairs that contain formic acid and histamine, the agents that cause the ‘stinging’. Dermatological reactions from exposure to the formic acid which is released with even gentle mechanical stress to the leaves can range from mild irritation to severe dermatitis. Despite this feature, the young shoots and leaves are harvested and blanched in boiling water, neutralizing the formic acid, to yield a tasty vegetable dish and as an additive to soups. The plant produces high quality fibers and is being cultivated for this use in Europe.

The root and leaves of nettle are used in herbal medicine. The mineral-rich leaves are used mainly for their diuretic properties, in the treatment of anemia, as a blood tonic and purifier and an infusion relieves high blood pressure and cystitis. A decoction of the root is astringent and indicated for diarrhea and dysentery. Homoeopaths use a fresh plant tincture for eczema. As an infusion, nettle leaves are taken in doses of 2 fl oz (56 mL). The typical dose of the powdered herb is 5-10 grains (325-650 mg).

Nettle has been used for hundreds of years to treat rheumatism (disorders of the muscles and joints), eczema, arthritis, gout, and anemia. Today, many people use it to treat urinary problems during the early stages of an enlarged prostate (called benign prostatic hyperplasia or BPH), for urinary tract infections, for kidney stones, for hay fever (allergic rhinitis), or in compresses or creams for treating joint pain, sprains and strains, tendonitis, and insect bites. In fact, some small but well designed studies are begging to confirm that certain traditional uses have scientific validity, particularly osteoarthritis especially when used in conjunction with anti-inflammatory medications. Recent laboratory studies are offering plausible explanations for why stinging nettles might help rheumatoid arthritis as well as several of the conditions already mentioned (R. L. Thurmond, E. W. Gelfand and P. J. Dunford, 2008. The role of histamine H1 and H4 receptors in allergic inflammation: the search for new antihistamines, Nat. Rev. Drug Discov. 7:41-53; J. E. Chrubasik, B. D. Roufogalis, H. Wagner and S. A. Chrubasik, 2007. A comprehensive review on nettle effect and efficacy profiles, Part J: herba urticae, Phytomedicine. 14:423-435; H. Pajouhesh and G. R. Lenz, 2005. Medicinal chemical properties of successful central nervous system drugs, NeuroRx. 2:541-553).

Recent studies have shown nettle leaf activity against certain inflammatory markers and related disorders (H. Tunon, C. Olavsdoter and L. Bohin, 1995. Evaluation of anti-inflammatory activity of some Swedish medicinal plants. Inhibition of prostaglandin biosynthesis and PAF-induced, J. Ethnopharmacol. 48:61-76; A. Konrad, M. Mahler, S. Ari, B. Flogerzi and S. Klingelhofer, 2005. Ameliorative effect of IDS 30, a stinging nettle leaf extract, on chronic colitis, Int. J. Colorectal Dis. 20:9-17; R. Miesel, M. Kurpisz and H. Kroger, 1995. Modulation of inflammatory arthritis by inhibition of poly(ADP ribose) polymerase, Inflammation. 19:379-387; T. Ozen and H. Korkmaz, 2003. Modulatory effect of Urtica dioica L. (Urticaceae) leaf extract on biotransformation enzyme systems, antioxidant enzymes, lactate dehydrogenase and lipid peroxidation in mice, Phytomedicine. 10:405-415; G. A. FitzGerald, 2003. COX-2 and beyond: Approaches to prostaglandin inhibition in human disease, Nat. Rev. Drug Discov. 2:879-890).

Nettle has been shown to possess benefits for allergies (e.g., allergic rhinitis), as it has anti-allergenic properties. Nettle treats hay fever, asthma, itchy skin conditions, and insect bites. The juice can be used to treat nettle stings. Decongestants, antihistamines, allergy shots and even prescription medications such as Allegra® and Claritin® treat only the symptoms of allergies and tend to lose effectiveness over a period of time. They can also cause drowsiness, dry sinuses, insomnia and high blood pressure. Nettle has none of these side effects. It can be used on a regular basis and has an impressive number of other benefits most notably as a treatment for prostate enlargement. In a double-blind placebo-controlled randomized study of 98 patients with allergic rhinitis, the effect of a freeze-dried preparation of Urtica dioica was compared against placebo. Based on daily symptom diaries and the global response recorded at the follow-up visit after one week of therapy, Urtica dioica was rated higher than placebo in relieving symptoms.

Key control points in allergic rhinitis, an inflammatory response to particulates like pollen, dust and related allergins, include the enzymes that control the flow of arachidonic acid into an inflammatory cascade that generates prostaglandins and leukotrienes (see FIG. 1 and FIG. 2). The major players in the cascade (see FIG. 2) are histamine production and release (H₁ receptors), prostaglandin D₂ Synthase responsible for the production of certain pro-inflammatory prostaglandins, the Leukotriene Receptor that controls pro-inflammatory leukotriene release, and Typtase. Tryptase, in particular, controls the degranulation of Mast cells and certain Basophils that that contain a broad diversity of cytokines and chemokines that drive the inflammatory manifestation of allergic rhinitis; these include, runny nose, itchy and watery eyes, sneezing, itchy skin, and issue swelling (P. Edwards, 2006. Combinatorial approach towards the discovery of tryptase inhibitors, Drug Discov. Today. 11: 181-182; W. Cookson, 2002. Genetics and genomics of asthma and allergic diseases, Immunol Rev. 190:195-206; J. W. Steinke, S. S. Rich and L. Borish, 2008. Genetics of allergic disease, J. Allergy Clin. Immunol 121:S384-S387). Nettle has been studied and has shown promise in treating Alzheimer's disease, arthritis, asthma, bladder infections, bronchitis, bursitis, gingivitis, gout, hives, kidney stones, laryngitis, multiple sclerosis, PMS, prostate enlargement, sciatica, and tendonitis. Externally it has been used to improve the appearance of the hair, and is said to be a remedy against oily hair and dandruff.

Nettle has been shown to have anti-inflammatory effects and to boost the immune system. Aerial parts have been used historically to treat muscle pain and arthritis. Taken orally, products (specifically magnonol) made from nettle aerial parts may interfere with the body production of inflammation-causing chemicals specifically tumor necrosis factor-alpha (TNF-α). Consequently, the aerial parts of nettle may have the primary anti-inflammatory effect. They may also enhance responses of the immune system. Chemicals in nettle aerial parts are also thought to reduce the feeling of pain or interfere with the way that nerves send pain signals. All of these effects may reduce the pain and stiffness of arthritis and similar conditions. They may also have some value for relieving other inflammatory conditions such as colitis. Lastly nettle possesses astringent properties and has been shown to slow or stop bleeding from wounds and nosebleeds, and is good for heavy menstrual bleeding.

Nettle has a long history of use as an anti-inflammatory in homeopathic medicine. The Homeopathic Pharmacopeia includes a monograph on Urtica dioica leaves that describes its uses for seasonal allergies and upper respiratory maladies. Toxicology screens on nettle extracts show little to no toxicity, mutagenicity and carcinogenicity (W. Cookson, 2002. Genetics and genomics of asthma and allergic diseases, Immunol. Rev. 190:195-206). Ethanolic extracts of nettle show increased hepatic biotransformation and antioxidant enzymes in rats with no evidence of liver damage (J. W. Steinke, S. S. Rich and L. Borish, 2008. Genetics of allergic disease, J. Allergy Clin. Immunol. 121:S384-S387).

Based on the above, there is a need for novel nettle extract compositions having certain medically beneficial chemical constituents.

### SUMMARY OF THE INVENTION

The present disclosure provides in part certain extracts of nettle, which contain certain compounds that are active against one or more inflammatory-related endpoints, such as COX, LOX, HPDGS, Tryptase and H₁ Receptor. For example, one embodiment relates to a nettle extract comprising at least one compound selected from the group consisting of 6-azacytosine, levulinic acid, threonine, niacinamide, DL-methyl-m-tyrosine, 4-methyl-7-ethoxy coumarin, vitamin B5, isopropyl-B-D-thiogalactopyranoside, osthole, phosphatidylcholine, 4-shogaol, piperine/cocluarine/laurifoline, 8-dehydrogingerdione, sinomenin/deoxyharringtonine, and picrocrocin/carnosol.

In some embodiments, the aforementioned nettle extracts may further comprise at least one of resorcinol, proline, leucine, adenine, levoglucosan/glycogen/laminarin, synephrine, or shikimic acid. In other embodiments, the aforementioned extracts further comprise 3,4-dimethoxychalcone.

In another aspect, the present invention relates to nettle (Urtica dioica) extracts comprising a fraction having a Direct Analysis in Real Time (DART) Time-of-Flight (TOF) mass spectrometry chromatogram as shown in any of FIGS. 3-10.

In another aspect, the present invention relates to a pharmaceutical composition comprising an extract of nettle and a pharmaceutically acceptable carrier. In another aspect, the invention relates to a food or medicament comprising a nettle extract.

The extracts of the present invention are useful for treating or preventing seasonal allergies or allergic rhinitis. As disclosed herein, preferred extracts are enriched in a range of bioactives that address several important and key allergic rhinitis endpoints, including (1) H1 receptor inactivation/inhibition, blocking histamine function; (2) prostaglandin D₂ synthase inhibition, blocking prostaglandin production by mast cells and basophils; (3) COX-1 and COX-2 inhibition, blocking prostaglandin formation; (4) 5-LOX inhibition which blocks leukotriene production: and, (5) tryptase inhibition blocking mast cell degranulation and release of allergenic and immune mediators that cause a range of allergy symptoms. The extracts down-regulate or mitigate these known key immune and inflammatory responses to air-borne allergens that constitute allergic rhinitis, or hay fevers.

As such, the allergic responses that include sneezing, nasal congestion, itchy and watery eyes and related discomforts will be minimized or mitigated.

Another aspect of the invention relates to methods of making the extracts.

Further features and advantages of the disclosed extracts will become apparent from the description, drawings and claims that follow.

### BRIEF DESCRIPTION OF THE DRAWINGS

FIG. 1 depicts the role of Arachidonic Acid, COX and Prostaglandins in inflammatory cascades. The inflammatory pathway for Allergic Rhinitis is highlighted in dashed red lines (R. L. Thurmond, E. W. Gelfand and P. J. Dunford, 2008. The role of histamine H1 and H4 receptors in allergic inflammation: the search for new antihistamines, Nat. Rev. Drug Discov. 7:41-53).

FIG. 2 depicts a flow diagram showing the steps in Allergic Rhinitis where an allergen activates a Dendritic cell/T cell and this triggers mast cell degrandulation leading to the production of inflammatory mediators (Histamine, leukotrienes, prostaglandins and tryptase) and a cytokine cascade (F. Oliver, E. U. Amon, A. Breathnach, D. M. Francis, P. Sarathchandra, A. K. Black and M. W. Greaves, 1991. Contact urticaria due to the common stinging nettle (Urtica dioica): histological, ultrastructural and pharmacological studies, Clin. Exp. Dermatol. 16:1-7).

FIG. 3 depicts a DART TOF-MS fingerprint of showing the relative abundances of each of the chemical species (Y-axis) and the mass distribution (X-axis) for Extract 1.

FIG. 4 depicts a DART TOF-MS fingerprint of showing the relative abundances of each of the chemical species (Y-axis) and the mass distribution (X-axis) for Extract 2.

FIG. 5 depicts a DART TOF-MS fingerprint of showing the relative abundances of each of the chemical species (Y-axis) and the mass distribution (X-axis) for Extract 3.

FIG. 6 depicts a DART TOF-MS fingerprint of showing the relative abundances of each of the chemical species (Y-axis) and the mass distribution (X-axis) for Extract 4.

FIG. 7 depicts a DART TOF-MS fingerprint of showing the relative abundances of each of the chemical species (Y-axis) and the mass distribution (X-axis) for Extract 5.

FIG. 8 depicts a DAT TOF-MS fingerprint of showing the relative abundances of each of the chemical species (Y-axis) and the mass distribution (X-axis) for Extract 6.

FIG. 9 depicts a DART TOF-MS fingerprint of showing the relative abundances of each of the chemical species (Y-axis) and the mass distribution (X-axis) for Extract 7.

FIG. 10 depicts a DART TOF-MS fingerprint of showing the relative abundances of each of the chemical species (Y-axis) and the mass distribution (X-axis) for Extract 8.

FIG. 11 depicts the pharmacokinetic profile of key bioactives in nettle extract 2 present in serum after ingestion of 200 mg of nettle extract 2. Values are the average of serum samples from 5 adults and data are presented as relative intensity from the DART TOF mass spectra.

FIG. 12 depicts the pharmacokinetic profile of key bioactives in nettle extract 2 present in urine after ingestion of 200 mg of nettle extract 2. Values are the average of serum samples from 5 adults and data are presented as relative intensity from the DART TOF mass spectra.

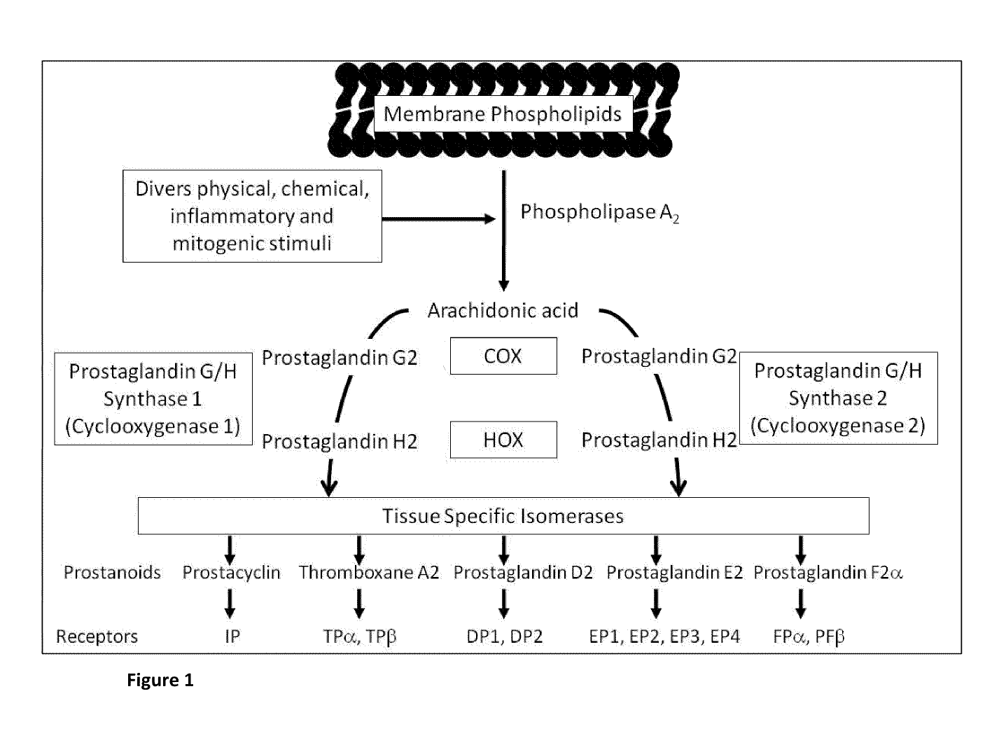

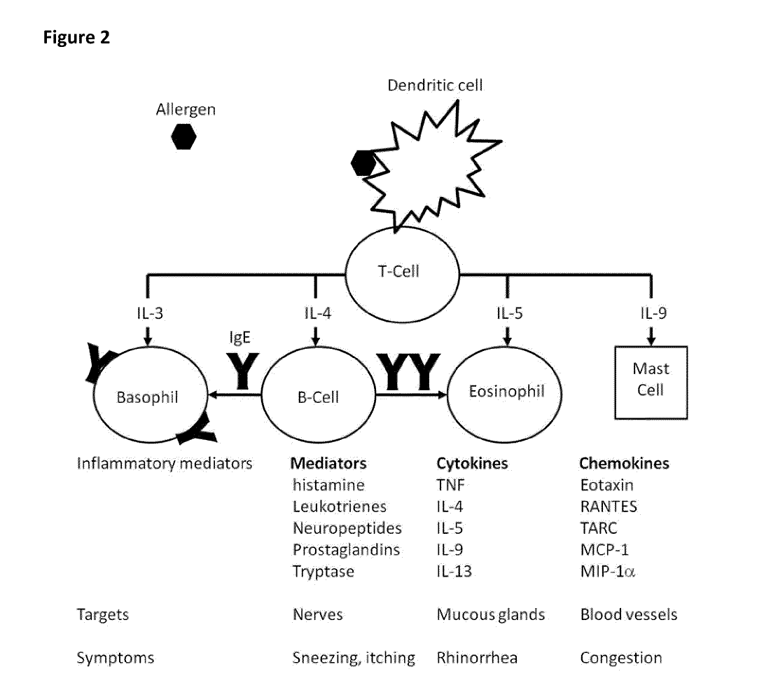

### DETAILED DESCRIPTION OF THE INVENTION

### Definitions

The term “effective amount” as used herein refers to the amount necessary to elicit the desired biological response. As will be appreciated by those of ordinary skill in this art, the effective amount of a composite or bioactive agent may vary depending on such factors as the desired biological endpoint, the bioactive agent to be delivered, the composition of the encapsulating matrix, the target tissue, etc.

As used herein, the term “extract” refers to a product prepared by extraction. The extract may be in the form of a solution in a solvent, or the extract may be a concentrate or essence which is free of, or substantially free of solvent. The extract also may be formulated into a pharmaceutical composition or food product, as described further below. The term extract may be a single extract obtained from a particular extraction step or series of extraction steps, or the extract also may be a combination of extracts obtained from separate extraction steps. Such combined extracts are thus also encompassed by the term “extract.”

As used herein, “feedstock” generally refers to raw plant material, comprising whole plants alone, or in combination with on or more constituent parts of a plant comprising leaves, roots, including, but not limited to, main roots, tail roots, and fiber roots, stems, bark, leaves, berries, seeds, and flowers, wherein the plant or constituent parts may comprise material that is raw, dried, steamed, heated or otherwise subjected to physical processing to facilitate processing, which may further comprise material that is intact, chopped, diced, milled, ground or otherwise processed to affected the size and physical integrity of the plant material. Occasionally, the term “feedstock” may be used to characterize an extraction product that is to be used as feed source for additional extraction processes.

As used herein, the term “fraction” means the extraction composition comprising a specific group of chemical compounds characterized by certain physical, chemical properties or physical or chemical properties.

A “patient,” “subject” or “host” to be treated by the subject method may be a primate (e.g. human), bovine, ovine, equine, porcine, rodent, feline, or canine.

The term “pharmaceutically-acceptable salts” is art-recognized and refers to the relatively non-toxic, inorganic and organic acid addition salts of compounds, including, for example, those contained in compositions of the present invention.

The term “synergistic” is art recognized and refers to two or more components working together so that the total effect is greater than the sum of the components.

The term “treating” is art-recognized and refers to curing as well as ameliorating at least one symptom of any condition or disorder.

As used herein, the term “cyclooxygenase” or “COX” refers to an enzyme that is responsible for the formation of biological molecules call prostanoids, including prostaglandins, thromboxane, and prostacyclin.

As used herein, the term “allergy” refers to a disorder (or improper reaction) of the immune system often also referred to as atopy. Allergic reactions occur to normally harmless environmental substances known as allergens; these reactions are acquired, predictable, and rapid. Strictly, allergy is one of four forms of hypersensitivity and is called type I (or immediate) hypersensitivity. It is characterized by excessive activation of certain white blood cells called mast cells and basophils by a type of antibody known as IgE, resulting in an extreme inflammatory response. Common allergic reactions include eczema, hives, hay fever, asthma, food allergies, and reactions to the venom of stinging insects such as wasps and bees. Mild allergies like hay fever are highly prevalent in the human population and cause symptoms such as allergic conjunctivitis, itchiness, and runny nose. Allergies can play a major role in conditions such as asthma. In some people, severe allergies to environmental or dietary allergens or to medication may result in life-threatening anaphylactic reactions and potentially death.

As used herein, the term “HPGDS” refers to hematopoietic Prostaglandin-D synthase, a sigma class glutathione-S-transferase family member. The enzyme catalyzes the conversion of PGH2 to PGD2 and plays a role in the production of prostanoids in the immune system and mast cells. The presence of this enzyme can be used to identify the differentiation stage of human megakaryocytes.

As used herein, the term “tryptase” refers to the most abundant secretory granule-derived serine proteinase contained in mast cells that has recently been used as a marker for mast cell activation. It is involved with allergenic response and is suspected to act as a mitogen for fibroblast lines. Elevated levels of serum tryptase occur in both anaphylactic and anaphylactoid reactions, but a negative test does not exclude anaphylaxis.

As used herein, the term “mast cell” refers to a resident cell of several types of tissues and contains many granules rich in histamine and heparin. Although best known for their role in allergy and anaphylaxis, mast cells play an important protective role as well, being intimately involved in wound healing and defense against pathogens.

As used herein, the term “histamine” refers to a biogenic amine involved in local immune responses as well as regulating physiological function in the gut and acting as a neurotransmitter. Histamine triggers the inflammatory response. As part of an immune response to foreign pathogens, histamine is produced by basophils and by mast cells found in nearby connective tissues. Histamine increases the permeability of the capillaries to white blood cells and other proteins, in order to allow them to engage foreign invaders in the affected tissues.

As used herein, the term “histamine receptor” refers to a class of G-protein coupled receptors with histamine as their endogenous ligand. There are several splice variants of H3 present in various species. Though all of the receptors are 7-transmembrane g protein coupled receptors, H1 and H2 are quite different from H3 and H4 in their activities. H1 causes an increase in PIP2 hydrolysis, H2 stimulates gastric acid secretion, and H3 mediates feedback inhibition of histamine.

As used herein, the term “inhibition” or “enzyme inhibition” refers to the function of reducing enzymatic activity.

As used herein, the term “antagonist” or “receptor antagonist” refers to a type of receptor ligand or drug that does not provoke a biological response itself upon binding to a receptor, but blocks or dampens agonist-mediated responses. In pharmacology, antagonists have affinity but no efficacy for their cognate receptors, and binding will disrupt the interaction and inhibit the function of an agonist or inverse agonist at receptors. Antagonists mediate their effects by binding to the active site or to allosteric sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist receptor binding. The majority of drug antagonists achieve their potency by competing with endogenous ligands or substrates at structurally-defined binding sites on receptors.

As used herein, the term “agonist” or “receptor agonist” refers to a type of ligand or drug that binds and alters the activity of a receptor. The ability to alter the activity of a receptor, also known as the agonist's efficacy, is a property that distinguishes it from antagonists, a type of receptor ligand which also binds a receptor but which does not alter the activity of the receptor. The efficacy of an agonist may be positive, causing an increase in the receptor's activity or negative causing a decrease in the receptor's activity.

As used herein, the term “inhibitor” refers to molecules that bind to enzymes and decrease their activity. The binding of an inhibitor can stop a substrate from entering the enzyme's active site and/or hinder the enzyme from catalyzing its reaction. Inhibitor binding is either reversible or irreversible. Irreversible inhibitors usually react with the enzyme and change it chemically. These inhibitors modify key amino acid residues needed for enzymatic activity. Reversible inhibitors bind non-covalently and different types of inhibition are produced depending on whether these inhibitors bind the enzyme, the enzyme-substrate complex, or both.

As used herein, the term “mast cell” refers to a resident cell of several types of tissues containing many granules rich in histamine and heparin. Although best known for their role in allergy and anaphylaxis, mast cells play an important protective role as well, being intimately involved in wound healing and defense against pathogens.

As used herein, the term “degranulation” refers to a cellular process that releases antimicrobial cytotoxic molecules from secretory vesicles called granules found inside some cells. It is used by several different cells involved in the immune system, including granulocytes (neutrophils, basophils and eosinophils) and mast cells, and certain lymphocytes such as natural killer (NK) cells and cytotoxic T cells, whose main purpose is to destroy invading microorganisms.

As used herein, the term “allergy” refers to a disorder of the immune system also referred to as atopy. Allergic reactions occur to environmental substances known as allergens; these reactions are acquired, predictable and rapid. Allergy is one of four forms of hypersensitivity and is called type I (or immediate) hypersensitivity. It is characterized by excessive activation of certain white blood cells called mast cells and basophils by a type of antibody known as IgE, resulting in an extreme inflammatory response. Common allergic reactions include eczema, hives, hay fever, asthma, food allergies, and reactions to the venom of stinging insects such as wasps and bees.

As used herein, the term “anaphylaxis” refers to an acute systemic (multi-system) and severe Type I Hypersensitivity allergic reaction in humans and other mammals causing anaphylactic shock due to the release of large quantities of immunological mediators (histamines, prostaglandins, leukotrienes) from mast cells leading to systemic vasodilation (associated with a sudden drop in blood pressure) and edema of bronchial mucosa (resulting in bronchoconstriction and difficulty breathing).

As used herein, the term “Arthritis” refers to an inflammatory disorder that includes osteoarthritis and rheumatoid arthritis. The most common form of arthritis, osteoarthritis (degenerative joint disease) is a result of trauma to the joint, infection of the joint, or age. Other arthritis forms are rheumatoid arthritis and psoriatic arthritis, autoimmune diseases in which the body attacks itself. Septic arthritis is caused by joint infection. Gouty arthritis is caused by deposition of uric acid crystals in the joint, causing inflammation.

As used herein, the term “Cyclooxygenase” (COX) refers to an enzyme that is responsible for formation of important biological mediators called prostanoids (e.g. prostaglandins, prostacyclin and thromboxane). These include COX-1 and COX-2 cyclooxygenases.

As used herein, the term “Prostanoid” refers to a subclass of eicosanoids consisting of: the prostaglandins (mediators of inflammatory and anaphylactic reactions), the thromboxanes (mediators of vasoconstriction) and the prostacyclins (active in the resolution phase of inflammation).

As used herein, the term “Eicosanoids” refers to signaling molecules made by oxygenation of twenty-carbon essential fatty acids. There are four families of eicosanoids—the prostaglandins, prostacyclins, the thromboxanes and the leukotrienes.

As used herein, the term “Lipoxygenases” (LOX) refers to a family of iron-containing enzymes that catalyze the dioxygenation of polyunsaturated fatty acids in lipids containing a cis,cis-1,4-pentadiene structure. These include 5-LOX, 12-LOX, and 15-LOX enzymes.

As used herein, the term “Leukotrienes” refers to naturally produced eicosanoid lipid mediators responsible for the effects an inflammatory response. Leukotrienes use both autocrine and paracrine signaling to regulate the body's response. Leukotrienes are produced in the body from arachidonic acid by the enzyme 5-lipoxygenase.

As used herein, the term “Autocrine” refers to a form of signaling in which a cell secretes a hormone, or chemical messenger (called the autocrine agent) that binds to autocrine receptors on the same cell, leading to changes in the cell.

As used herein the term “Paracrine” refers to a form of cell signaling in which the target cell is different, but near (“para”=near) the signal-releasing cell.

As used herein the term “Arachidonic acid” (AA, sometimes ARA) refers to an omega-6 fatty acid 20:4 (ω-6).

### Extracts

The present disclosure provides in part certain extracts of nettle. The nettle extracts contain certain compounds that are active against one or more inflammatory-related endpoints, such as COX, LOX, HPDGS, Tryptase and H₁ Receptor. For example, one embodiment relates to a nettle extract comprising at least one compound selected from the group consisting of 6-azacytosine, levulinic acid, threonine, niacinamide, DL-methyl-m-tyrosine, 4-methyl-7-ethoxy coumarin, vitamin B5, isopropyl-B-D-thiogalactopyranoside, osthole, phosphatidylcholine, 4-shogaol, piperine/cocluarine/laurifoline, 8-dehydrogingerdione, sinomenin/deoxyharringtonine, and picrocrocin/carnosol. The presence of a slash “/” between compound names in the Chemical composition lists for each extract indicates that the compounds have the same molecular weight, and either one, both or all of the compounds may be present in the extract. For example, “piperine/cocluarine/laurifoline” indicates that the compound identified as present in the extract and having bioactivity against certain therapeutic endpoints may be piperine, coclaurine, laurifoline, or any combination of two of these compounds or all three of these compounds.

In some embodiments, the extracts comprise certain amounts of the aforementioned compounds. The amounts of the compounds are described, solely by way of example and for convenience, in micrograms (μg) per 100 mg of the extracts. These embodiments, therefore, are not in any limited to the absolute quantities of compounds or the absolute quantities of the extract. In one embodiment, the extract comprises at least one compound selected from: about 1 to 3000 μg 6-azacytosine, about 5 to 5000 μg levulinic acid, about 5 to 1000 μg threonine, about 5 to 1000 μg niacinamide, about 10 to 1000 μg DL-methyl-m-tyrosine, about 10 to 2500 μg 4-methyl-7-ethoxy coumarin, about 50 to 3000 μg vitamin B5, about 5 to 250 μg isopropyl-B-D-thiogalactopyranoside, about 10 to 1000 μg osthole, about 10 to 500 μg phosphatidylcholine, about 10 to 1000 μg 4-shogaol, about 10 to 1000 μg piperine/cocluarine/laurifoline, about 10 to 750 μg 8-dehydrogingerdione, about 10 to 500 μg sinomenin/deoxyharringtonine, and about 10 to 500 μg picrocrocin/carnosol, per 100 mg of the extract. The extract may contain one, two, three, or more of the aforementioned compounds, or it may contain all of the aforementioned compounds. The aforementioned compounds may individually impart therapeutic activity to the extract, for example by inhibition one or more therapeutic endpoints as described below, or the compounds may impart therapeutic activity to the extract by a synergistic interaction with another compound present in the extract. Additionally, the extract may contain additional compounds. The additional compounds may or may not contribute to the overall therapeutic properties of the extract individually, or synergistically.

In another embodiment, the aforementioned extract comprises at least one compound selected from: about 1 to 1750 μg 6-azacytosine, about 10 to 2000 μg levulinic acid, about 10 to 500 μg threonine, about 10 to 300 μg niacinamide, about 30 to 300 μg DL-methyl-m-tyrosine, about 50 to 1500 μg 4-methyl-7-ethoxy coumarin, about 100 to 2000 μg vitamin B5, to 100 μg isopropyl-B-D-thiogalactopyranoside, about 50 to 500 μg osthole, about 50 to 200 μg phosphatidylcholine, about 50 to 500 μg 4-shogaol, about 50 to 400 μg piperine/cocluarine/laurifoline, about 50 to 400 μg 8-dehydrogingerdione, about 30 to 250 μg sinomenin/deoxyharringtonine, and about 50 to 250 μg picrocrocin/carnosol, per 100 mg of the extract.

In certain embodiments, the extract comprises about 1 to 1750 μg, about 500 to 1750 μg, about 1000 to 1750 μg, or about 1500 to 1750 μg of 6-azacytosine, per 100 mg of the extract. In other embodiments, the extract comprises about 1250, 1350, 1450, 1550, 1650, or 1750 μg of 6-azacytosine per 100 mg of the extract.

In other embodiments, the aforementioned nettle extracts comprise nettle extract of about 20 to 90 μg, 20 to 80 μg, 20 to 70 μg, or 30 to 60 μg isopropyl-B-D-thiogalactopyranoside per 100 mg of the extract. In other embodiments, the extract comprises about 30, 40, 50, 60, 70 or 80 μg isopropyl-B-D-thiogalactopyranoside per 100 mg of the extract.

In other embodiments, the aforementioned nettle extracts comprise about 30 to 200 μg, 30 to 150 μg, 30 to 100 μg, or 40 to 90 μg sinomenin/deoxyharringtonine per 100 mg of the extract. In other embodiments, the nettle extract comprises about 30, 40, 50, 60, 70, 80, 90 to 100 μg sinomenin/deoxyharringtonine per 100 mg of the extract.

In other embodiments, any of the aforementioned the nettle extracts comprise about 500 to 2500 μg, 500 to 2000 μg, 1000 to 2500 μg, or 1500 to 2500 μg levulinic acid per 100 mg of the extract. In other embodiments, the extract comprises about 1500, 1600, 1700, 1800, 1900, or 2000 μg levulinic acid per 100 mg of the extract.

In other embodiments, the aforementioned nettle extracts comprise about 10 to 500, to 250, or 10 to 100 μg threonine per 100 mg of the extract. In other embodiments, the extract comprises about 10, 20, 30, 40, 50, 60, 70, 80, 90 or 100 μg threonine per 100 mg of the extract.

In other embodiments, the aforementioned nettle extracts comprise about 50 to 300, 100 to 300, 150 to 300, or 200 to 300 μg niacinamide per 100 mg of the extract. In another embodiment, the extract comprises about 180, 190, 200, 210, 220, 230, 240, or 250 μg niacinamide per 100 mg of the extract.

In another embodiments, the aforementioned extracts comprise about 30, to 500, 30 to 300, 50 to 300, 100 to 300, 150 to 300, or 200 to 300 μg DL-methyl-m-tyrosine per 100 mg of the extract. In other embodiments, the extract comprises about 200, 210, 220, 230, 240, 250, 260, 270, 280, 290, or 300 μg DL-methyl-m-tyrosine per 100 mg of the extract.

In other embodiments, any of the aforementioned extracts comprise about 50 to 1500, 500 to 1500, 750 to 1500, or 1000 to 1500 μg of 4-methyl-7-ethoxy coumarin per 100 mg of the extract. In other embodiments, the extract comprises about 1000, 1050, 1100, 1150, 1200, or 1250 μg of 4-methyl-7-ethoxy coumarin per 100 mg of the extract.

In other embodiments, the aforementioned extracts comprise about 100 to 2000, 100 to 1500, 100 to 1000, 100 to 500 or 100 to 250 μg vitamin B5 per 100 mg of the extract. In other embodiments, the extracts comprise about 100, 110, 120, 130, 140, 150, 160, 170, 180, 190, or 200 μg of vitamin B5 per 100 mg of the extract.

In other embodiments, the aforementioned nettle extracts comprise about 50 to 500, 100 to 500, 200 to 500, 250 to 500 or 200 to 400 μg osthole per 100 mg of the extract. In other embodiments, the extracts comprise about 300, 310, 320, 330, 340, 350, 360, 370, 380, 390, or 400 μg osthole per 100 mg of the extract.

In other embodiments, the aforementioned nettle extracts comprise about 50 to 200, 75 to 200, 100 to 200 or 100 to 150 μg phosphatidylcholine per 100 mg of the extract. In other embodiments, the extracts comprise about 100, 110, 120, 130, 140 150, 160, 170, 180, 190 or 200 μg phosphatidylcholine per 100 mg of the extract.

In some embodiments, the aforementioned nettle extracts comprises about 50 to 500, 50 to 250, 50 to 200, 50 to 150 or 50 to 100 μg 4-shogaol per 100 mg of the extract. In other embodiments, the extracts comprise about 50, 60, 70, 80, 90, or 100 μg 4-shogaol per 100 mg of the extract.

In other embodiments, the aforementioned nettle extracts comprise 50 to 400, 50 to 250, 50 to 200, 50 to 150, or 50 to 100 μg piperine/cocluarine/laurifoline per 100 mg of the extract. In other embodiments, the extracts comprise about 50, 60, 70, 80, 90, or 100 μg piperine/cocluarine/laurifoline per 100 mg of the extract.

In some embodiments, the aforementioned extracts comprise about 50 to 400, 50 to 250, 50 to 200, 50 to 150 or 50 to 100 μg 8-dehydrogingerdione per 100 mg of the extract. In other embodiments, the extracts comprise about 50, 60, 70, 80, 90, or 100 μg 8-dehydrogingerdione per 100 mg of the extract.

In other embodiments, the aforementioned nettle extracts comprise about 50 to 250, 50 to 200, 50 to 150 or 50 to 100 μg picrocrocin/camosol per 100 mg of the extract. In other embodiments, the extracts comprise about 50, 60, 70, 80, 90, or 100 μg picrocrocin/carnosol per 100 mg of the extract.

In some embodiments, the nettle extract comprises about 1 to 1750 μg 6-azacytosine, about 10 to 2000 μg levulinic acid, about 10 to 500 μg threonine, about 10 to 300 μg niacinamide, about 30 to 300 μg DL-methyl-m-tyrosine, about 50 to 1500 μg 4-methyl-7-ethoxy coumarin, about 100 to 2000 μg vitamin B5, 20 to 100 μg isopropyl-B-D-thiogalactopyranoside, about 50 to 500 μg osthole, about 50 to 200 μg phosphatidylcholine, about 50 to 500 μg 4-shogaol, about 50 to 400 μg piperine/cocluarine/laurifoline, about 50 to 400 μg 8-dehydrogingerdione, about 30 to 250 μg sinomenin/deoxyharringtonine, and about 50 to 250 μg picrocrocin/camosol, per 100 mg of the extract.

In other embodiments, the nettle extract comprises about 1500 to 1750 μg 6-azacytosine, about 1800 to 2000 μg levulinic acid, about 50 to 75 μg threonine, about 150 to 250 μg niacinamide, about 200 to 300 μg DL-methyl-m-tyrosine, about 1000 to 1300 μg 4-methyl-7-ethoxy coumarin, about 100 to 200 μg vitamin B5, 30 to 60 μg isopropyl-B-D-thiogalactopyranoside, about 250 to 400 μg osthole, about 100 to 200 μg phosphatidylcholine, about 50 to 100 μg 4-shogaol, about 75 to 150 μg piperine/cocluarine/laurifoline, about 50 to 150 μg 8-dehydrogingerdione, about 30 to 100 μg sinomenin/deoxyharringtonine, and about 75 to 150 μg picrocrocin/camosol, per 100 mg of the extract.

In other embodiments, any of the aforementioned nettle extracts may further comprise at least one of resorcinol, proline, leucine, adenine, levoglucosan/glycogen/-laminarin, synephrine, or shikimic acid. In other embodiments, the extract further comprises at least one of about 10 to 1500 μg of resorcinol, about 50 to 1500 μg of proline, about 5 to 1500 μg of leucine, about 10 to 5000 μg of adenine, about 300 to 10,000 μg of levoglucosan/glycogen/laminarin, about 100 to 3,000 μg of synephrine, or about 50 to 1000 μg of shikimic acid per 100 mg of the extract.

In some embodiments, any of the aforementioned nettle extracts may further comprise 3,4-dimethoxy chalcone, for example, about 25 to 200 μg of 3,4-dimethoxychalcone per 100 mg of the extract.

In some embodiments, the invention relates to a nettle extract comprising about 1500 to 1750 μg 6-azacytosine, about 1800 to 2000 μg levulinic acid, about 50 to 75 μg threonine, about 150 to 250 μg niacinamide, about 200 to 300 μg DL-methyl-m-tyrosine, about 1000 to 1300 μg 4-methyl-7-ethoxy coumarin, about 100 to 200 μg vitamin B5, 30 to 60 μg isopropyl-B-D-thiogalactopyranoside, about 250 to 400 μg osthole, about 100 to 200 μg phosphatidylcholine, about 50 to 100 μg 4-shogaol, about 75 to 150 μg piperine/cocluarine/laurifoline, about 50 to 150 μg 8-dehydrogingerdione, about 30 to 100 μg sinomenin/deoxyharringtonine, about 75 to 150 μg picrocrocin/carnosol, 300 to 600 μg of resorcinol, about 100 to 250 μg of proline, about 150 to 350 μg of leucine, about 2000 to 3000 μg of adenine, about 4000 to 6000 μg of levoglucosan/glycogen/laminarin, about 1500 to 2,000 μg of synephrine, about 250 to 700 μg of shikimic acid, and about 50 to 150 μg of 3,4-dimethoxychalcone per 100 mg of the extract.

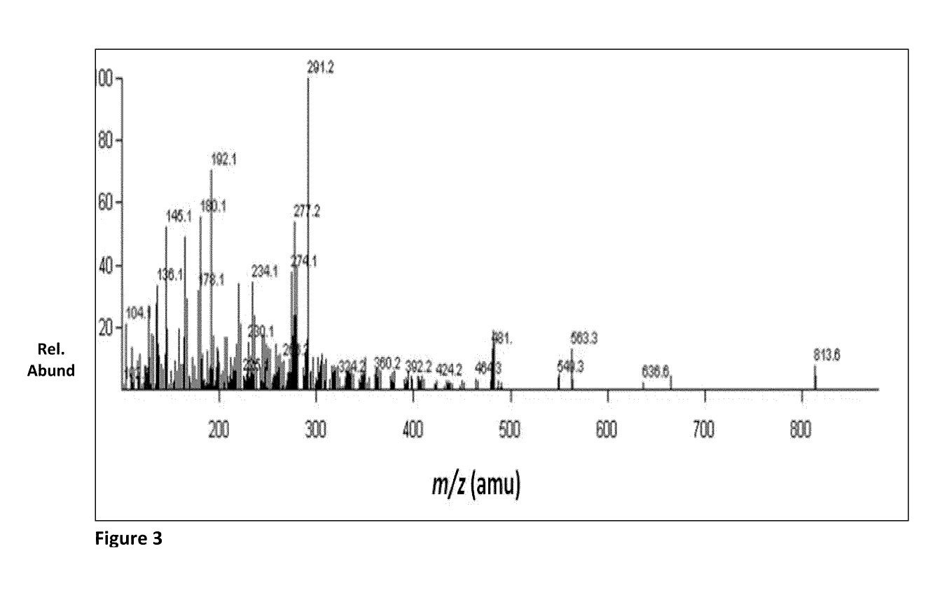

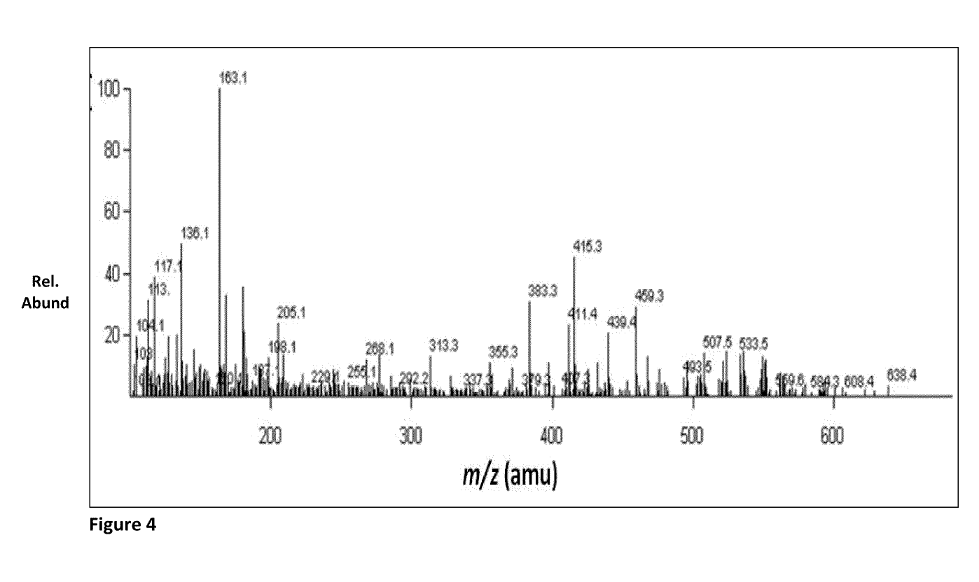

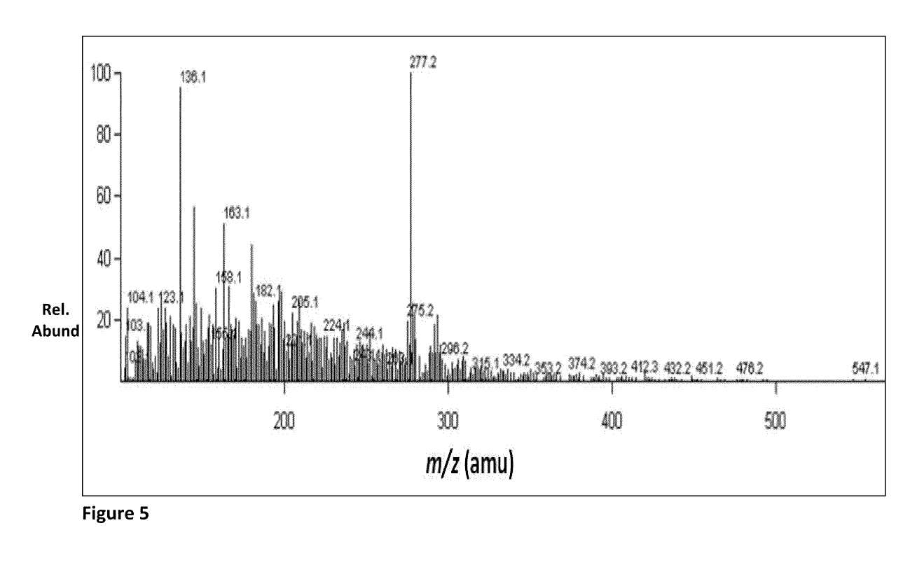

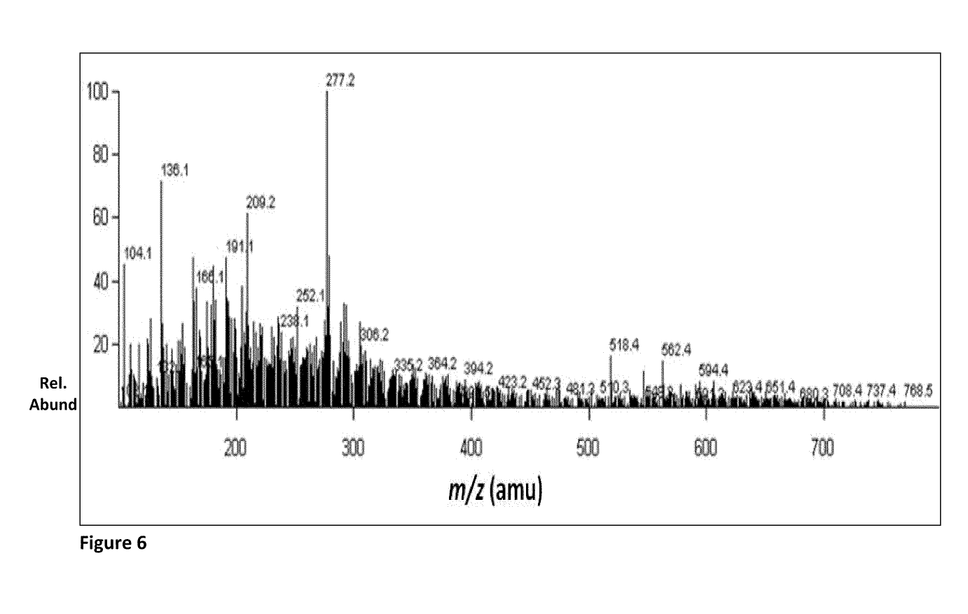

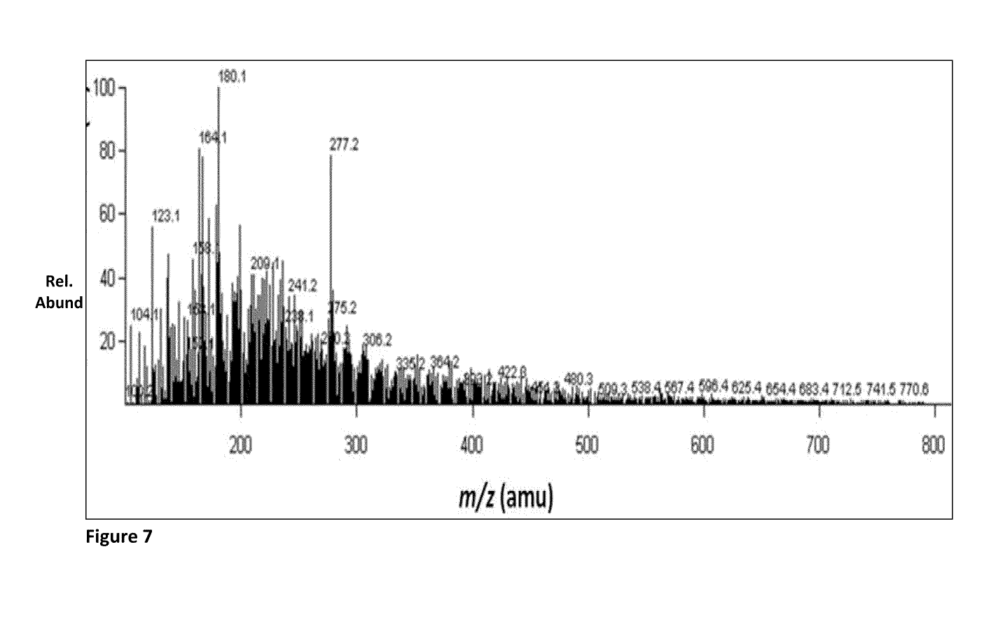

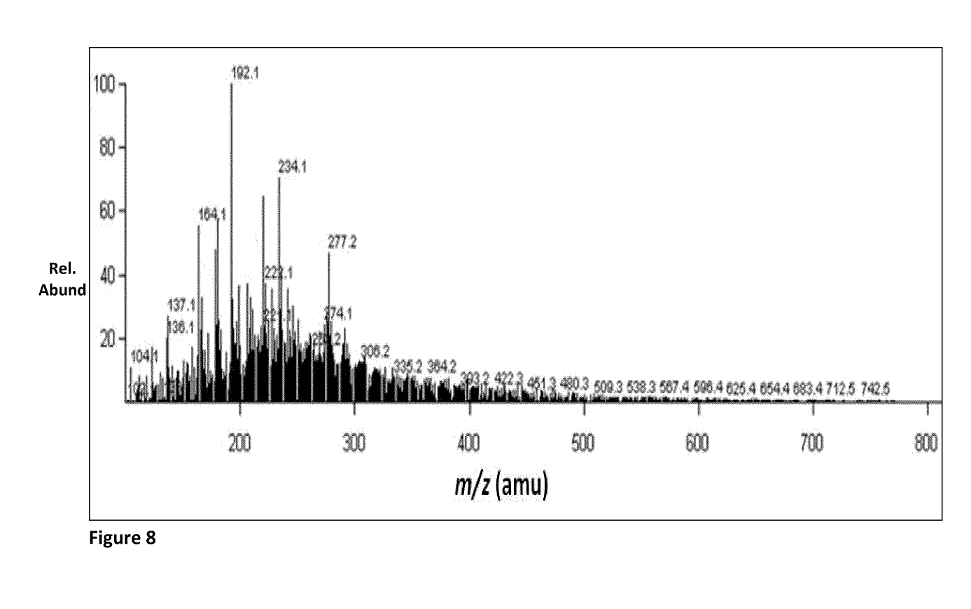

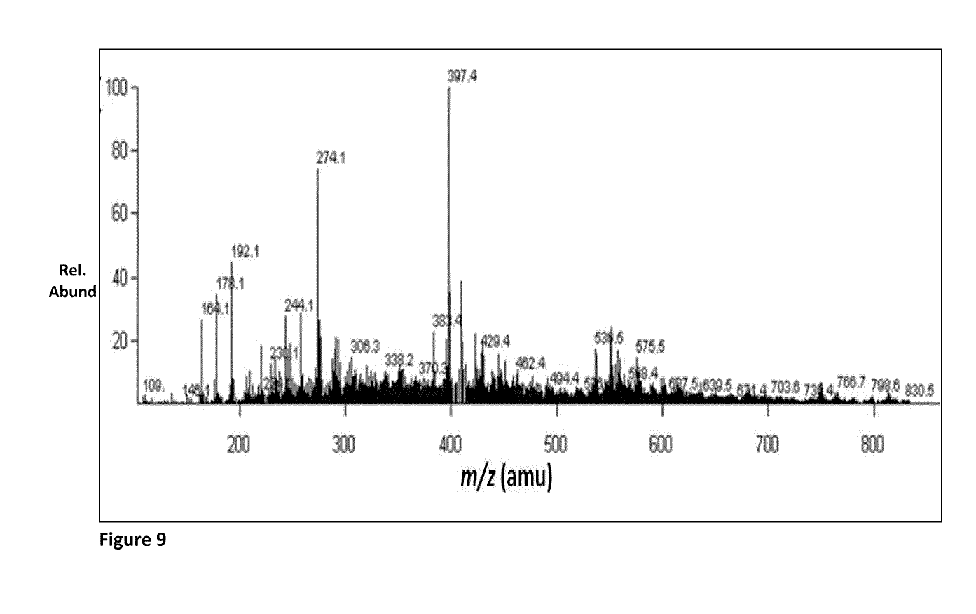

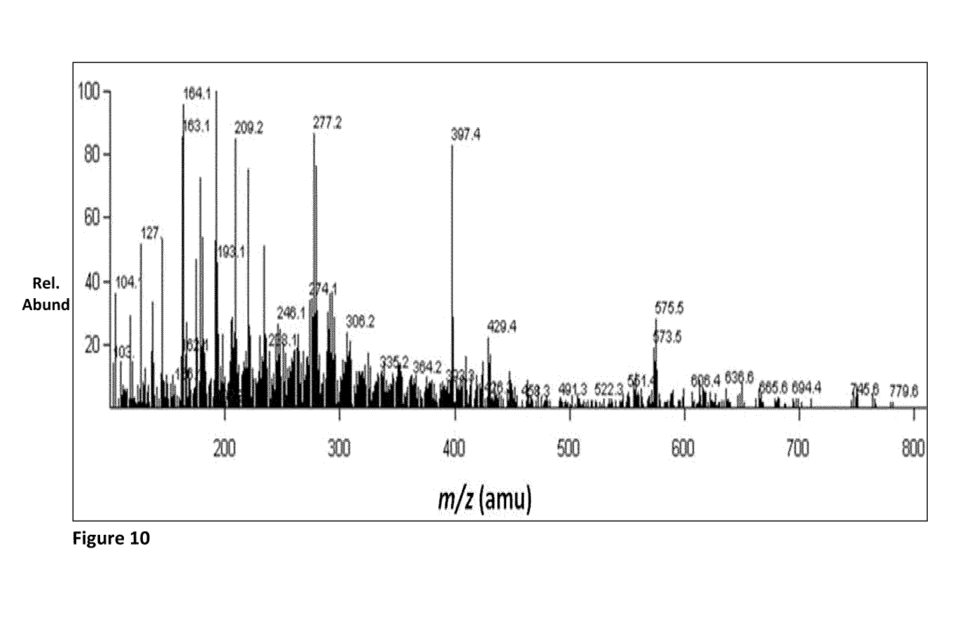

In certain embodiments, the invention relates to a nettle extract comprising a fraction having a Direct Analysis in Real Time (DART) TOF mass spectrometry chromatogram of any of FIGS. 3 to 10.

In certain embodiments, the aforementioned nettle extracts are active against several therapeutic endpoints relating to allergies and inflammation. In certain embodiments, the nettle extract has an IC₅₀ value for COX-1 inhibition of less than 1000 μg/mL. In other embodiments, the extract has an IC₅₀ value for COX-1 inhibition is about 1 μg/mL to 500 μg/mL, 5 μg/mL to 400 μg/mL, or 50 μg/mL to 350 μg/mL.

In other embodiments, any of the aforementioned extracts have an IC₅₀ value for COX-2 inhibition of less than 1000 μg/mL. In other embodiments, the IC₅₀ value for COX-2 inhibition is about 1 μg/mL to 500 μg/mL, 5 μg/mL to 400 μg/mL, or 50 μg/mL to 300 μg/mL.

In some embodiments, any of the aforementioned nettle extracts has an IC₅₀ value for 5-LOX inhibition of less than 1000 μg/mL. In other embodiments, the IC₅₀ for 5-LOX inhibition is about 1 μg/mL to 1000 μg/mL, 50 μg/mL to 750 μg/mL, or 100 μg/mL to 500 μg/mL.

In other embodiments, any of the aforementioned extracts has an IC₅₀ for HPGDS of less than 1000 μg/mL. In other embodiments, the IC₅₀ for HPGDS is about 1 to 1000 μg/mL, 1 to 500 μg/mL, or 10 to 300 μg/mL.

In some embodiments, any of the aforementioned extracts has an IC₅₀ for H₁ antagonism of less than 1000 μg/mL. In other embodiments, the IC₅₀ for H₁ antagonism is about 1 to 900 μg/mL, 1 to 750 μg/mL, 50 to 500 μg/mL or 50 to 250 μg/mL.

In certain embodiments, the nettle extract has an IC₅₀ for H₁ negative agonism of less than 1000 μg/mL. In other embodiments, the IC₅₀ for H₁ negative agonism is about 1 to 900 μg/mL, 1 to 750 μg/mL, 50 to 500 μg/mL, or 50 to 250 μg/mL.

In some embodiments, any of the aforementioned nettle extracts has an IC₅₀ for tryptase inhibition of less than 1000 μg/mL. In other embodiments, the IC₅₀ for tryptase is about 1 to 500 μg/mL, 1 to 250 μg/mL, 10 to 200 μg/mL or about 20 to 150 μg/mL.

### Methods of Treatment and Prevention

The aforementioned extracts are useful in treating a variety of disease and conditions associated with different inflammatory and allergic endpoints. Accordingly, one aspect of the invention provides a method of treating or preventing an inflammatory disorder in a subject comprising administering to a subject in need thereof a therapeutically effective amount of the aforementioned extracts. The extract may be administered alone as the isolated extract itself, or it may be administered as a pharmaceutical composition comprising the extract and a pharmaceutically acceptable carrier. In another embodiment, the invention relates to a method of treating or preventing symptoms of an inflammatory disorder in a subject comprising administering to a subject in need thereof a therapeutically effective amount the aforementioned extracts. The inflammatory condition may be either chronic or acute. In some embodiments, the condition is allergic rhinitis (hay fever). In other embodiments, the condition is rheumatism (disorders of the muscles and joints), osteoarthritis, eczema, arthritis (e.g. rheumatoid arthritis or osteoarthritis), gout, anemia, enlarged prostate, joint pain, tendonitis, sprains, insect bites, asthma, or itchy skin conditions. The aforementioned nettle extracts also may be useful in treating a variety of other disorders, such as joint and muscle pain in arthritis and other inflammatory conditions.

### Formulations

Compositions of the disclosure comprise extracts of nettle plant materials in forms such as a paste, powder, oils, liquids, suspensions, solutions, or other forms, comprising, one or more fractions or sub-fractions to be used as dietary supplements, nutraceuticals, or such other preparations that may be used to prevent or treat various human ailments. The extracts can be processed to produce such consumable items, for example, by mixing them into a food product, in a capsule or tablet, or providing the paste itself for use as a dietary supplement, with sweeteners or flavors added as appropriate. Accordingly, such preparations may include, but are not limited to, nettle extract preparations for oral delivery in the form of tablets, capsules, lozenges, liquids, emulsions, dry flowable powders and rapid dissolve tablet. Based on the anti-allergic activities described herein, patients would be expected to benefit from daily dosages in the range of from about 50 mgs to about 1000 mg. For example, a lozenge comprising about 50, 55, 60, 65, 70, 75, 80, 85, 90, 95, 100, 105, 110, 115, 120, 125, 130, 135, 140, 145 or 150 mgs of the extract can be administered once or twice a day to a subject as a prophylactic. Alternatively, in response to a severe allergic reaction, two lozenges may be needed every 4 to 6 h.

In one embodiment, a dry extracted nettle species composition is mixed with a suitable solvent, such as but not limited to water or ethyl alcohol, along with a suitable food-grade material using a high shear mixer and then spray air-dried using conventional techniques to produce a powder having grains of very small nettle extract particles combined with a food-grade carrier.

In a particular example, an extracted nettle extract composition is mixed with about twice its weight of a food-grade carrier such as maltodextrin having a particle size of between 100 to about 150 micrometers and an ethyl alcohol solvent using a high shear mixer. Inert carriers, such as silica, preferably having an average particle size on the order of about 1 to about 50 micrometers, can be added to improve the flow of the final powder that is formed. Preferably, such additions are up to 2% by weight of the mixture. The amount of ethyl alcohol used is preferably the minimum needed to form a solution with a viscosity appropriate for spray air-drying. Typical amounts are in the range of between about 5 to about 10 liters per kilogram of extracted material. The solution of extract, maltodextrin and ethyl alcohol is spray air-dried to generate a powder with an average particle size comparable to that of the starting carrier material.

In another embodiment, an extract and food-grade carrier, such as magnesium carbonate, a whey protein, or maltodextrin are dry mixed, followed by mixing in a high shear mixer containing a suitable solvent, such as water or ethyl alcohol. The mixture is then dried via freeze drying or refractive window drying. In a particular example, extract material is combined with food grade material about one and one-half times by weight of the extract, such as magnesium carbonate having an average particle size of about 20 to 200 micrometers. Inert carriers such as silica having a particle size of about 1 to about 50 micrometers can be added, preferably in an amount up to 2% by weight of the mixture, to improve the flow of the mixture. The magnesium carbonate and silica are then dry mixed in a high speed mixer, similar to a food processor-type of mixer, operating at 100's of rpm. The extract is then heated until it flows like a heavy oil. Preferably, it is heated to about 50° C. The heated extract is then added to the magnesium carbonate and silica powder mixture that is being mixed in the high shear mixer. The mixing is continued preferably until the particle sizes are in the range of between about 250 micrometers to about 1 millimeter. Between about 2 to about 10 liters of cold water (preferably at about 4° C.) per kilogram of extract is introduced into a high shear mixer. The mixture of extract, magnesium carbonate, and silica is introduced slowly or incrementally into the high shear mixer while mixing. An emulsifying agent such as carboxymethylcellulose or lecithin can also be added to the mixture if needed. Sweetening agents such as Sucralose or Acesulfame K up to about 5% by weight can also be added at this stage if desired. Alternatively, extract of Stevia rebaudiana, a very sweet-tasting dietary supplement, can be added instead of or in conjunction with a specific sweetening agent (for simplicity, Stevia will be referred to herein as a sweetening agent). After mixing is completed, the mixture is dried using freeze-drying or refractive window drying. The resulting dry flowable powder of extract, magnesium carbonate, silica and optional emulsifying agent and optional sweetener has an average particle size comparable to that of the starting carrier and a predetermined extract.

According to another embodiment, an extract is combined with approximately an equal weight of food-grade carrier such as whey protein, preferably having a particle size of between about 200 to about 1000 micrometers. Inert carriers such as silica having a particle size of between about 1 to about 50 micrometers, or carboxymethylcellulose having a particle size of between about 10 to about 100 micrometers can be added to improve the flow of the mixture. Preferably, an inert carrier addition is no more than about 2% by weight of the mixture. The whey protein and inert ingredient are then dry mixed in a food processor-type of mixer that operates over 100 rpm. The extract can be heated until it flows like a heavy oil (preferably heated to about 50° C.). The heated extract is then added incrementally to the whey protein and inert carrier that is being mixed in the food processor-type mixer. The mixing of the extract and the whey protein and inert carrier is continued until the particle sizes are in the range of about 250 micrometers to about 1 millimeter. Next, 2 to 10 liters of cold water (preferably at about 4° C.) per kilogram of the paste mixture is introduced in a high shear mixer. The mixture of extract, whey protein, and inert carrier is introduced incrementally into the cold water containing high shear mixer while mixing. Sweetening agents or other taste additives of up to about 5% by weight can be added at this stage if desired. After mixing is completed, the mixture is dried using freeze drying or refractive window drying. The resulting dry flowable powder of extract, whey protein, inert carrier and optional sweetener has a particle size of about 150 to about 700 micrometers and a unique predetermined extract.

In the embodiments where the extract is to be included into an oral fast dissolve tablet as described in U.S. Pat. No. 5,298,261, the unique extract can be used “neat,” that is, without any additional components which are added later in the tablet forming process as described in the patent cited. This method obviates the necessity to take the extract to a dry flowable powder that is then used to make the tablet.

Once a dry extract powder is obtained, such as by the methods discussed herein, it can be distributed for use, e.g., as a dietary supplement or for other uses. In a particular embodiment, the novel extract powder is mixed with other ingredients to form a tableting composition of powder that can be formed into tablets. The tableting powder is first wet with a solvent comprising alcohol, alcohol and water, or other suitable solvents in an amount sufficient to form a thick doughy consistency. Suitable alcohols include, but not limited to, ethyl alcohol, isopropyl alcohol, denatured ethyl alcohol containing isopropyl alcohol, acetone, and denatured ethyl alcohol containing acetone. The resulting paste is then pressed into a tablet mold. An automated tablet molding system, such as described in U.S. Pat. No. 5,407,339, can be used. The tablets can then be removed from the mold and dried, preferably by air-drying for at least several hours at a temperature high enough to drive off the solvent used to wet the tableting powder mixture, typically between about 70° to about 85° C. The dried tablet can then be packaged for distribution

Compositions can be in the form of a paste, resin, oil, powder or liquid. Liquid preparations for oral administration may take the form of, for example, solutions, syrups or suspensions, or they may be presented as a dry product for reconstitution with water or other suitable vehicle prior to administration. Such liquid preparations may be prepared by conventional means with pharmaceutically acceptable additives such as suspending agents (e.g., sorbitol syrup, methyl cellulose, or hydrogenated edible fats); emulsifying agents (e.g., lecithin or acacia); non-aqueous vehicles (e.g., almond oil, oily esters or ethyl alcohol); preservatives (e.g., methyl or propyl p-hyroxybenzoates or sorbic acid); and artificial or natural colors and/or sweeteners. Compositions of the liquid preparations can be administered to humans or animals in pharmaceutical carriers known to those skilled in the art. Such pharmaceutical carriers include, but are not limited to, capsules, lozenges, syrups, sprays, rinses, and mouthwash.

Dry powder compositions may be prepared according to methods disclosed herein and by other methods known to those skilled in the art such as, but not limited to, spray air drying, freeze drying, vacuum drying, and refractive window drying. The combined dry powder compositions can be incorporated into a pharmaceutical carrier such, but not limited to, tablets or capsules, or reconstituted in a beverage such as a tea.

The described extracts may be combined with extracts from other plants such as, but not limited to, varieties of Gymnemia, turmeric, boswellia, guarana, cherry, lettuce, Echinacia, piper betel leaf, Areca catechu, Muira puama, ginger, willow, suma, kava, horny goat weed, Ginko bilboa, mate, garlic, puncture vine, arctic root astragalus, eucommia, gastropodia, and uncaria, or pharmaceutical or nutraceutical agents.

A tableting powder can be formed by adding about 1% to 40% by weight of the powdered extract, with between 30% to about 80% by weight of a dry water-dispersible absorbent such as, but not limited to, lactose. Other dry additives such as, but not limited to, one or more sweetener, flavoring and/or coloring agents, a binder such as acacia or gum arabic, a lubricant, a disintegrant, and a buffer can also be added to the tableting powder. The dry ingredients are screened to a particle size of between about 50 to about 150 mesh. Preferably, the dry ingredients are screened to a particle size of between about 80 to about 100 mesh.

Preferably, the tablet exhibits rapid dissolution or disintegration in the oral cavity. The tablet is preferably a homogeneous composition that dissolves or disintegrates rapidly in the oral cavity to release the extract content over a period of about 2 sec or less than 60 sec or more, preferably about 3 to about 45 sec, and most preferably between about 5 to about 15 sec.

Various rapid-dissolve tablet formulations known in the art can be used. Representative formulations are disclosed, for example, in U.S. Pat. Nos. 5,464,632; 6,106,861; 6,221,392; 5,298,261; and 6,200,604; the entire contents of each are expressly incorporated by reference herein. For example, U.S. Pat. No. 5,298,261 teaches a freeze-drying process. This process involves the use of freezing and then drying under a vacuum to remove water by sublimation. Preferred ingredients include hydroxyethylcellulose, such as Natrosol from Hercules Chemical Company, added to between 0.1% and 1.5%. Additional components include maltodextrin (Maltrin, M-500) at between 1% and 5%. These amounts are solubilized in water and used as a starting mixture to which is added the Ginger species extraction composition, along with flavors, sweeteners such as Sucralose or Acesulfame K, and emulsifiers such as BeFlora and BeFloraPlus which are extracts of mung bean. A particularly preferred tableting composition or powder contains about 10% to 60% by of the extract powder and about 30% to about 60% of a water-soluble diluent.

In a preferred implementation, the tableting powder is made by mixing in a dry powdered form the various components as described above, e.g., active ingredient (extract), diluent, sweetening additive, and flavoring, etc. An overage in the range of about 10% to about 15% of the active extract can be added to compensate for losses during subsequent tablet processing. The mixture is then sifted through a sieve with a mesh size preferably in the range of about 80 mesh to about 100 mesh to ensure a generally uniform composition of particles.

The tablet can be of any desired size, shape, weight, or consistency. The total weight of the extract in the form of a dry flowable powder in a single oral dosage is typically in the range of about 40 mg to about 1000 mg. The tablet is intended to dissolve in the mouth and should therefore not be of a shape that encourages the tablet to be swallowed. The larger the tablet, the less it is likely to be accidentally swallowed, but the longer it will take to dissolve or disintegrate. In a preferred form, the tablet is a disk or wafer of about 0.15 inch to about 0.5 inch in diameter and about 0.08 inch to about 0.2 inch in thickness, and has a weight of between about 160 mg to about 1,500 mg. In addition to disk, wafer or coin shapes, the tablet can be in the form of a cylinder, sphere, cube, or other shapes. Although the tablet is preferably an extract composition separated by non-nettle species extract regions in periodic or non-periodic sequences, which can give the tablet a speckled appearance with different colors or shades of colors associated with the extract and the non-extract region.

Compositions of unique extract compositions may also comprise extract compositions in an amount between about 10 mg and about 2000 mg per dose. Based on the anti-allergenic and anti-inflammatory activities described in the examples below, the dose of extract would be about 50-2000 mg per day, for example in a lozenge form, as a prophylactic. In some embodiments, the dosage may be about 50-1000 mg/day, 50-500 mg/day, 50-250 mg/day, or about 100 mg/day. In response to a severe allergic response, two lozenges every 4 to 6 h may be needed.

### EXEMPLIFICATION

The disclosure now being generally described, it will be more readily understood by reference to the following examples, which are included merely for purposes of illustration of certain aspects and embodiments of the disclosure, and are not intended to limit the disclosure.

### Methods

A. Nettle (Urtica dioica) Feedstock

Dried Nettle (Stinging Nettle; Urtica dioica) leaves were obtained from U.S. commercial sources. The species was certified by the suppler as Urtica dioica L.

B. Extraction Procedure

Nettle leaves were ground to powder with particle size at around 20-40 mesh. Approximately 15 g of ground nettle leaf were extracted by leaching with water or aqueous ethanol at different temperature of 20, 40 and 60° C. respectively. The leaching was performed using 2 stages at solvent/feed ratio of 15 and 10 respectively and 2 hours in each stage. After extraction, the extracted slurry was filtered off with P4 filter paper with pore size of 4-8 μm and centrifuged at 2000 rpm for 20 min. The supernatants were collected and evaporated to dryness at 50° C. in a vacuum oven overnight. All extracts were lyophilized and were utilized as dried powders for DART TOF-MS analyses, as well as for all in vitro bioassay evaluations.

C. DART TOF-MS Characterization of Extracts

The JEOL DART AccuTOF-mass spectrometer (JMS-T100LC; Jeol USA, Peabody, Mass.) was used for chemical analysis of the nettle extracts and was executed in positive ion mode [M+H]⁺. The needle voltage was set to 3500V, heating element to 300° C., electrode 1 to 150V, electrode 2 to 250V, and helium gas flow to 3.98 L/min. For the mass spectrometer, the following settings were loaded: orifice 1 set to 20V, ring lens voltage set to 5V, and orifice 2 set to 5V. The peak voltage was set to 1000V in order to give peak resolution begging at 100 m/z. The microchannel plate detector (MCP) voltage was set at 2550V. Calibrations were performed internally with each sample using a 10% (w/v) solution of PEG 600 (Ultra Chemical, North Kingston, R1) that provided mass markers throughout the required mass range 100-1000 m/z. Calibration tolerances were held to 10 mmu. Nettle extracts were introduced into the DART helium plasma using the closed end of a borosilicate glass melting point capillary tube until a signal was achieved in the total-ion chromatogram (TIC). The next sample was introduced when the TIC returned to baseline levels. Candidate molecular formulae were identified using elemental composition and isotope matching programs in the Jeol MassCenterMain Suite software (JEOL USA, Peabody, Mass.).

D. COX-1 and COX-2 Enzyme Inhibition

All reagents and solutions were prepared according to the protocols established by Cayman Chemicals (Ann Arbor, Mich.) for the COX-1 and COX-2 inhibition assays. Two procedures were utilized to assess the COX-1/2-specific and non-specific activities.

Prostaglandin Production Inhibition: Nettle extracts were dissolved in neat dimethylsulfoxide (DMSO), and then diluted in reaction buffer to a final DMSO concentration of 1% (v/v). Reactions were run with COX-1 (ovine) or COX-2 (human recombinant) enzymes in the presence of Heme. Wells containing nettle extracts, 100% enzyme activity, background wells (heat inactivated enzymes), and the appropriate blanks were prepared. Solutions were incubated at 37° C. for 15 min prior to running the reaction. Arachidonic acid was added and the reaction proceeded for 2 min. The reaction was stopped by addition of 1 M HCl. The Prostaglandin F₂ product was quantified using EIA.

Quantification of Prostaglandin with EIA: The assay plate (EIA) was provided in the Cayman Chemicals screening kit. Aliquots (50 μL) of the reaction products (PGF₂) from prostaglandin production were added to their respective wells. Total activity and blank wells received 150 μL of EIA buffer, non-specific binding wells received 100 μL of EIA buffer, and maximum binding wells received 50 μL of EIA buffer. COX 100% activity wells, non-specific binding, background, maximum binding, standards, and nettele extract wells received 50 μL of tracer. COX 100% activity, background, maximum binding, standards, and nettle extract wells also received 50 μL of antiserum. The EIA plate reactions were run for 18 h at room temperature. Plates were washed with wash buffer and 200 μL Ellman's Reagent was added to all wells, followed by 5 μL of tracer to the total activity well. The color development was quantified by absorbance at 409 nm using a BioTek Synergy microplate reader.

E. Hematopoietic Prostaglandin D Synthase (HPGDS) Inhibition Assays

All reagents and solutions were prepared according to the protocols established by Cayman Chemicals (Ann Arbor, Mich.) for the H-PGDS and L-PGDS inhibition assays. Two procedures were utilized to assess the PGDS-specific and non-specific activities.

Prostaglandin Production Inhibition: Nettle extracts were dissolved in neat dimethylsulfoxide (DMSO), and then diluted in reaction buffer to a final DMSO concentration of 1% (v/v). Wells containing nettle extract, 100% enzyme activity, and background wells (no enzyme) and the appropriate blanks were prepared. To determine the H-PGDS activity, H-PGDS enzyme was added to wells with Glutathione (GSH) and incubated for 2 min. To determine the L-PGDS activity, L-PGDS enzyme was added to wells with Dithiothreitol (DTT) and incubated for 2 min. The PGDS enzymes were both inactivated by addition of 1 M HCl. The Prostaglandin D₂ product was diluted with EIA kit buffer provided and quantified using EIA as described by the manufacturer.

Quantification of Prostaglandin with EIA: The assay plate (EIA) was provided in the Cayman Chemicals screening kit. Aliquots (50 μL) of the reaction products (PGD₂) from prostaglandin production were added to their respective wells. Total activity and blank wells received 150 μL of EIA buffer, non-specific binding wells received 100 μL of EIA buffer, and maximum binding wells received 50 μL of EIA buffer. PGDS 100% activity wells, non-specific binding, background, maximum binding, standards, and nettle extract wells received 50 μL of tracer. PGDS 100% activity, background, maximum binding, standards, and nettle extract wells also received 50 μL of antiserum. The EIA plate reactions were run for 2 h at room temperature. Plates were washed with wash buffer and 200 μL Ellman's Reagent was added to all wells, followed by 5 μL of tracer to the total activity well. The color development was quantified at 409 nm using a BioTek Synergy microplate reader.

F. Histamine Receptor (H₁) Activity Assays

Histamine Receptor (H₁) activity was determined using Geneblazer H1 HEK 293T Division Arrested Cells (Invitrogen, Calif.). Cells were seeded onto a tissue culture treated 384 well plate according to manufacturer's specifications using DMEM (Dulbecco's modified Eagle medium) with FBS (Fetal Bovine Serum) 10% Penicillin (100 U mL⁻¹), Streptomycin (100 μg mL⁻¹), non-essential amino acids (0.1 mM), and HEPES buffer. Cells were incubated overnight for 16-20 h in a CO₂ incubator (5% CO₂) at 37° C. allowing them to adhere to the plate. To determine if the nettle extracts were H1-receptor agonists, cells were exposed to serial dilutions of the nettle extracts for 5 h. For antagonist screening, cells were exposed to serial of dilutions of the nettle extracts for 30 min and then exposed to histamine (0.5 μM) at 37° C. for 4.5 h in a CO₂ incubator (5% CO₂). After the 5-h incubation period, CCF4-AM substrate (Invitrogen) was loaded in each well and incubated for 1 h at room temperature, according to the manufacturer's protocols. Plates were then excited at 409 nm and the emission read at 460 and 530 nm. The background subtracted fluorescence emission ratio (Em 460/530 nm) was obtained on a Biotek Synergy 4 plate reader (Winooski, Vt.) and percent inhibition of H₁-receptor activity in the presence of the nettle extracts as an agonist and antagonist was determined relative to histamine and triprolidine activity.

Antagonistic (competes for normal ligand) and negative agonistic (binds irreversibly to receptor blocking function) activities of Nettle for the Histamine (H₁) Receptor were examined. The extracts showed both H₁ receptor antagonism and negative agonist activities.

G. Tryptase Enzyme Inhibition Assays

Tryptase activity triggers mast cell degranulation which is requisite for release of cytokines and other factors that initiate allergy symptoms. Tryptase, a protease, activity was determined by monitoring the production of chromophore p-nitroaniline (pNA) generated by the cleavage of tosyl-gly-pro-lys-pNA by the tryptase enzyme according to the manufacturer's protocol (Millipore Inc., Westbury, Mass.). In a 96-well microtiter format, tryptase was added to the extract, followed by tosyl-gly-pro-lys-pNA and reaction buffer and incubated for 2 h at 37° C. After the incubation, absorbance at 40 nm was measured in each well using a BioTek Synergy 4 (BioTek, Winooski, Vt.) plate reader.

H. Human Pharmacokinetic Studies

Five healthy consenting adults ranging in age from 18 to 50 were instructed not to consume foods rich in polyphenolics 24 hr prior to the initiation of the study. A certified individual collected blood samples at several time intervals between 0 and 480 minutes after 2 lozenges of nettle Extract 2 were ingested. Immediately after the time zero time point, blood samples were collected, two 100-mg doses of nettle Extract 2 were administered and allowed to dissolve slowly in the oral cavity of the subjects. Blood samples were handled with approved protocols and precautions, centrifuged to remove cells and the serum fraction was collected and frozen. Blood was not treated with heparin to avoid any analytical interference. Urine samples were collected from the same 5 subjects on a time course (0 to 480 minutes) and frozen. Serum samples were stored frozen until analysis. The serum was extracted with an equal volume of neat ethanol (USP) to minimize background of proteins, peptides, and polysaccharides present in serum. The ethanol extract was centrifuged for 10 minutes at 4° C., the supernatant was removed, concentrated to 200 μL volume which was then used for DART TOF-MS analyses. Urine samples were stored at −80° C. until DART MS analysis. The samples were introduced by placing the closed end of a borosilicate glass capillary tube into the samples, and the coated capillary tube was placed into the DIP-it™ sample holder providing an even surface exposure for ionization in the He plasma. The sample was allowed to remain in the He plasma stream until signal was observed in the total-ion-chromatogram (TIC). The sample was removed and the TIC was brought down to baseline levels before the next sample was introduced. A polyethylene glycol 600 (Ultra Chemicals, Kingston R.I. ) was used as an internal calibration standard giving mass peaks throughout the desired range of 100-1000 amu.

### Results

A. Summary of in vitro Biological Activities of the Nettle Extracts

The nettle extracts were evaluated for 6 therapeutic endpoints related to seasonal allergies and associated inflammation. The extracts demonstrated dose-dependent inhibition for all endpoints. Nettle Extract 2, for example, possessed strong anti-allergenic activity across all the endpoints analyzed.

The IC₅₀ values for the selective inhibition of COX1 and COX2 by the nettle extracts 1 to 8 are depicted in Tables 1 and 2 respectively. The IC₅₀ values for Extract 2 for the selective inhibition of COX-1 and COX-2 are 294 and 228 μg mL⁻¹, respectively. Additionally, Table 3 contains the in vitro summary of inhibition activity against the HPGDS enzyme, another prostaglandin synthase. Multiple nettle extracts are active inhibitors of HPGDS with IC₅₀ values ranging from 191 to 524 μg mL⁻¹ (Table 3).

TABLE 1
Sumary of the COX1 inhibitory activity of the 8 nettle extracts described
herein with IC₅₀ values (μg mL⁻¹), correlation
coefficients (R²), and number of samples (N) provided
(NA = IC₅₀ value not achieved).

| Extract | COX-1 | | |
|---|---|---|---|
| No. | (IC₅₀ μg mL⁻¹) | R² | N |
| 1 | NA | NA | NA |
| 2 | 294 | 0.56 | 9 |
| 3 | NA | NA | NA |
| 4 | NA | NA | NA |
| 5 | NA | NA | NA |
| 6 | NA | 0.94 | 9 |
| 7 | NA | 0.85 | 9 |
| 8 | NA | 0.81 | 9 |

TABLE 2
Sumary of the COX-2 inhibitory activity of the 8 nettle extracts described
herein with IC₅₀ values (μg mL⁻¹), correlation coefficients
(R²), and number of samples (N) provided (NA = IC₅₀ value
not achieved).

| Extract | COX-2 | | |
|---|---|---|---|
| No. | (IC₅₀ μg mL⁻¹) | R² | N |
| 1 | NA | NA | NA |
| 2 | 228 | 0.81 | 9 |
| 3 | 2181 | 0.99 | 9 |
| 4 | 1499 | 0.9 | 9 |
| 5 | 2166 | 0.84 | 9 |
| 6 | NA | NA | NA |
| 7 | 2853 | 0.88 | 9 |
| 8 | NA | NA | NA |

TABLE 3
Sumary of the HPGDS inhibitory activity of the 8 nettle extracts
described herein with IC₅₀ values (μg mL⁻¹), correlation coefficients
(R²), and number of samples (N) provided (NA = IC⁵⁰ value not
achieved).

| Extract | HPGDS | | |
|---|---|---|---|
| No. | (IC₅₀ μg mL⁻¹) | R² | N |
| 1 | NA | NA | NA |
| 2 | 524 | 0.71 | 5 |
| 3 | 307 | 0.8 | 5 |
| 4 | 471 | 0.99 | 5 |
| 5 | 191 | 0.55 | 5 |
| 6 | NA | NA | NA |
| 7 | NA | NA | NA |
| 8 | NA | NA | NA |

Antagonistic (competes for normal ligand) and negative agonistic (binds irreversibly to receptor blocking function) activities of Nettle for the Histamine (H₁) Receptor were examined. The extracts showed both H₁ receptor antagonism and negative agonist activities, as seen in Tables 4 and 5. Nettle Extract 2, for example, possessed potent H₁ receptor activities with an IC₅₀ value of 250 μg mL⁻¹ for antagonism, and an IC₅₀ value of 190 μg mL⁻¹ for negative agonism. In both cases, IC₁₀₀ values were obtained (ca. 1000-1100 μg mL⁻¹).

TABLE 4
Sumary of H₁-receptor antagonism by the 8 nettle extracts described
herein with IC₅₀ values (μg mL⁻¹), correlation coefficients
(R²), and number of samples (N) provided (NA = IC₅₀ value
not achieved).

| Extract | H₁ Antagonism | | |
|---|---|---|---|
| No. | (IC₅₀ μg mL⁻¹) | R² | N |
| 1 | NA | NA | NA |
| 2 | 250 | 0.71 | 4 |
| 3 | NA | NA | NA |
| 4 | NA | NA | NA |
| 5 | 550 | 0.71 | 4 |
| 6 | 330 | 0.81 | 4 |
| 7 | 180 | 0.83 | 4 |
| 8 | 260 | 0.86 | 4 |

TABLE 5
Sumary of H₁-receptor negative agonism by the 8 nettle extracts
described herein with IC₅₀ values (μg mL⁻¹), correlation
coefficients (R²), and number of samples (N) provided (NA = IC₅₀ value
not achieved).

| | H₁ Negative | | |
|---|---|---|---|
| Extract | Agonism | | |
| No. | (IC₅₀ μg mL⁻¹) | R² | N |
| 1 | NA | NA | NA |
| 2 | 190 | 0.96 | 4 |
| 3 | NA | NA | NA |
| 4 | 1240 | 0.69 | 4 |
| 5 | 350 | 0.99 | 4 |
| 6 | 750 | 0.79 | 4 |
| 7 | 280 | 0.79 | 4 |
| 8 | 320 | 0.92 | 4 |

Tryptase activity triggers mast cell degranulation which is requisite for release of cytokines and other factors that initiate allergy symptoms. The IC₅₀ values for the selective inhibition of Tryptase by the nettle extracts are provided in Table 6. Nettle Extract 2, for example, showed a dose-dependent inhibition of Tryptase with an IC₅₀ value of 143 μg mL⁻¹.

TABLE 6
Sumary of tryptase enzyme inhibition activity by the 8 nettle extracts
described herein with IC₅₀ values (μg mL⁻¹), correlation
coefficients (R²), and number of samples (N) provided (NA = IC₅₀ value
not achieved).

| Extract | Tryptase | | |
|---|---|---|---|
| No. | (IC₅₀ μg mL⁻¹) | R² | N |
| 1 | 108 | 0.95 | 8 |
| 2 | 143 | 0.69 | 8 |
| 3 | 103 | 0.88 | 8 |
| 4 | 109 | 0.95 | 8 |
| 5 | 66 | 0.8 | 8 |
| 6 | 129 | 0.91 | 8 |
| 7 | 100% inhibition | | 8 |
| | achieved at all | | |
| | concentration | | |
| 8 | 48 | 0.82 | 8 |

Table 7 below provides a summary of the key bioactives present in any of the nettle extracts 1 through 8 analyzed along with their molecular mass, range in relative abundances throughout the 8 nettle extracts, and weight (in μg) per 100 mg of extract derived from the range in relative abundances.

TABLE 7
Summary of anti-inflammatory/anti-allergenic bioactive compounds
present in the nettle extracts described here listing the compound
name, molecular mass, range of relative abundance in the nettle
extracts, and range of weight (in μg) per 100 mg of nettle extract.

| | | Relative | Weight |
|---|---|---|---|
| | Molecular | Abundance | (μg) per |
| Compound Name | Mass | (%) | 100 mg |
| Resorcinol | 110.037 | 2.08-26.42 | 2,080-26,420 |
| 6-azacytosine | 112.039 | 0.15-50.53 | 150-50,530 |
| Proline | 115.063 | 1.98-45.09 | 1,980-45,090 |
| Levulinic acid | 116.047 | 0.87-38.59 | 870-38,590 |
| Threonine | 119.058 | 0.80-3.42 | 800-3,420 |
| Vitamin B3 (niacinamide) | 122.048 | 0.68-31.41 | 680-31,410 |
| Leucine | 131.095 | 3.66-27.96 | 3,660-27,960 |
| Adenine | 135.055 | 2.49-100 | 2,490-100,000 |
| Levoglucosan/Glyogen/ | 162.053 | 2.71-100 | 2,710-100,000 |
| Laminarin | | | |
| Synephrine | 167.095 | 1.62-33.58 | 1,620-33,580 |
| Shikimic acid | 174.053 | 3.94-17.25 | 3,940-17,250 |
| DL-methyl-m-tyrosine | 195.090 | 1.71-39.83 | 1,710-39,830 |
| 4-methyl-7-ethoxycoumarin | 204.079 | 2.02-38.08 | 2,020-38,080 |
| Vitamin B5 | 219.111 | 1.81-75.21 | 1,810-75,210 |
| isopropyl-B-D- | 238.088 | 0.97-2.43 | 970-2,430 |
| thiogalactopyranoside | | | |
| phosphatidylcholine | 242.115 | 1.82-14.13 | 1,820-14,130 |
| osthole | 244.110 | 1.81-22.55 | 1,810-22,550 |
| 4-shogaol | 248.121 | 1.56-28.20 | 1,560-28,200 |
| piperine/cocluarine/laurifoline | 285.137 | 0.61-16.22 | 610-16,220 |
| 8-dehydrogingerdione | 318.183 | 0.36-13.25 | 360-13,250 |
| sinomenin/deoxyharringtonine | 329.163 | 0.32-8.56 | 320-8,560 |
| picrocrocin/carnosol | 330.183 | 0.46-14.60 | 450-14,680 |

B. Human Pharmacokinetics

Key bioactives in nettle extract 2 appeared in serum within 10 minutes from 5 healthy adults who injested two 100-mg lozenges at time zero (FIG. 11). This initial rapid uptake is most likely ascribed to oral cavity uptake. The key bioactives included vitamin B3, leucine, adenine, levoglucosan, synpherine, and osthole. The levels of the bioactives increased through about 45 minutes and declined thereafter, though detectable levels persisted in serum through 2 hours. Interestingly, there was a second peak in bioactive levels at about 60 minutes post-consumption that probably indicates small intestine uptake (FIG. 11). The key bioactives in nettle Extract 2 appear in urine by the first 1-hour time point and persist through the 8-hour sampling time (FIG. 12). The data show that the key nettle Extract 2 bioactives appear in serum within minutes not hours.

C. DART TOF-MS Characterization of the Nettle Extracts

Tables 8 through 15 below indicate the compounds characterized by DART TOF-MS in each of the respective nettle Extracts 1 to 8. Tables 3 through 10 list the compound name (as determined by a searchable database of exact masses), the calculated mass of the compound, and the relative abundance (%) of the compound in each extract.

TABLE 8
DART TOF-MS characterization of Nettle Extract 1.

| | | Rel. |
|---|---|---|
| | Calc. | Abund. |
| Compound Name | Mass | (%) |
| 2-Methyl-2-butenoic | 100.0762 | 1.9648 |
| acid Amide | | |
| 3-Aminodihydro- | 102.0555 | 1.5637 |
| 2(3H)-furanone | | |
| N,N- | 104.0711 | 20.6499 |
| Dimethylglycine | | |
| 5-Methyl-3- | 109.0402 | 4.5826 |
| isoxazolecarboxylic | | |
| acid: Nitrile | | |
| 1H-Pyrrole-3- | 110.0718 | 13.4511 |
| carboximidamide | | |
| Resorcinol | 111.0446 | 2.0828 |
| 2,3-Dihydro-1H- | 112.0762 | 2.9012 |
| pyrrole: N—Ac | | |
| Levulinic acid | 116.0711 | 9.117 |
| 4,5-Dihydro-2- | 117.0612 | 4.2947 |
| methylthiazole: N- | | |
| Me | | |
| 2- | 118.0868 | 11.1257 |
| Methylaminoacetic | | |
| acid: Et ester | | |
| Threonine | 120.066 | 1.6426 |
| Trimethylvinylammonium | 122.0736 | 2.099 |
| Chloride | | |
| 2,3-Dimethyl-5- | 123.081 | 7.583 |
| methylene-2- | | |
| cyclopenten-1-one | | |
| 2- | 124.0762 | 7.4542 |
| Hydroxybenzylamine | | |
| 1H-Pyrrole-2- | 125.0715 | 5.3107 |
| carboxaldehyde N- | | |
| Me, oxime | | |
| 2-Amino-4- | 126.0667 | 6.0202 |
| hydroxypyrimidine | | |
| 1-Me | | |
| 5-Hydroxy-3-vinyl- | 127.0395 | 26.7256 |
| 2(5H)-furanone | | |
| 1-Nonene | 127.1487 | 0.3958 |
| 5,6-Dihydro-5- | 129.0551 | 1.6286 |
| hydroxy-6-methyl- | | |
| 2H-pyran-2-one | | |
| 2-Amino-4-hexenoic | 130.0868 | 17.7356 |
| acid | | |
| Leucine | 132.1024 | 17.0822 |
| 2-Amino-4-hydroxy- | 134.0817 | 1.3897 |
| 3-methylbutanoic | | |
| acid | | |
| Adenine | 136.061 | 33.2384 |
| 4-Methylbenzoic | 137.0602 | 14.4814 |
| acid | | |
| 5-Ethyl-2- | 138.0919 | 10.4585 |
| methylpyridine: N- | | |
| Oxide | | |
| Choline: Chloride | 140.0842 | 7.7662 |
| 2-Aminoethanol: N, | 142.1232 | 6.1354 |
| N-Di-2-propenyl | | |
| 4-Phenyl-3-butenoic | 144.0813 | 10.9701 |
| acid Nitrile | | |
| 2,3-Dihydro-3,6- | 145.0501 | 52.2885 |
| dihydroxy-2-methyl- | | |
| 4H-pyran-4-one | | |
| 2-Amino-3- | 146.0817 | 19.3727 |
| hydroxymethyl-3- | | |
| pentenoic acid | | |
| 2-Amino-5- | 148.0973 | 1.2195 |
| hydroxyhexanoic | | |
| acid | | |
| 4-Methylbenzoic | 150.0919 | 5.6078 |
| acid Methylamide | | |
| Ethyl-1,4- | 152.0711 | 1.7088 |
| benzoquinone 4- | | |
| Oxime | | |
| 1,4-Benzenediol | 153.0915 | 2.7635 |
| Me-Et ether | | |
| Scopine: 3-Ketone | 154.0868 | 9.2018 |
| 7-Isothiocyanato-1- | 156.0847 | 5.8973 |
| heptene | | |
| Diptocarpilidine: S- | 158.1003 | 19.0104 |
| Deoxo | | |
| 4-Hydroxy-1,1- | 160.0973 | 8.0789 |
| dimethylpyrrolidinium- | | |
| 2-carboxylate | | |
| 3-Aminotetrahydro- | 162.0766 | 7.9005 |
| 5-(hydroxymethyl)- | | |
| 3-furancarboxylic | | |
| acid | | |
| Levoglucosan/Glyogen/ | 163.0615 | 16.4584 |
| Laminarin | | |
| N-[2-(4- | 164.0711 | 49.0504 |
| Hydroxyphenyl)ethenyl]formamide | | |
| 2-Amino-2- | 166.0868 | 29.0305 |
| phenylpropanoic | | |
| acid | | |
| Synephrine | 168.1024 | 4.0846 |
| Tetramethylpyrazine: | 169.0977 | 3.6595 |
| 1,4-Dioxide | | |
| 3(5)- | 170.0718 | 1.9273 |
| Pyrazolecarboxylic | | |
| acid N-Ph, nitrile | | |
| 2-Aminoethanol: N, | 172.1337 | 10.1959 |
| N-Di-Et, 2- | | |
| propenoyl | | |
| 6-Hydroxy-4,8- | 174.0919 | 7.5932 |
| dimethylquinoline | | |
| 1H-Indole-2,3- | 176.0711 | 3.1787 |
| dione: N-Et | | |
| N-[2-(4- | 178.0868 | 31.7493 |
| Hydroxyphenyl)ethenyl]formamide: | | |
| Me | | |
| ether | | |
| 1,2,3,4-Tetrahydro- | 180.1024 | 55.2555 |
| 4,8-dihydroxy-2- | | |
| methylisoquinoline | | |
| 2-Amino-3-(4- | 181.0977 | 9.4717 |
| aminophenyl)propanoic | | |
| acid | | |
| (2- | 182.0946 | 10.9694 |
| Aminoethyl)phosphonic | | |
| acid: Di-Et | | |
| ester | | |
| Carcinine | 183.1246 | 2.4224 |
| 2-Naphthol: 2- | 185.0966 | 3.1673 |
| Propenyl ether | | |
| 1-(1-Hydroxyethyl)- | 186.0766 | 1.1299 |
| 3-isocyano-4- | | |
| cyclopentene-1,2,3- | | |
| triol | | |
| Ecgonine | 186.113 | 1.8416 |
| 2-(1-Aminopropyl)- | 187.0541 | 1.2617 |
| 4-thiazolecarboxylic | | |
| acid | | |
| 2-Amino-4- | 188.0923 | 12.2713 |
| propylidenepentanedioic | | |
| acid | | |
| 2-Ethyl-4- | 190.098 | 6.5012 |
| hydroxyquinazoline | | |
| N-Amino | | |
| Carnitine Et ester | 191.1521 | 7.3563 |
| Hydrohydrastinine | 192.1024 | 70.3935 |
| 1,2,3,4- | 193.0501 | 0.9148 |
| Naphthalenetetrol | | |
| (N- | 193.0977 | 6.1562 |
| Phenylacetyl)glycine | | |
| Amide | | |
| 1,2,3,4-Tetrahydro- | 194.1181 | 16.9515 |
| 7-hydroxy-6- | | |
| methoxy-1- | | |
| methylisoquinoline | | |
| DL-methyl-m- | 196.0821 | 2.5659 |
| tyrosine | | |
| 2-Acetyl-4,4,6- | 197.1177 | 6.9083 |
| trimethyl-1,3- | | |
| cyclohexanedione | | |
| Buddamine | 198.113 | 13.5659 |
| Pedatisectine D | 199.1082 | 12.1825 |
| N-(2- | 200.1035 | 8.2982 |
| Cyanoethyl)glutamine | | |
| 2,6- | 202.1079 | 4.5907 |
| Piperidinedicarboxylic | | |
| acid Di-Me ester | | |
| 1H-Indole-3- | 204.1137 | 5.9177 |
| propanoic acid: | | |
| Hydrazide | | |
| 4-methyl-7- | 205.0725 | 2.0175 |
| ethoxycoumarin | | |
| N-(2- | 206.1181 | 16.6926 |
| Hydroxyethyl)camamide | | |
| N-Me | | |
| Indole: N-Benzyl | 208.1126 | 16.6407 |
| 6,10-Dimethyl-5,9- | 209.1541 | 4.131 |
| undecadiene-2,8- | | |
| dione | | |
| 4-Methylbenzoic | 212.1075 | 10.3063 |
| acid: Anilide | | |
| Linderazulene: 2,3- | 213.1279 | 3.1458 |
| Dihydro | | |
| 2-Aminobenzyl | 214.1232 | 7.3705 |
| alcohol: N-Benzyl | | |
| 2-(2,4- | 215.1072 | 5.5426 |
| Hexadiynylidene)- | | |
| 1,6- | | |
| dioxaspiro[4.5]dec- | | |
| 3-ene | | |
| a-Amino-?- | 216.1236 | 10.233 |
| oxooxiraneoctanoic | | |
| acid | | |
| 2,3,4,9-Tetrahydro- | 217.0977 | 5.5286 |
| 1H-pyrido[3,4-b]indol- | | |
| 1-one: 7- | | |
| Methoxy | | |
| Glycylglycylglycine: | 218.1141 | 14.1975 |
| Et ester | | |
| Vitamin B5 | 220.1185 | 33.6049 |
| 2-Amino-2- | 222.1341 | 21.0243 |
| deoxygalactose | | |
| 3,4,6-Tri-Me | | |
| Murexine | 225.1477 | 3.8679 |
| 2-Amino-4-hydroxy- | 226.1079 | 3.4389 |
| 4-(4- | | |
| hydroxyphenyl)-3- | | |
| methylbutanoic acid | | |
| 2-Amino-4-hexynoic | 228.1236 | 4.6069 |
| acid N-tert- | | |
| Butyloxycarbonyl | | |
| 5,8-Dihydroxy-2,6- | 229.144 | 6.9223 |
| dodecadienoic acid | | |
| Kinetin; 7H-form: | 230.1042 | 15.0782 |
| 7-Me | | |
| 9H-Pyrido[3,4-b]indol- | 231.1133 | 3.3789 |
| 7-ol: 1,2,3,4- | | |
| Tetrahydro, Me | | |
| ether, N9-formyl | | |
| 1H-Indole-3- | 232.1337 | 10.4005 |
| butanoic acid: Et | | |
| ester | | |
| Arabinitol 2,3:4,5- | 233.1389 | 4.99 |
| Di-O- | | |
| isopropylidene | | |
| 4-(3-Indolyl)-2- | 234.1494 | 34.194 |
| methyl-1-butanol N- | | |
| Methoxy | | |
| 7-Isopropyl-1- | 235.1487 | 4.914 |
| methylphenanthrene | | |
| Anhalonine N-Me | 236.1286 | 23.562 |
| 2-Amino-3- | 238.1079 | 7.0831 |
| hydroxypentanoic | | |
| acid N-Benzoyl | | |
| 2-Hydroxybenzoic | 242.1392 | 6.7807 |
| acid: Choline ester | | |
| Lapachenol 3,4- | 243.1385 | 2.2979 |
| Dihydro | | |
| 4-Oxo-2- | 244.1185 | 17.7923 |
| piperidinecarboxylic | | |
| acid N-tert- | | |
| Butyloxycarbonyl | | |
| Osthole | 245.0926 | 1.9912 |
| 3-Deoxy-3- | 246.1341 | 17.4536 |
| (methylamino)arabinose; | | |
| β-L-Furanose- | | |
| form: N—Ac, 1,2-O- | | |
| isopropylidene | | |
| Hypaphorine | 247.1446 | 7.0083 |
| 3-(3,4- | 248.1286 | 14.4311 |
| Methylenedioxyphenyl)- | | |
| 2-propenoic | | |
| acid 2- | | |
| Methylpropylamide | | |
| Anhalonine N-Et | 250.1443 | 13.5051 |
| N-2-Phenylethylcamamide | 252.1388 | 12.6985 |
| Ichthyopterin | 254.0889 | 2.0294 |
| a,a′-Diamino-2- | 255.1345 | 3.7563 |
| methylenecycloheptene- | | |
| 1,3-diacetic acid | | |
| Kainic acid N—Ac | 256.1185 | 4.7498 |
| Pachysontol | 257.1389 | 4.2106 |
| Elaeagin | 259.1334 | 5.3854 |
| 3-(Dimethylamino)- | 260.1498 | 10.6096 |
| 2,3,6-trideoxy-lyxo- | | |
| hexose Di-Ac | | |
| Cyclo(leucylphenylalanyl) | 261.1603 | 6.6423 |
| 8-Methyl-8- | 262.1443 | 11.1775 |
| azabicyclo[3.2.1]octane- | | |
| 3,6-diol 3- | | |
| Benzoyl | | |
| 2-Acetamido-2- | 264.1447 | 8.3279 |
| deoxyglucose 3,4,6- | | |
| Tri-Me | | |
| 2-Acetyl-3,5,6,8- | 265.0348 | 0.7374 |
| tetrahydroxy-1,4- | | |
| naphthoquinone | | |
| Siamenol | 266.1545 | 9.0859 |
| Showdomycin: | 268.0821 | 1.048 |
| 2′,3′-Isopropylidene | | |
| 3,4,5- | 269.1177 | 2.5666 |
| Phenanthrenetriol: | | |
| Tri-Me ether | | |
| 1,3- | 270.113 | 5.1355 |
| Dihydroxyacridone: | | |
| Di-Me ether, N-Me | | |
| 2-Hydroxybenzoic | 271.1334 | 5.4463 |
| acid: 4-tert- | | |
| Butylphenyl ester | | |
| Cynometrine: N1- | 272.1399 | 9.5354 |
| De-Me | | |
| Verimol F | 273.149 | 5.3731 |
| Otanthusic acid | 274.1265 | 37.4133 |
| Piperidide | | |
| 12,18- | 275.18 | 17.1547 |
| Heneicosadiene- | | |
| 1,3,8,10,20-pentayne | | |
| 3,4-Dihydro | | |
| 8-Methyl-8- | 276.1599 | 23.2952 |
| azabicyclo[3.2.1]octane- | | |
| 3,6-diol 3-O- | | |
| Phenylacetyl | | |
| 9,11,13,15- | 277.2167 | 53.776 |
| Octadecatetraenoic | | |
| acid | | |
| Stenine | 278.212 | 23.828 |
| 7-Hydroxy-14,15- | 279.2324 | 39.6541 |
| dinor-8(17)-labden- | | |
| 13-one | | |
| Poranthericine: Ac | 280.2276 | 14.2487 |
| piperine/cocluarine/laurifoline | 286.1443 | 6.6875 |
| Komaroine | 287.1548 | 4.2332 |
| Trichostatic acid | 288.1599 | 11.133 |
| chiro-Inositol 1,4- | 289.1651 | 12.6858 |
| Di-Me, 2,3:5,6-di-O- | | |
| isopropylidene | | |
| Oridine | 290.1756 | 15.7679 |
| 8-Hydroxy-17- | 291.196 | 100 |
| octadecene-9,11- | | |
| diynoic acid | | |
| Ecklonialactone A: | 293.2116 | 5.112 |
| 6,7-Dihydro | | |
| 2-Amino-2- | 294.1916 | 5.6438 |
| deoxyglucose Di-Et | | |
| acetal, 5,6-O- | | |
| isopropylidene | | |
| 2-Amino-4,8,10- | 296.2589 | 10.1082 |
| octadecatriene-1,3- | | |
| diol | | |
| 1,10:4,5-Diepoxy- | 299.1494 | 1.3089 |
| 3,6,8-trihydroxy-11- | | |
| germacren-9-one | | |
| Saxitoxin | 300.142 | 3.7823 |
| N,N′-Bis(3- | 301.1552 | 2.3699 |
| methoxybenzyl)urea | | |
| Pulchelline | 302.1756 | 10.146 |
| Trichostatin A | 303.1708 | 5.4059 |
| Schoberidine | 304.1813 | 7.5654 |
| HapalindoleA: | 305.2017 | 8.8716 |
| Dechloro | | |
| Amicetamine | 306.1916 | 10.917 |
| Spermidine: N ?- | 308.1974 | 7.9522 |
| (3,4-Dihydroxyc | | |
| amoyl) | | |
| Taberpsychine | 309.1967 | 2.461 |
| Atherosperminine | 310.1807 | 9.5657 |
| Angustine | 314.1293 | 3.188 |
| Ipanguline D3: 3″- | 316.176 | 7.3577 |
| Ac | | |
| Franklinol | 317.1753 | 5.3081 |
| Blastmycetin B: 3- | 318.1817 | 7.1186 |
| Epimer, 3-deoxy | | |
| 8- | 319.1909 | 5.8511 |
| dehydrogingerdione | | |
| Calabacine | 320.1974 | 6.8342 |
| 8-Methyl-8- | 322.2018 | 7.2431 |
| azabicyclo[3.2.1]octane- | | |
| 3,6-diol | | |
| Ditigloyl | | |
| Inomycin | 324.2049 | 3.8006 |
| Glucaric acid 2,4-O- | 327.108 | 0.3972 |
| Benzylidene,di-Me | | |
| ester | | |
| Lomatin O-(3- | 329.1389 | 0.7476 |
| Methyl-2-butenoyl) | | |
| sinomenin/deoxyharringtonine | 330.1705 | 3.7125 |
| picrocrocin/carnosol | 331.1757 | 4.9942 |
| 4-(5-Phenyl-2,4- | 333.1854 | 5.0803 |
| pentadien-1- | | |
| yl)tetracyclo[5.4.0.02, | | |
| 5.03,9]undec-10- | | |
| ene-8-carboxylic | | |
| acid | | |
| 4-[2- | 334.2018 | 6.6536 |
| (Acetylmethylamino)ethyl]- | | |
| 4-(4- | | |
| hydroxy-3- | | |
| methoxyphenyl)-2,4- | | |
| cyclohexadien-1- | | |
| one: 2,3,5,6- | | |
| Tetrahydro, Me | | |
| ether | | |
| 2,6-Dimethyl-7- | 335.207 | 4.5348 |
| octene-1,6-diol: 8-O- | | |
| β-D- | | |
| Glucopyranoside | | |
| 11-Hydroxy-4,12- | 336.1997 | 6.2704 |
| dimethyl-13-(2- | | |
| methyl-4-thiazolyl)- | | |
| 8,12-tridecadien-3- | | |
| one | | |
| Daphnicadine | 338.212 | 4.9174 |
| Cataline: O1-De- | 344.1498 | 2.9456 |
| Me,N-de-Me | | |
| 3,7,11,15(17)- | 345.1702 | 2.1457 |
| Cembratetraene- | | |
| 16,2:19,6-diolide | | |
| 11,12-Epoxide | | |
| Suaveoline N—Ac | 346.1919 | 4.4611 |
| Pseudophrynamine | 347.1971 | 3.9829 |
| 258A 5,6- | | |
| Dimethoxy, 4′- | | |
| carboxylic acid, Me | | |
| ester | | |
| Mahanine | 348.1963 | 5.156 |
| Amorphastilbol | 349.2167 | 4.4141 |
| 2-Pentadecyl-4(1H)- | 350.2484 | 9.9014 |
| quinolinone: | | |
| 6′,7′,9′,10′,12′,13′- | | |
| Hexadehydro | | |
| Manzamine C: | 352.2752 | 2.0068 |
| 1?,2,3,4-Tetrahydro | | |
| ribo-Hexos-3-ulose | 354.1916 | 3.5209 |
| 1,2:5,6-Di-O- | | |
| cyclohexylidene, | | |
| oxime | | |
| Lincosamine 7-Me, | 360.2022 | 4.8493 |
| 1,2:3,4-di-O- | | |
| isopropylidene, N—Ac | | |
| Cypholophine: O—Ac | 361.2127 | 6.3829 |
| Murrayamine F | 362.212 | 6.2372 |
| 12-Hydroxy- | 363.2171 | 3.5193 |
| 8(17),13-labdadien- | | |
| 15,12-olid-19-oic | | |
| acid12-Me ether | | |
| Euchrestine C: 2-Me | 364.2276 | 6.0717 |
| ether | | |
| 3,17,20- | 366.2644 | 5.6894 |
| Trihydroxypregnan- | | |
| 11-one Oxime | | |
| Acsine | 376.2124 | 4.3936 |
| Estra-1,3,5(10)- | 377.2116 | 2.7753 |
| triene-3,17-diol 3- | | |
| Benzoyl | | |
| Heteratisine: O-De- | 378.228 | 4.9504 |
| Me | | |
| Pseudostrychnine: Et | 379.2021 | 1.7253 |
| ether | | |
| 2-Amino-4- | 380.2566 | 5.9847 |
| octadecene-1,3-diol | | |
| 1-Phosphate | | |
| Dioncophylline A: | 390.2069 | 3.2928 |
| 1,2-Didehydro, Me | | |
| ether | | |
| 1,6,14-Trihydroxy- | 391.212 | 1.669 |
| 16-kaurene-7,15- | | |
| dione 6-Ac | | |
| DioncophyllineA: | 392.2225 | 3.4152 |
| Me ether | | |
| Linderatone | 393.2066 | 2.3128 |
| Macrocentrine | 394.2593 | 5.6223 |
| 5-Hydroxy-9- | 397.4045 | 4.0735 |
| hexacosanone | | |
| Dammar-24-ene- | 404.2437 | 3.9317 |
| 3,20,26-triol | | |
| Aurachin E | 405.2542 | 3.5254 |
| 2-Phenyl-1,5,9- | 406.2494 | 2.5586 |
| triazacyclotridecan- | | |
| 4-one N9-E-C | | |
| amoyl | | |
| Linderatone: O7-Me | 407.2222 | 2.4232 |
| 5,13-Dihydroxy- | 408.2572 | 4.0573 |
| 2,4,6,14-tetramethyl- | | |
| 15-(2-methyl-4- | | |
| thiazolyl)-10,14- | | |
| pentadecadien-3-one | | |
| Fructigenine B | 410.2443 | 2.7878 |
| Delboxine: O8,O14- | 422.2179 | 1.2527 |
| Di-de-Me, 14- | | |
| ketone | | |
| Paxilline: 4b- | 422.2695 | 1.3341 |
| Deoxy, 3β-alcohol | | |
| Butirosamine | 424.2407 | 2.6755 |
| Coyhaiquine: 1″- | 432.1811 | 0.9409 |
| Aldehyde | | |
| Ipanguline D3: 7-O- | 434.2179 | 2.3382 |
| (Phenylacetyl), 3″- | | |
| Ac | | |
| Koumicine | 435.2284 | 1.5814 |
| Acophine | 436.2618 | 2.7454 |
| 1,4-Epoxy-1,8,13- | 437.1811 | 1.622 |
| trihydroxy-5,7(11)- | | |
| germacradien-12,6- | | |
| olide; (1a,4β,5E, | | |
| 8β,10βH)-form: 8- | | |
| O-(4-Hydroxy-2- | | |
| methyl-2E- | | |
| butenoyl), 13-Ac | | |
| 4-Methylaconit-2- | 438.2492 | 2.1446 |
| ene- | | |
| 1,6,7,8,14,16,18- | | |
| heptol; | | |
| (1a,5β,6β,14a,16β)- | | |
| form: O16,O18-Di- | | |
| Me, N-Et | | |
| Diploclidine | 440.2801 | 1.459 |
| 8-(4-Methyl-2- | 448.2124 | 0.9012 |
| oxopentyl)dihydrochelerythrine | | |
| Sarcotragin A: N- | 448.2699 | 0.6893 |
| De-(2-phenylethyl), | | |
| N-(carboxymethyl) | | |
| Gentamicin C; | 448.3135 | 0.6023 |
| Gentamicin C2b: 5- | | |
| Deoxy | | |
| Mutamicin 1B | 450.2564 | 2.7002 |
| 4-Methylaconitane- | 452.2648 | 2.2319 |
| 1,6,7,8,14,16,18- | | |
| heptol; | | |
| (1a,5β,6β,14a,16β)- | | |
| form: 14-Ketone, O6, | | |
| O16,O18-tri-Me, | | |
| N-Et | | |
| Ascochalasin: 21,22- | 464.2801 | 2.9735 |
| Didehydro(E-) | | |
| 4-Methylaconitane- | 466.2805 | 2.3096 |
| 1,6,7,8,14,16,18- | | |
| heptol; | | |
| (1a,5β,6β,14a,16β)- | | |
| form: 14-Ketone, O1, | | |
| O6,O16,O18- | | |
| tetra-Me, N-Et | | |
| Ethyl glucoside; a- | 479.2433 | 2.6944 |
| D-Pyranose-form: | | |
| 3,4,6-Tribenzyl | | |
| Fumitremorgin B | 480.2498 | 5.1852 |
| 6-Hydroxy-3,4- | 490.0985 | 1.8594 |
| methylenedioxy-10- | | |
| nitro-1- | | |
| phenanthrenecarboxylic | | |
| acid: 6-O-β-D- | | |
| Glucopyranoside | | |
| 2,3,5,11,14- | 491.2281 | 0.3587 |
| Pentahydroxy-12- | | |
| oxobufa-20,22- | | |
| dienolide; | | |
| (2β,3β,5β,11a,14β)- | | |
| form: 2-Ac | | |
| 3,14,19- | 549.27 | 3.5108 |
| Trihydroxycarda- | | |
| 5,20(22)-dienolide; | | |
| (3β,14β)-form: 19- | | |
| Aldehyde, 3-O-β-D- | | |
| glucopyranoside | | |
| Ergosine: 9,10a- | 550.3029 | 4.9219 |
| Dihydro | | |
| Cyasterone: 3-Ac | 563.322 | 12.9919 |
| CytochalasinB: Di- | 564.2961 | 2.8385 |
| Ac | | |
| 2-Amino-6,9- | 664.6244 | 4.1551 |
| heptacosadiene- | | |
| 1,3,5-triol; | | |
| (2?,3?,5?,6E,9E)- | | |
| form: N- | | |
| Pentadecanoyl | | |
| Pyrophaeophorbide | 813.5682 | 7.4569 |
| a: 3,7R,5,11R- | | |
| Tetramethyl-2E- | | |
| hexadecenyl ester | | |

TABLE 9
DART TOF-MS characterization of Nettle Extract 2.

| | | Rel. |
|---|---|---|
| | Calc. | Abund. |
| Compound Name | Mass | (%) |
| 3-Aminodihydro- | 102.0555 | 1.6586 |
| 2(3H)-furanone | | |
| 4-Hydroxy-2- | 103.0395 | 9.9254 |
| oxobutanal | | |
| N,N- | 104.0711 | 19.4027 |
| Dimethylglycine | | |
| 5-Methyl-3- | 109.0402 | 5.6214 |
| isoxazolecarboxylic | | |
| acid: Nitrile | | |
| 1H-Pyrrole-3- | 110.0718 | 9.1518 |
| carboximidamide | | |
| Resorcinol | 111.0446 | 9.5431 |
| Cytosine; OH-form | 112.0511 | 12.0251 |
| 6-azacytosine | 113.0425 | 31.2587 |
| 3-Azetidinone: N—Ac | 114.0555 | 6.2286 |
| 2-Amino-3- | 115.0507 | 7.8158 |
| cyanopropanoic | | |
| acid; (S)-form | | |
| Proline | 116.0711 | 3.4373 |
| Levulinic acid | 117.0612 | 38.5861 |
| 2-Amino-3,4- | 118.0504 | 3.0894 |
| dihydroxybutanoic | | |
| acid; (2R,3R)-form: | | |
| Lactone | | |
| Succinic acid | 119.0344 | 5.0119 |
| Threonine | 120.066 | 1.3511 |
| Nitrosofungin | 121.0613 | 6.5386 |
| Benzaldehyde, (E)- | 122.0606 | 2.1105 |
| Oxime | | |
| Vitamin B3 | 123.0657 | 4.2889 |
| (niacinamide) | | |
| 2- | 124.0762 | 6.7941 |
| Hydroxybenzylamine | | |
| 1H-Pyrrole-2- | 125.0715 | 12.0719 |
| carboxaldehyde: N- | | |
| Me, oxime | | |
| 2-Amino-4- | 126.0667 | 7.5932 |
| hydroxypyrimidine; | | |
| 1H-form: 1-Me | | |
| 5-Hydroxy-3-vinyl- | 127.0395 | 19.4107 |
| 2(5H)-furanone | | |
| 3- | 128.0711 | 4.1698 |
| Azabicyclo[3.1.0]hexane- | | |
| 2-carboxylic | | |
| acid; (1S,2S,5R)- | | |
| form | | |
| 3-Azetidinone: N—Ac, | 129.0664 | 6.7285 |
| oxime | | |
| 2-Amino-4-hexenoic | 130.0868 | 3.1848 |
| acid | | |
| Leucine | 132.1024 | 4.7604 |
| 3,4-Dihydroxy-2- | 133.0501 | 19.6285 |
| methylenebutanoicacid | | |
| 1H-Indol-3-ol | 134.0606 | 3.224 |
| 2,3-Dihydroxy-2- | 135.0657 | 1.3173 |
| methylbutanoicacid | | |
| Adenine | 136.061 | 49.4889 |
| 4-Methylbenzoic | 137.0602 | 10.9016 |
| acid | | |
| 1,4- | 139.0759 | 6.4353 |
| Dimethoxybenzene | | |
| Choline: Chloride | 140.0842 | 10.2012 |
| Norzooanemonin | 141.0664 | 3.813 |
| a-Amino-2- | 142.0868 | 4.2666 |
| cyclopentene-1- | | |
| acetic acid | | |
| 2- | 144.066 | 4.9263 |
| Hydroxymethylclavam | | |
| 2,3-Dihydro-3,6- | 145.0501 | 14.7854 |
| dihydroxy-2-methyl- | | |
| 4H-pyran-4-one | | |
| 2,6-Dimethyl-1- | 145.1592 | 0.634 |
| heptanol; (S)-form | | |
| 2-Amino-3- | 146.0817 | 5.824 |
| hydroxymethyl-3- | | |
| pentenoic acid | | |
| 1,4:3,6- | 147.0657 | 7.4541 |
| Dianhydromannitol | | |
| 2-Hydroxybenzoic | 148.0762 | 2.0246 |
| acid: Et ether, nitrile | | |
| O-Carbamoylserine | 149.0562 | 9.1354 |
| 4-Methylbenzoic | 150.0919 | 9.813 |
| acid: Methylamide | | |
| Diisopropyldisulfide | 151.0615 | 4.5401 |
| Ethyl-1,4- | 152.0711 | 7.5959 |
| benzoquinone: 4- | | |
| Oxime | | |
| Phenylmethanethiol: | 153.0738 | 8.4557 |
| S-Et | | |
| Scopine: 3-Ketone | 154.0868 | 7.7181 |
| 3,4- | 155.0708 | 4.8341 |
| Dihydroxybenzyl | | |
| alcohol: 4-Me ether | | |
| 7-Isothiocyanato-1- | 156.0847 | 5.5881 |
| heptene | | |
| 3-(2-Hydroxyethyl)- | 158.0817 | 2.5761 |
| 4-oxa-1- | | |
| azabicyclo[3.2.0]heptan- | | |
| 7-one | | |
| 2-Amino-4- | 159.0769 | 1.8336 |
| methylenepentanedioic | | |
| acid; (S)-form: | | |
| ?-Amide | | |
| N-Benzoylglycine: | 161.0715 | 1.0294 |
| Nitrile | | |
| 3-Aminotetrahydro- | 162.0766 | 4.2859 |
| 5-(hydroxymethyl)- | | |
| 3-furancarboxylic | | |
| acid | | |
| Levoglucosan/Glyogen/ | 163.0615 | 100 |
| Laminarin | | |
| N-[2-(4- | 164.0711 | 9.693 |
| Hydroxyphenyl)ethenyl]formamide | | |
| Rhamnose; D-form | 165.0763 | 8.4339 |
| 2-Amino-2- | 166.0868 | 10.241 |
| phenylpropanoic | | |
| acid | | |
| Amino-1,4- | 167.082 | 6.7397 |
| benzoquinone: N- | | |
| Me, 4-methyloxime | | |
| Iodoacetic acid: | 167.931 | 0.2402 |
| Nitrile | | |
| Synephrine | 168.1024 | 32.6556 |
| 2-Aminoethyl | 170.0582 | 0.9398 |
| dihydrogen | | |
| phosphate: Mono-Et | | |
| ester | | |
| 1-(3,4- | 171.0657 | 0.8038 |
| Dihydroxyphenyl)- | | |
| 1,2-ethanediol; (?)- | | |
| form | | |
| 2-Amino-4-hexenoic | 172.0973 | 2.3814 |
| acid; (±)-(Z)-form: | | |
| N—Ac | | |
| 3,7-Dimethyl-1H- | 173.0966 | 2.6395 |
| indene-5- | | |
| carboxaldehyde | | |
| 1,2,3- | 174.0878 | 2.0807 |
| Propanetricarboxylic | | |
| acid: Triamide | | |
| Shikimic acid | 175.0606 | 10.1962 |
| 3- | 176.0923 | 5.3511 |
| Aminoheptanedioic | | |
| acid; (±)-form | | |
| Chloroiodomethane | 176.8968 | 0.1241 |
| N4-(2- | 177.0875 | 4.0369 |
| Hydroxyethyl)asparagine; | | |
| (S)-form | | |
| N-[2-(4- | 178.0868 | 4.8626 |
| Hydroxyphenyl)ethenyl]formamide: | | |
| Me | | |
| ether | | |
| myo-Inosose 2 | 179.0555 | 3.0288 |
| 6-Deoxymannonic | 180.0872 | 35.5968 |
| acid; L-form: | | |
| Amide | | |
| 2-(4- | 181.0864 | 21.0621 |
| Hydroxyphenyl)propanoic | | |
| acid; (R)- | | |
| form: Me ester | | |
| (2- | 182.0946 | 11.9684 |
| Aminoethyl)phosphonic | | |
| acid: Di-Et ester | | |
| Talitol | 183.0868 | 6.8052 |
| 4,6-Dimethyl- | 185.0814 | 1.9223 |
| 1,2,3,5-benzenetetrol: | | |
| 1-Me ether | | |
| 1-(1-Hydroxyethyl)- | 186.0766 | 2.1198 |
| 3-isocyano-4- | | |
| cyclopentene-1,2,3- | | |
| triol | | |
| a-Amino-5-oxo- | 187.0719 | 4.6277 |
| 2(5H)- | | |
| isoxazolebutanoic | | |
| acid | | |
| 2-Amino-4- | 188.0923 | 3.4936 |
| propylidenepentanedioic | | |
| acid | | |
| 4-Hydroxy-3-(3- | 189.0915 | 2.7638 |
| methyl-3-buten-1- | | |
| ynyl)benzoic acid: | | |
| 1′-Alcohol | | |
| 2-Ethyl-4- | 190.098 | 3.2006 |
| hydroxyquinazoline; | | |
| 3H-form: N-Amino | | |
| 7-Hydroxy-2H-1- | 191.0708 | 0.7431 |
| benzopyran-2-one: | | |
| Et ether | | |
| 4-Cyclohexylphenol: | 191.1436 | 9.7378 |
| Me ether | | |
| Hydrohydrastinine | 192.1024 | 8.8975 |
| 2,6,8,10- | 193.1228 | 8.4651 |
| Dodecatetraenoicacid | | |
| 2-Amino-2- | 194.1028 | 5.6635 |
| deoxygulose; D- | | |
| form: N-Me | | |
| 3,4-Dihydro-3,8- | 195.0657 | 1.1773 |
| dihydroxy-3-methyl- | | |
| 1H-2-benzopyran-1- | | |
| one | | |
| DL-methyl-m- | 196.0973 | 4.9624 |
| tyrosine | | |
| 2-Acetyl-4,4,6- | 197.1177 | 6.3062 |
| trimethyl-1,3- | | |
| cyclohexanedione | | |
| Stizolamine | 198.0991 | 12.3894 |
| 5-(1-Hydroxybutyl)- | 199.097 | 2.7235 |
| 6-hydroxymethyl-2H- | | |
| pyran-2-one | | |
| Anticapsin | 200.0923 | 2.4112 |
| 4-Hydroxy-3-(3- | 201.0915 | 1.7105 |
| methyl-3-buten-1- | | |
| ynyl)benzoic acid: | | |
| 1′-Aldehyde, Me | | |
| ether | | |
| 3-(1H-Indol-3-yl)-2- | 202.0868 | 1.1834 |
| propenoic acid; (E)- | | |
| form: Me ester | | |
| 3-Hydroxy-5- | 203.0708 | 0.9981 |
| methyl-1- | | |
| naphthalenecarboxylic | | |
| acid | | |
| Glycylglycylglycine: | 204.0984 | 3.3994 |
| Me ester | | |
| 4-methyl-7- | 205.0725 | 23.7605 |
| ethoxycoumarin | | |
| Evernitrose | 206.1028 | 5.5967 |
| 6-Deoxyglucose; β- | 207.1232 | 4.2583 |
| L-Pyranose-form: 3- | | |
| Me, Et glycoside | | |
| (N- | 208.1086 | 5.89 |
| Phenylacetyl)glycine: | | |
| Hydrazide | | |
| 6,10-Dimethyl-5,9- | 209.1541 | 13.1682 |
| undecadiene-2,8- | | |
| dione | | |
| Linderazulene | 211.1123 | 2.1584 |
| 4-Methylbenzoic | 212.1075 | 3.9971 |
| acid: Anilide | | |
| Viomycidine: Ac | 213.0987 | 1.9492 |
| 9H-Pyrido[3,4-b]indol- | 214.1106 | 2.0882 |
| 6-ol: Me | | |
| ether, N2-Me | | |
| Cyclo(aspartylleucyl) | 215.1032 | 2.2987 |
| 3-Hydroxy-5- | 216.1024 | 3.5608 |
| methyl-1- | | |
| naphthalenecarboxylic | | |
| acid: Me ether, | | |
| amide | | |
| 3-Hydroxy-5- | 217.0864 | 2.2753 |
| methyl-1- | | |
| naphthalenecarboxylic | | |
| acid: Me ether | | |
| Glycylglycylglycine: | 218.1141 | 2.9292 |
| Et ester | | |
| 5,7-Dimethoxy-2-(1- | 219.1021 | 2.4573 |
| methylethenyl)benzofuran | | |
| Vitamin B5 | 220.1185 | 3.1573 |
| 1H-Indole-3- | 221.1078 | 4.382 |
| carboxaldehyde: | | |
| Anil | | |
| Anhalonine; (±)- | 222.113 | 6.8038 |
| form | | |
| Cystathionine | 223.0752 | 0.9831 |
| 1,2,3,4-Tetrahydro- | 224.0923 | 1.951 |
| 6,7-dihydroxy-1- | | |
| methyl-3- | | |
| isoquinolinecarboxylic | | |
| acid | | |
| Choline: O-(4- | 225.1365 | 3.4441 |
| Hydroxybenzoyl) | | |
| 2-Amino-4-hydroxy- | 226.1079 | 2.7388 |
| 4-(4- | | |
| hydroxyphenyl)-3- | | |
| methylbutanoic acid | | |
| Carnosine | 227.1144 | 3.3847 |
| 2-Amino-4-hexynoic | 228.1236 | 2.6078 |
| acid; (R)-form: N- | | |
| tert- | | |
| Butyloxycarbonyl | | |
| Xanthocidin | 229.1076 | 2.6494 |
| Kinetin; 7H-form: | 230.1042 | 3.2376 |
| 7-Me | | |
| 3-Hydroxy-5- | 231.1021 | 1.6417 |
| methyl-1- | | |
| naphthalenecarboxylic | | |
| acid: Et ester | | |
| Coryneine: Chloride | 232.1104 | 2.6988 |
| Sorbicillin | 233.1177 | 1.9648 |
| 2-Amino-4-hexenoic | 234.113 | 3.1196 |
| acid; (±)-(E)-form: | | |
| N-Benzoyl | | |
| 5-O-Methyl-myo- | 235.1181 | 3.455 |
| inositol: 1,2-O- | | |
| Isopropylidene | | |
| Anhalonine; (S)- | 236.1286 | 3.9295 |
| form: N-Me | | |
| 2-Amino-3- | 238.1079 | 3.1075 |
| hydroxypentanoic | | |
| acid; (2RS,3RS)- | | |
| form: N-Benzoyl | | |
| isopropyl-B-D- | 239.1032 | 0.9677 |
| thiogalactopyranoside | | |
| 9H-Carbazole-3- | 240.1024 | 1.6589 |
| carboxylicacid: Et | | |
| ester | | |
| 3,5- | 241.1228 | 4.0087 |
| Dihydroxystilbene; | | |
| (E)-form: Di-Me | | |
| ether | | |
| Flindersine: N-Me | 242.1181 | 2.8507 |
| phosphatidylcholine | 243.1232 | 2.7201 |
| Biotin,, Amide | 244.1119 | 8.621 |
| Osthole | 245.0926 | 6.9901 |
| Riedelianine | 246.113 | 3.3601 |
| 4,5-Dihydroxy-2,6- | 247.1181 | 3.3368 |
| octadienoicacid: 2,3- | | |
| Dihydroxypropyl | | |
| ester | | |
| 3-(3,4- | 248.1286 | 3.4006 |
| Methylenedioxyphenyl)- | | |
| 2-propenoic acid; | | |
| (E)-form: 2- | | |
| Methylpropylamide | | |
| 4-shogaol | 249.1338 | 1.5636 |
| 2-Acetamido-2- | 250.129 | 2.7988 |
| deoxyglucose; D- | | |
| form: 3,4-Di-Me | | |
| N-2-Phenylethylcamamide | 252.1388 | 4.4605 |
| 5-Deoxy-3-C- | 255.1232 | 4.0396 |
| hydroxymethyllyxose; | | |
| a-L-Furanose- | | |
| form: Benzyl | | |
| glycoside | | |
| Kainic acid,, JAN: | 256.1185 | 2.6832 |
| N—Ac | | |
| Cordiachrome G | 257.1177 | 2.8696 |
| Cyclo(alanyltryptophyl); | 258.1242 | 2.4553 |
| (3?,6?)-form | | |
| arabino-Hexos-2- | 259.1181 | 3.0235 |
| ulose; β-D-2,6- | | |
| Pyranose-form: | | |
| 2,3:4,5-Di-O- | | |
| isopropylidene | | |
| Peganidine: Oxime | 260.1399 | 2.3945 |
| myo-Inositol: | 261.1338 | 2.8952 |
| 1,2:4,5-Di-O- | | |
| isopropylidene | | |
| 8-Methyl-8- | 262.1443 | 2.575 |
| azabicyclo[3.2.1]octane- | | |
| 3,6-diol; (3R, | | |
| 6R)-form: 3- | | |
| Benzoyl | | |
| Furodysinin; (−)- | 263.1469 | 0.7281 |
| form: 14- | | |
| (Methylthio) | | |
| 2-Acetamido-2- | 264.1447 | 2.627 |
| deoxyglucose; D- | | |
| form: 3,4,6-Tri-Me | | |
| 3-Hydroxy-1-oxo- | 265.144 | 1.6699 |
| 7(11)-eudesmen- | | |
| 12,6-olide; | | |
| (3β,4a,6β)-form | | |
| Anisomycin | 266.1392 | 2.9833 |
| 2′- | 268.1046 | 11.7278 |
| Deoxyribofuranosyl | | |
| guanine | | |
| 3,4,5- | 269.1177 | 3.4104 |
| Phenanthrenetriol: | | |
| Tri-Me ether | | |
| 1,2,3,4-Tetrahydro- | 270.1341 | 1.0772 |
| 5-hydroxy-1- | | |
| hydroxymethyl-6,7- | | |
| dimethoxy-2- | | |
| methylisoquinoline; | | |
| (?)-form: N-Oxide | | |
| 5,8-Dihydroxy-2,6- | 271.1545 | 3.3025 |
| dodecadienoic acid; | | |
| (2Z,5?,6Z,8?)- | | |
| form: 8-Ac | | |
| Cynometrine: N1- | 272.1399 | 4.0352 |
| De-Me | | |
| 9H-Pyrido[3,4-b]indol- | 273.1239 | 2.2258 |
| 7-ol: 1,2,3,4- | | |
| Tetrahydro, Me | | |
| ether, N9-formyl, | | |
| Ac | | |
| Galanthamine‡; (−)- | 274.1443 | 2.2013 |
| form: O-De-Me | | |
| 13,14-Dihydroxy- | 275.1647 | 3.8764 |
| 8,11,13- | | |
| podocarpatrien-7- | | |
| one | | |
| 8-Methyl-8- | 276.1599 | 2.762 |
| azabicyclo[3.2.1]octane- | | |
| 3,6-diol; (3R, | | |
| 6R)-form: 3-O- | | |
| Phenylacetyl | | |
| 9,11,13,15- | 277.21671 | 13.5288 |
| Octadecatetraenoic | | |
| acid; (9E,11E,13E, | | |
| 15E)-form | | |
| C-Alkaloid J | 278.1783 | 3.5572 |
| 7-Hydroxy-14,15- | 279.2324 | 0.974 |
| dinor-8(17)-labden- | | |
| 13-one | | |
| Clausenapin | 280.1701 | 2.8152 |
| Bharatamine; (±)- | 282.1494 | 2.0434 |
| form | | |
| Decarbomethoxytetrahydrosecodine | 285.233 | 6.5609 |
| piperine/cocluarine/laurifoline | 286.1443 | 2.132 |
| Villostyrene; (Z)- | 287.1283 | 2.4398 |
| form: 2′-O-De-Me | | |
| Nauclechine; | 288.15 | 2.4507 |
| Natural-form: | | |
| De(methoxycarbonyl), | | |
| 19-deoxy, 18,19- | | |
| didehydro | | |
| chiro-Inositol; D- | 289.1651 | 2.7001 |
| form: 1,4-Di-Me, | | |
| 2,3:5,6-di-O- | | |
| isopropylidene | | |
| 4-Amino-4,6- | 290.1603 | 2.2723 |
| dideoxy-3-C- | | |
| methylmannose; β- | | |
| D-Pyranose-form: | | |
| Me glycoside, N- | | |
| Me, N,2-di-Ac | | |
| 8-Hydroxy-17- | 291.196 | 4.1135 |
| octadecene-9,11- | | |
| diynoic acid | | |
| 1,2,3,5- | 292.1661 | 2.1896 |
| Benzenetetrol: | | |
| Tris(ethylamide) | | |
| Ecklonialactone A: | 293.2116 | 3.2256 |
| 6,7-Dihydro | | |
| 2-Amino-2- | 294.1916 | 2.0099 |
| deoxyglucose; D- | | |
| form: Di-Et acetal, | | |
| 5,6-O- | | |
| isopropylidene | | |
| 16-Hydroxy- | 295.2273 | 2.5341 |
| 9,12,14- | | |
| octadecatrienoic acid | | |
| N,N- | 296.1359 | 0.7118 |
| Dimethyladenosine | | |
| 8-Methyl-8- | 296.1862 | 0.3645 |
| azabicyclo[3.2.1]octane- | | |
| 3,6-diol; (3R, | | |
| 6R)-form: 3- | | |
| Tigloyl, 6-propanoyl | | |
| 16-Hydroxy-6- | 299.2222 | 5.4836 |
| hexadecenoicacid: | | |
| Formyl | | |
| Saxitoxin | 300.142 | 1.0573 |
| Febrifugine‡; (+)- | 301.1664 | 2.1951 |
| form: Oxime | | |
| 3-Amino-3- | 302.1603 | 2.4105 |
| deoxyglucose; a-D- | | |
| Furanose-form: | | |
| 1,2:5,6-Di-O- | | |
| Isopropylidene, N—Ac | | |
| Trichostatin A | 303.1708 | 2.5476 |
| Cocaine,,; (−)-form | 304.1549 | 1.794 |
| 2,4,6-Trihydroxy-3- | 305.1753 | 1.9572 |
| prenylacetophenone; | | |
| 4-O-(3-Methyl-2- | | |
| butenyl) | | |
| 8-Methyl-8- | 306.1705 | 1.7648 |
| azabicyclo[3.2.1]octane- | | |
| 3,6-diol; (3RS, | | |
| 6RS)-form: 3-O- | | |
| (4- | | |
| Methoxyphenylacetyl) | | |
| Spartocytisine: N,O- | 308.1498 | 1.4375 |
| Di-Ac | | |
| [6]-Gingerol; (S)- | 309.2066 | 2.9901 |
| form: 4′-Me ether | | |
| 3-Amino-3- | 310.1654 | 2.407 |
| deoxyxylose; a-D- | | |
| Furanose-form: Me | | |
| glycoside, 5-benzyl, | | |
| N-Me, N—Ac | | |
| Conkurchine | 313.2643 | 12.8033 |
| Salutaridine; (+)- | 314.1392 | 1.0393 |
| form: N-De-Me | | |
| Prosopine‡: 11′- | 314.2695 | 2.4944 |
| Ketone | | |
| Flourensianol: | 315.1596 | 1.9245 |
| Tigloyl | | |
| Ipanguline D3: 3″- | 316.176 | 2.635 |
| Ac | | |
| Franklinol | 317.1753 | 2.0994 |
| 8-Methyl-8- | 318.1705 | 1.6343 |
| azabicyclo[3.2.1]octane- | | |
| 3,6-diol; (3R, | | |
| 6R)-form: 3-O- | | |
| Phenylacetyl, 6-Ac | | |
| 8- | 319.1909 | 1.5626 |
| dehydrogingerdione | | |
| Nitraricine | 320.1763 | 1.8601 |
| Cycloprodigiosin | 322.1919 | 1.5265 |
| 12,14-Labdadiene- | 323.2586 | 0.949 |
| 1,8,18-triol | | |
| 2-Dodecyl-4(1H)- | 328.264 | 2.4596 |
| quinolinone: N-Me | | |
| 2-Dodecyl-3- | 329.2692 | 2.4692 |
| methylbutanedioicacid; | | |
| (2R,3S)-form: | | |
| Di-Me ester | | |
| Salutaridine; (+)- | 330.1705 | 1.1885 |
| form: 5,6?-Dihydro | | |
| sinomenin/deoxyharringtonine | 330.2644 | 0.7867 |
| picrocrocin/carnosol | 331.1909 | 1.885 |
| Alkaloid K5 | 332.1862 | 1.8215 |
| 4-(5-Phenyl-2,4- | 333.1854 | 1.5829 |
| pentadien-1- | | |
| yl)tetracyclo[5.4.0.02, | | |
| 5.03,9]undec-10- | | |
| ene-8-carboxylic | | |
| acid | | |
| 4-[2- | 334.2018 | 1.6921 |
| (Acetylmethylamino)ethyl]- | | |
| 4-(4- | | |
| hydroxy-3- | | |
| methoxyphenyl)-2,4- | | |
| cyclohexadien-1- | | |
| one: 2,3,5,6- | | |
| Tetrahydro, Me ether | | |
| Lysinomycin: De-N- | 335.2294 | 1.8367 |
| lysyl, 4′,5′-dihydro | | |
| Aquaticine | 336.1811 | 0.7261 |
| 5,9- | 337.3106 | 1.5479 |
| Docosadienoicacid | | |
| 5-(8- | 339.2899 | 2.2887 |
| Heptadecenyl)dihydro- | | |
| 3-hydroxy-2(3H)- | | |
| furanone | | |
| 20-Aminopregna- | 342.2797 | 1.881 |
| 1,4-dien-3-one; (20S)- | | |
| form: N,N-Di-Me | | |
| Secodine: | 343.2385 | 2.5378 |
| 15,16,17,20- | | |
| Tetrahydro | | |
| Pregn-4-ene-3,20- | 345.2542 | 1.1326 |
| dione: Dioxime | | |
| Mahanine; (?)-form: | 346.1807 | 0.9097 |
| Deoxy, 1′-oxo | | |
| Broussonetinine A | 346.2593 | 0.8685 |
| Bacchalejin 1 | 348.2175 | 1.974 |
| Pregna-5,20-diene- | 349.2379 | 2.1386 |
| 3,7,11,19-tetrol | | |
| Ankorine; (−)-form: | 350.1967 | 1.3556 |
| Carboxylic acid | | |
| 20-Methylpregnane- | 351.2899 | 1.692 |
| 2,3,6-triol | | |
| 2-(Aminomethyl)-2- | 354.2644 | 2.4212 |
| propenoic acid: N- | | |
| (2- | | |
| Oxopentadecanoyl), | | |
| Me ester | | |
| Glycerol 1- | 355.2848 | 10.5819 |
| alkanoates; Glycerol | | |
| 1-(9Z,12Z- | | |
| octadecadienoate) | | |
| CyclobuxophyllineO | 356.2953 | 2.6124 |
| 13-Docosenoic acid; | 357.2924 | 6.6591 |
| (Z)-form: Chloride | | |
| Buxandonine L | 358.311 | 1.1337 |
| Angustoline; (?)- | 360.1712 | 0.5245 |
| form: Et ether | | |
| Atidine | 360.2538 | 0.6974 |
| 3-Hydroxy-8- | 361.2379 | 1.3224 |
| kempen-6-one; | | |
| (3β,7β)-form: 2β- | | |
| Acetoxy | | |
| 13(16),14- | 365.2692 | 0.9937 |
| Labdadiene-3,6,8- | | |
| triol; (ent-3β,6a,8a)- | | |
| form: 6-Ac | | |
| ConioidineA | 366.3008 | 1.1779 |
| Tetrahydro-2- | 367.3212 | 2.3801 |
| furanmethanol; (±)- | | |
| form: 9Z- | | |
| Octadecenoyl | | |
| Corynoline; (+)- | 368.1498 | 0.5496 |
| form: 14-Epimer | | |
| CyclosuffrobuxineK | 368.2953 | 1.7116 |
| 26,27-Dinorergosta- | 369.3157 | 5.3195 |
| 4,22-dien-3-one | | |
| Cyclobuxoviramine | 370.311 | 3.4542 |
| 3-Hydroxy-19- | 371.2222 | 9.2131 |
| oxocarda-14,20(22)- | | |
| dienolide | | |
| 4-(2-Amino-3- | 372.1294 | 0.5911 |
| hydroxyphenyl)-4- | | |
| oxobutanoic acid: O- | | |
| β-D- | | |
| Glucopyranoside | | |
| Hetisan-2,11,13-triol; | 372.2175 | 0.3884 |
| (2a,11a,13R)- | | |
| form: 13-Ac | | |
| Daphniphyllum | 372.2538 | 0.3204 |
| Alkaloid A1 | | |
| Sakyomicin B; (+)- | 373.0923 | 0.1611 |
| form | | |
| 3-(2,3,5- | 373.1498 | 0.1661 |
| Trihydroxyphenyl)- | | |
| 2-propen-1-ol; (E)- | | |
| form; 2′,5′-Di-Me | | |
| ether, 3′-O-β-D- | | |
| glucopyranoside | | |
| Smalogenin | 373.2379 | 1.2078 |
| Myxalamides; | 374.2695 | 2.3863 |
| Myxalamide D | | |
| Wedeliasecokaurenolide: | 375.2171 | 0.931 |
| 3β-Acetoxy | | |
| Cadiamine: O8-(2- | 376.2236 | 1.515 |
| Pyrrolecarbonyl) | | |
| 1,5,6- | 377.2328 | 1.1637 |
| Vouacapanetriol; | | |
| (1a,5a,6β)-form: 6- | | |
| Ac | | |
| Cryptopleurine; (R)- | 378.2069 | 0.9019 |
| form | | |
| Seconorrlandin C | 379.2484 | 1.3981 |
| Psylloborine A | 381.3269 | 3.9419 |
| 2-(Aminomethyl)-2- | 382.3321 | 2.0091 |
| propenoic acid: N- | | |
| Octadecanoyl, Me | | |
| ester | | |
| 1,2-Dihydroxy-5- | 383.3161 | 30.8393 |
| heneicosen-4-one; | | |
| (2?,5E)-form: 1-Ac | | |
| CyclobuxophyllineO: | 384.3266 | 7.6396 |
| N,N-Di-Me | | |
| Cholest-7-en-3-one | 385.347 | 3.0758 |
| Protoemetine; (−)- | 390.228 | 1.6697 |
| form: Alcohol, O9- | | |
| de-Me, di-O-Ac | | |
| 3-(11- | 395.3525 | 4.3813 |
| Eicosenyl)dihydro- | | |
| 4-hydroxy-5-methyl- | | |
| 2(3H)-furanone | | |
| 2-(Aminomethyl)-2- | 396.3477 | 0.2859 |
| propenoic acid: N- | | |
| Nonadecanoyl, Me | | |
| ester | | |
| 3(20)-Phytene-1,2- | 397.3318 | 10.5496 |
| diol; (2?,7?,11?)(1)- | | |
| form: Di-Ac | | |
| Cholesta-4,6-dien-3- | 398.3423 | 4.7657 |
| one: Z-Oxime | | |
| 3-Hydroxycholest-5- | 401.3419 | 3.0589 |
| en-24-one | | |
| Baleabuxaline I | 407.3273 | 2.1615 |
| Antibiotic U 106305 | 408.3266 | 1.6297 |
| Stigmasta- | 409.347 | 7.3283 |
| 3,5,24(28)-trien-7- | | |
| one | | |
| 1-Azasterol: 3- | 410.3423 | 2.2782 |
| Ketone | | |
| Stigmasta- | 411.3627 | 23.2523 |
| 5,7,24(28)-trien-3- | | |
| ol; (3β,24Z)-form | | |
| 15-Azasterol | 412.3579 | 7.6401 |
| 1,21- | 413.3631 | 1.1378 |
| Heneicosanediol: Di- | | |
| Ac | | |
| Crispatone | 415.2484 | 45.3266 |
| Pregna-5,20-diene- | 417.2641 | 2.8874 |
| 3,7,11-triol; | | |
| (3a,7a,11a)-form: | | |
| 3,7-Di-Ac | | |
| 3-Amino-12- | 418.2957 | 2.1911 |
| hydroxypregnan-20- | | |
| one; (3β,5a,12β)- | | |
| form: N,O-Di-Ac | | |
| Axinellamine B‡ | 419.3426 | 0.9147 |
| Aflavinine: 13-Oxo, | 420.2902 | 1.7187 |
| 10,11-dihydro, | | |
| 24,25-didehydro | | |
| 5-Methylene-3- | 421.3106 | 1.9945 |
| (6,9,12,15,18,21- | | |
| tetracosahexaenyl)- | | |
| 2(5H)-furanone | | |
| Ancistrocladine: 1- | 422.2331 | 0.6991 |
| And/or 3-epimer, Me | | |
| ether | | |
| 6,12,20(30)- | 423.3627 | 4.7064 |
| Ursatrien-3-ol | | |
| Ircinamine B | 424.3613 | 2.3661 |
| a-Tocotrienol | 425.3419 | 8.6566 |
| Moenjodaramine | 427.3688 | 0.3501 |
| Solaspiralidine | 428.3164 | 0.8918 |
| Cholest-5-ene- | 431.3161 | 0.9077 |
| 3,16,22,26-tetrol; | | |
| (3β,25S)-form: | | |
| 16,22-Diketone | | |
| Proxiphomin | 432.2902 | 10.3917 |
| Tryptoquivaline G | 433.1512 | 0.0286 |
| 8,11′;12,12′- | 433.3106 | 2.7311 |
| Bi[1(10),7- | | |
| eremophiladien-9- | | |
| one] | | |
| 3,6-Diiodo-9H- | 433.8902 | 0.0572 |
| carbazole: N-Me | | |
| 4-Methylaconitane- | 434.2906 | 1.0873 |
| 1,8,14,16-tetrol; | | |
| (1a,14a,16β)-form: | | |
| O1,O16-Di-Me, N- | | |
| Et, O14-Ac | | |
| 4,15,26- | 435.3263 | 1.9869 |
| Triacontatriene- | | |
| 1,12,18,29-tetrayne- | | |
| 3,28-diol; (3?,4E, | | |
| 15Z,26E,28?)- | | |
| form: 12,13- | | |
| Dihydro(Z-) | | |
| 4-Methylaconitane- | 436.3063 | 1.4246 |
| 1,8,14,16,18-pentol; | | |
| (1a,14a,16β)-form: | | |
| O1,O8,O16,O18- | | |
| Tetra-Me, N-Et | | |
| Antibiotic PB 5266A | 438.0931 | 0.0523 |
| Amphiasterin B3 | 439.3787 | 20.091 |
| Fibrostatins; | 440.1015 | 0.0344 |
| Fibrostatin F | | |
| 4,4-Dimethyl-15- | 440.3892 | 5.5662 |
| azasterol | | |
| Buxamine I: N3,N3, | 441.3845 | 3.73 |
| N20-Tri-Me, N20- | | |
| Ac | | |
| Alkaloid LE1 | 442.3797 | 2.4348 |
| Metachromin G | 448.2851 | 2.1567 |
| Ergost-24(28)-ene- | 449.3631 | 1.5263 |
| 1,3,5,6-tetrol; | | |
| (1a,3β,5a,6β)-form | | |
| Ergostane-1,3,5,6- | 451.3787 | 1.9523 |
| tetrol | | |
| 3,5- | 453.3944 | 4.4256 |
| Dioxooctacosanoicacid | | |
| Deacylcylindrol: 4′- | 459.2746 | 29.1391 |
| Butanoyl | | |
| Piericidin; Piericidin | 460.3063 | 6.6987 |
| A2: 10′-Me ether, | | |
| 11′,12′-epoxide | | |
| Lysinomycin | 461.3087 | 2.8422 |
| Isolankacidinol | 462.2492 | 0.7048 |
| 25-Methylergostane- | 465.3944 | 1.8686 |
| 2,3,6,15-tetrol | | |
| 4-Dotriacontanone | 465.5035 | 0.356 |
| 24- | 467.4253 | 12.5903 |
| Isopropylcycloart- | | |
| 25-en-3-one | | |
| Spirosolane-3,23- | 474.3583 | 4.0266 |
| diol; (3β,5a,22R, | | |
| 23S,25R)-form: | | |
| 23-Ac | | |
| 8- | 479.4828 | 4.3734 |
| Dotriacontenoicacid | | |
| CoriacenineA: | 481.4481 | 2.7933 |
| Didehydro | | |
| Rhodopeptin; | 482.3706 | 1.6518 |
| Rhodopeptin C1 | | |
| 12- | 493.4984 | 5.5375 |
| Oxotritriacontanal | | |
| 28-Hydroxy- | 495.4413 | 10.8746 |
| 7,16,18- | | |
| hentriacontanetrione; | | |
| (S)-form | | |
| Nemorosone | 503.3161 | 6.1665 |
| Majusculamide A | 504.3437 | 5.9789 |
| Epothilone B | 508.2733 | 0.6451 |
| 31-Hydroxy-7- | 509.4933 | 2.2627 |
| hentriacontanone: | | |
| Ac | | |
| 7,18,19-Trihydroxy- | 510.2855 | 0.3108 |
| 16,18-dimethyl-10- | | |
| phenyl[11]cytochalasa- | | |
| 6(12),13-diene-: | | |
| 19-Ac | | |
| Fenestin B | 520.3499 | 4.3996 |
| Quaesitol | 521.3267 | 0.7642 |
| 3-Methyl-3-buten-1- | 521.5297 | 11.2842 |
| ol: Triacontanoyl | | |
| 2,3,14,20,22,25- | 523.3271 | 0.262 |
| Hexahydroxycholest- | | |
| 7-en-6-one; | | |
| (2β,3β,5β,20R,22R)- | | |
| form: 2-Ac | | |
| Tetrahydro-2-(1- | 523.4726 | 14.2872 |
| hydroxy-9-nonenyl)- | | |
| 5-pentyl-3-furanol: | | |
| 1′-O-Tetradecanoyl | | |
| Budmunchiamine | 525.5107 | 0.7379 |
| L5‡: 6′-Hydroxy, N1, | | |
| N9,N13-tri-Me | | |
| 4-Methylaconitane- | 526.3168 | 1.649 |
| 1,8,14,16,18-pentol; | | |
| (1a,14a,16β)-form: | | |
| O1,O16,O18-Tri- | | |
| Me, N-Et, 14- | | |
| benzoyl | | |
| 9-Octadecenyl 9- | 533.5297 | 13.078 |
| octadecenoate | | |
| Roehybridine | 534.2968 | 2.6939 |
| Borrelidin: Ac, Me | 546.3431 | 2.771 |
| ester | | |
| 1,2,7,7′,8,8′,11,11′,12, | 547.5243 | 5.3902 |
| 12′- | | |
| Decahydrolycopene | | |
| Artemoin A | 551.5039 | 11.4632 |
| Antibiotic SH 50 | 554.3666 | 2.1227 |
| O- | 564.3689 | 5.1536 |
| Deethyltalaroconvolutin | | |
| C: 4′,5′- | | |
| Isopropylidene | | |
| Manzamine A; (+)- | 565.3542 | 0.7125 |
| form: 2-N-Oxide | | |
| 12-Hydroxy-9- | 565.5196 | 2.8153 |
| octadecenoic acid; | | |
| (R,Z)-form: 12- | | |
| Hydroxy-9- | | |
| octadecenyl ester | | |
| 2-Amino-9-methyl- | 566.5148 | 1.132 |
| 4,8-octadecadiene- | | |
| 1,3-diol; (2S,3R,4E, | | |
| 8E)-form: N-(2R- | | |
| Hydroxyhexadecanoyl) | | |
| Penitrem C: | 568.3427 | 1.9267 |
| Dechloro | | |
| 3,22,24-Trihydroxy- | 569.3842 | 1.8922 |
| 12-oleanen-28-oic | | |
| acid; (3β,22β)-form: | | |
| 3-Ketone, 22- | | |
| angeloyl | | |
| Brasilicardin A: 4′- | 570.3642 | 2.3481 |
| Deacyl, 3′- | | |
| deglycosyl | | |
| Veratramine: 20- | 572.3587 | 0.6945 |
| Epimer, 23-O-β-D- | | |
| glucopyranoside | | |
| 3-Methyl-3-buten-1- | 577.5923 | 1.0139 |
| ol: | | |
| Tetratriacontanoyl | | |
| DestruxinA | 578.3554 | 2.4956 |
| Atrataphloroglucinol A | 579.2958 | 0.7716 |
| Tonkinelin | 579.5352 | 3.5729 |
| Penitrem C: | 584.3376 | 0.6812 |
| Dechloro, 23a,24a- | | |
| epoxide | | |
| Rhinolidine: 3-O-β- | 592.4213 | 4.3349 |
| D-Glucopyranoside | | |
| Santiaguine | 593.3855 | 1.1838 |
| Uvariamicin IV | 593.5145 | 1.1315 |
| Apicidin: N- | 594.3655 | 5.5917 |
| Demethoxy | | |
| Stercobilin | 595.3495 | 2.6401 |
| DestruxinE; | 608.3659 | 0.9896 |
| DestruxinE1 | | |
| 3,4,12,14,16,17,20- | 622.3591 | 1.8543 |
| Cevaneheptol; | | |
| (3β,4a,5a,16β)-form: | | |
| 3,4,16-Tri-Ac | | |
| Destruxin D; | 638.3765 | 2.8275 |
| Destruxin D1 | | |
| 2-Amino-4- | 684.6142 | 1.5028 |
| nonacosene-1,3,6- | | |
| triol; (2R,3R,4E, | | |
| 6R)-form: N-(3R, | | |
| 4S- | | |
| Dihydroxydodecanoyl) | | |

TABLE 10
DART TOF-MS characterization of Nettle Extract 3.

| | | Rel. |
|---|---|---|
| | Calc. | Abund. |
| Compound Name | Mass | (%) |
| 2-Methyl-2-butenoic | 100.0762 | 6.5887 |
| 3-Ketone, 22- | | |
| angeloyl | | |
| 3-Aminodihydro- | 102.0555 | 3.8973 |
| 2(3H)-furanone | | |
| 4-Hydroxy-2- | 103.0395 | 14.1567 |
| oxobutanal | | |
| N,N- | 104.0711 | 23.6558 |
| Dimethylglycine | | |
| 2,3- | 105.0664 | 1.0443 |
| Diaminopropanoicacid | | |
| Diethanolamine | 106.0868 | 0.8812 |
| Butanoic acid: | 107.0263 | 0.2779 |
| Chloride | | |
| 1H-Imidazole-4(5)- | 108.0561 | 0.6541 |
| acetic acid: Nitrile | | |
| 1-Chloro-2-propanol; | 109.042 | 7.0953 |
| (±)-form: Me ether | | |
| 1H-Pyrrole-3- | 110.0718 | 12.836 |
| carboximidamide | | |
| Resorcinol | 111.0558 | 11.1465 |
| 2,3-Dihydro-1H- | 112.0762 | 11.1938 |
| pyrrole: N—Ac | | |
| 6-azacytosine | 113.0602 | 10.2505 |
| Creatinine | 114.0667 | 6.9471 |
| 2-Amino-3- | 115.0507 | 6.7441 |
| cyanopropanoic | | |
| acid; (S)-form | | |
| Proline | 116.0711 | 18.4725 |
| Levulinic acid | 117.0612 | 18.5221 |
| 2- | 118.0868 | 17.8505 |
| Methylaminoacetic | | |
| acid: Et ester | | |
| Succinic acid | 119.0344 | 5.6792 |
| Threonine | 120.066 | 3.4248 |
| Nitrosofungin | 121.0613 | 8.0398 |
| 1H-Imidazole-4(5)- | 122.0718 | 2.6259 |
| acetic acid; 1H- | | |
| form: N-Me, nitrile | | |
| Vitamin B3 | 123.0657 | 23.4603 |
| (niacinamide) | | |
| 2- | 124.0762 | 12.2319 |
| Hydroxybenzylamine | | |
| 1H-Pyrrole-2- | 125.0715 | 26.1712 |
| carboxaldehyde: N- | | |
| Me, oxime | | |
| 2-Amino-4- | 126.0667 | 16.4043 |
| hydroxypyrimidine; | | |
| 1H-form: 1-Me | | |
| 5-Hydroxy-3-vinyl- | 127.0395 | 23.3354 |
| 2(5H)-furanone | | |
| 3-Azetidinone: N—Ac, | 129.0664 | 7.8633 |
| oxime | | |
| 2-Amino-4-hexenoic | 130.0868 | 21.0204 |
| acid | | |
| 3-Amino-2- | 131.082 | 1.2862 |
| pyrrolidinecarboxylic | | |
| acid; (2S,3R)- | | |
| form | | |
| N-(2-Methoxy-2- | 131.131 | 0.6635 |
| propenyl)trimethylammonium(1+) | | |
| Leucine | 132.1024 | 18.2491 |
| Asparagine | 133.0613 | 16.9519 |
| 2-Amino-4-hydroxy- | 134.0817 | 5.9095 |
| 3-methylbutanoic | | |
| acid | | |
| 2-Amino-4- | 135.0769 | 3.9953 |
| (aminooxy)butanoic | | |
| acid; (±)-form | | |
| Adenine | 136.061 | 95.3699 |
| 2-Methyl-1,2,3,4- | 137.0814 | 15.5276 |
| butanetetrol | | |
| 5-Ethyl-2- | 138.0919 | 10.7429 |
| methylpyridine: N- | | |
| Oxide | | |
| 3-Hydroxy-2,5- | 139.0871 | 12.9538 |
| dimethylpyrazine; | | |
| OH-form: Me ether | | |
| Choline: Chloride | 140.0842 | 18.386 |
| Norzooanemonin | 141.0664 | 5.4871 |
| 2-Aminoethanol: N, | 142.1232 | 21.0533 |
| N-Di-2-propenyl | | |
| 2-Hydroxy-2- | 143.0344 | 0.3138 |
| hydroxymethyl-4- | | |
| cyclopentene-1,3- | | |
| dione | | |
| 1-Undecene-5,7,9- | 143.0861 | 12.9739 |
| triyne | | |
| 4-Phenyl-3-butenoic | 144.0813 | 16.9409 |
| acid; (E)-form: | | |
| Nitrile | | |
| 2,3-Dihydro-3,6- | 145.0501 | 56.3027 |
| dihydroxy-2-methyl- | | |
| 4H-pyran-4-one | | |
| 2-Amino-3- | 146.0817 | 24.9366 |
| hydroxymethyl-3- | | |
| pentenoic acid | | |
| 2,3- | 147.0769 | 10.5397 |
| Diaminopropanoic | | |
| acid; (S)-form: N3- | | |
| Ac | | |
| 2-Amino-5- | 148.0973 | 4.795 |
| hydroxyhexanoic | | |
| acid | | |
| O-Carbamoylserine | 149.0562 | 23.3632 |
| 4-Methylbenzoic | 150.0919 | 12.6544 |
| acid: Methylamide | | |
| 4-Methylbenzoic | 151.0759 | 8.5414 |
| acid: Me ester | | |
| 2-Aminobenzoic | 152.0824 | 13.5084 |
| acid: Hydrazide | | |
| 1,4-Benzenediol: | 153.0915 | 16.8559 |
| Me-Et ether | | |
| Scopine: 3-Ketone | 154.0868 | 21.3642 |
| 1H-Imidazole-1- | 155.082 | 11.722 |
| acetic acid: Et ester | | |
| Scopine | 156.1024 | 18.1748 |
| Tetrahydro-2- | 157.0864 | 2.069 |
| furanmethanol; (±)- | | |
| form: 2-Propenoyl | | |
| Diptocarpilidine: S- | 158.1003 | 29.8134 |
| Deoxo | | |
| 2-Amino-4- | 159.0769 | 3.9811 |
| methylenepentanedioic | | |
| acid; (S)-form: | | |
| ?-Amide | | |
| 4-Hydroxy-1,1- | 160.0973 | 15.7335 |
| dimethylpyrrolidinium- | | |
| 2-carboxylate; (2S, | | |
| 4R)-form | | |
| 2,6-Diamino-4- | 161.0926 | 3.7707 |
| oxohexanoicacid | | |
| 3-Aminotetrahydro- | 162.0766 | 9.9068 |
| 5-(hydroxymethyl)- | | |
| 3-furancarboxylic | | |
| acid | | |
| Levoglucosan/Glyogen/ | 163.0615 | 51.0657 |
| Laminarin | | |
| 2-Thioxo-4- | 163.984 | 0.0623 |
| thiazolidinecarboxylic | | |
| acid; (S)-form | | |
| 2-Amino-4,5- | 164.0923 | 13.0644 |
| dihydroxy-4- | | |
| methylpentanoic | | |
| acid | | |
| 1,2,4,5- | 165.1127 | 14.9895 |
| Pentanetetrol; (2RS, | | |
| 4RS)-form: 1,5-Di- | | |
| Me ether | | |
| 2-Amino-2- | 166.0868 | 30.3233 |
| phenylpropanoic | | |
| acid | | |
| 6-Deoxyaltritol; D- | 167.0919 | 17.9438 |
| form | | |
| Synephrine | 168.1024 | 16.2865 |
| Tetramethylpyrazine: | 169.0977 | 14.8874 |
| 1,4-Dioxide | | |
| 2-Amino-3-(3- | 170.0817 | 20.4081 |
| furanyl)propanoic | | |
| acid; (S)-form: N- | | |
| Me | | |
| Viomycidine | 171.0882 | 4.3224 |
| 2-Propylquinoline | 172.1126 | 19.0778 |
| Caprazamycin; | 173.1038 | 7.2348 |
| CaprazamycinG | | |
| 8-Methyl-8- | 174.113 | 13.9203 |
| azabicyclo[3.2.1]octane- | | |
| 3,6,7-triol | | |
| Shikimic acid | 175.0606 | 3.9357 |
| Muscarine‡ | 175.1572 | 10.9628 |
| 3- | 176.0923 | 14.1133 |
| Aminoheptanedioic | | |
| acid; (±)-form | | |
| N4-(2- | 177.0875 | 7.3678 |
| Hydroxyethyl)asparagine; | | |
| (S)-form | | |
| N-[2-(4- | 178.0868 | 16.4242 |
| Hydroxyphenyl)ethenyl]formamide: | | |
| Me | | |
| ether | | |
| N-Benzoylglycine: | 179.082 | 16.0989 |
| Amide | | |
| 6-Deoxymannonic | 180.0872 | 44.0945 |
| acid; L-form: | | |
| Amide | | |
| 2-Amino-3-(4- | 181.0977 | 28.5038 |
| aminophenyl)propanoic | | |
| acid | | |
| (2- | 182.0946 | 25.8835 |
| Aminoethyl)phosphonic | | |
| acid: Di-Et ester | | |
| Dihydro-5-(5- | 183.1021 | 17.9999 |
| hydroxy-1,3- | | |
| hexadienyl)-2(3H)- | | |
| furanone | | |
| 6-(3-Methyl-1,3- | 184.1126 | 18.2093 |
| butadienyl)-1H- | | |
| indole; (Z)-form | | |
| 2-Naphthol: 2- | 185.0966 | 11.6116 |
| Propenyl ether | | |
| 5-Methyldodecane | 185.2269 | 1.0841 |
| 4-(1H-Indol-3-yl)-3- | 186.0919 | 20.3856 |
| buten-2-one | | |
| 4-Hydroxy-3-(3- | 187.0759 | 9.2112 |
| methyl-3-buten-1- | | |
| ynyl)benzoic acid: | | |
| 1′-Aldehyde | | |
| 2-Amino-4- | 188.0923 | 15.8631 |
| propylidenepentanedioic | | |
| acid | | |
| 1H-Indole-3- | 189.1028 | 6.0942 |
| methanamine: 3-N—Ac | | |
| 2-Ethyl-4- | 190.098 | 11.0712 |
| hydroxyquinazoline; | | |
| 3H-form: N-Amino | | |
| 4-Cyclohexylphenol: | 191.1436 | 18.9308 |
| Me ether | | |
| Garosamine; β-L- | 192.1236 | 18.0072 |
| Pyranose-form: Me | | |
| glycoside | | |
| 6-Deoxyglucose; a- | 193.1076 | 24.8605 |
| D-Pyranose-form: 3- | | |
| Me, Me glycoside | | |
| 1,2,3,4-Tetrahydro- | 194.1181 | 17.2918 |
| 7-hydroxy-6- | | |
| methoxy-1- | | |
| methylisoquinoline; | | |
| (S)-form | | |
| Tagatose; a-D- | 195.0868 | 3.3666 |
| Pyranose-form: Me | | |
| glycoside | | |
| DL-methyl-m- | 196.1099 | 25.598 |
| tyrosine | | |
| 2-Acetyl-4,4,6- | 197.1177 | 27.815 |
| trimethyl-1,3- | | |
| cyclohexanedione | | |
| Buddamine | 198.113 | 29.0171 |
| Pedatisectine D | 199.1082 | 13.1948 |
| N-(2- | 200.1035 | 19.5123 |
| Cyanoethyl)glutamine; | | |
| (±)-form | | |
| 4-Hydroxy-3-(3- | 201.0915 | 9.4948 |
| methyl-3-buten-1- | | |
| ynyl)benzoic acid: | | |
| 1′-Aldehyde, Me | | |
| ether | | |
| 2,6- | 202.1079 | 14.4653 |
| Piperidinedicarboxylic | | |
| acid; (2RS,6RS)- | | |
| form: Di-Me ester | | |
| 1,2,3,4-Tetrahydro- | 203.1184 | 6.7914 |
| 1-methyl-β- | | |
| carboline; (?)-form: | | |
| Nb-Oxide | | |
| Carnitine,; (±)-form: | 204.1236 | 12.6812 |
| O—Ac | | |
| Indeno[1,2,3-ij][2,7]naphthyridine | 205.0765 | 22.1322 |
| 4-methyl-7- | 206.1181 | 13.1582 |
| ethoxycoumarin | | |
| Eremopetasinorol: 3- | 207.1385 | 13.3263 |
| Ketone | | |
| (N- | 208.1086 | 19.2251 |
| Phenylacetyl)glycine: | | |
| Hydrazide | | |
| 6,10-Dimethyl-5,9- | 209.1541 | 26.3414 |
| undecadiene-2,8- | | |
| dione | | |
| Muscarine‡; (2S,4R, | 210.1261 | 16.3733 |
| 5S)-form: Chloride | | |
| Antibiotic A 41-89; | 211.1334 | 12.3925 |
| Antibiotic A 41-89I | | |
| 4-Methylbenzoic | 212.1075 | 15.807 |
| acid: Anilide | | |
| Linderazulene: 2,3- | 213.1279 | 7.3943 |
| Dihydro | | |
| 2-Aminobenzyl | 214.1232 | 15.2011 |
| alcohol: N-Benzyl | | |
| Cyclo(aspartylleucyl) | 215.1032 | 8.8031 |
| a-Amino-?- | 216.1236 | 18.7089 |
| oxooxiraneoctanoic | | |
| acid | | |
| 2-Oxohexanedioic | 217.1076 | 6.5608 |
| acid: Di-Et ester | | |
| Glycylglycylglycine: | 218.1141 | 17.7816 |
| Et ester | | |
| 5,7-Dimethoxy-2-(1- | 219.1021 | 15.0242 |
| methylethenyl)benzofuran | | |
| Vitamin B5 | 220.1185 | 13.3746 |
| Penienone | 221.1541 | 13.9132 |
| Anhalonine; (±)- | 222.113 | 13.8136 |
| form | | |
| Cystathionine | 223.0752 | 4.3901 |
| 14-Nor-5- | 223.1698 | 2.7406 |
| protoilludene-7,8- | | |
| diol; (7a,8β)-form | | |
| Cerulenin | 224.1286 | 14.2891 |
| Murexine | 225.1477 | 10.4311 |
| 10-(3-Pyridinyl)- | 226.1232 | 14.2499 |
| 3,5,7,9-decatetraen- | | |
| 2-one; (all-E)-form | | |
| 1-(2,5- | 227.1283 | 6.6621 |
| Dihydroxyphenyl)- | | |
| 1,2-propanediol; (1R*, | | |
| 2R*)-form: 1-Et, | | |
| 5′-Me ether | | |
| 2-Pentyl-4(1H)- | 228.1388 | 8.9417 |
| quinolinone; OH- | | |
| form: 1′,2′- | | |
| Didehydro, Me ether | | |
| Faramol | 229.1228 | 7.3971 |
| Haplofoline | 230.1181 | 14.1165 |
| Ribose; a-D- | 231.1232 | 5.3563 |
| Pyranose-form: | | |
| 1,2:3,4-Di-O- | | |
| isopropylidene | | |
| 1H-Indole-3- | 232.1337 | 13.7294 |
| butanoic acid: Et | | |
| ester | | |
| Cytisine; (−)-form: | 233.129 | 7.9896 |
| N—Ac | | |
| 2-Amino-4-hexenoic | 234.113 | 11.9623 |
| acid; (±)-(E)-form: | | |
| N-Benzoyl | | |
| 8-Hydroxy-13-nor- | 235.1334 | 16.7557 |
| 4(15),7(11)- | | |
| eudesmadien-12,8- | | |
| olide | | |
| Anhalonine; (S)- | 236.1286 | 18.5747 |
| form: N-Me | | |
| Theophylline,, JAN, | 237.1351 | 11.1256 |
| N-(2-Methylpropyl) | | |
| 2,3-Dihydro-2- | 238.1232 | 12.5889 |
| methylindole; (R)- | | |
| form: N-Benzoyl | | |
| isopropyl-B-D- | 239.1032 | 1.7528 |
| thiogalactopyranoside | | |
| Mescaline: N- | 240.1236 | 7.7477 |
| Formyl | | |
| 3,5- | 241.1228 | 6.8563 |
| Dihydroxystilbene; | | |
| (E)-form: Di-Me | | |
| ether | | |
| 2-Hydroxybenzoic | 242.1392 | 10.0822 |
| acid: Choline ester | | |
| phosphatidylcholine | 243.1232 | 4.4636 |
| Sceletenone | 244.1337 | 11.7282 |
| Osthole | 245.0926 | 6.6145 |
| 3-Deoxy-3- | 246.1341 | 12.8987 |
| (methylamino)arabinose; | | |
| β-L-Furanose- | | |
| form: N—Ac, 1,2-O- | | |
| isopropylidene | | |
| N-?- | 247.1294 | 11.1566 |
| Glutamylvaline; L- | | |
| L-form | | |
| 3-(3,4- | 248.1286 | 11.5418 |
| Methylenedioxyphenyl)- | | |
| 2-propenoic acid; | | |
| (E)-form: 2- | | |
| Methylpropylamide | | |
| 4-shogaol | 249.1338 | 9.4141 |
| Anhalonine; (S)- | 250.1443 | 11.837 |
| form: N-Et | | |
| Lachnumfuran A | 251.1283 | 5.9858 |
| 1,2,3,4-Tetrahydro- | 252.1236 | 16.7941 |
| 6,7-dihydroxy-1- | | |
| methyl-3- | | |
| isoquinolinecarboxylic | | |
| acid; (1S,3S)- | | |
| form: Et ester | | |
| 1,2,3-Benzenetriol: | 253.0712 | 1.1024 |
| Tri-Ac | | |
| 1,2,3,4-Tetrahydro- | 253.1678 | 1.577 |
| 6,7,8-trihydroxy-1- | | |
| methylisoquinoline; | | |
| (?)-form: 6,7-Di-Me | | |
| ether, N,N-di-Me | | |
| 1,2,3,4-Tetrahydro- | 254.1392 | 9.8924 |
| 5,6,7-trimethoxy-2- | | |
| methylisoquinoline: | | |
| N-Oxide | | |
| a,a′-Diamino-2- | 255.1345 | 6.789 |
| methylenecycloheptene- | | |
| 1,3-diacetic acid | | |
| 2-Amino-3-phenyl- | 256.1337 | 8.2473 |
| 1-propanol; (S)- | | |
| form: N-Benzoyl | | |
| Parazoanthoxanthin | 257.1514 | 5.3277 |
| A: 3,N6,N6-Tri-Me | | |
| Elaeocarpine | 258.1494 | 6.8569 |
| Cyclo(leucylphenylalanyl); | 259.1446 | 9.0353 |
| (3S,6S)- | | |
| form: 1′,3- | | |
| Didehydro(Z-) | | |
| Peganidine: Oxime | 260.1399 | 11.7146 |
| myo-Inositol: | 261.1338 | 8.2878 |
| 1,2:4,5-Di-O- | | |
| isopropylidene | | |
| 8-Methyl-8- | 262.1443 | 10.0096 |
| azabicyclo[3.2.1]octane- | | |
| 3,6-diol; (3R, | | |
| 6R)-form: 3- | | |
| Benzoyl | | |
| Furodysinin; (−)- | 263.1469 | 3.7533 |
| form: 14- | | |
| (Methylthio) | | |
| 2-Acetamido-2- | 264.1447 | 8.1885 |
| deoxyglucose; D- | | |
| form: 3,4,6-Tri-Me | | |
| Eupomatenoid 6 | 265.1228 | 4.9287 |
| Anisomycin | 266.1392 | 10.5068 |
| 2,3-Dihydroxy-3- | 267.0868 | 0.5291 |
| phenylpropanoicacid; | | |
| (2RS,3SR)-form: | | |
| Di-Ac | | |
| Peyoruvic acid | 268.1185 | 10.0439 |
| 8,8a-Dihydro-8a- | 269.1389 | 3.8001 |
| hydroxy-3,3,6,6,8,8- | | |
| hexamethyl-1,2- | | |
| benzodioxin-5,7(3H, | | |
| 6H | | |
| 1,2,3,4-Tetrahydro- | 270.1341 | 9.6314 |
| 5-hydroxy-1- | | |
| hydroxymethyl-6,7- | | |
| dimethoxy-2- | | |
| methylisoquinoline; | | |
| (?)-form: N-Oxide | | |
| Glucitol; D-form: | 271.1181 | 6.1825 |
| 4,6-Benzylidene | | |
| Cynometrine: N1- | 272.1399 | 8.0191 |
| De-Me | | |
| Verimol F | 273.149 | 4.9913 |
| Galanthamine‡; (−)- | 274.1443 | 7.3932 |
| form: O-De-Me | | |
| 8,11,13- | 275.2011 | 19.4817 |
| Podocarpatriene- | | |
| 12,13-diol: 12-Me | | |
| ether | | |
| Calabatine | 276.1963 | 11.4381 |
| 9,11,13,15- | 277.2167 | 100 |
| Octadecatetraenoic | | |
| acid; (9E,11E,13E, | | |
| 15E)-form | | |
| Stenine | 278.212 | 25.1911 |
| 7-Hydroxy-14,15- | 279.2324 | 21.6319 |
| dinor-8(17)-labden- | | |
| 13-one | | |
| Brevicolline; (±)- | 280.1813 | 13.3009 |
| form: N-Me | | |
| 2,3-Dibromo-5- | 280.8849 | 0.0165 |
| hydroxy-1,4- | | |
| benzoquinone | | |
| Thiarubrin E: 1- | 280.9861 | 0.033 |
| Deoxy, 1-chloro | | |
| Damirone A: 6- | 281.0125 | 0.0158 |
| Bromo, N5-de-Me | | |
| Methyl β-D- | 281.0872 | 0.1702 |
| glucopyranoside: 6- | | |
| O-Malonyl | | |
| Bharatamine; (±)- | 282.1494 | 7.6593 |
| form | | |
| 3-O-β-L- | 283.1029 | 0.9866 |
| Arabinopyranosyl-L- | | |
| arabinose | | |
| (N- | 284.1286 | 2.4864 |
| Phenylacetyl)glycine: | | |
| Benzyl ester | | |
| 2-Phenylethanol: O- | 285.1338 | 2.4427 |
| β-D- | | |
| Glucopyranoside | | |
| piperine/cocluarine/laurifoline | 286.1654 | 5.1809 |
| 4-(3,7-Dimethyl-2,6- | 287.1647 | 2.9295 |
| octadienyl)-5- | | |
| hydroxy-2(3H)- | | |
| benzofuranone | | |
| Trichostatic acid | 288.1599 | 9.1375 |
| 2,3,9-Trihydroxy- | 289.144 | 11.1623 |
| 1,3,5,7,9- | | |
| cadinapentaen-14-al: | | |
| 2,3-Di-Me ether | | |
| 4-Amino-4,6- | 290.1603 | 8.7716 |
| dideoxy-3-C- | | |
| methylmannose; β- | | |
| D-Pyranose-form: | | |
| Me glycoside, N- | | |
| Me, N,2-di-Ac | | |
| 8-Hydroxy-17- | 291.196 | 18.2243 |
| octadecene-9,11- | | |
| diynoic acid | | |
| (4-Aminobutyl)urea; | 292.1872 | 8.8096 |
| (2S,3′S)-form | | |
| Ecklonialactone A: | 293.2116 | 21.2953 |
| 6,7-Dihydro | | |
| Capsaicin: Nonanoyl | 294.2069 | 9.1679 |
| analogue | | |
| Tribromoacetic acid | 294.8205 | 0.0614 |
| 16-Hydroxy- | 295.2273 | 11.6354 |
| 9,12,14- | | |
| octadecatrienoic acid | | |
| 2-(3,7-Dimethyl-2,6- | 296.2014 | 6.798 |
| octadienyl)-3- | | |
| methyl-4(1H)- | | |
| quinolinone | | |
| Delesserine | 297.0974 | 0.2651 |
| Gindaricine | 298.1443 | 5.0583 |
| 1,10:4,5-Diepoxy- | 299.1494 | 1.5652 |
| 3,6,8-trihydroxy-11- | | |
| germacren-9-one; | | |
| (1β,3a,4a,5a,6a,8a,10β)- | | |
| form | | |
| Erythratine: 2-Deoxy | 300.1599 | 3.637 |
| N,N′-Bis(3- | 301.1552 | 2.08 |
| methoxybenzyl)urea | | |
| Pulchelline‡ | 302.1756 | 5.8463 |
| Trichostatin A | 303.1708 | 3.3365 |
| Schoberidine | 304.1813 | 4.3523 |
| 2,4,6-Trihydroxy-3- | 305.1753 | 5.1984 |
| prenylacetophenone; | | |
| 4-O-(3-Methyl-2- | | |
| butenyl) | | |
| 8-Methyl-8- | 306.1705 | 6.6871 |
| azabicyclo[3.2.1]octane- | | |
| 3,6-diol; (3RS, | | |
| 6RS)-form: 3-O- | | |
| (4- | | |
| Methoxyphenylacetyl) | | |
| 8-Hydroxy-4-oxo- | 307.1545 | 3.7462 |
| 7(11)-pseudoguaien- | | |
| 12,6-olide; | | |
| (1a,5β,6β,8a,10a H)- | | |
| form: Ac | | |
| 2-Benzamidobenzoic | 308.165 | 6.4758 |
| acid | | |
| Taberpsychine | 309.1967 | 7.79 |
| Brevicarine: N—Ac | 310.1919 | 6.1321 |
| Sarolactone | 311.0919 | 0.2493 |
| 1,3,5-Trihydroxy-4- | 312.1236 | 0.8349 |
| prenylacridone | | |
| Antibiotic PI 091 | 312.2175 | 0.2556 |
| 5-[(4- | 313.144 | 2.3166 |
| Hydroxyphenyl)ethenyl]- | | |
| 2-(3-methyl-1- | | |
| butenyl)-1,3- | | |
| benzenediol: 3′- | | |
| Hydroxy | | |
| Salutaridine; (+)- | 314.1392 | 3.9948 |
| form: N-De-Me | | |
| 3,4-Epoxy-2,5,8,9- | 315.1444 | 1.7579 |
| tetrahydroxy-11(13)- | | |
| germacren-12,6- | | |
| olide | | |
| Franklinol | 317.1753 | 3.4206 |
| 8-Methyl-8- | 318.1705 | 3.9781 |
| azabicyclo[3.2.1]octane- | | |
| 3,6-diol; (3R, | | |
| 6R)-form: 3-O- | | |
| Phenylacetyl, 6-Ac | | |
| 8- | 319.1909 | 3.7919 |
| dehydrogingerdione | | |
| Calabacine | 320.1974 | 4.9283 |
| Diptocarpidine: S,S′- | 321.2034 | 2.9424 |
| Dideoxy | | |
| 8-Methyl-8- | 322.2018 | 4.7881 |
| azabicyclo[3.2.1]octane- | | |
| 3,6-diol; (3R, | | |
| 6R)-form: Ditigloyl | | |
| Bavachromene | 323.1283 | 0.8856 |
| C-Alkaloid M | 324.1838 | 4.0024 |
| Venusol; (E)-form | 325.0923 | 0.2918 |
| Ergometrine,, | 326.1868 | 3.1266 |
| Plicane | 327.1345 | 1.0241 |
| ChondriamideA: | 328.145 | 1.1816 |
| (10′Z)-Isomer | | |
| Lomatin; (R)-form: | 329.1389 | 0.8851 |
| O-(3-Methyl-2- | | |
| butenoyl) | | |
| sinomenin/deoxyharringtonine | 330.1705 | 2.2984 |
| picrocrocin/carnosol | 331.1757 | 2.1252 |
| 4-Amino-4,6- | 332.1709 | 3.8324 |
| dideoxy-3-C- | | |
| methylmannose; β- | | |
| D-Pyranose-form: | | |
| Me glycoside, N, | | |
| 2,3-tri-Ac | | |
| 4-(5-Phenyl-2,4- | 333.1854 | 3.0627 |
| pentadien-1- | | |
| yl)tetracyclo[5.4.0.02, | | |
| 5.03,9]undec-10- | | |
| ene-8-carboxylic | | |
| acid | | |
| Nb-(3- | 334.1919 | 3.1566 |
| Indolylmethyl)-5- | | |
| methoxy-Nb- | | |
| methyltryptamine | | |
| Lasiodiplodin; (R)- | 335.1858 | 2.2175 |
| form: Ac | | |
| 2-Benzyl-3-tropanol; | 336.1963 | 3.766 |
| (1RS,2SR,3RS)- | | |
| form: Benzoyl | | |
| 8-Methyl-8- | 338.1967 | 2.6492 |
| azabicyclo[3.2.1]octane- | | |
| 3,6,7-triol; (1R*, | | |
| 3S*,6S*,7R*)- | | |
| form: 3,6-Di-O- | | |
| tigloyl | | |
| 2- | 340.1912 | 2.6523 |
| Methoxyatherosperminine | | |
| Picrasidine E | 341.1137 | 0.4527 |
| Tecleaverdoornine: | 342.1341 | 0.5939 |
| Me ether | | |
| Arginine,,; (±)- | 343.144 | 0.8372 |
| form: N2-Me, N2- | | |
| (4- | | |
| methylbenzenesulfonyl) | | |
| Mazethramycin A: | 344.161 | 1.8097 |
| Me ether | | |
| 3,7,11,15(17)- | 345.1702 | 1.637 |
| Cembratetraene- | | |
| 16,2:19,6-diolide; (1R, | | |
| 2S,3E,6R,11E)- | | |
| form: 11,12-Epoxide | | |
| Suaveoline‡: N—Ac | 346.1919 | 2.7445 |
| Wederegiolide: 15a- | 347.1858 | 2.0179 |
| Hydroxy | | |
| Mahanine; (?)-form: | 348.1963 | 2.3454 |
| Deoxy, ? | | |
| 1,6,14-Trihydroxy- | 349.2015 | 1.5481 |
| 16-kaurene-7,15- | | |
| dione | | |
| Fawcettiine; (−)- | 350.2331 | 3.7526 |
| form: Ac | | |
| Stemospironine | 352.2124 | 2.5345 |
| Ajmalicine; (−)- | 353.1865 | 0.5579 |
| form: 20-Epimer | | |
| ribo-Hexos-3-ulose; | 354.1916 | 2.2102 |
| a-D-Furanose- | | |
| form: 1,2:5,6-Di-O- | | |
| cyclohexylidene, | | |
| oxime | | |
| Isocorydine; (R)- | 358.1654 | 1.0817 |
| form: a-N-Oxide | | |
| Phyllocryptonine | 359.1732 | 1.4391 |
| Lincosamine; a- | 360.2022 | 2.4469 |
| Pyranose-form: 7- | | |
| Me, 1,2:3,4-di-O- | | |
| isopropylidene, N—Ac | | |
| Furanoeremophilane- | 361.2015 | 2.3715 |
| 3,6-diol; (3β,6β)- | | |
| form: 3-(2-Methyl-2- | | |
| propenoyl), 6-Ac | | |
| 8-Methyl-8- | 362.1967 | 2.7438 |
| azabicyclo[3.2.1]octan- | | |
| 3-ol; (1RS,3RS)- | | |
| form: O-(3,4,5- | | |
| Trimethoxyc amoyl) | | |
| 23-Oxo- | 363.196 | 1.2519 |
| 3,5,7,9,11,13,15,17,19, | | |
| 21- | | |
| tetracosadecaenoicacid | | |
| Adenosine,, N6- | 364.1985 | 1.9342 |
| Cyclohexyl, 2′-O- | | |
| Me | | |
| 3,14,17,21- | 365.2328 | 0.1964 |
| Tetrahydroxypregn- | | |
| 5-en-20-one; | | |
| (3β,14β,17βOH)- | | |
| form | | |
| Crosemperine | 368.2073 | 1.6157 |
| Dendrowardine: | 374.2098 | 1.7199 |
| Chloride | | |
| Wedeliasecokaurenolide: | 375.2171 | 1.3298 |
| 3β-Acetoxy | | |
| Acsine | 376.2124 | 1.6893 |
| Lincomycin,,: 1- | 377.2288 | 1.31 |
| De(methylthio), 1- | | |
| hydroxy | | |
| Heteratisine: O-De- | 378.228 | 2.0617 |
| Me | | |
| Pseudostrychnine: Et | 379.2021 | 0.702 |
| ether | | |
| 3- | 380.2589 | 2.6762 |
| Greenwayodendrinol; | | |
| 3β-form: Ac | | |
| Kayawongine | 382.2382 | 1.5283 |
| Eudesmin | 387.1807 | 0.7209 |
| Saxoguattine | 388.176 | 0.6596 |
| Wederegiolide: 15β- | 389.1964 | 0.9539 |
| Acetoxy | | |
| DioncophyllineA: | 390.2069 | 1.833 |
| 1,2-Didehydro, Me | | |
| ether | | |
| Antibiotic LL-Z | 391.204 | 0.8112 |
| 1272β: 5-Chloro | | |
| Roquefortine: 3a,12- | 392.2086 | 1.6989 |
| Dihydro | | |
| Linderatone | 393.2066 | 0.6291 |
| Macrocentrine | 394.2593 | 2.1276 |
| 12- | 396.2413 | 0.8436 |
| Methoxyaffinisine: | | |
| 17-Carboxylic acid, | | |
| N4-Me, Et ester | | |
| Dehydronapelline: | 398.2331 | 0.8571 |
| 12-Ketone, 15-Ac | | |
| 4,11-Eudesmanediol; | 403.2696 | 0.7783 |
| (ent-4a,5a)-form: 4- | | |
| O-β-D- | | |
| Glucopyranoside | | |
| Dammar-24-ene- | 404.2437 | 1.1227 |
| 3,20,26-triol; | | |
| Danomycin B: 11S- | | |
| Hydroxy, 14,15- | | |
| didehydro | | |
| 2-Phenyl-1,5,9- | 406.2494 | 1.5382 |
| triazacyclotridecan- | | |
| 4-one; (S)-form: N9- | | |
| E-C amoyl | | |
| Shamixanthone | 407.1858 | 0.5479 |
| 5,13-Dihydroxy- | 408.2572 | 1.2535 |
| 2,4,6,14-tetramethyl- | | |
| 15-(2-methyl-4- | | |
| thiazolyl)-10,14- | | |
| pentadecadien-3-one | | |
| Aconitane- | 410.2542 | 0.9739 |
| 1,4,8,9,14,16-hexol; | | |
| (1a,5β,14a,16β)- | | |
| form: O14,O16-Di- | | |
| Me, N-Et | | |
| Saliniketal A: 18- | 412.2699 | 0.7261 |
| Hydroxy | | |
| Trachyone | 414.3008 | 0.8356 |
| Paxilline: 4b-Deoxy | 420.2538 | 2.2841 |
| Kazinol L | 421.2379 | 0.7335 |
| Graciline: 6β,16β- | 422.2542 | 0.8736 |
| Dimethoxy | | |
| Lincomycin,,: S- | 423.2165 | 0.2786 |
| Oxide | | |
| Aconitane- | 424.2699 | 0.9551 |
| 1,4,8,9,14,16-hexol; | | |
| (1a,5β,14a,16β)- | | |
| form: O1,O14,O16- | | |
| Tri-Me, N-Et | | |
| Aurachin G: 3?- | 426.2644 | 0.3278 |
| Methoxy, 1′,2′- | | |
| dihydro | | |
| Coyhaiquine: 1″- | 432.1811 | 0.2871 |
| Aldehyde | | |
| Estra-1,3,5(10)- | 434.2331 | 0.6025 |
| triene-3,17-diol; | | |
| 17β-form: O17-(3- | | |
| Pyridinecarboxylate), | | |
| 3-propanoyl | | |
| Koumicine | 435.2284 | 0.3642 |
| Acophine | 436.2618 | 0.8427 |
| Antibiotic SI 4228C: | 437.2175 | 0.3332 |
| 8-Hydroxy | | |
| 4-Methylaconit-2- | 438.2492 | 0.7215 |
| ene- | | |
| 1,6,7,8,14,16,18- | | |
| heptol; | | |
| (1a,5β,6β,14a,16β)- | | |
| form: O16,O18-Di- | | |
| Me, N-Et | | |
| Riboflavine,,: | 439.1019 | 0.1019 |
| 4′,5′-Cyclic | | |
| phosphate | | |
| Dehydronapelline: | 442.2593 | 0.4108 |
| Di-Ac | | |
| Sarcotragin A: N- | 448.2699 | 1.4498 |
| De-(2-phenylethyl), | | |
| N-(carboxymethyl) | | |
| 3,7-Dimethyl-2,6- | 449.2387 | 0.323 |
| octadien-1-o1; (E)- | | |
| form: O-[β-D- | | |
| Apiofuranosyl- | | |
| (1?6)-β-D- | | |
| glucopyranoside] | | |
| Heliotropium | 450.273 | 0.6182 |
| europaeum Alkaloid | | |
| 7,20-Epoxy-16- | 451.2332 | 0.2312 |
| kaurene-1,3,6,7,15- | | |
| pentol; (ent- | | |
| 1a,3a,6a,15a)-form: | | |
| 6,15-Di-Ac | | |
| Trideacetylpyripyropene | 452.2437 | 0.5319 |
| A: 7,11,19- | | |
| Trideoxy, 3-Ac | | |
| Lasiocarpine: O3′- | 454.2441 | 0.4599 |
| Ac | | |
| Ascochalasin: 21,22- | 464.2801 | 0.719 |
| Didehydro(E-) | | |
| Trideacetylpyripyropene | 466.2593 | 0.5651 |
| A: 7,11,19- | | |
| Trideoxy, 3- | | |
| propanoyl | | |
| CyclopiamineA | 468.2498 | 0.4097 |
| Meleagrin: O—Ac | 476.1934 | 0.1157 |
| Spectinabilin | 478.2229 | 0.3649 |
| Nigakilactone N: | 479.2281 | 0.2109 |
| 11,12-Di-Ac | | |
| CytochalasinB | 480.275 | 0.3587 |
| Trideacetylpyripyropene | 482.2542 | 0.2933 |
| A: 7,19- | | |
| Dideoxy, 3- | | |
| propanoyl | | |
| Zygosporin G | 492.275 | 0.3425 |
| 4-Methylaconitane- | 494.2754 | 0.4492 |
| 1,5,6,7,8,14,16- | | |
| heptol; | | |
| (1a,5β,6β,14a,16β)- | | |
| form: 7,8-Methylene | | |
| ether, O1,O14,O16- | | |
| tri-Me, N-Me, 6- | | |
| Ac | | |
| Ustilaginoidin A; (R)- | 547.0876 | 0.1364 |
| form: 1″,1′″- | | |
| Dihydroxy | | |
| Antibiotic A 80915C | 547.1421 | 0.1481 |
| Auriculatin: Tri-Ac | 547.1968 | 0.1405 |
| Vignatic acid A | 554.2866 | 0.181 |
| 4-Methylaconitane- | 568.3274 | 0.2192 |
| 1,8,14,16,18-pentol; | | |
| (1a,14a,16β)-form: | | |
| O1,O16,O18-Tri- | | |
| Me, N-Et, 8- | | |
| benzoyl, 14-Ac | | |

TABLE 11
DART TOF-MS characterization of Nettle Extract 4.

| | | Rel. |
|---|---|---|
| | Calc. | Abund. |
| Compound Name | Mass | (%) |
| 2-Methyl-2-butenoic | 100.0762 | 2.7054 |
| acid; (E)-form: | | |
| Amide | | |
| Acetaldehyde: | 102.0667 | 1.5384 |
| Semicarbazone | | |
| 4-Hydroxy-2- | 103.0395 | 5.6413 |
| oxobutanal | | |
| N,N- | 104.0711 | 45.256 |
| Dimethylglycine | | |
| 5-Methyl-3- | 109.0402 | 9.8949 |
| isoxazolecarboxylic | | |
| acid: Nitrile | | |
| 1H-Pyrrole-3- | 110.0718 | 19.5914 |
| carboximidamide | | |
| Resorcinol | 111.0446 | 11.6489 |
| 2,3-Dihydro-1H- | 112.0762 | 10.3376 |
| pyrrole: N—Ac | | |
| 6-azacytosine | 113.0425 | 8.7773 |
| 3-Azetidinone: N—Ac | 114.0555 | 7.9021 |
| 2-Amino-3- | 115.0507 | 1.0476 |
| cyanopropanoic | | |
| acid; (S)-form | | |
| Proline | 116.0824 | 3.2818 |
| Levulinic acid | 117.0612 | 19.697 |
| Guanidinoaceticacid | 118.0616 | 2.699 |
| Succinic acid | 119.0344 | 4.3296 |
| Threonine | 120.066 | 2.2831 |
| Nitrosofungin | 121.0613 | 7.4962 |
| Benzaldehyde, (E)- | 122.0606 | 3.8447 |
| Oxime | | |
| Vitamin B3 | 123.0657 | 6.2933 |
| (niacinamide) | | |
| 2- | 124.0762 | 21.6506 |
| Hydroxybenzylamine | | |
| 1H-Pyrrole-2- | 125.0715 | 19.7444 |
| carboxaldehyde: N- | | |
| Me, oxime | | |
| 2-Amino-4- | 126.0667 | 11.2498 |
| hydroxypyrimidine; | | |
| 1H-form: 1-Me | | |
| 5-Hydroxy-3-vinyl- | 127.0395 | 27.7184 |
| 2(5H)-furanone | | |
| 1-Nonene | 127.1487 | 2.0384 |
| 3- | 128.0711 | 9.5384 |
| Azabicyclo[3.1.0]hexane- | | |
| 2-carboxylic | | |
| acid; (1S,2S,5R)- | | |
| form | | |
| 5,6-Dihydro-5- | 129.0551 | 6.2993 |
| hydroxy-6-methyl- | | |
| 2H-pyran-2-one; | | |
| (5R,6S)-form | | |
| 2-Amino-4-hexenoic | 130.0868 | 5.8132 |
| acid | | |
| Leucine | 132.1024 | 9.1289 |
| Asparagine | 133.0613 | 6.16 |
| 2-Amino-4-hydroxy- | 134.0817 | 3.8415 |
| 3-methylbutanoic | | |
| acid | | |
| Adenine | 136.061 | 71.5949 |
| 4-Methylbenzoic | 137.0602 | 26.3881 |
| acid | | |
| 5-Ethyl-2- | 138.0919 | 18.5322 |
| methylpyridine: N- | | |
| Oxide | | |
| 1,4- | 139.0759 | 11.3104 |
| Dimethoxybenzene | | |
| Choline: Chloride | 140.0842 | 19.9966 |
| 1,2,3-Benzenetriol: | 141.0551 | 4.6247 |
| 1-Me ether | | |
| a-Amino-2- | 142.0868 | 10.0521 |
| cyclopentene-1- | | |
| acetic acid | | |
| 2- | 144.066 | 11.4152 |
| Hydroxymethylclavam | | |
| 2,3-Dihydro-3,6- | 145.0501 | 18.1507 |
| dihydroxy-2-methyl- | | |
| 4H-pyran-4-one | | |
| 2-Amino-3- | 146.0817 | 10.503 |
| hydroxymethyl-3- | | |
| pentenoic acid | | |
| 2,3- | 147.0769 | 6.4382 |
| Diaminopropanoic | | |
| acid; (S)-form: N3- | | |
| Ac | | |
| 2-Amino-5- | 148.0973 | 5.34 |
| hydroxyhexanoic | | |
| acid | | |
| O-Carbamoylserine | 149.0562 | 13.122 |
| 4-Methylbenzoic | 150.0919 | 21.0777 |
| acid: Methylamide | | |
| 4-Methylbenzoic | 151.0759 | 11.162 |
| acid: Me ester | | |
| 2-Aminobenzoic | 152.0824 | 20.8611 |
| acid: Hydrazide | | |
| Phenylmethanethiol: | 153.0738 | 16.5004 |
| S-Et | | |
| Scopine: 3-Ketone | 154.0868 | 26.0969 |
| 2,3-Dichloro-2- | 154.9666 | 0.1638 |
| propenoicacid; (E)- | | |
| form: Me ester | | |
| 3,4- | 155.0708 | 13.2058 |
| Dihydroxybenzyl | | |
| alcohol: 4-Me ether | | |
| Scopine | 156.1024 | 18.4662 |
| 3-(2-Hydroxyethyl)- | 158.0817 | 7.5253 |
| 4-oxa-1- | | |
| azabicyclo[3.2.0]heptan- | | |
| 7-one | | |
| 4-Hydroxy-1,1- | 160.0973 | 6.0987 |
| dimethylpyrrolidinium- | | |
| 2-carboxylate; (2S, | | |
| 4R)-form | | |
| 2,6-Diamino-4- | 161.0926 | 2.6787 |
| oxohexanoicacid | | |
| Boschniakine; (R)- | 162.0919 | 11.9221 |
| form | | |
| Levoglucosan/Glyogen/ | 163.0615 | 47.4427 |
| Laminarin | | |
| N-[2-(4- | 164.0711 | 33.2933 |
| Hydroxyphenyl)ethenyl]formamide | | |
| 1,2,4,5- | 165.1127 | 10.5128 |
| Pentanetetrol; (2RS, | | |
| 4RS)-form: 1,5-Di- | | |
| Me ether | | |
| Amino-1,4- | 167.082 | 12.3499 |
| benzoquinone: N- | | |
| Me, 4-methyloxime | | |
| Synephrine | 168.1024 | 23.9798 |
| Tetramethylpyrazine: | 169.0977 | 21.7227 |
| 1,4-Dioxide | | |
| 2-Amino-3-(3- | 170.0817 | 10.9813 |
| furanyl)propanoic | | |
| acid; (S)-form: N- | | |
| Me | | |
| 2-Amino-4-hexenoic | 172.0973 | 7.686 |
| acid; (±)-(Z)-form: | | |
| N—Ac | | |
| Caprazamycin; | 173.1038 | 8.4747 |
| CaprazamycinG | | |
| 8-Methyl-8- | 174.113 | 10.0865 |
| azabicyclo[3.2.1]octane- | | |
| 3,6,7-triol | | |
| 4,5,6-Trihydroxy-6- | 175.0606 | 7.3282 |
| (hydroxymethyl)-2- | | |
| cyclohexen-1-one | | |
| Shikimic acid | 175.1572 | 33.2836 |
| 4-Amino-1-phenyl- | 176.1075 | 18.5863 |
| 1-penten-3-one | | |
| Canavanine; (S)- | 177.0987 | 13.6874 |
| form | | |
| N-[2-(4- | 178.0868 | 32.3401 |
| Hydroxyphenyl)ethenyl]formamide: | | |
| Me | | |
| ether | | |
| N-Benzoylglycine: | 179.082 | 14.8109 |
| Amide | | |
| 1,2,3,4-Tetrahydro- | 180.1024 | 44.4995 |
| 4,8-dihydroxy-2- | | |
| methylisoquinoline | | |
| Herbipoline | 181.0963 | 27.1535 |
| (2- | 182.0946 | 33.9499 |
| Aminoethyl)phosphonic | | |
| acid: Di-Et ester | | |
| Dihydro-5-(5- | 183.1021 | 13.5207 |
| hydroxy-1,3- | | |
| hexadienyl)-2(3H)- | | |
| furanone | | |
| Pyridoxine,,: 5-Me | 184.0973 | 11.6646 |
| ether | | |
| 2-Naphthol: 2- | 185.0966 | 5.6601 |
| Propenyl ether | | |
| 4-Hydroxy-3-(3- | 187.0759 | 9.9549 |
| methyl-3-buten-1- | | |
| ynyl)benzoic acid: | | |
| 1′-Aldehyde | | |
| 2-Amino-4- | 188.0923 | 15.2387 |
| propylidenepentanedioic | | |
| acid | | |
| 1H-Indole-3- | 189.1028 | 7.4048 |
| methanamine: 3-N—Ac | | |
| 2-Ethyl-4- | 190.098 | 15.5806 |
| hydroxyquinazoline; | | |
| 3H-form: N-Amino | | |
| Carnitine,; (R)- | 191.1521 | 47.1298 |
| form: Et ester | | |
| Garosamine; β-L- | 192.1236 | 34.3625 |
| Pyranose-form: Me | | |
| glycoside | | |
| 2,6,8,10- | 193.1228 | 33.0626 |
| Dodecatetraenoicacid | | |
| 1,2,3,4-Tetrahydro- | 194.1181 | 28.3158 |
| 7-hydroxy-6- | | |
| methoxy-1- | | |
| methylisoquinoline; | | |
| (S)-form | | |
| Tagatose; a-D- | 195.0868 | 4.2152 |
| Pyranose-form: Me | | |
| glycoside | | |
| DL-methyl-m- | 196.1099 | 27.7271 |
| tyrosine | | |
| 2-Acetyl-4,4,6- | 197.1177 | 17.2172 |
| trimethyl-1,3- | | |
| cyclohexanedione | | |
| Buddamine | 198.113 | 28.0628 |
| Pedatisectine D | 199.1082 | 24.7936 |
| N-(2- | 200.1035 | 15.3585 |
| Cyanoethyl)glutamine; | | |
| (±)-form | | |
| Mackinazolinone | 201.1028 | 3.7051 |
| 2,6- | 202.1079 | 10.474 |
| Piperidinedicarboxylic | | |
| acid; (2RS,6RS)- | | |
| form: Di-Me ester | | |
| 1,2,3,4-Tetrahydro- | 203.1184 | 8.828 |
| 1-methyl-β- | | |
| carboline; (?)-form: | | |
| Nb-Oxide | | |
| Carnitine,; (±)-form: | 204.1236 | 18.5191 |
| O—Ac | | |
| 4-methyl-7- | 205.0937 | 38.0808 |
| ethoxycoumarin | | |
| N-(2- | 206.1181 | 23.4827 |
| Hydroxyethyl)camamide: | | |
| N-Me | | |
| 6-Deoxyglucose; β- | 207.1232 | 19.8402 |
| L-Pyranose-form: 3- | | |
| Me, Et glycoside | | |
| 1,2,3,4-Tetrahydro- | 208.1337 | 29.9995 |
| 7-hydroxy-6- | | |
| methoxy-1- | | |
| methylisoquinoline; | | |
| (±)-form: N-Me | | |
| 6,10-Dimethyl-5,9- | 209.1541 | 61.5097 |
| undecadiene-2,8- | | |
| dione | | |
| Elaeokanine E | 210.1494 | 25.51 |
| Antibiotic A 41-89; | 211.1334 | 15.0694 |
| Antibiotic A 41-89I | | |
| 4-Methylbenzoic | 212.1075 | 18.8445 |
| acid: Anilide | | |
| Linderazulene: 2,3- | 213.1279 | 11.8085 |
| Dihydro | | |
| 2-Aminobenzyl | 214.1232 | 14.0147 |
| alcohol: N-Benzyl | | |
| Cyclo(aspartylleucyl) | 215.1032 | 26.6912 |
| a-Amino-?- | 216.1236 | 23.711 |
| oxooxiraneoctanoic | | |
| acid | | |
| 2-Oxohexanedioic | 217.1076 | 12.7927 |
| acid: Di-Et ester | | |
| Glycylglycylglycine: | 218.1141 | 18.4871 |
| Et ester | | |
| 2-Amino-3-(3- | 219.1133 | 14.2809 |
| indolyl)butanoic acid | | |
| Vitamin B5 | 220.1185 | 26.2087 |
| 5-Aminopentanoic | 221.129 | 22.3453 |
| acid: N-Benzoyl, | | |
| amide | | |
| 2-Amino-2- | 222.1341 | 25.1993 |
| deoxygalactose; a- | | |
| D-Pyranose-form: | | |
| 3,4,6-Tri-Me | | |
| Cystathionine | 223.0752 | 2.0921 |
| 14-Nor-5- | 223.1698 | 1.4354 |
| protoilludene-7,8- | | |
| diol; (7a,8β)-form | | |
| Cerulenin | 224.1286 | 15.7003 |
| Choline: O-(4- | 225.1365 | 14.8582 |
| Hydroxybenzoyl) | | |
| 10-(3-Pyridinyl)- | 226.1232 | 12.8264 |
| 3,5,7,9-decatetraen- | | |
| 2-one; (all-E)-form | | |
| 1-(2,5- | 227.1283 | 13.6756 |
| Dihydroxyphenyl)- | | |
| 1,2-propanediol; (1R*, | | |
| 2R*)-form: 1-Et, | | |
| 5′-Me ether | | |
| 2-Amino-4-hexynoic | 228.1236 | 13.5397 |
| acid; (R)-form: N- | | |
| tert- | | |
| Butyloxycarbonyl | | |
| 1-Pentadecylamine | 228.2691 | 0.8688 |
| Faramol | 229.1228 | 13.2682 |
| Haplofoline | 230.1181 | 25.1032 |
| 9H-Pyrido[3,4-b]indol- | 231.1133 | 12.4554 |
| 7-ol: 1,2,3,4- | | |
| Tetrahydro, Me | | |
| ether, N9-formyl | | |
| 2-Oxohexanedioic | 232.1185 | 22.0306 |
| acid: Di-Et ester, | | |
| oxime | | |
| Cytisine; (−)-form: | 233.129 | 15.7436 |
| N—Ac | | |
| Garosamine; β-L- | 234.1341 | 19.2561 |
| Pyranose-form: Me | | |
| glycoside,N—Ac | | |
| 8-Hydroxy-13-nor- | 235.1334 | 28.2953 |
| 4(15),7(11)- | | |
| eudesmadien-12,8- | | |
| olide | | |
| Anhalonine; (S)- | 236.1286 | 26.4652 |
| form: N-Me | | |
| Theophylline,, JAN, | 237.1351 | 14.778 |
| N-(2-Methylpropyl) | | |
| 2,3-Dihydro-2- | 238.1232 | 23.3293 |
| methylindole; (R)- | | |
| form: N-Benzoyl | | |
| Mescaline: N- | 240.1236 | 14.3996 |
| Formyl | | |
| 3,5- | 241.1228 | 10.5323 |
| Dihydroxystilbene; | | |
| (E)-form: Di-Me | | |
| ether | | |
| 2-Hydroxybenzoic | 242.1392 | 14.2344 |
| acid: Choline ester | | |
| phosphatidylcholine | 243.1385 | 11.5762 |
| Sceletenone | 244.1337 | 17.6913 |
| Osthole | 245.1177 | 16.2482 |
| 3-Deoxy-3- | 246.1341 | 21.3628 |
| (methylamino)arabinose; | | |
| β-L-Furanose- | | |
| form: N—Ac, 1,2-O- | | |
| isopropylidene | | |
| 8-Hydroxy- | 247.1334 | 17.9893 |
| 1,4,7(11)-guaiatrien- | | |
| 12,8-olide | | |
| 3-(3,4- | 248.1286 | 22.1968 |
| Methylenedioxyphenyl)- | | |
| 2-propenoic acid; | | |
| (E)-form: 2- | | |
| Methylpropylamide | | |
| 4-shogaol | 249.1338 | 13.7241 |
| Anhalonine; (S)- | 250.1443 | 18.4483 |
| form: N-Et | | |
| 9,10-Dihydro-2- | 251.1436 | 10.2669 |
| hydroxy-1,6- | | |
| dimethyl-7- | | |
| vinylphenanthrene | | |
| N-2-Phenylethylcamamide | 252.1388 | 31.7119 |
| Nebularine | 253.0937 | 1.9597 |
| 1,2,3,4-Tetrahydro- | 253.1678 | 2.6339 |
| 6,7,8-trihydroxy-1- | | |
| methylisoquinoline; | | |
| (?)-form: 6,7-Di-Me | | |
| ether, N,N-di-Me | | |
| 1,2,3,4-Tetrahydro- | 254.1392 | 12.6489 |
| 5,6,7-trimethoxy-2- | | |
| methylisoquinoline: | | |
| N-Oxide | | |
| a,a′-Diamino-2- | 255.1345 | 13.4967 |
| methylenecycloheptene- | | |
| 1,3-diacetic acid | | |
| 2-Amino-3-phenyl- | 256.1337 | 15.5157 |
| 1-propanol; (S)- | | |
| form: N-Benzoyl | | |
| Cordiachrome G | 257.1177 | 15.3092 |
| Carbazole: N- | 258.1282 | 14.7579 |
| Benzyl | | |
| Elaeagin | 259.1334 | 15.5814 |
| Peganidine: Oxime | 260.1399 | 18.7145 |
| myo-Inositol: | 261.1338 | 17.4333 |
| 1,2:4,5-Di-O- | | |
| isopropylidene | | |
| 8-Methyl-8- | 262.1443 | 19.6041 |
| azabicyclo[3.2.1]octane- | | |
| 3,6-diol; (3R, | | |
| 6R)-form: 3- | | |
| Benzoyl | | |
| Furodysinin; (−)- | 263.1469 | 13.0596 |
| form: 14- | | |
| (Methylthio) | | |
| 2-Acetamido-2- | 264.1447 | 16.8209 |
| deoxyglucose; D- | | |
| form: 3,4,6-Tri-Me | | |
| 3-Hydroxy-1-oxo- | 265.144 | 9.3063 |
| 7(11)-eudesmen- | | |
| 12,6-olide; | | |
| (3β,4a,6β)-form | | |
| Anisomycin | 266.1392 | 19.1666 |
| 1,5-Bis(4- | 267.1021 | 1.5206 |
| hydroxyphenyl)-1,4- | | |
| pentadien-3-one; | | |
| (E,E)-form | | |
| Peyoruvic acid | 268.1185 | 21.7645 |
| 8,8a-Dihydro-8a- | 269.1389 | 11.8384 |
| hydroxy-3,3,6,6,8,8- | | |
| hexamethyl-1,2- | | |
| benzodioxin-5,7(3H, | | |
| 6H | | |
| Coccuvine; Natural- | 270.1494 | 12.7514 |
| form | | |
| 2-Hydroxybenzoic | 271.1334 | 14.9108 |
| acid: 4-tert- | | |
| Butylphenyl ester | | |
| Cynometrine: N1- | 272.1399 | 17.3954 |
| De-Me | | |
| Verimol F | 273.149 | 15.4373 |
| Galanthamine‡; (−)- | 274.1443 | 17.2935 |
| form: O-De-Me | | |
| Hydrostatin A | 275.1971 | 27.2056 |
| Physostigmine,, | 276.1712 | 22.6507 |
| 9,11,13,15- | 277.2167 | 100 |
| Octadecatetraenoic | | |
| acid; (9E,11E,13E, | | |
| 15E)-form | | |
| Stenine | 278.212 | 31.7045 |
| 3′-Methoxy- | 278.9972 | 0.0651 |
| 2,2′;5′,2″- | | |
| terthiophene | | |
| 7-Hydroxy-14,15- | 279.2324 | 48.0068 |
| dinor-8(17)-labden- | | |
| 13-one | | |
| Dendrobine: N- | 280.1912 | 22.2332 |
| Oxide | | |
| 1,7-Diphenyl-3,5- | 281.1541 | 4.8297 |
| heptanediol; (3S,5S)- | | |
| form: 1,2- | | |
| Didehydro(E-), 3- | | |
| ketone | | |
| Bharatamine; (±)- | 282.1494 | 14.2731 |
| form | | |
| Colletodiol: 9,10- | 283.1181 | 3.5774 |
| Dihydro, 11,12- | | |
| diketone | | |
| (N- | 284.1286 | 9.5816 |
| Phenylacetyl)glycine: | | |
| Benzyl ester | | |
| 2,3-Dihydro-2-(4- | 285.149 | 8.7719 |
| hydroxyphenyl)-5- | | |
| (2-hydroxypropyl)- | | |
| 3-methylbenzofuran | | |
| piperine/cocluarine/laurifoline | 286.1443 | 12.3437 |
| Komaroine | 287.1548 | 10.9939 |
| Trichostatic acid | 288.1599 | 16.9523 |
| chiro-Inositol; D- | 289.1651 | 26.6707 |
| form: 1,4-Di-Me, | | |
| 2,3:5,6-di-O- | | |
| isopropylidene | | |
| 4-Amino-4,6- | 290.1603 | 20.4136 |
| dideoxy-3-C- | | |
| methylmannose; β- | | |
| D-Pyranose-form: | | |
| Me glycoside, N- | | |
| Me, N, 2-di-Ac | | |
| 8-Hydroxy-17- | 291.196 | 32.4864 |
| octadecene-9,11- | | |
| diynoic acid | | |
| 1,2,3,5- | 292.1661 | 17.3442 |
| Benzenetetrol: | | |
| Tris(ethylamide) | | |
| Ecklonialactone A: | 293.2116 | 32.2169 |
| 6,7-Dihydro | | |
| 3-Amino-2,3,6- | 294.1705 | 16.1121 |
| trideoxy-3-C- | | |
| methyl-lyxo-hexose; | | |
| a-L-Pyranose-form: | | |
| Et glycoside, N- | | |
| benzoyl | | |
| 16-Hydroxy- | 295.2273 | 20.8926 |
| 9,12,14- | | |
| octadecatrienoic acid | | |
| 8-Methyl-8- | 296.1862 | 15.6973 |
| azabicyclo[3.2.1]octane- | | |
| 3,6-diol; (3R, | | |
| 6R)-form: 3- | | |
| Tigloyl, 6-propanoyl | | |
| Tanshindiol A: 18- | 297.1127 | 0.8377 |
| Deoxy | | |
| Verpacamide D | 298.1515 | 9.8978 |
| 1,10:4,5-Diepoxy- | 299.1494 | 7.5993 |
| 3,6,8-trihydroxy-11- | | |
| germacren-9-one; | | |
| (1β,3a,4a,5a,6a,8a,10β)- | | |
| form | | |
| Erythratine: 2-Deoxy | 300.1599 | 11.1325 |
| Febrifugine‡; (+)- | 301.1664 | 10.9477 |
| form: Oxime | | |
| Pulchelline‡ | 302.1756 | 13.1594 |
| Trichostatin A | 303.1708 | 11.4194 |
| Schoberidine | 304.1813 | 12.8898 |
| Jaborandine | 305.2229 | 26.6795 |
| Amicetamine | 306.1916 | 18.9976 |
| Eburnamenine; (−)- | 307.181 | 13.4678 |
| form: 11-Methoxy, | | |
| 14,15-didehydro | | |
| C-Alkaloid B | 308.1888 | 16.4869 |
| Taberpsychine | 309.1967 | 17.6889 |
| Atherosperminine | 310.1807 | 14.3538 |
| 1-O—C | 311.1131 | 1.5881 |
| amoylglucose; β-D- | | |
| Pyranose-(E)-form | | |
| Clausenamide; (±)- | 312.1599 | 7.0243 |
| form: 1′-Me ether | | |
| 5-[(4- | 313.144 | 6.1193 |
| Hydroxyphenyl)ethenyl]- | | |
| 2-(3-methyl-1- | | |
| butenyl)-1,3- | | |
| benzenediol: 3′- | | |
| Hydroxy | | |
| Salutaridine; (+)- | 314.1392 | 14.8831 |
| form: N-De-Me | | |
| Flourensianol: | 315.1596 | 8.7956 |
| Tigloyl | | |
| Ipanguline D3: 3″- | 316.176 | 12.3184 |
| Ac | | |
| 2-Amino-16-methyl- | 316.3215 | 0.3113 |
| 1,3-octadecanediol | | |
| Franklinol | 317.1753 | 11.6255 |
| 8-Methyl-8- | 318.1705 | 12.7919 |
| azabicyclo[3.2.1]octane- | | |
| 3,6-diol; (3R, | | |
| 6R)-form: 3-O- | | |
| Phenylacetyl, 6-Ac | | |
| 8- | 319.1909 | 12.0366 |
| dehydrogingerdione | | |
| Calabacine | 320.1974 | 12.6277 |
| 3-Hydroxy-17-nor- | 321.2066 | 10.7653 |
| 8-oxo-13-labden-15- | | |
| oic acid; (ent-3β,13Z)- | | |
| form: 3-Ketone | | |
| 8-Methyl-8- | 322.2018 | 14.7353 |
| azabicyclo[3.2.1]octane- | | |
| 3,6-diol; (3R, | | |
| 6R)-form: Ditigloyl | | |
| Colletochlorin A; (E)- | 323.1858 | 7.8177 |
| form: Dechloro | | |
| Inomycin | 324.2049 | 14.2824 |
| Talpinine | 325.1916 | 4.215 |
| Ergometrine,, | 326.1868 | 10.9175 |
| Plicane | 327.1345 | 2.4325 |
| Cryptostyline I; (R)- | 328.1549 | 4.4556 |
| form | | |
| 5-Isopropyl-2- | 329.16 | 5.7401 |
| methyl-1,3- | | |
| benzenediol: Mono- | | |
| O-β-D- | | |
| glucopyranoside | | |
| sinomenin/deoxyharringtonine | 330.1705 | 8.5624 |
| picrocrocin/carnosol | 331.1757 | 8.8042 |
| Alkaloid K5 | 332.1862 | 9.5233 |
| 4-(5-Phenyl-2,4- | 333.1854 | 11.4302 |
| pentadien-1- | | |
| yl)tetracyclo[5.4.0.02, | | |
| 5.03,9]undec-10- | | |
| ene-8-carboxylic | | |
| acid | | |
| Nb-(3- | 334.1919 | 10.1235 |
| Indolylmethyl)-5- | | |
| methoxy-Nb- | | |
| methyltryptamine | | |
| 2,6-Dimethyl-7- | 335.207 | 9.6611 |
| octene-1,6-diol: 8-O- | | |
| β-D- | | |
| Glucopyranoside | | |
| 2-Benzyl-3-tropanol; | 336.1963 | 11.9508 |
| (1RS,2SR,3RS)- | | |
| form: Benzoyl | | |
| Pseudovincadifformine: | 337.1916 | 4.8866 |
| 15,20-Didehydro | | |
| 8-Methyl-8- | 338.1967 | 9.988 |
| azabicyclo[3.2.1]octane- | | |
| 3,6,7-triol; (1R*, | | |
| 3S*,6S*,7R*)- | | |
| form: 3,6-Di-O- | | |
| tigloyl | | |
| ribo-Hexos-3-ulose; | 339.1807 | 6.1217 |
| a-D-Furanose- | | |
| form: 1,2:5,6-Di-O- | | |
| cyclohexylidene | | |
| 4-[2-(3,5-Dimethyl- | 340.176 | 9.4625 |
| 2-oxocyclohexyl)-2- | | |
| hydroxyethyl]-2,6- | | |
| piperidinedione9C,I; | | |
| (1S,3S,5S,aR)- | | |
| form: 5R-Acetoxy | | |
| 3,4-Dihydro-3,4,5,6- | 341.1389 | 3.32 |
| tetramethoxy-2- | | |
| phenyl-2H-furo[2,3- | | |
| h]-1-benzopyran; | | |
| (2R*,3S*,4R*)- | | |
| form: 3-Demethoxy | | |
| Isocorypalmine | 342.1705 | 6.7069 |
| Sorbistin C | 343.1716 | 5.4612 |
| Gelsedine: 14R- | 345.1814 | 7.4848 |
| Hydroxy | | |
| Suaveoline‡: N—Ac | 346.1919 | 7.0088 |
| Pseudophrynamine | 347.1971 | 7.9188 |
| 258A; (−)-form: 5,6- | | |
| Dimethoxy, 4′- | | |
| carboxylic acid, Me | | |
| ester | | |
| Mahanine; (?)-form: | 348.1963 | 8.1986 |
| Deoxy, ? | | |
| 1,6,14-Trihydroxy- | 349.2015 | 10.1267 |
| 16-kaurene-7,15- | | |
| dione | | |
| Fawcettiine; (−)- | 350.2331 | 13.3597 |
| form: Ac | | |
| Cryptocaryic acid | 351.2324 | 9.0765 |
| Stemospironine | 352.2124 | 12.3883 |
| Ajmalicine; (−)- | 353.1865 | 5.9774 |
| form: 20-Epimer | | |
| ribo-Hexos-3-ulose; | 354.1916 | 8.7548 |
| a-D-Furanose- | | |
| form: 1,2:5,6-Di-O- | | |
| cyclohexylidene, | | |
| oxime | | |
| Colletochlorin A; (E)- | 355.1312 | 0.5732 |
| form: 6′-Ketone | | |
| Rutacridone: 1′,2′- | 356.1498 | 3.3166 |
| Dihydro, 1′- | | |
| hydroxy, 2′-methoxy | | |
| Xanthoascin | 357.1603 | 3.8544 |
| Indicine‡: 3′-Ac, N- | 358.1866 | 5.4354 |
| oxide | | |
| Kouminidine | 359.1971 | 8.2548 |
| Lincosamine; a- | 360.2022 | 8.4871 |
| Pyranose-form: 7- | | |
| Me, 1,2:3,4-di-O- | | |
| isopropylidene, N—Ac | | |
| Cypholophine: O—Ac | 361.2127 | 10.7672 |
| Murrayamine F | 362.212 | 9.3582 |
| 23-Oxo- | 363.196 | 6.911 |
| 3,5,7,9,11,13,15,17,19, | | |
| 21- | | |
| tetracosadecaenoicacid | | |
| Consiculine | 364.2124 | 9.8174 |
| 3-(1,1-Dimethyl-2- | 365.2116 | 7.1545 |
| propenyl)-8-(3- | | |
| methyl-2- | | |
| butenyl)xanthyletin | | |
| Tetrahydropentoxyline | 367.1505 | 1.4747 |
| 4,18:8,13-Diepoxy- | 367.212 | 0.2823 |
| 6,19-dihydroxy- | | |
| 15,16-clerodanolide | | |
| Ajmalicine; (−)- | 368.21 | 7.0659 |
| form: 20-Epimer, Nb- | | |
| Me | | |
| Madindoline A | 370.2018 | 5.9655 |
| Sucrose: 1′,6′-Di- | 371.1553 | 2.5129 |
| Me | | |
| Adenosine,, N6-(2- | 372.1672 | 2.4323 |
| Methylbenzyl) | | |
| 3,4-Bis(3,4- | 373.2015 | 5.3175 |
| dimethoxybenzyl)tetrahydrofuran | | |
| Dendrowardine: | 374.2098 | 6.8755 |
| Chloride | | |
| Wedeliasecokaurenolide: | 375.2171 | 9.416 |
| 3β-Acetoxy | | |
| Acsine | 376.2124 | 7.4235 |
| 1,5,6- | 377.2328 | 8.233 |
| Vouacapanetriol; | | |
| (1a,5a,6β)-form: 6- | | |
| Ac | | |
| Heteratisine: O-De- | 378.228 | 9.7266 |
| Me | | |
| Isohumulinone A | 379.212 | 6.3474 |
| 16-Pentacosene-2,4- | 379.3576 | 0.5805 |
| dione | | |
| Julandine | 380.2225 | 10.3363 |
| 2-Hydroxy-1(10)- | 381.2066 | 3.5597 |
| oplopene-4,9-dione; | | |
| 2a-form: O—C amoyl | | |
| Septicine; (R)-form: | 382.2018 | 5.7302 |
| 7-Demethoxy, N- | | |
| oxide(β-) | | |
| 11,12-Epoxy- | 383.207 | 1.3188 |
| 2,5,7,9,10- | | |
| pentahydroxy-4(20)- | | |
| taxen-13-one | | |
| Syneilesine | 384.2022 | 5.0981 |
| 1-O- | 385.1498 | 0.9517 |
| Coumaroylglycerol; | | |
| (S,E)-form: 4′- | | |
| Deoxy, 2-O-β-D- | | |
| glucopyranoside | | |
| Glaucamine: 8- | 386.1603 | 4.433 |
| Epimer | | |
| 21-Hydroxypregn-4- | 387.2171 | 7.7489 |
| ene-3,11,20-trione: | | |
| Ac | | |
| Gynocardin: Hexa- | 388.1971 | 6.152 |
| Me ether | | |
| 3,14,19- | 389.2328 | 7.3322 |
| Trihydroxycarda- | | |
| 5,20(22)-dienolide; | | |
| (3β,14β)-form | | |
| Protoemetine; (−)- | 390.228 | 7.3171 |
| form: Alcohol, O9- | | |
| de-Me, di-O—Ac | | |
| 1,6,14-Trihydroxy- | 391.212 | 3.9417 |
| 16-kaurene-7,15- | | |
| dione; (ent- | | |
| 1β,6a,14a)-form: 6- | | |
| Ac | | |
| Roquefortine: 3a,12- | 392.2086 | 6.7164 |
| Dihydro | | |
| Kopsanol: 22- | 393.2178 | 6.0697 |
| Epimer, Na,O-di- | | |
| Ac | | |
| Aurachin G | 394.2382 | 8.5441 |
| 3,10-Epoxy-1,3,8- | 395.207 | 3.9923 |
| trihydroxy-4,11(13)- | | |
| germacradien-12,6- | | |
| olide; (1β,3a,4Z, | | |
| 6a,8β)-form: 8-(2- | | |
| Methylpropanoyl), | | |
| 1,3-di-Me ether | | |
| Melovinone | 396.2175 | 5.8852 |
| Karatavic acid | 397.2015 | 2.3048 |
| Sorbistin A; | 398.2138 | 4.4772 |
| Sorbistin A1 | | |
| N,N- | 400.1621 | 0.8917 |
| Dimethyladenosine: | | |
| 2′-Benzoyl | | |
| 1,4,6,12,18,19- | 401.2175 | 5.3913 |
| Hexahydroxy-13- | | |
| cleroden-15,16-olide | | |
| Alpinigenine; (+)- | 402.1916 | 4.6572 |
| form: 8-Epimer, Me | | |
| ether, N-de-Me | | |
| Zuelanin | 403.2484 | 7.5035 |
| Dammar-24-ene- | 404.2437 | 6.3414 |
| 3,20,26-triol; | | |
| Danomycin B: 11S- | | |
| Hydroxy, 14,15- | | |
| didehydro | | |
| Aurachin E | 405.2542 | 7.5976 |
| 2-Phenyl-1,5,9- | 406.2494 | 7.7306 |
| triazacyclotridecan- | | |
| 4-one; (S)-form: N9- | | |
| E-C amoyl | | |
| 1,3,5,14- | 407.2433 | 5.8619 |
| Tetrahydroxycard- | | |
| 20(22)-enolide | | |
| Butirosamine: 5- | 408.2458 | 6.7659 |
| Deoxy | | |
| 7,20-Epoxy-16- | 409.2226 | 2.6605 |
| kaurene-1,6,7,15,19- | | |
| pentol; (ent- | | |
| 1β,6a,7aOH,15a)- | | |
| form: 19-Ac | | |
| Acidissiminol: 6′,7′- | 410.2331 | 4.4289 |
| Epoxide | | |
| Cudratricusxanthone | 411.1807 | 1.0203 |
| A: 2-Me ether | | |
| Sorbistin A; | 412.2295 | 4.2085 |
| Sorbistin A2 | | |
| Austalide K | 413.2328 | 1.8991 |
| Tryptophyllin L 1.1 | 414.2505 | 5.7426 |
| 1,3,6,7- | 415.1757 | 2.0103 |
| Tetrahydroxy-2,8- | | |
| diprenylxanthone: | | |
| 2″,3″-Dihydro, 3″- | | |
| hydroxy | | |
| Narceine: 8- | 416.1709 | 1.9574 |
| Demethoxy | | |
| 2-Methyl-6- | 417.2124 | 5.5841 |
| methylene-7-octene- | | |
| 2,3-diol: 2-O-(2-O- | | |
| Acetyl-β-D- | | |
| glucopyranoside), 3- | | |
| Ac | | |
| Rinamycin | 418.2441 | 5.5064 |
| 6,19-Dihydroxy- | 419.2433 | 5.36 |
| 4(18),13- | | |
| clerodadien-16,15- | | |
| olide; (ent-6β)- | | |
| form: 6,19-Di-Ac | | |
| Antibiotic U 62162 | 420.2386 | 4.778 |
| Kazinol L | 421.2379 | 6.2263 |
| Graciline: 6β,16β- | 422.2542 | 5.6972 |
| Dimethoxy | | |
| N—(N-L-Arginyl-D- | 423.2356 | 4.1607 |
| allothreonyl)-L- | | |
| phenylalanine | | |
| Butirosamine | 424.2407 | 5.0541 |
| Alviminine | 425.2076 | 1.4163 |
| Aconitane- | 426.2492 | 4.1277 |
| 1,4,6,7,8,14,16- | | |
| heptol; | | |
| (1a,5β,6β,14a,16β)- | | |
| form: O6,O16-Di- | | |
| Me, N-Et | | |
| Lunariamine | 428.2549 | 3.4515 |
| Jacmaia incana | 430.1866 | 1.2255 |
| Alkaloid | | |
| Piericidin; Piericidin | 430.2957 | 3.3447 |
| A2 | | |
| Nummularine F: | 431.2658 | 6.3972 |
| Dihydro | | |
| Higenamine; (S)- | 432.2022 | 2.1527 |
| form: O | | |
| Agerasanin | 433.2379 | 3.8125 |
| Sisomicin B | 434.2615 | 5.5761 |
| 7,11-Dihydroxy-16- | 435.2383 | 4.0529 |
| oxo-17-spongianal; | | |
| (7β,11β)-form: Di- | | |
| Ac | | |
| Graciline: 7-Deoxy, | 436.2699 | 5.1114 |
| 6β,16β,18- | | |
| trimethoxy | | |
| Minovincinine; (−)- | 437.244 | 2.4406 |
| form: O-(3-Methyl- | | |
| 2-butenoyl) | | |
| 4-Methylaconit-2- | 438.2492 | 4.1883 |
| ene- | | |
| 1,6,7,8,14,16,18- | | |
| heptol; | | |
| (1a,5β,6β,14a,16β)- | | |
| form: O16,O18-Di- | | |
| Me, N-Et | | |
| Malyngamide F | 440.2567 | 3.2813 |
| Antibiotic SF 1306B | 442.2414 | 3.6923 |
| Quinine,: O-(2- | 445.2127 | 0.5441 |
| Hydroxybenzoyl) | | |
| 2,3- | 445.2954 | 1.507 |
| Dihydroxyspirost- | | |
| 9(11)-en-12-one | | |
| Apiosporamide: N- | 446.2179 | 1.633 |
| Hydroxy | | |
| Piericidin; Piericidin | 446.2906 | 1.1425 |
| A1: 10′-Me ether, | | |
| 11′,12′-epoxide | | |
| 16-Kaurene-3,7,18- | 447.2746 | 4.5053 |
| triol; (ent-3β,7a)- | | |
| form: Tri-Ac | | |
| Sarcotragin A: N- | 448.2699 | 4.9899 |
| De-(2-phenylethyl), | | |
| N-(carboxymethyl) | | |
| Retinoic acid; (13Z)- | 449.2692 | 5.2138 |
| form: 2-(4- | | |
| Methoxyphenyl)-2- | | |
| oxoethyl ester | | |
| 4-Methylaconitane- | 450.2492 | 5.3558 |
| 1,5,6,7,8,14,16- | | |
| heptol; | | |
| (1a,5β,6β,14a,16β)- | | |
| form: 6-Ketone, 7,8- | | |
| methylene ether, O1, | | |
| O14,O16-tri-Me, | | |
| N-Me | | |
| Encecanescin | 451.2484 | 2.9735 |
| 4-Methylaconitane- | 452.2648 | 3.404 |
| 1,6,7,8,14,16,18- | | |
| heptol; | | |
| (1a,5β,6β,14a,16β)- | | |
| form: 14-Ketone, O6, | | |
| O16,O18-tri-Me, | | |
| N-Et | | |
| Ajmalinimine: Ac | 453.2389 | 1.9355 |
| Seldomycin 3 | 454.2513 | 4.2423 |
| Deoxymalyngamide | 456.2517 | 2.252 |
| C: 4a,9a-Epoxide | | |
| CryptoechinulinG | 458.2807 | 3.5949 |
| CaleahymenoneA | 461.1964 | 2.0709 |
| Aeruginosin 298A: | 462.2604 | 4.1614 |
| N1-De(argininolyl) | | |
| 3,7-Dimethyl-2,6- | 463.2543 | 3.6228 |
| octadien-1-ol; (E)- | | |
| form: O-[a-L- | | |
| Rhamnopyranosyl-(1?6)- | | |
| β-D- | | |
| glucopyranoside] | | |
| Antibiotic I-SKB2 | 464.272 | 5.2055 |
| Erinacine G | 465.2488 | 2.2185 |
| 4-Methylaconitane- | 466.2805 | 3.3487 |
| 1,6,7,8,14,16,18- | | |
| heptol; | | |
| (1a,5β,6β,14a,16β)- | | |
| form: 14-Ketone, O1, | | |
| O6,O16,O18- | | |
| tetra-Me, N-Et | | |
| Fissistin | 467.2433 | 1.7129 |
| Nebmycin T | 468.2669 | 3.2133 |
| Hetidine: 3-O-(2- | 470.2542 | 2.7493 |
| Methylpropanoyl), | | |
| 2-Ac | | |
| 4,6′- | 472.3063 | 5.3925 |
| Anhydrooxysporidinone | | |
| Piericidin; Piericidin | 474.3219 | 7.899 |
| A3: 10′-Me ether, | | |
| 11′,12′-epoxide | | |
| 17,28- | 475.3212 | 5.1775 |
| Dotriacontadiene- | | |
| 2,4,6,15,31- | | |
| pentayne-1,8,30-triol | | |
| Ipanguline D3: 7-O- | 476.2284 | 1.0796 |
| (Phenylacetyl), | | |
| 2″,3″-di-Ac | | |
| Austalide H | 477.2488 | 1.4291 |
| Epothilone C | 478.2627 | 2.7856 |
| 4,18:8,13-Diepoxy- | 479.2645 | 2.2494 |
| 6,19-dihydroxy- | | |
| 15,16-clerodanolide; | | |
| (ent-4β,6β,8a,12R)- | | |
| form: 6- | | |
| (Methylpropanoyl), | | |
| 19-Ac | | |
| CytochalasinB | 480.275 | 3.077 |
| Mitraspecine | 481.2702 | 2.0038 |
| Malyngamide F: O—Ac | 482.2673 | 3.1706 |
| GomisinR: Angeloyl | 483.2019 | 0.5452 |
| Discodermide | 483.2495 | 0.3196 |
| Kanamycin B | 484.2618 | 2.1103 |
| Roseotoxin S | 486.2815 | 2.5199 |
| 4-Methylaconit-2- | 490.2805 | 5.1248 |
| ene-1,6,7,8,14,16- | | |
| hexol; | | |
| (1a,6β,14a,16β)- | | |
| form: 7,8-Methylene | | |
| ether, O1,O14,O16- | | |
| tri-Me, N-Et, 6- | | |
| Ac | | |
| Mauritine C | 491.2658 | 2.5406 |
| 4-Methylaconitane- | 492.2961 | 4.601 |
| 1,8,14,16,18-pentol; | | |
| (1a,14a,16β)-form: | | |
| O16,O18-Di-Me, N- | | |
| Et, 8,14-di-Ac | | |
| Ethyl glucoside; a- | 493.259 | 1.8592 |
| D-Pyranose-form: | | |
| 3,4,6-Tribenzyl, 2- | | |
| Me | | |
| 4-Methylaconitane- | 494.2754 | 2.4603 |
| 1,5,6,7,8,14,16- | | |
| heptol; | | |
| (1a,5β,6β,14a,16β)- | | |
| form: 7,8-Methylene | | |
| ether, O1,O14,O16- | | |
| tri-Me, N-Me, 6- | | |
| Ac | | |
| Cinchophyllamine: | 495.276 | 1.5647 |
| 3-Epimer, 4′,17- | | |
| didehydro | | |
| 4-Methylaconitane- | 496.291 | 2.3981 |
| 1,6,7,8,14,16,18- | | |
| heptol; (1a,5β,6β, | | |
| 14a,16β)-form: O6, | | |
| O16,O18-Tri-Me, | | |
| N-Et, 14-Ac | | |
| Pregn-5-ene- | 497.2903 | 2.1408 |
| 3,12,14,17,20- | | |
| pentol; | | |
| (3β,12β,14β,17βOH, | | |
| 20S)-form: 12-C | | |
| amoyl | | |
| Spermidine: N,N″- | 498.2604 | 2.5626 |
| Bis(4-hydroxy-3- | | |
| methoxyc amoyl) | | |
| Bisvertinoquinol | 499.2332 | 0.7776 |
| 3,7- | 500.3046 | 2.8394 |
| Dihydroxycholan- | | |
| 24-oicacid; | | |
| (3a,5β,7a)-form: N- | | |
| (2-Sulfoethyl)amide | | |
| Nephilatoxin 501 | 502.3142 | 5.0507 |
| Patrinoside: 10-Ac | 505.2285 | 0.8792 |
| Epothilone I1 | 506.294 | 3.5276 |
| Marine | 507.2859 | 2.1846 |
| Streptomyces | | |
| C30H38N2O5 | | |
| lactam | | |
| Antibiotic FR | 508.291 | 2.9851 |
| 901465: 2-Deoxy | | |
| Spirost-25(27)-ene- | 509.275 | 1.7586 |
| 1,2,3,4,5,6,7-heptol; | | |
| (1β,2β,3β,4β,5β,6β,7a)- | | |
| form: 6-Ketone | | |
| Penochalasin C | 510.2756 | 2.5594 |
| Spirost-25(27)-ene- | 511.2907 | 2.3855 |
| 1,2,3,4,5,6,7-heptol | | |
| Cyclozoanthamine | 512.3012 | 2.4028 |
| Achyroclinopyrone; | 513.2852 | 1.3431 |
| 3″- | | |
| Methylachyroclinopyrone | | |
| Bisparthenolidine | 514.3168 | 2.9085 |
| Ciliatocholicacid | 516.309 | 2.8308 |
| 3,14-Dihydroxycard- | 518.3481 | 16.2294 |
| 20(22)-enolide; | | |
| (3β,5a,14β,17a)- | | |
| form: 3-O-(4- | | |
| Amino-2,4,6- | | |
| trideoxy-3-O- | | |
| methyl-a-L-arabino- | | |
| hexopyranoside) | | |
| Swertiabisxanthone I | 519.0563 | 0.299 |
| Cosmosiin: 6″-O- | 519.1138 | 0.2977 |
| Malonyl | | |
| Spirost-5-en-3-ol; | 519.3474 | 7.8037 |
| (3β,25R)-form: | | |
| Benzoyl | | |
| Cadabicine: N16,O- | 520.2447 | 0.8086 |
| Di-Ac | | |
| 6,9-Dihydroxy-4,7- | 521.2598 | 1.6031 |
| megastigmadien-3- | | |
| one; (6S,7E,9R)- | | |
| form: 7,8-Dihydro, | | |
| 9-O-[β-D- | | |
| apiofuranosyl-(1?6)- | | |
| β-D- | | |
| glucopyranoside] | | |
| Epothilone B: 21- | 522.2889 | 2.8695 |
| Methyl | | |
| Nummularine E | 523.292 | 2.9878 |
| Cephaeline; (−)- | 524.3124 | 3.5311 |
| form: N- | | |
| (Methylaminocarbonyl) | | |
| Nummularine E: | 525.3077 | 2.4599 |
| Dihydro | | |
| Malyngamide I: Ac | 526.2935 | 3.9897 |
| Thaimycin B | 527.3121 | 1.9051 |
| Chlamydocin: | 529.3026 | 0.1844 |
| Deepoxy, 7′R- | | |
| hydroxy, | | |
| stereoisomer(?) | | |
| 5,6-Epoxy-3,11- | 529.3529 | 0.4017 |
| dihydroxyergost-22- | | |
| en-1-one; | | |
| (3β,5β,6β,11a,22E, | | |
| 24R)-form: Di-Ac | | |
| Nemorosone: 13- | 533.3267 | 1.0196 |
| Hydroxy, 4-Me ether | | |
| 2,3,11,14,20,22- | 535.2907 | 2.898 |
| Hexahydroxy-6- | | |
| oxostigmasta- | | |
| 7,24(28)-dien-29,25- | | |
| olide | | |
| 4-Methylaconitane- | 536.3223 | 3.7055 |
| 1,6,8,14,16,18- | | |
| hexol; | | |
| (1a,5β,6a,14a,16β)- | | |
| form: O1,O6,O16, | | |
| O18-Tetra-Me, | | |
| N-Et, 8,14-di-Ac | | |
| 2,3,5,14,20,29- | 537.3063 | 2.5902 |
| Hexahydroxy-6- | | |
| oxostigmast-7-en- | | |
| 26,22-olide | | |
| Butirosin A: 3′,4′- | 538.3088 | 3.6418 |
| Dideoxy, 6′-N-Me | | |
| Crocacin A | 539.3121 | 3.309 |
| 4-Methylaconitane- | 541.3277 | 2.8934 |
| 1,8,14,16,18-pentol; | | |
| (1a,14a,16β)-form: | | |
| O1,O14,O16-Tri- | | |
| Me, N-Et, 18-O-(2- | | |
| aminobenzoyl) | | |
| Thiobinupharidine: | 543.2892 | 0.939 |
| 6R,6′R-Dihydroxy, | | |
| S-oxide (S-) | | |
| Viopurpurin | 545.1084 | 0.1237 |
| Riboflavine,,: | 545.1883 | 0.2773 |
| 2′,3′,4′,5′-Tetra-Ac | | |
| Plastatin | 546.1036 | 0.1091 |
| 2-Amino-6- | 546.1683 | 0.1531 |
| (hydroxymethyl)- | | |
| 4(1H)-pteridinone: | | |
| O-[4-O-Methyl-a- | | |
| D- | | |
| glucuronopyranosyl- | | |
| (1?6)-β-D- | | |
| galactopyranoside] | | |
| Borrelidin: Ac, Me | 546.3431 | 11.1601 |
| ester | | |
| CytochalasinL | 548.2648 | 1.0871 |
| Ingenol: 3- | 549.3216 | 3.0298 |
| (2,4,6,8,10- | | |
| Tetradecapentaenoyl) | | |
| Petunianine B | 550.3168 | 4.8564 |
| 3,5,14- | 551.322 | 3.1431 |
| Trihydroxycard- | | |
| 20(22)-enolide; | | |
| (3β,5β,14β)-form: 3- | | |
| O-(3-O-Methyl-β- | | |
| D-fucopyranoside) | | |
| 4-Methylaconitane- | 552.3325 | 5.0089 |
| 1,8,14,16,18-pentol; | | |
| (1a,14a,16β)-form: | | |
| O1,O16,O18-Tri- | | |
| Me, N-Et, 14-O-E- | | |
| c amoyl | | |
| Toxiferine I: | 553.3331 | 2.2655 |
| Bis(deoxy), di-N- | | |
| de-Me | | |
| Acosmine: O- | 554.323 | 3.6419 |
| (3,4,5- | | |
| Trimethoxybenzoyl) | | |
| Stypotriol: Tri-Ac | 555.3322 | 2.9543 |
| 4-Methylaconitane- | 556.3274 | 2.989 |
| 1,8,14,16,18-pentol; | | |
| (1a,14a,16β)-form: | | |
| O8,O14,O16-Tri- | | |
| Me, N-Et, 18-O-(2- | | |
| methoxybenzoyl) | | |
| Integerressine: | 557.3128 | 0.9621 |
| Dihydro | | |
| 12-Hydroxy-20,24- | 557.3842 | 0.3375 |
| dimethyl-24-oxo-16- | | |
| scalaren-25-al; | | |
| (12a)-form: 12-(3- | | |
| Propanoyloxybutanoyl) | | |
| Nummularine P | 558.3291 | 1.7272 |
| 22,25-Epoxy-24- | 563.3584 | 6.8147 |
| methylfurostane- | | |
| 2,3,11,20-tetrol; | | |
| (2a,3a,5a,11β,16β,20R, | | |
| 22S,24S)-form: | | |
| 2,3-Di-Ac | | |
| CytochalasinB: Di- | 564.2961 | 2.3745 |
| Ac | | |
| Nummularine E: O—Ac | 565.3026 | 1.8598 |
| Protoverine: 14,15-O- | 566.3329 | 3.0186 |
| Isopropylidene | | |
| Milbemycin IV: 27- | 567.3322 | 2.6834 |
| Oxo, 5-ketone | | |
| Penitrem C: | 568.3427 | 5.1785 |
| Dechloro | | |
| Spirostane-2,3,5,6- | 569.3478 | 4.0927 |
| tetrol; | | |
| (2a,3β,5a,6β,25R)- | | |
| form: O2-Benzoyl | | |
| Sativanine D | 570.3291 | 4.1282 |
| Chamone I | 571.3787 | 3.6748 |
| Veratramine: 20- | 572.3587 | 4.0824 |
| Epimer, 23-O-β-D- | | |
| glucopyranoside | | |
| Antibiotic IC 202A | 573.3975 | 1.2652 |
| DiscarineA | 574.3393 | 3.3874 |
| 3,6-Cevanediol; | 576.39 | 2.3749 |
| (3β,5a,6β,17β,25β)- | | |
| form: 3-Ketone, 6-O- | | |
| β-D- | | |
| glucopyranoside | | |
| 4-Methylaconitane- | 578.3329 | 6.6494 |
| 1,6,7,8,10,14,16- | | |
| heptol; | | |
| (1a,6β,14a,16β)- | | |
| form: 7,8- | | |
| Methylene, 1,16-di- | | |
| Me ether, N-Et, 14- | | |
| O-(2R- | | |
| methylbutanoyl), 6- | | |
| Ac | | |
| 3,5,14- | 579.3169 | 2.2603 |
| Trihydroxycard- | | |
| 20(22)-enolide; | | |
| (3β,5β,14β)-form: 3- | | |
| O-(6-O-Acetyl-2- | | |
| deoxy-β-D-lyxo- | | |
| hexopyranoside) | | |
| Apicidin: Pyrrolidine | 580.3499 | 3.366 |
| analogue, N- | | |
| demethoxy | | |
| 3,14,16- | 581.3326 | 2.3822 |
| Trihydroxycard- | | |
| 20(22)-enolide; | | |
| (3β,5β,14β,16β)- | | |
| form: 3-O-(2,3-Di- | | |
| O-methyl-β-D- | | |
| glucopyranoside) | | |
| Nodulisporic acid D: | 582.3583 | 4.5452 |
| 7-Ketone | | |
| Thiobinupharidine: | 583.3569 | 4.5121 |
| 6?,6′?-Diethoxy | | |
| Penitrem C: | 584.3376 | 3.2776 |
| Dechloro, 23a,24a- | | |
| epoxide | | |
| Sampsonione I | 585.358 | 4.5873 |
| Nummularine P: N?- | 586.3241 | 2.2162 |
| Formyl | | |
| Matopensine: 18- | 587.3386 | 0.5469 |
| Hydroxy | | |
| Nidohottin: 6- | 587.3948 | 0.3703 |
| Hydroxy | | |
| 6,14,22,30,32- | 587.4523 | 0.6341 |
| Pentahydroxy- | | |
| 8,16,24- | | |
| tritriacontanetrione | | |
| Angiolam A | 588.39 | 2.7501 |
| Annodienin | 589.4832 | 1.8561 |
| Petisine: 3-O-β-D- | 590.3693 | 6.8908 |
| Glucopyranosyl | | |
| Sciadoline | 591.2495 | 0.3323 |
| Germine: | 592.4002 | 4.9335 |
| 3,4,7,15,16-Penta- | | |
| Ac | | |
| Santiaguine | 593.3855 | 2.4706 |
| Rhizoxin: 2,3:11,12- | 594.3431 | 7.7813 |
| Bis(deepoxy), | | |
| 2,3,11,12- | | |
| tetradehydro | | |
| Stercobilin | 595.3495 | 4.7078 |
| Kahalalide D | 596.356 | 3.753 |
| 7-Labdene-2,15-diol; | 597.3427 | 2.044 |
| (2a,13?)-form: 2-Ac, | | |
| 15-O-(3,4- | | |
| diacetoxyc amoyl) | | |
| CirramycinA1 | 598.3591 | 2.9365 |
| Tenuilobine | 598.5747 | 0.2121 |
| Toxiferine I: | 599.375 | 3.3652 |
| Monodeoxy | | |
| Icadamide B | 600.3747 | 2.0696 |
| Vilangin | 601.374 | 2.4659 |
| Tetronasin,, | 602.3818 | 2.0796 |
| 12-Oleanene-3,28- | 603.4049 | 0.3264 |
| diol; 3β-form: 28- | | |
| Aldehyde, 3-(3,4- | | |
| dihydroxy-E-c | | |
| amoyl) | | |
| 3,16-Dihydroxy-24- | 605.3842 | 1.826 |
| methylenelanosta- | | |
| 7,9(11)-dien-21- | | |
| oicacid; (3β,16a)- | | |
| form: 3-O-(4- | | |
| Hydroxybenzoyl) | | |
| Roxaticin | 607.3846 | 3.4576 |
| Chymostatin; | 608.3196 | 1.9662 |
| Chymostatin A | | |
| Jolantinine | 609.3328 | 2.0671 |
| Boophiline | 610.3777 | 2.9036 |
| 3,14,22,25- | 611.3795 | 2.9019 |
| Tetrahydroxycholest- | | |
| 7-en-6-one; | | |
| (3β,5a,22R)-form: | | |
| 3-O-β-D- | | |
| Glucopyranoside | | |
| Zizyphine A | 612.3761 | 4.3315 |
| Eisenia Inhibitory | 613.3826 | 4.099 |
| peptapeptides; EIPP3 | | |
| Toxiferine I | 615.3699 | 2.7267 |
| Calebassine | 617.3855 | 1.334 |
| Nodulisporic acid | 618.3794 | 3.1538 |
| D1: 3″,4′-Dihydro, | | |
| 3″-hydroxy (3′R, | | |
| 4′S-) | | |
| 7,26- | 620.4162 | 2.5329 |
| Dihydroxycholest-4- | | |
| en-3-one; (7a,25R)- | | |
| form: 7-O-(2- | | |
| Acetamido-2-deoxy- | | |
| β-D- | | |
| glucopyranoside) | | |
| Capuvosidine: N- | 621.3804 | 2.3758 |
| De-Me | | |
| 3,4,12,14,16,17,20- | 622.3591 | 4.4564 |
| Cevaneheptol; | | |
| (3β,4a,5a,16β)-form: | | |
| 3,4,16-Tri-Ac | | |
| Ceanothic acid: 3- | 623.3584 | 2.6021 |
| (3,4- | | |
| Dihydroxybenzoyl) | | |
| DestruxinD | 624.3608 | 3.0057 |
| 3,3′,4′,5,7- | 625.374 | 2.3722 |
| Pentahydroxyflavone: | | |
| 3′,4′,5,7-Tetra-Me | | |
| ether, 3-O- | | |
| octadecanoyl | | |
| Apicidin: 8′S- | 626.3917 | 3.4222 |
| Alcohol | | |
| Gentinone A: 7-Ac | 627.3897 | 3.2757 |
| 2,2′,3,3′- | 629.3842 | 2.8746 |
| Tetrahydroxy-β,β- | | |
| carotene-4,4′-dione | | |
| Macrospegatrine | 630.357 | 1.0703 |
| 2,3,19,24- | 631.3846 | 2.0784 |
| Tetrahydroxy-12- | | |
| oleanen-28-oic acid; | | |
| (2a,3β,19a)-form: | | |
| 2,3,23-Tri-Ac | | |
| 3,13-Dihydroxy-11- | 633.4002 | 1.0385 |
| oleanen-28-oicacid; | | |
| (3β,13β)-form: | | |
| 11a,12a-Epoxide, | | |
| 28?13 lactone, 3-O- | | |
| β-D-glucopyranoside | | |
| Aurantinin A | 637.374 | 2.7065 |
| Destruxin D; | 638.3765 | 6.4736 |
| Destruxin D1 | | |
| 1,10(14)- | 639.3744 | 3.9051 |
| Fusicoccadiene- | | |
| 8,9,12,16,19-pentol; | | |
| (3β,6a,7β,8β,9a,11β, | | |
| 12a,18S)-form: O16- | | |
| Me, 12-Ac, 9-O- | | |
| [1,1-dimethyl-2- | | |
| propenyl-(?6)-β-D- | | |
| glucopyranoside] | | |
| Apicidin: 9′- | 640.371 | 3.9872 |
| Hydroxy | | |
| 2,3,23-Trihydroxy- | 641.3689 | 3.1723 |
| 12-oleanen-28-oic | | |
| acid; (2a,3β)-form: | | |
| 23-O-(3,4,5- | | |
| Trihydroxybenzoyl) | | |
| GlabrinA | 643.3819 | 1.95 |
| Ganefromycine | 644.3798 | 1.7634 |
| Gentinone A: | 645.4002 | 1.8989 |
| Deepoxy, 24R,25- | | |
| dihydroxy, 7-Ac | | |
| Taiwaniadduct I: 12- | 647.4311 | 1.01 |
| Me ether, Me ester | | |
| 20,24-Epoxy-6,25- | 649.3952 | 1.7477 |
| dihydroxycycloartane- | | |
| 3,16-dione; | | |
| (6a,20R,24S)-form: | | |
| 6-O-β-D- | | |
| Glucopyranoside | | |
| GlycolipidB | 651.3955 | 2.3026 |
| 6(17),11- | 653.3901 | 1.9195 |
| Jatrophadiene- | | |
| 3,5,7,8,9,14,15- | | |
| heptol; | | |
| (3β,5a,7β,8a,9a,11E, | | |
| 14β,15β)-form: 5,7- | | |
| Bis(2- | | |
| methylpropanoyl), 8- | | |
| (2-methylbutanoyl), | | |
| 14-Ac | | |
| Antibiotic VM | 655.3846 | 2.4633 |
| 44866: 22-Deoxy, | | |
| 13β-(2- | | |
| methylpropanoyloxy) | | |
| Antibiotic WF | 656.3798 | 2.9036 |
| 1360E | | |
| Pregn-5-ene- | 657.385 | 3.3696 |
| 3,14,20-triol; | | |
| (3β,14β,20S)-form: | | |
| 3-O-(6-Deoxy-3-O- | | |
| methyl-β-D- | | |
| galactopyranoside), | | |
| 20-O-β-D- | | |
| glucopyranoside | | |
| Pregna-5,20-dien-3- | 658.3955 | 3.3429 |
| ol; 3β: 3-O-(2- | | |
| Acetamido-3,6-di- | | |
| O-acetyl-2-deoxy-4- | | |
| O-butanoyl-β-D- | | |
| galactopyranoside) | | |
| 22,23- | 659.3795 | 2.5236 |
| Dihydroxymilbemycinal: | | |
| O5-Me, 23-O- | | |
| (2-methylbutanoyl) | | |
| Oleanolic acid 3- | 660.4475 | 3.3584 |
| glycosides; | | |
| Monoglycosides: 3- | | |
| O-(2-Acetamido-2- | | |
| deoxy-β-D- | | |
| glucopyranoside) | | |
| Neurosporaxanthin: | 661.4104 | 1.5448 |
| β-D-Glucopyranosyl | | |
| ester | | |
| 7,26- | 662.4268 | 2.5949 |
| Dihydroxycholest-4- | | |
| en-3-one; (7a,25R)- | | |
| form: 26-Ac, 7-O- | | |
| (2-acetamido-2- | | |
| deoxy-β-D- | | |
| glucopyranoside) | | |
| 3,11,14- | 665.3901 | 2.1564 |
| Trihydroxycard- | | |
| 20(22)-enolide; | | |
| (3β,5β,11a,14β)- | | |
| form: 3-O-[2,6- | | |
| Dideoxy-3-O- | | |
| methyl-β-D-ribo- | | |
| hexopyranosyl- | | |
| (1?4)-2,6-dideoxy-β- | | |
| D-ribo- | | |
| Rosamicin: 2′,3-Di- | 666.3853 | 4.0851 |
| Ac | | |
| Rodiasine: Me | 667.3747 | 1.989 |
| ether, N2, N2′-di- | | |
| Me | | |
| Pyloricidin D: N5- | 668.387 | 1.8976 |
| L-Valyl-L-isoleucyl- | | |
| L-leucyl | | |
| Miraziridine A | 669.3935 | 1.9899 |
| Amphibine E | 671.3921 | 2.4179 |
| 22,23- | 673.3952 | 1.5919 |
| Dihydroxymilbemycinal: | | |
| 23-O-(2- | | |
| Methylhexanoyl) | | |
| 1(19),7,10,12- | 673.5407 | 0.7009 |
| Xenicatetraene- | | |
| 6,14,17,18-tetrol; | | |
| (6a,7Z,10E,12E)- | | |
| form: 17-Eicosanoyl, | | |
| 18-Ac | | |
| Germine: 3,15-Bis-O- | 674.3904 | 2.2429 |
| (2-methyl-2- | | |
| butenoyl) | | |
| Conoduramine: | 675.391 | 1.0494 |
| Demethoxy | | |
| Antascomicin; | 676.406 | 1.701 |
| Antascomicin E | | |
| Ervahanine A: 3′- | 677.3703 | 0.5352 |
| Hydroxy, N-de-Me | | |
| Lanost-9(11)-ene- | 677.4628 | 0.7625 |
| 2,3,24,25-tetrol; | | |
| (2a,3β,5a,24R)- | | |
| form: 2-O-(Methyl | | |
| 3-hydroxy-3- | | |
| methylglutaroyl), 3- | | |
| Ac | | |
| Monamycins; | 678.419 | 2.2302 |
| Monamycin A | | |
| Thiazinotrienomycin | 680.3369 | 0.3971 |
| F: 32,33-Dihydro | | |
| 3,23,28-Trihydroxy- | 680.4373 | 1.5308 |
| 30-norlanost-8-en- | | |
| 29-oic acid; | | |
| (3β,23?)-form: 3-(N- | | |
| Acetyl-β-D- | | |
| glucosaminide) | | |
| GingerglycolipidA: | 681.4061 | 1.8394 |
| 12′,13′,15′,16′- | | |
| Tetrahydro | | |
| Mycinamycin VI: | 682.4166 | 2.8944 |
| 2″-Me ether | | |
| 12-Oleanene- | 683.4006 | 2.9337 |
| 3,16,21,22,23,28- | | |
| hexol; | | |
| (3β,16β,21β,22a)- | | |
| form: 3-O-β-D- | | |
| Glucuronopyranoside | | |
| 3,14-Dihydroxybufa- | 685.4176 | 2.4786 |
| 20,22-dienolide; | | |
| (3β,5β,14β)-form: 3- | | |
| (Arginylpimeloyl) | | |
| a-Substance IB | 686.3989 | 1.1126 |
| Neofolitispate 1 | 686.5108 | 1.284 |
| Ingenol: 13- | 687.4472 | 2.0243 |
| Dodecanoyloxy, 3- | | |
| (2,3- | | |
| dimethylbutanoyl), | | |
| 20-Ac | | |
| Oleandomycin,, | 688.4272 | 2.292 |
| Kabicidin | 689.4112 | 2.6711 |
| Conodurine: N-De- | 691.3859 | 1.6913 |
| Me | | |
| Monamycins; | 692.4347 | 2.4802 |
| Monamycin C | | |
| 16,23- | 693.4214 | 3.0691 |
| Epoxycycloart-7- | | |
| ene-3,15,16,24,25- | | |
| pentol; | | |
| (3β,15a,16aOH,23R, | | |
| 24S)-form: 25-Me | | |
| ether, 24-Ac, 3-O-β- | | |
| D-xylopyranoside | | |
| Melianin B | 695.4159 | 1.3239 |
| Mycinamycin VI: | 696.4323 | 1.4783 |
| 2″,3″-Di-Me ether | | |
| 3,20,25,28- | 697.4163 | 1.6256 |
| Tetrahydroxystigmastane- | | |
| 6,16-dione; | | |
| (3β,5a,20?,24S, | | |
| 28?)-form: 3-O-β- | | |
| D-Glucopyranoside, | | |
| 28-Ac | | |
| Mycinamycin II: 14- | 698.4115 | 1.0581 |
| Deoxy, 3B-O-de- | | |
| Me | | |
| Laidlomycin,, | 699.4319 | 1.7665 |
| Jurubine: N- | 700.4424 | 2.4277 |
| Salicylidene | | |
| 22,23- | 701.4265 | 1.9487 |
| Dihydroxymilbemycina3: | | |
| O5-Me, 23-O- | | |
| (2-methylhexanoyl) | | |
| Antibiotic SUAM | 702.4289 | 2.2276 |
| 20010 | | |
| Antibiotic M 6124 | 704.4023 | 0.3262 |
| Monamycins; | 706.4503 | 0.4297 |
| Monamycin F | | |
| Melianin A: 23- | 709.4315 | 1.2357 |
| Deoxy, 24a-methoxy | | |
| (24R-) | | |
| 3-Hydroxychol-5- | 711.3955 | 1.0622 |
| en-24-oicacid; | | |
| (3β,20S)-form: 3-O- | | |
| (a-L- | | |
| Rhamnopyranosyl- | | |
| β-D- | | |
| glucuronopyranoside), | | |
| Me ester | | |
| Avermectin B1a: 3′- | 715.4057 | 1.4618 |
| O-De-Me, 4′-O- | | |
| deglycosyl | | |
| Glycylmethionylglycylalanylleucylarginyl | 716.4241 | 1.4223 |
| leucin9eCI,: Amide | | |
| Phepropeptin A: 1- | 717.4339 | 1.444 |
| L-Phenylalanine | | |
| analogue | | |
| Spirostane-3,6,23- | 727.4268 | 1.5096 |
| triol; (3β,5a,6a,20S, | | |
| 22S,23R,25S)- | | |
| form: 6-O-[β-D- | | |
| Xylopyranosyl- | | |
| (1?3)-6-deoxy-β-D- | | |
| glucopyranoside] | | |
| Hibispeptin A | 731.3768 | 0.476 |
| Aspochalamin A: | 734.4281 | 1.0298 |
| 17-Deoxy | | |
| Cabufiline: Deepoxy | 735.4121 | 0.5119 |
| Antibiotic 14A | 736.4483 | 1.3835 |
| Conodurine: 3′- | 737.4278 | 1.5477 |
| Methoxy, 19,20β- | | |
| dihydro | | |
| Pepstatins; Pepstatin I | 742.533 | 1.2115 |
| Cholestane- | 745.4738 | 1.4005 |
| 3,6,8,15,24-pentol; | | |
| (3β,5a,6a,15β,24S)- | | |
| form: 24-O-[2,4-Di- | | |
| O-methyl-β-D- | | |
| xylopyranosyl-(1?2)- | | |
| a-L- | | |
| arabinofuranoside] | | |
| Incarvillateine: 5″- | 749.4377 | 1.0874 |
| Methoxy | | |
| Acylsucroses: | 763.4116 | 0.3229 |
| 2,3′,4′,6′-Tetrakis(3- | | |
| methylbutanoyl), 1′- | | |
| (2S- | | |
| methylbutanoyl) | | |
| 3,27-Dihydroxy-12- | 765.4366 | 0.7911 |
| oleanen-28-oic acid; | | |
| 3β: 3-(4-Hydroxy- | | |
| E-c amoyl), 27-(4- | | |
| hydroxy-Z-c amoyl) | | |

TABLE 12
DART TOF-MS characterization of Nettle Extract 5.

| | | Rel |
|---|---|---|
| | Calc. | Abund. |
| Compound Name | Mass | (%) |
| 2-Methyl-2-butenoic | 100.0762 | 2.8895 |
| acid; (E)-form: | | |
| Amide | | |
| 3-Aminodihydro- | 102.0555 | 0.2293 |
| 2(3H)-furanone | | |
| N,N- | 104.0711 | 24.4221 |
| Dimethylglycine | | |
| Diethanolamine | 106.0868 | 0.4015 |
| 2-Amino-4- | 109.0765 | 5.3635 |
| methylpyridine | | |
| Cyclohexanecarboxylicacid: | 110.0969 | 7.9647 |
| Nitrile | | |
| Resorcinol | 111.0922 | 4.4254 |
| Histamine | 112.0874 | 22.5742 |
| Tetramethylammonium(1+): | 114.1094 | 3.2081 |
| Hydrogen | | |
| difluoride | | |
| Proline | 116.0711 | 18.4047 |
| 2- | 118.0868 | 11.6656 |
| Methylaminoacetic | | |
| acid: Et ester | | |
| Threonine | 120.066 | 0.9594 |
| 5-Ethyl-2- | 122.0969 | 0.7951 |
| methylpyridine | | |
| Vitamin B3 | 123.0922 | 56.1574 |
| (niacinamide) | | |
| 6-Methyl-3,5- | 125.0966 | 10.1983 |
| heptadien-2-one; (E)- | | |
| (?)-form | | |
| 6-Acetyl-1,2,3,4- | 126.0919 | 12.45642 |
| tetrahydropyridine | | |
| 4,5- | 127.0871 | 2.5368 |
| Dimethylimidazole: | | |
| N-Me,N′-oxide | | |
| Dichloroacetic acid: | 127.967 | 0.0579 |
| Amide | | |
| Cyclohexanecarboxylicacid: | 128.1075 | 14.0756 |
| Amide | | |
| 2-Amino-4-hexenoic | 130.0868 | 30.122 |
| acid | | |
| N-(2-Methoxy-2- | 131.131 | 4.4992 |
| propenyl)trimethylammonium(1+) | | |
| Leucine | 132.1024 | 11.8343 |
| Thiourea: N,N-Di- | 133.0799 | 1.4143 |
| Et | | |
| 2-Amino-4-hydroxy- | 134.0817 | 3.9575 |
| 3-methylbutanoic | | |
| acid | | |
| 3-Phenyl-2-propen- | 135.081 | 1.5323 |
| 1-ol | | |
| Adenine | 136.061 | 40.0157 |
| Tetramethylpyrazine | 137.1078 | 47.5387 |
| 4,5,6,7-Tetrahydro- | 138.103 | 21.021 |
| 1H-imidazo[4,5-c]pyridine: | | |
| N6-Me | | |
| 2-Pentylfuran | 139.1123 | 23.9565 |
| 9- | 140.1075 | 25.0821 |
| Azabicyclo[3.3.1]nonan- | | |
| 3-one | | |
| Pyracrimycin A; (E)- | 141.1028 | 6.6859 |
| form: 1,2-Dihydro | | |
| 2-Aminoethanol: N,N- | 142.1232 | 24.7655 |
| Di-2-propenyl | | |
| 1-Undecene-5,7,9- | 143.0861 | 8.4831 |
| triyne | | |
| 4-tert-Butyl-2- | 144.1024 | 13.7779 |
| oxazolidinone | | |
| 2,3-Dihydro-3,6- | 145.0501 | 6.7626 |
| dihydroxy-2-methyl- | | |
| 4H-pyran-4-one | | |
| 2-Amino-3- | 146.0817 | 32.1256 |
| hydroxymethyl-3- | | |
| pentenoic acid | | |
| 2-Amino-2- | 147.0922 | 6.6805 |
| phenylpropanoic | | |
| acid; (±)-form: | | |
| Nitrile | | |
| 2-Amino-5- | 148.0973 | 6.9103 |
| hydroxyhexanoic | | |
| acid | | |
| 4-(1- | 149.0966 | 7.2186 |
| Propenyl)phenol; (E)- | | |
| form: Me ether | | |
| 4-Methylbenzoic | 150.0919 | 13.5254 |
| acid: Methylamide | | |
| 2,5-Dimethyl-3- | 151.1235 | 27.5783 |
| propylpyrazine | | |
| 2-Hydroxy-3- | 153.1028 | 26.3931 |
| isopropylpyrazine; | | |
| OH: Me ether | | |
| Histamine: N?-Ac | 154.098 | 21.1022 |
| 4-Hydroxy-2- | 155.1072 | 10.1843 |
| cyclopenten-1-one; | | |
| (R)-form: tert-Butyl | | |
| ether | | |
| Scopine | 156.1024 | 16.9105 |
| 1,3-Dimethyl-2,9- | 157.1228 | 5.7196 |
| dioxabicyclo[3.3.1]nonane; | | |
| (1R,3R)- | | |
| form | | |
| 8-Methyl-8- | 158.1181 | 45.6075 |
| azabicyclo[3.2.1]octane- | | |
| 3,6-diol | | |
| 5-Methyl-2,3- | 159.1133 | 1.9881 |
| hexanedione: | | |
| Dioxime | | |
| 4-Hydroxy-1,1- | 160.0973 | 35.8201 |
| dimethylpyrrolidinium- | | |
| 2-carboxylate; (2S, | | |
| 4R)-form | | |
| 2-Aminobutanoic | 161.1078 | 6.6515 |
| acid; (±)-form: N- | | |
| Ph, nitrile | | |
| Boschniakine; (R)- | 162.0919 | 8.5297 |
| form | | |
| Levoglucosan/Glyogen/ | 163.1082 | 15.8049 |
| Laminarin | | |
| N-[2-(4- | 164.0711 | 80.8028 |
| Hydroxyphenyl)ethenyl]formamide | | |
| 3-Butyl-2,5- | 165.1391 | 40.7331 |
| dimethylpyrazine | | |
| 2-Amino-2- | 166.0868 | 78.0251 |
| phenylpropanoic | | |
| acid | | |
| 1,1,2,2- | 166.8989 | 0.174 |
| Tetrachloroethane | | |
| Synephrine | 168.1137 | 19.6177 |
| Tetramethylpyrazine: | 169.0977 | 29.1022 |
| 1,4-Dioxide | | |
| 9- | 170.1181 | 15.0048 |
| Azabicyclo[3.3.1]nonan- | | |
| 3-one: N-Me, | | |
| N-oxide | | |
| Fumaric acid: | 171.1133 | 5.2675 |
| Bis(dimethylamide) | | |
| 2-Aminoethanol: N, | 172.1337 | 58.4378 |
| N-Di-Et, 2- | | |
| propenoyl | | |
| Octanedioic acid: | 173.129 | 10.0507 |
| Diamide | | |
| 8-Methyl-8- | 174.113 | 19.6385 |
| azabicyclo[3.2.1]octane- | | |
| 3,6,7-triol | | |
| Shikimic acid | 175.1235 | 3.3402 |
| 4-Amino-1-phenyl- | 176.1075 | 14.6922 |
| 1-penten-3-one | | |
| 1,2,6-Trihydroxy-2- | 177.1127 | 11.9515 |
| methyl-4-heptanone | | |
| N-[2-(4- | 178.0868 | 63.145 |
| Hydroxyphenyl)ethenyl]formamide: | | |
| Me | | |
| ether | | |
| 1,2-Bis(2- | 179.1283 | 44.8577 |
| methoxyethoxy)ethane | | |
| 1,2,3,4-Tetrahydro- | 180.1024 | 100 |
| 4,8-dihydroxy-2- | | |
| methylisoquinoline | | |
| 4-Isopropyl-1,2- | 181.1228 | 47.8371 |
| benzenediol: Di-Me | | |
| ether | | |
| 6-Nitro-p-mentha- | 182.1181 | 28.4865 |
| 1(7),2-diene | | |
| Carcinine | 183.1246 | 34.9827 |
| 8-Methyl-8- | 184.1337 | 19.5673 |
| azabicyclo[3.2.1]octan- | | |
| 3-ol; (1RS,3RS)- | | |
| form: Ac | | |
| 5-(Methylthio)- | 184.9587 | 0.0303 |
| 1,2,3-trithiane | | |
| 3,7-Dimethyl-1- | 185.1364 | 13.2568 |
| (methylthio)-2,6- | | |
| octadiene; (E)-form | | |
| 7-(3-Methyl-2- | 186.1282 | 16.9657 |
| butenyl)-1H-indole | | |
| Dihydro-5-(4- | 187.1334 | 9.9905 |
| hydroxy-4- | | |
| methylpentyl)-2(3H)- | | |
| furanone | | |
| 2-Amino-4- | 188.0923 | 28.0381 |
| propylidenepentanedioic | | |
| acid | | |
| Glutamine,,; (S)- | 189.1239 | 6.7908 |
| form: N5-Isopropyl | | |
| 3- | 190.1079 | 16.37 |
| Aminoheptanedioic | | |
| acid; (±)-form: | | |
| Mono-Me ester | | |
| 2,6-Dideoxy-3-C- | 191.1283 | 13.3159 |
| methyl-ribo-hexose; | | |
| a-D-Pyranose-form: | | |
| Me glycoside, 3-Me | | |
| 2-Amino-4- | 192.1058 | 38.1808 |
| mercaptobutanoic | | |
| acid; (±)-form: S- | | |
| Butyl | | |
| 1,2,3,4-Tetrahydro- | 194.1181 | 35.5158 |
| 7-hydroxy-6- | | |
| methoxy-1- | | |
| methylisoquinoline; | | |
| (S)-form | | |
| 11,12,13-Trinor-3,8- | 195.1385 | 32.3332 |
| eudesmanedione | | |
| DL-methyl-m- | 196.1337 | 34.9074 |
| tyrosine | | |
| 2-Isopropyl-3,5- | 197.129 | 40.448 |
| dimethoxy-6- | | |
| methylpyrazine | | |
| Noformicin; (±)- | 198.1355 | 23.7732 |
| form | | |
| 2-(2-Butenylidene)- | 199.1334 | 56.4378 |
| 5-heptene-1,3,4- | | |
| triol; (2Z,3R*,4S*, | | |
| 6Z,8E)-form | | |
| 8-Methyl-8- | 200.1286 | 36.2365 |
| azabicyclo[3.2.1]octane- | | |
| 3,6-diol; (3R, | | |
| 6R)-form: 3-Ac | | |
| 4,6,12- | 201.1279 | 15.8469 |
| Tetradecatriene- | | |
| 8,10-diyn-1-ol; | | |
| (E,E,E)-form | | |
| 9-Mercaptononanoic | 202.1265 | 22.441 |
| acid: S-Me, nitrile, | | |
| S-oxide | | |
| 1,2,3,4-Tetrahydro- | 203.1184 | 9.4108 |
| 1-methyl-β- | | |
| carboline; (?)-form: | | |
| Nb-Oxide | | |
| Carnitine,; (±)-form: | 204.1236 | 13.9749 |
| O—Ac | | |
| 4-methyl-7- | 205.1341 | 12.0857 |
| ethoxycoumarin | | |
| N-(2- | 206.1181 | 29.7676 |
| Hydroxyethyl)camamide: | | |
| N-Me | | |
| Eremopetasinorol: 3- | 207.1385 | 19.2982 |
| Ketone | | |
| 1,2,3,4-Tetrahydro- | 208.1337 | 31.2335 |
| 7-hydroxy-6- | | |
| methoxy-1- | | |
| methylisoquinoline; | | |
| (±)-form: N-Me | | |
| 6,10-Dimethyl-5,9- | 209.1541 | 40.6392 |
| undecadiene-2,8- | | |
| dione | | |
| Elaeokanine E | 210.1494 | 25.0933 |
| Antibiotic A 41-89; | 211.1334 | 41.0746 |
| Antibiotic A 41-89I | | |
| Mescaline | 212.1286 | 22.3478 |
| Cucurbic acid | 213.149 | 29.8308 |
| Hexahydro-7a- | 214.1443 | 34.1362 |
| hydroxy-3H- | | |
| pyrrolizin-3-one; | | |
| (±)-form: (1- | | |
| Ethoxyethyl) ether | | |
| Tsitsikammafuran | 215.1436 | 26.4307 |
| a-Amino-?- | 216.1236 | 34.268 |
| oxooxiraneoctanoic | | |
| acid | | |
| 1,2,3,4-Tetrahydro- | 217.1341 | 13.9593 |
| 1-methyl-β- | | |
| carboline; (R)-form: | | |
| 5-Methoxy | | |
| 1H-Indole-3- | 218.1293 | 39.4872 |
| butanoic acid: | | |
| Hydrazide | | |
| 4,6-Tetradecadiene- | 219.1385 | 21.9967 |
| 8,10-diyne-1,12-diol | | |
| Vitamin B5 | 220.1185 | 39.3615 |
| Penienone | 221.1541 | 25.2139 |
| 2-Amino-2- | 222.1341 | 41.7206 |
| deoxygalactose; a- | | |
| D-Pyranose-form: | | |
| 3,4,6-Tri-Me | | |
| 14-Nor-5- | 223.1698 | 27.0164 |
| protoilludene-7,8- | | |
| diol; (7a,8β)-form | | |
| 6-Amino-7,8- | 224.1511 | 26.4799 |
| dihydro-2- | | |
| hydroxypurine: 6-N- | | |
| (3-Methylbutyl) | | |
| Murexine | 225.1477 | 37.4981 |
| Mescaline: N-Me | 226.1443 | 18.1983 |
| 4,7- | 227.1647 | 44.5658 |
| Megastigmadiene- | | |
| 3,6,9-triol | | |
| 8-Methyl-8- | 228.1599 | 19.7621 |
| azabicyclo[3.2.1]octane- | | |
| 3,6-diol; (3S, | | |
| 6S)-form: 3-O-(2- | | |
| Methylpropanoyl) | | |
| 5,8-Dihydroxy-2,6- | 229.144 | 20.2255 |
| dodecadienoic acid; | | |
| (2Z,5?,6Z,8?)-form | | |
| 2-Pyrrolidineacetic | 230.1392 | 22.7918 |
| acid; (S)-form: N-tert- | | |
| Butyloxycarbonyl | | |
| Verboccidentafuran: | 231.1385 | 12.6457 |
| 2-Oxo | | |
| N,N′-Bis(4- | 231.2548 | 0.4007 |
| aminobutyl)-1,4- | | |
| butanediamine | | |
| 2- | 232.1549 | 34.3479 |
| Aminoheptanedioic | | |
| acid; (±)-form: Di- | | |
| Et ester | | |
| Verboccidentafuran: | 233.1541 | 17.9284 |
| 4a,5a-Epoxide | | |
| 4-(3-Indolyl)-2- | 234.1494 | 39.1328 |
| methyl-1-butanol; (R)- | | |
| form: N-Methoxy | | |
| 7-Isopropyl-1- | 235.1487 | 25.9397 |
| methylphenanthrene | | |
| 2-Amino-2- | 236.1498 | 45.1781 |
| deoxymannose; β- | | |
| D-Furanose-form: | | |
| Me glycoside, 3,5,6- | | |
| tri-Me | | |
| 7-Hydroxy-13-nor- | 237.149 | 30.8446 |
| 3,9-bisaboladiene- | | |
| 2,11-dione | | |
| 1,2,3,4-Tetrahydro- | 238.1443 | 24.1561 |
| 5-hydroxy-6,7- | | |
| dimethoxy-1,2- | | |
| dimethylisoquinoline; | | |
| (±)-form | | |
| isopropyl-B-D- | 239.1647 | 20.1639 |
| thiogalactopyranoside | | |
| Heteromine H: N2, | 240.146 | 16.647 |
| N2-Di-Me | | |
| Putaminoxin B | 241.1803 | 33.6403 |
| 2,4-Undecadiene- | 242.1545 | 17.3149 |
| 8,10-diynoic acid; | | |
| (2E,4E)-form: | | |
| Piperidide | | |
| phosphatidylcholine | 243.1596 | 19.0676 |
| Peganidine: Deoxy, | 244.145 | 18.5463 |
| oxime | | |
| osthole | 245.1501 | 11.5445 |
| 8-Methyl-8- | 246.1494 | 34.5002 |
| azabicyclo[3.2.1]octan- | | |
| 3-ol; (1RS,3RS)- | | |
| form: O-Benzoyl | | |
| 1-Deoxygalactitol; | 247.1545 | 19.0319 |
| D-form: 2,3:4,5-Di- | | |
| O-isopropylidene | | |
| 4-Amino-4,6- | 248.1498 | 24.6995 |
| dideoxy-3-C- | | |
| methylmannose; β- | | |
| D-Pyranose-form: | | |
| Me glycoside, N- | | |
| Me, N—Ac | | |
| 4-shogaol | 249.149 | 23.0833 |
| 10- | 250.1477 | 29.1612 |
| (Methylsulfonyl)decanoic | | |
| acid: Amide | | |
| 1-Hydroxy-5,11(13)- | 251.1647 | 20.6126 |
| eudesmadien-12-oic | | |
| acid | | |
| 1,2,3,4-Tetrahydro- | 252.1599 | 29.104 |
| 5-hydroxy-6,7- | | |
| dimethoxy-1,2- | | |
| dimethylisoquinoline; | | |
| (±)-form: Me ether | | |
| 1,2,3,4-Tetrahydro- | 253.1678 | 14.8082 |
| 6,7,8-trihydroxy-1- | | |
| methylisoquinoline; | | |
| (?)-form: 6,7-Di-Me | | |
| ether, N,N-di-Me | | |
| N-2-Phenylethyic | 254.1545 | 16.2887 |
| amamide; (E)-form: | | |
| 2,3-Dihydro | | |
| Palitantin; (+)-form | 255.1596 | 16.6262 |
| 2- | 255.3052 | 0.9292 |
| Methylheptadecane | | |
| 8-Methyl-8- | 256.1549 | 18.6893 |
| azabicyclo[3.2.1]octane- | | |
| 3,6,7-triol; (1R*, | | |
| 3S*,6S*,7R*)- | | |
| form: 3-O-Tigloyl | | |
| Fumigaclavine B | 257.1654 | 15.9469 |
| 8-Methyl-8- | 258.1705 | 16.1807 |
| azabicyclo[3.2.1]octane- | | |
| 3,6,7-triol; (1R*, | | |
| 3S*,6S*,7R*)- | | |
| form: 3-O-(2- | | |
| Methylbutanoyl) | | |
| 1,8,16- | 259.1698 | 17.9634 |
| Heptadecatriene-4,6- | | |
| diyne-3,10-diol | | |
| Norchalciporol: O- | 260.165 | 17.3305 |
| Propanoyl | | |
| Narbosine A: 1-Me | 261.1702 | 21.6962 |
| glycoside | | |
| Axamide 1: 7,11- | 262.1629 | 19.7487 |
| Didehydro, | | |
| isothiocyanate | | |
| 10-Hydroxy-1,3,5- | 263.1647 | 16.7064 |
| cadinatrien-15-oic | | |
| acid; (7β,10a OH)- | | |
| form: Me ester | | |
| Alkaloid S—F | 264.1599 | 20.6236 |
| Guatambuine | 265.1704 | 13.8381 |
| Brevicolline; (S)- | 266.1657 | 21.6839 |
| form | | |
| 1,7-Diphenyl-6- | 267.1749 | 10.8117 |
| hepten-3-ol | | |
| Codonopsine | 268.1549 | 15.8619 |
| Borrelidin: 12,13- | 269.1654 | 20.2716 |
| Dihydro | | |
| 8-Methyl-8- | 270.1705 | 16.9053 |
| azabicyclo[3.2.1]octane- | | |
| 3,6-diol; (3RS, | | |
| 6RS)-form: 6-O- | | |
| (2- | | |
| Methylpropanoyl), | | |
| 3-Ac | | |
| 5,8-Dihydroxy-2,6- | 271.1545 | 11.5048 |
| dodecadienoic acid; | | |
| (2Z,5?,6Z,8?)- | | |
| form: 8-Ac | | |
| Ipalbidine; (±)-form: | 272.165 | 13.8185 |
| Ac | | |
| 2,3-Dihydro-3,3- | 273.1603 | 12.6858 |
| dimethyl-1H- | | |
| pyrrolo[1,2-a]indole- | | |
| 9-alanine | | |
| Ipanguline D3 | 274.1654 | 15.4241 |
| 12,18- | 275.18 | 26.9341 |
| Heneicosadiene- | | |
| 1,3,8,10,20- | | |
| pentayne; (Z,Z)- | | |
| form: 3,4-Dihydro | | |
| (Z-) | | |
| Calabatine | 276.1963 | 21.1354 |
| 9,11,13,15- | 277.2167 | 78.5458 |
| Octadecatetraenoic | | |
| acid; (9E,11E,13E, | | |
| 15E)-form | | |
| Stenine | 278.212 | 31.7159 |
| 7-Hydroxy-14,15- | 279.2324 | 36.1952 |
| dinor-8(17)-labden- | | |
| 13-one | | |
| Dendrobine: N- | 280.1912 | 22.2332 |
| Oxide | | |
| Brefeldin A | 281.1753 | 13.0586 |
| 8-Methyl-8- | 282.1705 | 15.8001 |
| azabicyclo[3.2.1]octane- | | |
| 3,6-diol; (3R, | | |
| 6R)-form: 3- | | |
| Tigloyl, 6-Ac | | |
| Randainal: 9- | 283.1334 | 2.6449 |
| Alcohol | | |
| 1,2- | 283.2174 | 1.3484 |
| Didehydropseudoaspidospermidine: | | |
| 20- | | |
| Epimer, dihydro | | |
| Amabiline‡ | 284.1862 | 11.5308 |
| 11,12-Epoxy-10(14)- | 285.1702 | 9.7681 |
| oplopene-2,3,4,9- | | |
| tetrol; | | |
| (2β,3a,4?,11?,9a)- | | |
| form | | |
| piperine/cocluarine/laurifoline | 286.1654 | 12.8675 |
| 1,2,8,9- | 287.1858 | 12.5145 |
| Tetrahydroxydihydro- | | |
| β-agarofuran | | |
| Tribenzylamine | 288.1752 | 17.4212 |
| 13,14-Dihydroxy- | 289.1803 | 17.206 |
| 8,11,13- | | |
| podocarpatrien-7- | | |
| one: 13-Me ether | | |
| Oridine | 290.1756 | 17.7556 |
| 8-Hydroxy-17- | 291.196 | 24.6973 |
| octadecene-9,11- | | |
| diynoic acid | | |
| Variotin, JAN | 292.1912 | 16.2422 |
| Ecklonialactone A: | 293.2116 | 20.5747 |
| 6,7-Dihydro | | |
| Capsaicin: Nonanoyl | 294.2069 | 15.8817 |
| analogue | | |
| Aristoteline; (+)- | 295.2174 | 15.4746 |
| form | | |
| 2-(3,7-Dimethyl-2,6- | 296.2014 | 12.1538 |
| octadienyl)-3- | | |
| methyl-4(1H)- | | |
| quinolinone | | |
| Methuenine: 19,20β- | 297.1967 | 3.4956 |
| Dihydro | | |
| Diphthine: 1′-Amide | 298.1879 | 11.1702 |
| Strychnochromine | 299.1759 | 7.3229 |
| Indicine‡: 3′-Epimer | 300.1811 | 11.7864 |
| 12- | 301.1803 | 9.5041 |
| Hydroxyandrosta- | | |
| 1,4-diene-3,17- | | |
| dione; 12β-form | | |
| Indolactam V | 302.1868 | 13.4892 |
| 16-Hydroxyandrost- | 303.196 | 9.715 |
| 4-ene-3,17-dione; | | |
| 16a-form | | |
| Joubertiamine; (R)- | 304.1912 | 15.4968 |
| form: 3′-Methoxy, | | |
| Me ether | | |
| 15,17- | 305.2116 | 18.7985 |
| Dihydroxyandrost-4- | | |
| en-3-one | | |
| Amicetamine | 306.1916 | 16.4049 |
| Azobenzene-4,4′- | 307.0041 | 0.0351 |
| dicarboxylic acid; | | |
| (E)-(?)-form, | | |
| Dichloride | | |
| Acanthoine: | 307.2134 | 15.0981 |
| Tetrahydro | | |
| Spermidine: N ?- | 308.1974 | 18.0838 |
| (3,4-Dihydroxycamoyl) | | |
| 1,6-Dibromo-1,3(8)- | 308.9889 | 0.0503 |
| ochtodadien-4-ol; | | |
| (1E,4S*,6S*)-form | | |
| 3,5-Dihydroxy-3- | 309.014 | 0.045 |
| methylpentanoicacid; | | |
| (R)-form: 5- | | |
| Diphosphate | | |
| Taberpsychine | 309.1967 | 14.0915 |
| Tombozine: N4-Me | 310.2045 | 13.9131 |
| 1-O-C | 311.1131 | 0.381 |
| amoylglucose; β-D- | | |
| Pyranose-(E)-form | | |
| Lachnelluloic acid | 311.2222 | 1.3785 |
| Hetisan-6,11-diol; | 312.1963 | 8.8947 |
| 11?-form: 11-Ketone | | |
| 4- | 313.1803 | 3.9476 |
| Hydroxymethylathrixianone: | | |
| 19- | | |
| Carboxylic acid, Me | | |
| ester | | |
| Martensine A: 10- | 314.1868 | 7.8185 |
| Ketone | | |
| Verecynarmin F: | 315.196 | 7.2568 |
| 11β-Hydroxy | | |
| Androst-4-ene- | 316.1912 | 11.3295 |
| 3,11,17-trione: 17- | | |
| Oxime | | |
| 3,7-Dimethyl-2,6- | 317.1964 | 9.6224 |
| octadien-1-ol; (E)- | | |
| form: O-β-D- | | |
| Glucopyranoside | | |
| Oridine; (−)-form: | 318.2069 | 11.8121 |
| Me ether, N-Me | | |
| 8- | 319.1909 | 10.4147 |
| dehydrogingerdione | | |
| Calabacine | 320.1974 | 13.0315 |
| 3-Hydroxy-17-nor- | 321.2066 | 10.3314 |
| 8-oxo-13-labden-15- | | |
| oic acid; (ent-3β,13Z)- | | |
| form: 3-Ketone | | |
| 8-Methyl-8- | 322.2018 | 13.8792 |
| azabicyclo[3.2.1]octane- | | |
| 3,6-diol; (3R, | | |
| 6R)-form: Ditigloyl | | |
| 4-(3,7-Dimethyl-2,6- | 323.2011 | 8.6985 |
| octadienyl)-3,5- | | |
| dihydroxybiphenyl | | |
| Tombozine: Me | 324.2201 | 10.936 |
| ether, N4-Me | | |
| Hymeglusin | 325.2015 | 4.4389 |
| C-Fluorocurine | 326.1994 | 12.0024 |
| Galeon | 327.1596 | 1.4183 |
| Fortuneine | 328.1912 | 5.0156 |
| Gelsedine | 329.1865 | 4.0555 |
| sinomenin/deoxyharringtonine | 330.2069 | 7.1922 |
| picrocrocin/carnosol | 331.1909 | 5.4425 |
| Bicyclomahanimbicine | 332.2014 | 8.4634 |
| Lysinomycin: De-N- | 333.2138 | 10.0442 |
| lysyl | | |
| 4-[2- | 334.2018 | 10.292 |
| (Acetylmethylamino)ethyl]- | | |
| 4-(4- | | |
| hydroxy-3- | | |
| methoxyphenyl)-2,4- | | |
| cyclohexadien-1- | | |
| one: 2,3,5,6- | | |
| Tetrahydro, Me ether | | |
| 2,6-Dimethyl-7- | 335.207 | 8.8805 |
| octene-1,6-diol: 8-O- | | |
| β-D- | | |
| Glucopyranoside | | |
| 3- | 336.2327 | 10.8856 |
| Greenwayodendrinol; | | |
| 3β-form: 3-Ketone | | |
| 4-(3,7-Dimethyl-2,6- | 337.2167 | 4.8622 |
| octadienyl)-3,5- | | |
| dihydroxybiphenyl; | | |
| (E)-form: Mono-Me | | |
| ether | | |
| Palustrine‡: N8- | 338.2443 | 11.2877 |
| Formyl | | |
| 21-Ethyl-2,6-epoxy- | 339.196 | 3.1213 |
| 1-oxa-2,5,14,17,20- | | |
| cycloheneicosapentaen- | | |
| 11-yn-4-one | | |
| Dysideabenzoxazole | 340.2276 | 11.0597 |
| 3,4-Dihydro-3,4,5,6- | 341.1389 | 0.7058 |
| tetramethoxy-2- | | |
| phenyl-2H-furo[2,3- | | |
| h]-1-benzopyran; | | |
| (2R*,3S*,4R*)- | | |
| form: 3-Demethoxy | | |
| 1,3(15),6- | 341.2116 | 0.7126 |
| Humulatrien-9-ol: | | |
| O-(4- | | |
| Hydroxybenzoyl) | | |
| Hetidine: 3-Deoxy | 342.2069 | 7.9129 |
| 12-Hydroxy-13- | 343.212 | 4.6026 |
| (hydroxymethyl)- | | |
| 3,5,7-trimethyl-2,4- | | |
| tetradecadiene-1,14- | | |
| dioic acid | | |
| Floridimine: 3′-Ac | 344.2073 | 8.8835 |
| Boviquinone 3 | 345.2066 | 7.2488 |
| 7-Isothiocyanato- | 346.2204 | 8.7763 |
| 11(20),14- | | |
| epiamphilectadien- | | |
| 12-ol | | |
| 5-Hydroxy-19- | 347.2222 | 7.2985 |
| vouacapanoic acid; | | |
| 5a-form: Me ester | | |
| Amorphastilbol | 349.2167 | 7.9064 |
| Cryptocaryic acid | 351.2324 | 5.5176 |
| Manzamine C: | 352.2752 | 15.2404 |
| 1?,2,3,4-Tetrahydro | | |
| Citreomontanin | 353.2116 | 3.62 |
| Suaveolindole | 354.2433 | 11.0871 |
| Estra-1,3,5(10)- | 357.2066 | 2.6209 |
| triene-3,17-diol; | | |
| 17a-form: Di-Ac | | |
| Ipanguline D3: 3″- | 358.2229 | 5.923 |
| (2-Methylbutanoyl) | | |
| Siphonodictyoic acid | 359.2222 | 4.8008 |
| Asperparaline A | 360.2287 | 8.8395 |
| 3-Hydroxy-8- | 361.2379 | 8.9316 |
| kempen-6-one; | | |
| (3β,7β)-form: 2β- | | |
| Acetoxy | | |
| Gomandonine | 362.2331 | 10.5555 |
| 12-Hydroxy- | 363.2171 | 5.6332 |
| 8(17),13-labdadien- | | |
| 15,12-olid-19-oic | | |
| acid; (12?)-form: 12- | | |
| Me ether | | |
| 4-Methylaconitane- | 364.2488 | 9.4563 |
| 1,7,8,14-tetrol; | | |
| (1a,5β,14a)-form: N- | | |
| Et | | |
| Neovasipyridone: N- | 365.244 | 6.6311 |
| (3- | | |
| Carbamoylpropyl) | | |
| 3,17,20- | 366.2644 | 11.909 |
| Trihydroxypregnan- | | |
| 11-one; | | |
| (3a,5β,17aOH,20R)- | | |
| form; Oxime | | |
| Kokanicin | 367.2273 | 2.3245 |
| Platenolide II: 9- | 369.2277 | 1.1275 |
| Ketone | | |
| Petrosaspongiolide L | 370.2746 | 9.3189 |
| Smalogenin | 373.2379 | 4.5136 |
| Curacin A | 374.2517 | 8.7807 |
| Picrionoside B | 375.2383 | 6.8971 |
| Phenylalaninylarginal: | 376.2348 | 8.6027 |
| N2-(2- | | |
| Methylpropanoyl) | | |
| Dendroxine: 6- | 377.2566 | 6.7457 |
| Hydroxy, N-(3- | | |
| methyl-2-butenyl) | | |
| Aconitane-1,8,14,16- | 378.2644 | 8.6936 |
| tetrol; | | |
| (1a,5β,14a,16β)- | | |
| form: O1,O16-Di- | | |
| Me, N-Et | | |
| Seconorrlandin C | 379.2484 | 4.8652 |
| 8(14)-Pimarene- | 381.2641 | 4.4493 |
| 2,15,16,19-tetrol; | | |
| (ent-2a,15S)-form: | | |
| 15-Ac | | |
| ConioidineA: 4′- | 382.2957 | 13.4768 |
| Hydroxy | | |
| 11,12-Epoxy- | 383.207 | 1.4654 |
| 2,5,7,9,10- | | |
| pentahydroxy-4(20)- | | |
| taxen-13-one | | |
| Sylvatine | 384.2538 | 5.4257 |
| Denudatine: O15- | 386.2695 | 7.5543 |
| Ac | | |
| 23-Hydroxy-3- | 387.2535 | 4.6472 |
| oxochola-4,6-dien- | | |
| 24-oic acid | | |
| Myxalamides; | 388.2851 | 7.7928 |
| Myxalamide C | | |
| 7,16-Seco- | 389.2692 | 6.1722 |
| 7,11,15(17)- | | |
| trinervitatriene-3,10- | | |
| diol; (3a,10a)-form: | | |
| Di-Ac | | |
| Dammar-24-ene- | 390.2644 | 7.0468 |
| 3,20,26-triol; | | |
| Danomycin B | | |
| Plakortolide F‡ | 391.2484 | 6.1016 |
| Vitamycin A | 392.2702 | 7.4168 |
| 3-Hydroxyandrost- | 393.2429 | 4.0029 |
| 11-en-17-one; | | |
| (3a,5a)-form: | | |
| Benzoyl | | |
| Undecylprodiginine | 394.2858 | 9.6102 |
| 16-Kaurene- | 395.2433 | 1.9809 |
| 1,3,6,11,15-pentol; | | |
| (ent- | | |
| 1β,3a,6β,11a,15a)- | | |
| form: 3-Ac | | |
| Aphidicolin: 17- | 396.275 | 7.8937 |
| Glycine ester | | |
| 3-Hydroxy-7(11),9- | 397.2226 | 1.4941 |
| eremophiladien-8- | | |
| one; 3a-form: 3-O- | | |
| β-D- | | |
| Glucopyranoside | | |
| 5-Hydroxy-9- | 397.4045 | 1.2456 |
| hexacosanone | | |
| Valilactone | 398.2906 | 10.8936 |
| Lystatin | 399.2971 | 0.679 |
| Bioxalomycin a1: | 400.1872 | 0.453 |
| 10,13-Quinone, N- | | |
| Me | | |
| Chalmicrin | 401.2903 | 7.5685 |
| Myriocin | 402.2855 | 7.4473 |
| 19,25-Dihydroxy- | 403.2848 | 6.1735 |
| 16-scalaren-24,25- | | |
| olide | | |
| Dammar-24-ene- | 404.2801 | 8.1509 |
| 3,20,26-triol; | | |
| Danomycin B: Me | | |
| ether | | |
| Iguesterin: ?20(29)- | 405.2793 | 5.0453 |
| Isomer | | |
| Cassaidine: 7- | 406.2957 | 6.4978 |
| Ketone | | |
| Amphibine I | 407.2658 | 5.3074 |
| CalicogorginA | 408.3114 | 8.2776 |
| Amorfrutin B | 409.2379 | 2.083 |
| Glutamicacid; (S)- | 410.2906 | 9.0546 |
| form: N-(9Z,12Z- | | |
| Octadecadienoyl) | | |
| Strongylophorine 2 | 411.2535 | 0.6485 |
| Antibiotic Sch | 412.2811 | 5.5018 |
| 382582: | | |
| De(methylthio) | | |
| 24,25-Epoxy- | 413.3055 | 1.274 |
| 17(24),18(25)- | | |
| scalaradien-12-ol; | | |
| 12a-form: Ac | | |
| Trachyone | 414.3008 | 10.7083 |
| 3,20-Diamino-18- | 417.3117 | 4.0112 |
| hydroxypregn-5-ene- | | |
| 11,16-dione; (3β,20S)- | | |
| form: N-Tetra-Me | | |
| 3-Amino-12- | 418.2957 | 6.9579 |
| hydroxypregnan-20- | | |
| one; (3β,5a,12β)- | | |
| form: N,O-Di-Ac | | |
| 9′-Hydroxy-9′-apoe- | 419.295 | 6.2263 |
| caroten-3-one | | |
| Aflavinine: 13-Oxo, | 420.2902 | 7.052 |
| 10,11-dihydro, | | |
| 24,25-didehydro | | |
| 8(17),13-Labdadien- | 421.2954 | 3.8949 |
| 15-ol; (ent-13E)- | | |
| form: 15-Carboxylic | | |
| acid, (3-acetoxy-2S- | | |
| hydroxypropyl) ester | | |
| Cassaidine: 19- | 422.2906 | 6.1815 |
| Hydroxy, 7-ketone | | |
| 15,17-Epoxy-7,17- | 423.2746 | 3.3078 |
| dihydroxy-16- | | |
| isocopalanoic acid; | | |
| (7a,13aH,14aH, | | |
| 17a)-form: 17-Me | | |
| ether, 7-Ac, Me ester | | |
| Asmarine B | 424.3076 | 8.5108 |
| 5,6-Epoxy-5,6,7,8- | 425.2692 | 1.3891 |
| tetrahydro-3- | | |
| hydroxy-8-oxo-10′- | | |
| apo-β,?-carotenal | | |
| Veralodine | 426.3008 | 5.7686 |
| Bufogenin,, JAN: | 427.2484 | 1.1857 |
| Ac | | |
| 2-Amino-3,5- | 428.3012 | 6.6673 |
| dihydroxy-2- | | |
| (hydroxymethyl)- | | |
| 6,8-eicosadienoic | | |
| acid; (+)-(E,E)- | | |
| form: 5-Ac | | |
| 3-Hydroxyergosta- | 429.3368 | 2.408 |
| 5,24(28)-dien-19-oic | | |
| acid | | |
| Lankamycin: | 431.3009 | 5.4945 |
| Aglycone, 8-deoxy, | | |
| O-de-Ac | | |
| Salviaethiopisolide | 433.2954 | 3.0086 |
| 22-Chlorocholesta- | 435.303 | 4.4358 |
| 8,14-diene-3,23-diol; | | |
| (3β,5a,22R,23S)- | | |
| form | | |
| Paspaline: 19-Oxo | 436.2851 | 5.4943 |
| Canarol: 7-Hydroxy, | 437.2692 | 1.7113 |
| 7,8-didehydro | | |
| Aflavinine: 20β,25- | 438.3008 | 6.91 |
| Dihydroxy | | |
| 3,11,17,20- | 439.2696 | 0.4052 |
| Tetrahydroxypregnan- | | |
| 21-oic acid; | | |
| (3a,5β,11β,17aOH,20R)- | | |
| form: Me ester, | | |
| 20-Ac | | |
| Agelasidine C; (−)- | 440.2947 | 6.0395 |
| form: 20-Hydroxy | | |
| Salsamine | 441.2753 | 1.6464 |
| Stigmast-5-ene- | 445.3681 | 5.4402 |
| 3,7,22-triol; | | |
| (3β,7a,22R,24R)- | | |
| form: 7-Ketone | | |
| Alginidine | 446.327 | 8.1319 |
| CNS 2103 | 447.3447 | 4.349 |
| Gentamicin C; | 448.3135 | 5.5202 |
| Gentamicin C2b: 5- | | |
| Deoxy | | |
| Spirostane-1,3,5- | 449.3267 | 3.3629 |
| triol | | |
| 3,6- | 450.3219 | 5.2658 |
| Dihydroxycholan- | | |
| 24-oicacid; | | |
| (3a,5β,6a)-form: | | |
| Glycine amide | | |
| Seldomycin 5 | 451.288 | 1.6185 |
| 4-Methylaconitane- | 452.3012 | 5.064 |
| 6,7,8,14,16,18- | | |
| hexol; | | |
| (5β,6β,14a,16β)- | | |
| form: O6,O14,O16, | | |
| O18-Tetra-Me, | | |
| N-Et | | |
| Withanolide N | 453.2641 | 1.0232 |
| Antibiotic BE 67251 | 454.2957 | 4.3395 |
| 5,17-Dihydroxy-1- | 455.2797 | 0.8682 |
| oxowitha-2,6,24- | | |
| trienolide | | |
| Piericidin; Antibiotic | 456.3114 | 5.4132 |
| IT 143B | | |
| 27(14?15)-Abeo-2,3- | 457.3318 | 1.6197 |
| dihydroxy-24-nor- | | |
| 4(23),12-ursadien- | | |
| 28-oic acid | | |
| Piericidin; Piericidin | 458.327 | 7.0951 |
| A4 | | |
| 3-Hydroxy-6′-apo-β- | 461.3419 | 1.5071 |
| caroten-6′-al: 6′- | | |
| Alcohol | | |
| Veramanine | 462.3219 | 5.795 |
| Lysinomycin: 4′,5′- | 463.3244 | 4.1328 |
| Dihydro | | |
| Gentamicin C; | 464.3084 | 4.1243 |
| Gentamicin C1: 4″- | | |
| Demethyl | | |
| Psychotrine | 465.2753 | 1.065 |
| 4-Methylaconitane- | 466.3168 | 4.6724 |
| 1,6,8,14,16,18- | | |
| hexol; | | |
| (1a,5β,6a,14a,16β)- | | |
| form: O1,O6,O8,O16, | | |
| O18-Penta-Me, | | |
| N-Et | | |
| 3- | 467.2645 | 0.796 |
| Hydroxyandrostan- | | |
| 17-one; (3a,5β)- | | |
| form: 3-(β-D- | | |
| Glucuronopyranoside) | | |
| Veralodine: Ac | 468.3114 | 2.8363 |
| Ochrolifuanines; (3S, | 469.2967 | 0.2152 |
| 17S,20R)-form: | | |
| 10-Hydroxy, N2′- | | |
| Me | | |
| Methymycin | 470.3118 | 4.7865 |
| 16,28-Dihydroxy-3- | 471.3474 | 1.6804 |
| oxo-30-friedelanoic | | |
| acid; 16a-form: | | |
| 30?28 Lactone | | |
| Reticulataxanthin | 473.3419 | 0.7418 |
| Piericidin; Piericidin | 474.3219 | 6.1058 |
| A3: 10′-Me ether, | | |
| 11′,12′-epoxide | | |
| Aerophobin 1 | 475.0016 | 0.02 |
| Spirostane-5,6-diol; | 475.3423 | 4.0232 |
| (5a,6β,25R)-form: | | |
| 6-Ac | | |
| 9,10-Secoergosta- | 477.3133 | 2.4012 |
| 5,7,10(19),22- | | |
| tetraen-3-ol; (3S,5Z, | | |
| 7E,22E)-form: O- | | |
| Phosphate | | |
| Gentamicin C; | 478.3241 | 4.442 |
| Gentamicin C1 | | |
| Fucofuroeckol B | 479.0614 | 0.076 |
| Spirost-5-ene- | 479.3009 | 1.3965 |
| 1,3,12,23,24-pentol | | |
| Cafamycin | 480.3114 | 3.9041 |
| CombimicinA2 | 481.2873 | 1.236 |
| Oreoline‡ | 482.3118 | 3.043 |
| Strychnofoline | 483.276 | 0.4566 |
| 3-Hydroxycholan- | 484.3096 | 2.766 |
| 24-oicacid; (3a,5β)- | | |
| form: N-(2- | | |
| Sulfoethyl)amide | | |
| 24-Methyl-27-nor- | 485.3478 | 1.4409 |
| 3,6,8,15,16,26- | | |
| cholestanehexol; | | |
| (3β,5a,6β,8β,15a,16β, | | |
| 24?)-form: 4β- | | |
| Hydroxy | | |
| 2-Amino-1,3,4- | 486.3431 | 5.0174 |
| octadecanetriol; (2S, | | |
| 3S,4R)-form: | | |
| O,O,O,N-Tetra-Ac | | |
| 16,28-Dihydroxy-3- | 489.358 | 2.8542 |
| oxo-30-friedelanoic | | |
| acid | | |
| AAL Toxin; AAL | 490.338 | 5.8206 |
| Toxin TA1: 4,5- | | |
| Dideoxy | | |
| 13,15-Ethano-3,8- | 491.3174 | 2.4204 |
| diethyl-2,7,12,18- | | |
| tetramethyl-17- | | |
| propylporphyrin | | |
| 4-Methylaconitane- | 492.3325 | 4.7037 |
| 1,8,14,16,18-pentol; | | |
| (1a,14a,16β)-form: | | |
| O8-Et, O1,O16,O18- | | |
| tri-Me, N-Et, 14- | | |
| Ac | | |
| 3,3′,4′,5,7- | 492.9747 | 0.075 |
| Pentahydroxy-6- | | |
| methoxyflavone: | | |
| 3,3′-Di-O-sulfate | | |
| Carmine red | 493.0982 | 0.0168 |
| Pseudothiobinupharidine | 493.2888 | 1.3538 |
| Sarcotragin A | 494.327 | 3.8594 |
| Thiobinupharidine | 495.3045 | 1.2588 |
| Syringolin A: 2,3- | 496.3135 | 2.1236 |
| Dihydro | | |
| 2,3,11,14,20,22,25- | 497.3114 | 0.9038 |
| Heptahydroxycholest- | | |
| 7-en-6-one | | |
| 4-Methylaconitane- | 498.3067 | 2.2071 |
| 1,6,7,8,10,14,16,18- | | |
| octol; | | |
| (1a,6β,14a,16β)- | | |
| form: O1,O6,O14, | | |
| O16,O18-Penta- | | |
| Me, N-Et | | |
| 2-Demethylvitamin | 499.3576 | 1.7581 |
| K2(25) | | |
| Ergosta-5,7,22-trien- | 501.3732 | 0.5347 |
| 3-ol; (3β,22E,24R)- | | |
| form: Benzoyl | | |
| Ambruticin VS 5: | 502.3532 | 4.7206 |
| N,N-Di-Me | | |
| Oteromycin: 4″- | 506.327 | 3.7616 |
| Hydroxy, 16,17- | | |
| dihydro | | |
| 3,12-Dioxo- | 507.311 | 2.1156 |
| 13,15,17(20),22,24- | | |
| isomalabaricapentaen- | | |
| 26,22-olide; | | |
| (8a,9β,13E,15E, | | |
| 17(20)E)-form: 3β- | | |
| Alcohol, 3-Ac | | |
| Indanomycin: | 508.3427 | 3.5171 |
| Homologue (R = CH2CH3) | | |
| 1(10),4- | 509.3114 | 1.1319 |
| Germacradien-6-ol; | | |
| (1(10)E,4E,6β)- | | |
| form: O-(2-O- | | |
| Angeloyl-6-O- | | |
| acetyl-β-O- | | |
| glucopyranoside) | | |
| Syringolin D: 2,3- | 510.3291 | 1.7801 |
| Dihydro | | |
| 2,3,5,14,20,22,25- | 511.3271 | 0.822 |
| Heptahydroxyergost- | | |
| 7-en-6-one | | |
| Peimisine: Di-Ac | 512.3376 | 2.1923 |
| Holost-8-ene-3,23- | 513.358 | 1.4652 |
| diol; (3β,23?)-form: | | |
| 25,26-Didehydro, | | |
| 23-Ac | | |
| Molliorin D | 514.3685 | 2.968 |
| Cholest-5-en-3-ol; | 517.4045 | 0.6884 |
| 3β-form: C amoyl | | |
| 3,14-Dihydroxycard- | 518.3481 | 3.8235 |
| 20(22)-enolide; | | |
| (3β,5a,14β,17a)- | | |
| form: 3-O-(4- | | |
| Amino-2,4,6- | | |
| trideoxy-3-O- | | |
| methyl-a-L-arabino- | | |
| hexopyranoside) | | |
| Spirost-5-en-3-ol; | 519.3474 | 1.9467 |
| (3β,25R)-form: | | |
| Benzoyl | | |
| Quaesitol | 521.3267 | 1.0643 |
| 4-Methylaconitane- | 522.3431 | 1.8519 |
| 1,6,7,8,14,16-hexol; | | |
| (1a,5β,6β,14a,16β)- | | |
| form: O1,O6,O16- | | |
| Tri-Me, N-Et, 14-O- | | |
| (2-methylbutanoyl) | | |
| 2,3,14,20,22,25- | 523.3271 | 1.138 |
| Hexahydroxycholest- | | |
| 7-en-6-one; | | |
| (2β,3β,5β,20R,22R)- | | |
| form: 2-Ac | | |
| O- | 524.3376 | 2.0106 |
| Deethyltalaroconvolutin C | | |
| N- | 525.3176 | 1.0801 |
| Deacetylkuanoniamine | | |
| D: O10- | | |
| Tetradecanoyl | | |
| Picromycin | 526.338 | 3.2714 |
| 2,3,7,11,15- | 527.3584 | 1.1269 |
| Pentahydroxy-18- | | |
| hydroxymethyl- | | |
| 2,6,10,14, | | |
| 16,20-hexamethyl- | | |
| 4,8,12,16- | | |
| docosatetraenoic | | |
| acid | | |
| Kayamycin | 528.3536 | 1.9566 |
| 3,16-Dihydroxy-24- | 529.3893 | 1.1691 |
| methyllanosta-8,25- | | |
| dien-21-oicacid; | | |
| (3a,16a,24?)-form: | | |
| 3-Ac | | |
| 17,24:20,24- | 533.3842 | 1.2099 |
| Diepoxydammarane- | | |
| 3,12,25-triol; | | |
| (3a,12β,17a,20S, | | |
| 24S)-form: 3-Ac | | |
| Milbemycin IV: 7- | 535.3423 | 1.363 |
| Deoxy, 2,5,6,7- | | |
| tetradehydro | | |
| Oxysporidinone: 4′- | 536.3587 | 2.6989 |
| Di-Me acetal | | |
| Scutianine C: | 537.3441 | 1.1066 |
| Dihydro | | |
| Milnamide A: 1(2N)- | 538.3519 | 1.462 |
| Dehydro | | |
| Thiobinupharidine: | 539.3307 | 0.9249 |
| 6- or 6′-Ethoxy | | |
| Halichomycin | 540.3689 | 2.3319 |
| Antibiotic YM | 541.3529 | 1.4586 |
| 32890A | | |
| Antibiotic 3127 | 542.3554 | 1.5187 |
| 2,29-Dihydroxy-3- | 543.4049 | 1.4748 |
| friedelanone; 2a- | | |
| form: Di-Ac | | |
| Isarolide B | 544.375 | 1.7495 |
| Incarvine A: N- | 545.3954 | 0.726 |
| Oxide | | |
| Stacopin P1 | 546.3655 | 2.6604 |
| Ribazole; a-D-form: | 549.2753 | 1.2081 |
| 2,3,5-Tribenzyl | | |
| Strychnopentamine | 550.3546 | 2.0718 |
| Manzamine B | 551.375 | 1.5872 |
| O- | 552.3689 | 2.2301 |
| Deethyltalaroconvolutin | | |
| C: 4′-Et ether | | |
| 1,3,16,20,24- | 553.374 | 1.1865 |
| Pentahydroxy-24- | | |
| (hydroxymethyl)cycloartan- | | |
| 28-oic acid | | |
| Antibiotic SH 50 | 554.3666 | 1.8566 |
| Epoxomicin | 555.3758 | 1.8449 |
| Fellutamide A: 1″- | 556.371 | 2.1749 |
| Deoxy | | |
| Stigmasta-5,24(28)- | 559.4362 | 0.6189 |
| dien-3-ol; | | |
| (3β,24(28)E)-form: | | |
| 3-O-a-L- | | |
| Rhamnopyranoside | | |
| 8-Multiflorene- | 561.3944 | 0.6067 |
| 3,7,29-triol; (3a,7a)- | | |
| form: 7-Ketone, 3- | | |
| benzoyl | | |
| Stacopin P1: 4- | 562.3604 | 4.354 |
| Hydroxy | | |
| Thaimycin A | 563.3332 | 2.9633 |
| O- | 564.3689 | 2.8717 |
| Deethyltalaroconvolutin | | |
| C: 4′,5′- | | |
| Isopropylidene | | |
| Cholesta-7,22-diene- | 565.3376 | 1.0439 |
| 2,3,5,6,9,11,19- | | |
| heptol; | | |
| (2a,3β,5a,6β,11a)- | | |
| form: 11,19-Di-Ac | | |
| Repromicin,, | 566.3693 | 13864 |
| Cholest-5-ene- | 567.3897 | 1.3672 |
| 2,3,16,22-tetrol; | | |
| (2β,3β,16β,22S)- | | |
| form: 16-O-β-D- | | |
| Apiofuranoside | | |
| Cholestane- | 569.4053 | 2.2803 |
| 3,6,15,24-tetrol; | | |
| (3β,5a,6a,15β,24S)- | | |
| form: 24-O-β-D- | | |
| Xylopyranoside | | |
| Nidohottin | 571.3998 | 1.9658 |
| Antibiotic IC 202A | 573.3975 | 0.9848 |
| Jervine: 11-Deoxo, | 574.3744 | 2.3876 |
| 3-O-β-D- | | |
| glucopyranoside | | |
| Spirost-5-en-3-ol; | 577.374 | 0.9696 |
| (3β,25R)-form: 3-O- | | |
| β-D- | | |
| Glucopyranoside | | |
| Hugonone A: 3- | 579.3685 | 1.085 |
| Ketone | | |
| Destruxins; | 580.371 | 1.395 |
| Destruxin B: N Val- | | |
| De-Me | | |
| N- | 581.3842 | 1.5391 |
| Deacetylkuanoniamine | | |
| D: 12-Deoxy, 3- | | |
| (6-methyl-2,4- | | |
| octadienoyl)(E,E) | | |
| Isariins; Isariin F1 | 582.3867 | 1.4985 |
| Toxiferine I: | 583.38 | 1.1496 |
| Bis(deoxy) | | |
| Rosamicin: 20- | 584.3798 | 1.7654 |
| Alcohol | | |
| Cholestane- | 585.4002 | 1.3625 |
| 3,6,8,15,24-pentol; | | |
| (3β,5a,6a,15β,24S)- | | |
| form: 24-O-a-L | | |
| Arabinofuranoside | | |
| N- | 586.1567 | 0.0387 |
| Deacetylkuanoniamine | | |
| D: N-(9Z- | | |
| Hexadecenoyl) | | |
| Quercetin 3- | 587.1037 | 0.0798 |
| glycosides; | | |
| Monosaccharides: 3- | | |
| O-[3,4,5- | | |
| Trihydroxybenzoyl- | | |
| (?2)-a-L- | | |
| arabinopyranoside] | | |
| 4,9,13,20- | 587.4311 | 0.97 |
| Tetrahydroxy-1,6- | | |
| tigliadien-3-one; | | |
| (4β,9a,13a)-form: | | |
| 13-Hexadecanoyl | | |
| Dihydroxanthommatin; | 588.1465 | 0.0268 |
| (S)-form: 5-O- | | |
| β-D- | | |
| Glucopyranoside | | |
| 12-Oleanene- | 589.4104 | 0.7705 |
| 3,16,21,22,23,28- | | |
| hexol; | | |
| (3β,16a,21β,22a)- | | |
| form: 22-Angeloyl | | |
| Korsevine: 3-O-β- | 590.4057 | 2.0855 |
| D-Glucopyranoside | | |
| 2,2′,3,4,4′- | 590.7441 | 0.0393 |
| Pentabromo-6- | | |
| hydroxydiphenyl | | |
| ether: Me ether | | |
| 2-(3-Bromo-2-(2,3- | 590.8559 | 0.0338 |
| dibromo-4,5- | | |
| dihydroxybenzyl)- | | |
| 4,5- | | |
| dihydroxyphenyl)ethanol: | | |
| 1-Sulfate | | |
| Steffimycin,,: 7- | 591.2077 | 0.123 |
| Alcohol, 4′-Me ether | | |
| 3,6-Dihydroxypregn- | 592.2944 | 0.2896 |
| 9(11)-en-20-one; | | |
| (3β,5a,6a): 3-O- | | |
| Sulfate, salt with | | |
| BCB21-I | | |
| Antibiotic S 541; | 593.3478 | 0.8866 |
| Antibiotic S 541A: | | |
| 7-Deoxy, 2,5,6,7- | | |
| tetradehydro | | |
| Destruxins; | 594.3867 | 1.862 |
| Destruxin B | | |
| 3,14,22,25- | 595.3635 | 1.3806 |
| Tetrahydroxycholest- | | |
| 7-en-6-one; | | |
| (3β,5a,22R)-form: | | |
| 25-Benzoyl, 22-Ac | | |
| N- | 597.3791 | 1.2935 |
| Deacetylkuanoniamine | | |
| D: 3-(6-Methyl- | | |
| 2,4-octadienoyl)(E,E) | | |
| Mauritine D | 598.3968 | 2.8461 |
| 3,21,24-Trihydroxy- | | |
| 14-serraten-16-one; | | |
| (3β,21a)-form: Tri- | | |
| Ac | | |
| Cholestane- | 601.3952 | 1.0183 |
| 3,4,6,8,15,26-hexol; | | |
| (3β,4β,5a,6a,8β,15β, | | |
| 25S)-form: 26-O-β- | | |
| D-Xylopyranoside | | |
| 12-Oleanene-3,28- | 603.4049 | 0.3873 |
| diol; 3β-form: 28- | | |
| Aldehyde, 3-(3,4- | | |
| dihydroxy-E-c | | |
| amoyl) | | |
| Lolicine A | 604.4002 | 1.1833 |
| Lasalocid B | 605.4053 | 0.4493 |
| Destruxin A5 | 606.3867 | 2.9619 |
| Roxaticin | 607.3846 | 1.2738 |
| Malyngamide J | 608.3798 | 1.4109 |
| Antibiotic SB | 609.3751 | 0.8331 |
| 291071: Homologue | | |
| (R = —C15H29) | | |
| Juvenimicin B1: 3- | 610.3955 | 0.9888 |
| Ac | | |
| Halipeptin A: 5′- | 611.3842 | 0.8793 |
| Deoxy | | |
| 5- | 612.4111 | 1.242 |
| Hydroxydiscodermolicacid | | |
| A2 1: 3-Me ether | 613.4257 | 0.9842 |
| Cholestane- | 615.4108 | 0.8894 |
| 3,6,8,15,16,26- | | |
| hexol; | | |
| (3β,5a,6a,15a,16β,25S)- | | |
| form: 3-O-(2-O- | | |
| Methyl-β-D- | | |
| xylopyranoside) | | |
| Cholestane- | 617.3901 | 0.4337 |
| 3,4,6,8,15,16,26- | | |
| heptol; | | |
| (3β,4β,5a,6a,15β,16β, | | |
| 25S)-form: 26-O- | | |
| β-D-Xylopyranoside | | |
| Fla-P5 | 621.3944 | 1.0278 |
| Rubijervine: | 622.3896 | 1.585 |
| Dibenzoyl | | |
| N- | 623.3948 | 1.1354 |
| Deacetylkuanoniamine | | |
| D: 3-(8-Methyl- | | |
| 2,4,6- | | |
| decatrienoyl)(all-E) | | |
| Apicidin | 624.3761 | 2.118 |
| Ferulinolone | 625.3893 | 1.3177 |
| 5- | 626.4268 | 1.4675 |
| Hydroxydiscodermolicacid: | | |
| Me ester | | |
| Flexixanthin: 2′- | 627.4413 | 1.1764 |
| Hydroxy, 1′,3-di-Me | | |
| ether | | |
| Ferensimycin A | 629.4265 | 0.9296 |
| Argiopinin IV | 631.4408 | 0.6072 |
| Pupukeamide | 633.4227 | 0.7897 |
| Aurantinin A | 637.374 | 1.0714 |
| Rosamicin: 2′- | 638.3904 | 2.0451 |
| Propanoyl | | |
| Filipin,,; Filipin II | 639.4108 | 1.3027 |
| Antibiotic M 2845: | 640.406 | 1.161 |
| O3-Propanoyl | | |
| 8,9,15-Trihydroxy- | 641.4053 | 1.028 |
| 14-oxo- | | |
| 1(10),4,11(13)- | | |
| germacratrien-12,6- | | |
| olide; (1(10)E,4Z, | | |
| 6a,8β,9a)-form: 9- | | |
| (9,12- | | |
| Octadecadienoyl), 8- | | |
| (2-methylbutanoyl) | | |
| Ferensimycin B | 643.4421 | 0.9603 |
| CoriaheptocinA | 645.4578 | 0.8105 |
| 3,7-Dihydroxy-24- | 649.4315 | 0.8981 |
| methylenelanost-8- | | |
| en-21-oicacid; | | |
| (3β,7a)-form: 7-O- | | |
| β-D- | | |
| Glucopyranoside | | |
| 25-Amino-3,23- | 650.4268 | 2.3112 |
| dihydroxystigmast- | | |
| 5-ene-7,22-dione; | | |
| (3β,23R,24S)-form: | | |
| N-Me, 3-O-β-D- | | |
| glucopyranoside | | |
| Longisporin | 651.4108 | 1.3691 |
| Dysoxin3a: 3-Decamoyl, | 653.4053 | 0.9555 |
| 3-benzoyl | | |
| Heterostrophin | 655.4421 | 0.5893 |
| Globomycin | 656.4234 | 0.687 |
| 12-Oleanene- | 657.4366 | 0.5143 |
| 3,16,21,22,28- | | |
| pentol; | | |
| (3β,16a,21β,22a)- | | |
| form: 21-O- | | |
| (Methylbutenoyl), | | |
| 22,28-di-Ac | | |
| Argiopinin IV: | 659.4721 | 0.5967 |
| 4″,9′-Di-Me | | |
| 12,14-Labdadiene- | 659.5614 | 0.11 |
| 1,8,18-triol; | | |
| (1β,8a,12E)-form: | | |
| 18-Eicosanoyl, 1-Ac | | |
| 3,7,15-Trihydroxy- | 665.4265 | 0.6888 |
| 24-methylenelanost- | | |
| 8-en-21-oic acid; | | |
| (3β,7a,15a)-form: 7- | | |
| O-β-D | | |
| Glucopyranoside | | |
| Aabomycin S | 666.4217 | 1.5883 |
| Mycinamycin VI | 668.401 | 1.2934 |
| Ficus Latex peptide 1 | 669.4048 | 1.3466 |
| Antibiotic SF | 670.4391 | 0.7024 |
| 1902A4a | | |
| Bronianone | 671.4311 | 0.6537 |
| Pepstatins; Pepsidin A | 672.4547 | 1.0171 |
| 4,9,12,13,20- | 673.4679 | 0.5319 |
| Pentahydroxy-1,6- | | |
| tigliadien-3-one; | | |
| (4a,9a,12β,13a)- | | |
| form: 12,13- | | |
| Didecanoyl | | |
| Minalemines; | 677.4496 | 0.8573 |
| Minalemine A: N13- | | |
| Sulfate | | |
| 3,23,28-Trihydroxy- | 680.4373 | 0.6522 |
| 30-norlanost-8-en- | | |
| 29-oic acid; | | |
| (3β,23?)-form: 3-(N- | | |
| Acetyl-β-D- | | |
| glucosaminide) | | |
| Cholest-4-ene- | 681.4214 | 0.6649 |
| 3,7,12,26-tetrol; | | |
| (3a,7a,12a,25R)- | | |
| form: 26-Ac, 7-O- | | |
| (6-O-Acetyl-β-D- | | |
| galactopyranoside) | | |
| Mycinamycin VI: | 682.4166 | 0.9878 |
| 2″-Me ether | | |
| Laidlomycin,,: 26- | 683.437 | 0.861 |
| Deoxy | | |
| 4,21,27,45- | 683.5767 | 0.382 |
| Octatetracontatetraene- | | |
| 1,12,15,47- | | |
| tetrayne-3,14-diol | | |
| Antibiotic SF | 684.4547 | 0.9265 |
| 1902A5 | | |
| 3,16,21-Trihydroxy- | 685.4679 | 0.7131 |
| 12-oleanen-28-oic | | |
| acid; (3β,16a,21β)- | | |
| form: 21-(2- | | |
| Hydroxymethyl-6- | | |
| methoxy-6-methyl- | | |
| 2,7-octadienoyl) | | |
| Neofolitispate 1 | 686.5108 | 0.9624 |
| Pterulamide III | 687.4809 | 0.542 |
| 3,11,14- | 689.4125 | 0.1772 |
| Trihydroxycard- | | |
| 20(22)-enolide; | | |
| (3β,5β,11a,14β)- | | |
| form: 3-O- | | |
| (Argininylpimeloyl) | | |
| Fasciculic acid C: | 694.453 | 1.2396 |
| 21-Deoxy, 2-acyl | | |
| isomer | | |
| Aabomycin X | 695.4734 | 0.6416 |
| 20,24- | 697.4527 | 0.6597 |
| Epoxydammarane- | | |
| 3,12,17,25-tetrol; | | |
| (3a,12β,17aOH,20S, | | |
| 24R)-form: 12-Ac, | | |
| 3-O-β-D- | | |
| glucopyranoside | | |
| HeinsiageninA: 3-O- | 698.4632 | 0.8307 |
| β-D- | | |
| Xylopyranoside | | |
| Pepstatins; Pepstatin B | 700.486 | 0.4866 |
| 2,6,10,15,19,23- | 701.484 | 0.412 |
| Hexamethyl- | | |
| 6,18,22- | | |
| tetracosatriene- | | |
| 2,10,11,14,15- | | |
| pentol: 11-Ac, 14- | | |
| O-β-D- | | |
| mannopyranoside | | |
| 6,10-Diethyl-12- | 701.6236 | 0.1854 |
| phenyldodecanoic | | |
| acid: Cholesteryl | | |
| ester | | |
| Gymnasterone A | 702.5097 | 1.1088 |
| 2,16,20,25- | 703.3693 | 0.0868 |
| Tetrahydroxycucurbita- | | |
| 5,23-diene- | | |
| 3,11,22-trione; | | |
| (2β,16a,20R)-form: | | |
| 16-O-(2-O-Acetyl- | | |
| 6-deoxy-a-L-ribo- | | |
| hexopyranos-3- | | |
| uloside) | | |
| Kulomoopunalide 2 | 703.4646 | 0.2583 |
| Glycerol 1,2- | 703.536 | 0.1109 |
| dialkanoates; | | |
| !Glycerol 1- | | |
| hexadecanoate 2- | | |
| tetradecanoate: 3-O- | | |
| β-D- | | |
| Galactopyranoside | | |
| Minalemines; | 705.4809 | 0.8119 |
| Minalemine C: N13- | | |
| Sulfate | | |
| Reserpine,,, JAN,; | 708.386 | 0.1304 |
| (−)-form: 1-N- | | |
| (Diethylaminoethyl) | | |
| Lanost-5-ene-3,26- | 709.4679 | 0.4031 |
| diol; (3a,25?)-form: | | |
| 3-Ketone, 26-O-(4- | | |
| O-benzoyl-β-D- | | |
| glucopyranoside) | | |
| Spirolide E | 710.4632 | 1.1737 |
| Abereamine 2: ? | 711.4683 | 0.6684 |
| Tylosin,,: 23- | 712.4636 | 0.8783 |
| Deglucosyl, 23- | | |
| deoxy, 20-deoxo | | |
| Chlorobactene: | 713.4781 | 0.6448 |
| 1′,2′-Dihydro, 1′- | | |
| hydroxy, O-β-D- | | |
| glucopyranoside | | |
| Argiopinin II: 4- | 715.4731 | 0.1011 |
| Deoxy, N1-de-Me | | |
| Phosphatidylethanol | 718.5387 | 0.5922 |
| amine; Glycerol 1- | | |
| hexadecanoate 2-(9- | | |
| octadecenoate) 3- | | |
| phosphoethanolamine | | |
| Pentabromo-2- | 724.5043 | 0.6976 |
| propanone: Enol | | |
| tribromoacetate | | |
| Pepstatins; Pepstatin G | 728.5173 | 0.7656 |
| Tetrahexin | 736.4636 | 0.7348 |
| Virilemycin A | 738.4792 | 0.7869 |
| Stigmast-5-en-3-ol; | 739.4996 | 0.679 |
| (3β,24R)-form: O- | | |
| [β-D- | | |
| Fructofuranosyl- | | |
| (2?1)-a-D- | | |
| glucopyranos-3-yl]ether | | |
| Violaxanthin; (all-E): | 741.5094 | 0.7747 |
| 3,3′-Dibutanoyl | | |
| Pepstatins; Pepstatin I | 742.533 | 0.7192 |
| Symplostatin 3 | 747.4908 | 0.2641 |
| Trichodecenin I | 752.5286 | 0.7548 |
| DunaimycinD2S: 8- | 769.5102 | 0.7929 |
| Deglycosyloxy | | |
| 2-Amino-9- | 772.5938 | 0.8255 |
| methylene-4- | | |
| octadecene-1,3,8- | | |
| triol; (2S,3R,4E, | | |
| 8?)-form: N—(2R- | | |
| Hydroxyoctadecanoyl), | | |
| 1-O-β-D- | | |
| glucopyranoside | | |
| Glycerol 1,2- | 774.552 | 0.4074 |
| dialkanoates; | | |
| !Glycerol 1,2-di-(9Z, | | |
| 12Z,15Z- | | |
| octadecatrienoate): | | |
| 3-O-(6-Amino-6- | | |
| deoxy-a-D- | | |
| glucopyranoside) | | |
| Antibiotic PC 766B | 777.4789 | 0.1799 |
| Huratoxin: 5-Deoxy, | 777.5669 | 0.1163 |
| 6,7-deepoxy, 6,7- | | |
| didehydro, 20- | | |
| pentadecanoyl | | |
| Phosphatidylcholine; | 782.57 | 0.2763 |
| Glycerol 1,2-di-9,12- | | |
| octadecadienoate 3- | | |
| phosphocholine | | |
| Dammar-24-ene- | 785.5051 | 0.3575 |
| 3,20,26-triol; | | |
| (3β,20S,24Z)-form: | | |
| 3,26-Di-O-β-D- | | |
| glucopyranoside | | |
| Lycoperdin: O- | 787.4996 | 0.3323 |
| [2,3,6-Trideoxy-β-L- | | |
| erythro- | | |
| hexopyranosyl- | | |
| (1?4)-2,3,6-trideoxy- | | |
| a-L-erythro- | | |
| hexopyranosyl- | | |
| (1?4)-2,3,6-trideoxy- | | |
| β-L-threo- | | |
| hexopyranoside] | | |
| Phosphatidylethanol | 788.523 | 0.1206 |
| amine; Glycerol 1,2- | | |
| di-5Z,8Z,11Z,14Z- | | |
| eicosatetraenoate 3- | | |
| phosphoethanolamine | | |
| Stigmastane- | 789.5 | 0.1923 |
| 3,4,6,8,15,29-hexol; | | |
| (3β,4β,5a,6a,8β,15β, | | |
| 24R)-form: 29-O- | | |
| [2,4-Di-O-methyl-β- | | |
| D-xylopyranosyl- | | |
| (1?2)-a-L- | | |
| arabinofuranoside] | | |

TABLE 13
DART TOF-MS characterization of Nettle Extract 6.

| | | Rel. |
|---|---|---|
| | Calc. | Abund. |
| Compound Name | Mass | (%) |
| 2-Methyl-2-butenoic | 100.0762 | 0.9562 |
| acid; (E)-form: | | |
| Amide | | |
| 3-Aminodihydro- | 102.0555 | 0.4409 |
| 2(3H)-furanone | | |
| 4-Hydroxy-2- | 103.0395 | 0.2836 |
| oxobutanal | | |
| N,N- | 104.0711 | 10.8073 |
| pentadecanoyl | | |
| Diethanolamine | 106.0868 | 0.1421 |
| 4,6-Heptadiyn-3-ol; | 109.0653 | 2.524 |
| (±)-form | | |
| Tetramethylammonium(1+): | 110.0736 | 3.4535 |
| Chloride | | |
| Resorcinol | 111.0446 | 2.4856 |
| Histamine | 112.0874 | 8.1585 |
| 6-azacytosine | 113.0238 | 0.1465 |
| Creatinine | 114.0667 | 3.3797 |
| Proline | 116.0711 | 5.9474 |
| Levulinic acid | 117.0612 | 0.8731 |
| 2- | 118.0868 | 7.7801 |
| Methylaminoacetic | | |
| acid: Et ester | | |
| Threonine | 120.066 | 0.7962 |
| Nitrosofungin | 121.0613 | 0.6912 |
| Trimethylvinylammonium(1+): | 122.0736 | 0.9792 |
| Chloride | | |
| Vitamin B3 | 123.081 | 17.0422 |
| (niacinamide) | | |
| 4,5,6,7-Tetrahydro- | 124.0874 | 5.88 |
| 1H-imidazo[4,5-c]pyridine | | |
| 6-Methyl-3,5- | 125.0966 | 3.6614 |
| heptadien-2-one; (E)- | | |
| (?)-form | | |
| 6-Acetyl-1,2,3,4- | 126.0919 | 4.5065 |
| tetrahydropyridine | | |
| 5-Hydroxy-3-vinyl- | 127.0395 | 5.3489 |
| 2(5H)-furanone | | |
| 5,6-Dihydro-5- | 129.0551 | 0.196 |
| hydroxy-6-methyl- | | |
| 2H-pyran-2-one; | | |
| (5R,6S)-form | | |
| 2-Amino-4-hexenoic | 130.0868 | 9.2268 |
| acid | | |
| 3-Octanol; (S)-form | 131.1436 | 0.6276 |
| Leucine | 132.1024 | 7.3831 |
| Asparagine | 133.0613 | 0.6814 |
| 2-Amino-4-hydroxy- | 134.0817 | 1.7019 |
| 3-methylbutanoic | | |
| acid | | |
| 3-Phenyl-2-propen- | 135.081 | 0.4556 |
| 1-ol | | |
| Adenine | 136.061 | 19.6317 |
| Tetramethylpyrazine | 137.1078 | 26.9746 |
| 5-Ethyl-2- | 138.0919 | 10.5534 |
| methylpyridine: N- | | |
| Oxide | | |
| 2-Pentylfuran | 139.1123 | 7.5284 |
| 9- | 140.1075 | 11.1866 |
| Azabicyclo[3.3.1]nonan- | | |
| 3-one | | |
| Pyracrimycin A; (E)- | 141.1028 | 1.2324 |
| form: 1,2-Dihydro | | |
| 2-Aminoethanol: N, | 142.1232 | 6.6772 |
| N-Di-2-propenyl | | |
| 1-Undecene-5,7,9- | 143.0861 | 0.2827 |
| triyne | | |
| 2-Butanol; (±)- | 143.1072 | 0.2411 |
| form: 2-Methyl-2- | | |
| propenoyl | | |
| 4-tert-Butyl-2- | 144.1024 | 7.4734 |
| oxazolidinone | | |
| 2,3-Dihydro-3,6- | 145.0501 | 9.4344 |
| dihydroxy-2-methyl- | | |
| 4H-pyran-4-one | | |
| 2-Amino-3- | 146.0817 | 9.2944 |
| hydroxymethyl-3- | | |
| pentenoic acid | | |
| 4-Phenyl-2-butenal | 147.081 | 1.8667 |
| 2-Amino-5- | 148.0973 | 2.1481 |
| hydroxyhexanoic | | |
| acid | | |
| O-(2- | 149.0926 | 4.7566 |
| Aminoethyl)serine | | |
| 4-Methylbenzoic | 150.0919 | 7.5062 |
| acid: Methylamide | | |
| 2,6-Decadien-4-yn- | 151.1123 | 13.0008 |
| 1-ol | | |
| Carbon tetrachloride | 152.8832 | 0.0204 |
| 2-Hydroxy-3- | 153.1028 | 12.1689 |
| isopropylpyrazine; | | |
| OH: Me ether | | |
| Histamine: N?-Ac | 154.098 | 11.774 |
| 3,4-Dihydroxy-2- | 155.0946 | 3.9487 |
| methylpyridine; Di- | | |
| OH-form: 4-Me | | |
| ether, N-Me | | |
| Scopine | 156.1024 | 7.822 |
| Tetrahydro-2- | 157.0864 | 0.5157 |
| furanmethanol; (±)- | | |
| form: 2-Propenoyl | | |
| 8-Methyl-8- | 158.1181 | 16.9491 |
| azabicyclo[3.2.1]octane- | | |
| 3,6-diol | | |
| 5-Methyl-2,3- | 159.1133 | 0.1941 |
| hexanedione: | | |
| Dioxime | | |
| 4-Hydroxy-1,1- | 160.0973 | 10.8682 |
| dimethylpyrrolidinium- | | |
| 2-carboxylate; (2S, | | |
| 4R)-form | | |
| 2-Aminobutanoic | 161.1078 | 2.038 |
| acid; (±)-form: N- | | |
| Ph, nitrile | | |
| 3-Aminotetrahydro- | 162.0766 | 7.5119 |
| 5-(hydroxymethyl)- | | |
| 3-furancarboxylic | | |
| acid | | |
| Levoglucosan/Glyogen/ | 163.097 | 14.2924 |
| Laminarin | | |
| N-[2-(4- | 164.0711 | 55.5815 |
| Hydroxyphenyl)ethenyl]formamide | | |
| 3-Butyl-2,5- | 165.1391 | 22.6312 |
| dimethylpyrazine | | |
| 2-Amino-2- | 166.0868 | 32.6059 |
| phenylpropanoic | | |
| acid | | |
| 2-Methoxy-3-(1- | 167.1184 | 15.8438 |
| methylpropyl)pyrazine; | | |
| (±)-form | | |
| Synephrine | 168.1137 | 10.3192 |
| Tetramethylpyrazine: | 169.0977 | 15.9305 |
| 1,4-Dioxide | | |
| 3-Methylhistidine‡ | 170.0929 | 8.6172 |
| Tetrahydro-2- | 171.1021 | 4.1202 |
| furanmethanol; (±)- | | |
| form: 2-Methyl-2- | | |
| propenoyl | | |
| 2-Aminoethanol: N, | 172.1337 | 21.2796 |
| N-Di-Et, 2- | | |
| propenoyl | | |
| 2,4-Dihydroxy- | 173.1177 | 5.6442 |
| 2,6,6- | | |
| trimethylcyclohexanone | | |
| 8-Methyl-8- | 174.113 | 9.4089 |
| azabicyclo[3.2.1]octane- | | |
| 3,6,7-triol | | |
| Shikimic acid | 175.1446 | 7.1902 |
| 3- | 176.0923 | 8.6275 |
| Aminoheptanedioic | | |
| acid; (±)-form | | |
| 1,2,6-Trihydroxy-2- | 177.1127 | 6.4305 |
| methyl-4-heptanone | | |
| N-[2-(4- | 178.0868 | 48.1006 |
| Hydroxyphenyl)ethenyl]formamide: | | |
| Me | | |
| ether | | |
| Trichloroacetaldehyde: | 178.9433 | 0.0138 |
| Mono-Me acetal | | |
| 1,2-Bis(2- | 179.1283 | 24.125 |
| methoxyethoxy)ethane | | |
| 1,2,3,4-Tetrahydro- | 180.1024 | 57.8041 |
| 4,8-dihydroxy-2- | | |
| methylisoquinoline | | |
| 4-Isopropyl-1,2- | 181.1228 | 25.5395 |
| benzenediol: Di-Me | | |
| ether | | |
| 6-Nitro-p-mentha- | 182.1181 | 16.2139 |
| 1(7),2-diene | | |
| Carcinine | 183.1246 | 22.3192 |
| 6-(3-Methyl-1,3- | 184.1126 | 9.8481 |
| butadienyl)-1H- | | |
| indole; (Z)-form | | |
| 4-Hydroxy-2,6,6- | 185.1177 | 6.8687 |
| trimethyl-1- | | |
| cyclohexen-1- | | |
| carboxylic acid | | |
| 7-(3-Methyl-2- | 186.1282 | 9.3666 |
| butenyl)-1H-indole | | |
| 4-Amino-2- | 187.1082 | 7.6775 |
| piperidinecarboxylic | | |
| acid; (2S,4S)-form: | | |
| N—Ac | | |
| 2-Amino-4- | 188.0923 | 15.6375 |
| propylidenepentanedioic | | |
| acid | | |
| Glutamine,,; (S)- | 189.1239 | 3.7557 |
| form: N5-Isopropyl | | |
| 2-Ethyl-4- | 190.098 | 9.1367 |
| hydroxyquinazoline; | | |
| 3H-form: N-Amino | | |
| Boschniakine; (R)- | 191.131 | 11.9716 |
| form: N-Et | | |
| Hydrohydrastinine | 192.1024 | 100 |
| Sulfur, 11CI, ESA, | 192.8402 | 0.0259 |
| ISO, JMAF; Sulfur | | |
| (S6) | | |
| 2,6,8,10- | 193.1228 | 32.1312 |
| Dodecatetraenoicacid | | |
| 1,2,3,4-Tetrahydro- | 194.1181 | 23.2164 |
| 7-hydroxy-6- | | |
| methoxy-1- | | |
| methylisoquinoline; | | |
| (S)-form | | |
| 11,12,13-Trinor-3,8- | 195.1385 | 18.2253 |
| eudesmanedione | | |
| 4-(3-Aminopropyl)- | 196.1337 | 17.9585 |
| 1,2-benzenediol: Di- | | |
| Me ether | | |
| DL-methyl-m- | 196.9094 | 0.0445 |
| tyrosine | | |
| 2-Acetyl-4,4,6- | 197.1177 | 25.0479 |
| trimethyl-1,3- | | |
| cyclohexanedione | | |
| Noformicin; (±)- | 198.1355 | 13.4314 |
| form | | |
| 2-(2-Butenylidene)- | 199.1334 | 36.6104 |
| 5-heptene-1,3,4- | | |
| triol; (2Z,3R*,4S*, | | |
| 6Z,8E)-form | | |
| 8-Methyl-8- | 200.1286 | 17.5982 |
| azabicyclo[3.2.1]octane- | | |
| 3,6-diol; (3R, | | |
| 6R)-form: 3-Ac | | |
| 4,6,12- | 201.1279 | 8.6531 |
| Tetradecatriene- | | |
| 8,10-diyn-1-ol; | | |
| (E,E,E)-form | | |
| 2,6- | 202.1079 | 11.4601 |
| Piperidinedicarboxylic | | |
| acid; (2RS,6RS)- | | |
| form: Di-Me ester | | |
| 1,2,3,4-Tetrahydro- | 203.1184 | 7.9096 |
| 1-methyl-β- | | |
| carboline; (?)-form: | | |
| Nb-Oxide | | |
| Carnitine,; (±)-form: | 204.1236 | 10.6893 |
| O—Ac | | |
| 4-methyl-7- | 205.1341 | 12.7369 |
| ethoxycoumarin | | |
| N-(2- | 206.1181 | 36.9433 |
| Hydroxyethyl)camamide: | | |
| N-Me | | |
| 6-Deoxyglucose; β- | 207.1232 | 14.667 |
| L-Pyranose-form: 3- | | |
| Me, Et glycoside | | |
| 1,2,3,4-Tetrahydro- | 208.1337 | 22.7558 |
| 7-hydroxy-6- | | |
| methoxy-1- | | |
| methylisoquinoline; | | |
| (±)-form: N-Me | | |
| 6,10-Dimethyl-5,9- | 209.1541 | 32.8251 |
| undecadiene-2,8- | | |
| dione | | |
| Antibiotic A 41-89; | 211.1334 | 28.9171 |
| Antibiotic A 41-89I | | |
| 4-Methylbenzoic | 212.1075 | 15.8588 |
| acid: Anilide | | |
| Cucurbic acid | 213.149 | 21.1049 |
| Hexahydro-7a- | 214.1443 | 16.8587 |
| hydroxy-3H- | | |
| pyrrolizin-3-one; | | |
| (±)-form: (1- | | |
| Ethoxyethyl) ether | | |
| Tsitsikammafuran | 215.1436 | 20.858 |
| a-Amino-?- | 216.1236 | 19.4255 |
| oxooxiraneoctanoic | | |
| acid | | |
| 7-Methoxy-2,2- | 217.1228 | 15.5827 |
| dimethyl-6-vinyl-2H- | | |
| 1-benzopyran | | |
| 1H-Indole-3- | 218.1181 | 23.6114 |
| butanoic acid: Me | | |
| ester | | |
| 4,6-Tetradecadiene- | 219.1385 | 17.4297 |
| 8,10-diyne-1,12-diol | | |
| Vitamin B5 | 220.1185 | 64.7828 |
| 1,5- | 221.1389 | 23.6448 |
| Anhydromannitol; | | |
| D-form: Tetra-Me | | |
| 2-Amino-2- | 222.1341 | 37.1814 |
| deoxygalactose; a- | | |
| D-Pyranose-form: | | |
| 3,4,6-Tri-Me | | |
| 14-Nor-5- | 223.1698 | 21.3096 |
| protoilludene-7,8- | | |
| diol; (7a,8β)-form | | |
| 6-Amino-7,8- | 224.1511 | 16.758 |
| dihydro-2- | | |
| hydroxypurine: 6-N- | | |
| (3-Methylbutyl) | | |
| Murexine | 225.1477 | 25.6195 |
| Mescaline: N-Me | 226.1443 | 11.2347 |
| 4,7- | 227.1647 | 35.5599 |
| Megastigmadiene- | | |
| 3,6,9-triol | | |
| 8-Methyl-8- | 228.1599 | 13.5043 |
| azabicyclo[3.2.1]octane- | | |
| 3,6-diol; (3S, | | |
| 6S)-form: 3-O-(2- | | |
| Methylpropanoyl) | | |
| 2- | 229.1552 | 23.1611 |
| Piperidinecarboxylic | | |
| acid; (S)-form: | | |
| Amide, N-tert- | | |
| butyloxycarbonyl | | |
| Haplofoline | 230.1181 | 19.0429 |
| Verboccidentafuran: | 231.1385 | 12.2198 |
| 2-Oxo | | |
| 1H-Indole-3- | 232.1337 | 20.7829 |
| butanoic acid: Et | | |
| ester | | |
| Arabinitol; D-form: | 233.1389 | 16.4036 |
| 2,3:4,5-Di-O- | | |
| isopropylidene | | |
| 4-(3-Indolyl)-2- | 234.1494 | 70.584 |
| methyl-1-butanol; (R)- | | |
| form: N-Methoxy | | |
| 7-Isopropyl-1- | 235.1487 | 26.771 |
| methylphenanthrene | | |
| Anhalonine; (S)- | 236.1286 | 41.3261 |
| form: N-Me | | |
| 7-Hydroxy-13-nor- | 237.149 | 22.7002 |
| 3,9-bisaboladiene- | | |
| 2,11-dione | | |
| 2,3-Dihydro-2- | 238.1232 | 18.5986 |
| methylindole; (R)- | | |
| form: N-Benzoyl | | |
| isopropyl-B-D- | 239.1647 | 17.9269 |
| thiogalactopyranoside | | |
| Heteromine H: N2, | 240.146 | 12.0067 |
| N2-Di-Me | | |
| Putaminoxin B | 241.1803 | 35.2631 |
| 8-Methyl-8- | 242.1756 | 14.1085 |
| azabicyclo[3.2.1]octane- | | |
| 3,6-diol; (3S, | | |
| 6S)-form: 3-O-(3- | | |
| Methylbutanoyl) | | |
| phosphatidylcholine | 243.1596 | 22.6895 |
| Sceletenone | 244.1337 | 20.3643 |
| osthole | 245.1501 | 13.726 |
| 8-Methyl-8- | 246.1494 | 29.9016 |
| azabicyclo[3.2.1]octan- | | |
| 3-ol; (1RS,3RS)- | | |
| form: O-Benzoyl | | |
| 1-Deoxygalactitol; | 247.1545 | 19.5096 |
| D-form: 2,3:4,5-Di- | | |
| O-isopropylidene | | |
| 4-Amino-4,6- | 248.1498 | 22.1706 |
| dideoxy-3-C- | | |
| methylmannose; β- | | |
| D-Pyranose-form: | | |
| Me glycoside, N- | | |
| Me, N—Ac | | |
| 4-shogaol | 249.149 | 17.8249 |
| Anhalonine; (S)- | 250.1443 | 25.7044 |
| form: N-Et | | |
| 1-Hydroxy-5,11(13)- | 251.1647 | 16.1075 |
| eudesmadien-12-oic | | |
| acid | | |
| 1,2,3,4-Tetrahydro- | 252.1599 | 18.2221 |
| 5-hydroxy-6,7- | | |
| dimethoxy-1,2- | | |
| dimethylisoquinoline; | | |
| (±)-form: Me ether | | |
| 1,2,3,4-Tetrahydro- | 253.1678 | 15.5355 |
| 6,7,8-trihydroxy-1- | | |
| methylisoquinoline; | | |
| (?)-form: 6,7-Di-Me | | |
| ether, N,N-di-Me | | |
| 1,2,3,4-Tetrahydro- | 254.1392 | 12.468 |
| 5,6,7-trimethoxy-2- | | |
| methylisoquinoline: | | |
| N-Oxide | | |
| Palitantin; (+)-form | 255.1596 | 16.6463 |
| 8-Methyl-8- | 256.1549 | 13.3277 |
| azabicyclo[3.2.1]octane- | | |
| 3,6,7-triol; (1R*, | | |
| 3S*,6S*,7R*)- | | |
| form: 3-O-Tigloyl | | |
| Fumigaclavine B | 257.1654 | 18.8712 |
| Elaeocarpine | 258.1494 | 16.4126 |
| 1,8,16- | 259.1698 | 18.5481 |
| Heptadecatriene-4,6- | | |
| diyne-3,10-diol | | |
| Norchalciporol: O- | 260.165 | 17.5945 |
| Propanoyl | | |
| Cyclo(leucylphenylalanyl) | 261.1603 | 21.14 |
| Physostigmine,,: N8- | 262.1555 | 20.3091 |
| De-Me | | |
| 10-Hydroxy-1,3,5- | 263.1647 | 16.2677 |
| cadinatrien-15-oic | | |
| acid; (7β,10a OH)- | | |
| form: Me ester | | |
| 2-Acetamido-2- | 264.1447 | 19.5548 |
| deoxyglucose; D- | | |
| form: 3,4,6-Tri-Me | | |
| 3- | 265.1552 | 12.909 |
| (Dimethylaminomethyl)- | | |
| 5- | | |
| hydroxyindole: a r, | | |
| N-Dimethoxy, Me | | |
| ether | | |
| Brevicolline; (S)- | 266.1657 | 14.4124 |
| form | | |
| 1,7-Diphenyl-6- | 267.1749 | 13.0275 |
| hepten-3-ol | | |
| Codonopsine | 268.1549 | 14.8716 |
| Estra-1,3,5(10),16- | 269.1905 | 21.9446 |
| tetraen-3-ol: Me | | |
| ether | | |
| 8-Methyl-8- | 270.1705 | 15.0963 |
| azabicyclo[3.2.1]octane- | | |
| 3,6-diol; (3RS, | | |
| 6RS)-form: 6-O- | | |
| (2- | | |
| Methylpropanoyl), | | |
| 3-Ac | | |
| Chamanen | 271.1698 | 13.7498 |
| 3-Octadecanol; (−)- | 271.3001 | 0.2808 |
| form | | |
| Procerine‡ | 272.1498 | 12.41 |
| 2,3-Dihydro-3,3- | 273.1603 | 15.574 |
| dimethyl-1H- | | |
| pyrrolo[1,2-a]indole- | | |
| 9-alanine | | |
| 3-Amino-3- | 274.129 | 24.0227 |
| deoxyribose; a-D- | | |
| Furanose-form: 1,2- | | |
| O-Isopropylidene, N, | | |
| 5-di-Ac | | |
| 12,18- | 275.18 | 28.3218 |
| Heneicosadiene- | | |
| 1,3,8,10,20- | | |
| pentayne; (Z,Z)- | | |
| form: 3,4-Dihydro | | |
| (Z-) | | |
| Physostigmine,, | 276.1712 | 25.2019 |
| 9,11,13,15- | 277.2167 | 46.586 |
| Octadecatetraenoic | | |
| acid; (9E,11E,13E, | | |
| 15E)-form | | |
| 7-Hydroxy-14,15- | 279.2324 | 25.1537 |
| dinor-8(17)-labden- | | |
| 13-one | | |
| Dendrobine: N- | 280.1912 | 17.6832 |
| Oxide | | |
| 3,5-Dibromo-4- | 280.9213 | 0.051 |
| hydroxybenzyl | | |
| alcohol | | |
| 3-Bromo-5-hydroxy- | 281.0013 | 0.0292 |
| 2-methyl-1,4- | | |
| naphthoquinone: Me | | |
| ether | | |
| 4,13- | 281.1865 | 14.9216 |
| Dihydroxylupanine; | | |
| (4a,13β)-form | | |
| 8-Methyl-8- | 282.1705 | 12.5471 |
| azabicyclo[3.2.1]octane- | | |
| 3,6-diol; (3R, | | |
| 6R)-form: 3- | | |
| Tigloyl, 6-Ac | | |
| Carbonyl iodide | 282.8117 | 0.0335 |
| Gilbertine | 283.181 | 8.0339 |
| Hunteracine | 284.1888 | 11.4933 |
| 4- | 285.1854 | 11.5413 |
| Hydroxymethylathrixianone | | |
| piperine/cocluarine/laurifoline | 286.1654 | 11.9297 |
| 1,2,8,9- | 287.1858 | 12.0639 |
| Tetrahydroxydihydro- | | |
| β-agarofuran | | |
| Trichostatic acid | 288.1599 | 14.6306 |
| 13,14-Dihydroxy- | 289.1803 | 18.2861 |
| 8,11,13- | | |
| podocarpatrien-7- | | |
| one: 13-Me ether | | |
| 4-Amino-4,6- | 290.1603 | 18.1105 |
| dideoxy-3-C- | | |
| methylmannose; β- | | |
| D-Pyranose-form: | | |
| Me glycoside, N- | | |
| Me, N,2-di-Ac | | |
| 1-Octen-3-ol; (R)- | 291.1807 | 23.1906 |
| form: O-β-D- | | |
| Glucopyranoside | | |
| Variotin, JAN | 292.1912 | 13.1528 |
| Ecklonialactone A: | 293.2116 | 18.3191 |
| 6,7-Dihydro | | |
| 2-Amino-2- | 294.1916 | 13.2311 |
| deoxyglucose; D- | | |
| form: Di-Et acetal, | | |
| 5,6-O- | | |
| isopropylidene | | |
| Aristoteline; (+)- | 295.2174 | 14.7543 |
| form | | |
| 2-(3,7-Dimethyl-2,6- | 296.2014 | 11.0975 |
| octadienyl)-3- | | |
| methyl-4(1H)- | | |
| quinolinone | | |
| 2,4,6-Trichloro-1,3- | 296.9488 | 0.0221 |
| benzenediol: Di-Ac | | |
| 6- | 297.0215 | 0.0402 |
| Sulfoaminopenicillanic | | |
| acid | | |
| Frondosin B | 297.1854 | 5.4414 |
| Diphthine: 1′-Amide | 298.1879 | 10.2854 |
| Strychnochromine | 299.1759 | 10.6025 |
| Indicine‡: 3′-Epimer | 300.1811 | 11.7089 |
| 12- | 301.1803 | 10.7776 |
| Hydroxyandrosta- | | |
| 1,4-diene-3,17- | | |
| dione; 12β-form | | |
| Indolactam V | 302.1868 | 11.6164 |
| 16-Hydroxyandrost- | 303.196 | 11.9643 |
| 4-ene-3,17-dione; | | |
| 16a-form | | |
| Schoberidine | 304.1813 | 12.8046 |
| Malindine: 19- | 305.1892 | 12.0322 |
| Epimer | | |
| Amicetamine | 306.1916 | 12.3456 |
| 2,3,3-Tribromo-2- | 306.8205 | 0.0372 |
| propenoic acid | | |
| Di-2- | 306.8905 | 0.0265 |
| propenylheptasulfide | | |
| Eburnamenine; (−)- | 307.181 | 11.9976 |
| form: 11-Methoxy, | | |
| 14,15-didehydro | | |
| C-Alkaloid B | 308.1888 | 14.1681 |
| Taberpsychine | 309.1967 | 13.6038 |
| Tombozine: N4-Me | 310.2045 | 11.7679 |
| Eburnamenine; (−)- | 311.2123 | 3.6022 |
| form: Me ether | | |
| Retusine‡ | 312.1811 | 8.5415 |
| 4- | 313.1803 | 4.7269 |
| Hydroxymethylathrixianone: | | |
| 19- | | |
| Carboxylic acid, Me | | |
| ester | | |
| Schelhammerine: 2- | 314.1756 | 7.9969 |
| Deoxy | | |
| p-Mentha-1,8-dien- | 315.1807 | 8.9564 |
| 6-ol; (4S,6R)-form: | | |
| O-β-D- | | |
| Glucopyranoside | | |
| Androst-4-ene- | 316.1912 | 9.4689 |
| 3,11,17-trione: 17- | | |
| Oxime | | |
| 3,7-Dimethyl-2,6- | 317.1964 | 10.6678 |
| octadien-1-ol; (E)- | | |
| form: O-β-D- | | |
| Glucopyranoside | | |
| Suaveoline‡: N-Me | 318.197 | 10.1671 |
| 8- | 319.1909 | 10.1472 |
| dehydrogingerdione | | |
| Calabacine | 320.1974 | 10.1566 |
| 3-Hydroxy-17-nor- | 321.2066 | 8.7529 |
| 8-oxo-13-labden-15- | | |
| oic acid; (ent-3β,13Z)- | | |
| form: 3-Ketone | | |
| 8-Methyl-8- | 322.2018 | 9.8341 |
| azabicyclo[3.2.1]octane- | | |
| 3,6-diol; (3R, | | |
| 6R)-form: Ditigloyl | | |
| Neomycin A | 323.193 | 8.0696 |
| Inomycin | 324.2049 | 8.2235 |
| Hymeglusin | 325.2015 | 7.154 |
| C-Fluorocurine | 326.1994 | 10.5538 |
| Fluopsin B | 326.9468 | 0.1517 |
| Aspirochlorine: | 327.0109 | 0.1505 |
| Dechloro | | |
| Galeon | 327.1596 | 2.5814 |
| Fortuneine | 328.1912 | 6.6377 |
| Gelsedine | 329.1865 | 5.6427 |
| sinomenin/deoxyharringtonine | 330.1916 | 6.6593 |
| picrocrocin/carnosol | 331.1909 | 5.6214 |
| Alkaloid K5 | 332.1862 | 6.702 |
| Lysinomycin: De-N- | 333.2138 | 10.5502 |
| lysyl | | |
| 4-[2- | 334.2018 | 7.9316 |
| (Acetylmethylamino)ethyl]- | | |
| 4-(4- | | |
| hydroxy-3- | | |
| methoxyphenyl)-2,4- | | |
| cyclohexadien-1- | | |
| one: 2,3,5,6- | | |
| Tetrahydro, Me ether | | |
| 2,6-Dimethyl-7- | 335.207 | 7.4019 |
| octene-1,6-diol: 8-O- | | |
| β-D- | | |
| Glucopyranoside | | |
| Dendrine | 336.2175 | 8.6456 |
| 10,14-Epoxy-2,3- | 337.2015 | 4.9834 |
| seco-4(15)- | | |
| aromadendrene-2,3- | | |
| diol; | | |
| (1a,5a,6β,7β,10β)- | | |
| form: Di-Ac | | |
| Gramodendrine | 338.2232 | 7.935 |
| 21-Ethyl-2,6-epoxy- | 339.196 | 4.3992 |
| 1-oxa-2,5,14,17,20- | | |
| cycloheneicosapentaen- | | |
| 11-yn-4-one | | |
| 8-Methyl-8- | 340.2124 | 7.5393 |
| azabicyclo[3.2.1]octane- | | |
| 3,6,7-triol; (1R*, | | |
| 3S*,6S*,7R*)- | | |
| form: 3-O-Tigloyl, | | |
| 6-O-(3- | | |
| methylbutanoyl) | | |
| N- | 341.1865 | 3.0371 |
| Acetylphenylalanylphenylalaninol | | |
| Peraksine: 10- | 342.1943 | 6.9884 |
| Hydroxy, N4-Me | | |
| 12-Hydroxy-13- | 343.212 | 6.5209 |
| (hydroxymethyl)- | | |
| 3,5,7-trimethyl-2,4- | | |
| tetradecadiene-1,14- | | |
| dioic acid | | |
| Floridimine: 3′-Ac | 344.2073 | 6.7069 |
| Boviquinone 3 | 345.2066 | 7.3901 |
| 7-Isothiocyanato- | 346.2204 | 8.9294 |
| 11(20),14- | | |
| epiamphilectadien- | | |
| 12-ol | | |
| 3-Hydroxy-1,3,4- | 347.207 | 7.9533 |
| tetradecanetricarboxylic | | |
| acid | | |
| Bacchalejin 1 | 348.2175 | 6.6876 |
| Amorphastilbol | 349.2167 | 7.3606 |
| Fawcettiine; (−)- | 350.2331 | 7.8697 |
| form: Ac | | |
| 9,13,15-Trihydroxy- | 351.2171 | 6.1388 |
| 16-kauren-19-oic | | |
| acid | | |
| Pulchellidine‡ | 352.2488 | 7.7555 |
| Citreomontanin | 353.2116 | 3.9386 |
| Isostemonidine | 354.228 | 6.2434 |
| 1,3,9-Trihydroxy- | 355.1545 | 0.5701 |
| 10- | | |
| prenylpterocarpan: | | |
| 1-Me ether | | |
| Cyclopiperstachine; | 356.2225 | 5.4359 |
| (±)-form | | |
| Estra-1,3,5(10)- | 357.2066 | 3.5186 |
| triene-3,17-diol; | | |
| 17a-form: Di-Ac | | |
| Protostephanine | 358.2018 | 4.999 |
| Siphonodictyoic acid | 359.2222 | 5.1179 |
| Alkaloid K7 | 360.2175 | 6.8728 |
| Threonylthreonylargininal | 361.2199 | 5.9817 |
| Gomandonine | 362.2331 | 7.2314 |
| 12-Hydroxy- | 363.2171 | 4.8788 |
| 8(17),13-labdadien- | | |
| 15,12-olid-19-oic | | |
| acid; (12?)-form: 12- | | |
| Me ether | | |
| Nonylprodigiosin | 364.2389 | 7.2377 |
| 3,14,17,21- | 365.2328 | 5.8015 |
| Tetrahydroxypregn- | | |
| 5-en-20-one; | | |
| (3β,14β,17βOH)- | | |
| form | | |
| 4,18:8,13-Diepoxy- | 367.212 | 3.0121 |
| 6,19-dihydroxy- | | |
| 15,16-clerodanolide | | |
| Garceine | 367.4052 | 0.1155 |
| Brevicompanine A | 368.2338 | 5.4014 |
| Tortuosamine; (R)- | 369.2178 | 1.8921 |
| form: N—Ac | | |
| Calycine‡ | 370.2382 | 5.7492 |
| Ophiocarpine; (−)- | 371.1732 | 1.0343 |
| form: N-Me | | |
| Smalogenin | 373.2379 | 4.8759 |
| Napelline: 12- | 374.2331 | 6.0872 |
| Ketone, N-oxide | | |
| Wedeliasecokaurenolide: | 375.2171 | 6.1662 |
| 3β-Acetoxy | | |
| Cadiamine: O8-(2- | 376.2236 | 5.9478 |
| Pyrrolecarbonyl) | | |
| 1,5,6- | 377.2328 | 5.5106 |
| Vouacapanetriol; | | |
| (1a,5a,6β)-form: 6- | | |
| Ac | | |
| Aconitane-1,8,14,16- | 378.2644 | 6.7593 |
| tetrol; | | |
| (1a,5β,14a,16β)- | | |
| form: O1,O16-Di- | | |
| Me, N-Et | | |
| 6′-Oxo-6,5′-diapo-6- | 379.2273 | 4.0513 |
| carotenoic acid | | |
| 3- | 380.2589 | 6.8928 |
| Greenwayodendrinol; | | |
| 3β-form: Ac | | |
| 3,17,20-Trihydroxy- | 381.2277 | 3.3423 |
| 11-oxopregnan-21- | | |
| oic acid; | | |
| (3a,5β,17aOH,20S)- | | |
| form | | |
| Cyclomicrobuxeine | 382.2746 | 7.2197 |
| K: N-De-Me,N- | | |
| formyl | | |
| 11,12-Epoxy- | 383.207 | 2.4972 |
| 2,5,7,9,10- | | |
| pentahydroxy-4(20)- | | |
| taxen-13-one | | |
| Intermedine‡: 3′- | 384.2386 | 3.4612 |
| Epimer, O3′-(3- | | |
| methylbutanoyl) | | |
| N′,N″,N″-Trimethyl- | 384.3954 | 0.2425 |
| N-(3-methyl-2,4- | | |
| dodecadienoyl)spermidine; | | |
| (2′Z,4′E)- | | |
| form, 2′,3′,4′,5′- | | |
| Tetrahydro | | |
| Estra-1,3,5(10)- | 385.2379 | 1.2935 |
| triene-3,17-diol; | | |
| 17β-form: 3,17- | | |
| Dipropanoyl | | |
| 3,5,8,9,10,14,15- | 387.2383 | 4.6842 |
| Pepluaneheptol | | |
| Daphnezomine K: | 388.2488 | 4.6681 |
| 21-Deoxy | | |
| Arundarine | 389.2341 | 4.0654 |
| Phenylalaninylarginal: | 390.2505 | 4.4817 |
| N2-(3- | | |
| Methylbutanoyl) | | |
| Plakortolide F‡ | 391.2484 | 5.0108 |
| 4-Methylaconitane- | 392.2437 | 5.4772 |
| 1,8,14,16-tetrol; | | |
| (1a,14a,16β)-form: | | |
| O16-Me, O14-Ac | | |
| 3-Hydroxyandrost- | 393.2429 | 3.5894 |
| 11-en-17-one; | | |
| (3a,5a)-form: | | |
| Benzoyl | | |
| Macrocentrine | 394.2593 | 5.4532 |
| 2′,4′,7-Trihydroxy- | 395.2222 | 3.4428 |
| 3′,8- | | |
| diprenylisoflavan | | |
| Anopterimine | 396.2538 | 5.0694 |
| 3-Hydroxy-7(11),9- | 397.2226 | 1.5016 |
| eremophiladien-8- | | |
| one; 3a-form: 3-O- | | |
| β-D- | | |
| Glucopyranoside | | |
| 24-Nor-12-ursene | 397.3834 | 6.928 |
| Rhodocomatulin: | 399.1444 | 0.1477 |
| Tetra-Me ether | | |
| 1-Nonadecyl-1,3,5- | 399.3838 | 0.193 |
| cyclohexanetriol | | |
| Uoamine A | 400.2885 | 3.8276 |
| 12,25-Dihydroxy- | 401.2692 | 4.3886 |
| 15,17-scalaradien- | | |
| 24,25-olide | | |
| Napelline: 12-Ac | 402.2644 | 4.6193 |
| 4,11-Eudesmanediol; | 403.2696 | 4.8821 |
| (ent-4a,5a)-form: 4- | | |
| O-β-D- | | |
| Glucopyranoside | | |
| Crambescidin431: | 404.2549 | 4.713 |
| Parent acid | | |
| 1,3,7,11- | 405.2641 | 4.0496 |
| Cembratetraene- | | |
| 13,14-diol; (1Z,3E, | | |
| 7E,13S,14R)- | | |
| form: 11S,12R- | | |
| Epoxide, 13,14-di- | | |
| Ac | | |
| Fortimicin A | 406.2665 | 4.7484 |
| Amphibine I | 407.2658 | 4.1519 |
| Norerythrostachamine: | 408.275 | 5.0843 |
| 19-Aldehyde | | |
| Amorfrutin B | 409.2379 | 2.0423 |
| 11,13(18)- | 409.3834 | 2.2035 |
| Oleanadiene | | |
| 3,4-Dihydro-3,6,8,9- | 411.2171 | 1.1064 |
| tetrahydroxy-3- | | |
| methyl-1(2H)- | | |
| anthracenone; (S)- | | |
| form: 6-O-(3,7- | | |
| Dimethyl-2E,6- | | |
| octadienyl) | | |
| Saliniketal A: 18- | 412.2699 | 2.9957 |
| Hydroxy | | |
| Epopromycin A | 413.2651 | 2.0076 |
| 1,3,6,7- | 415.1757 | 0.3017 |
| Tetrahydroxy-2,8- | | |
| diprenylxanthone: | | |
| 2″,3″-Dihydro, 3″- | | |
| hydroxy | | |
| Aszonalenin: N5-Ac | 416.1974 | 0.6586 |
| Pregna-5,20-diene- | 417.2641 | 4.3574 |
| 3,7,11-triol; | | |
| (3a,7a,11a)-form: | | |
| 3,7-Di-Ac | | |
| Integramine | 418.2746 | 4.276 |
| 3,20- | 419.2797 | 3.8218 |
| Dihydroxypregnan- | | |
| 11-one; (3a,5β,20R)- | | |
| form: Di-Ac | | |
| Norerythrostachamide: | 420.275 | 4.0481 |
| 3-Deoxy, 7-ketone | | |
| Iguesterin: 7-Oxo, | 421.2742 | 3.1301 |
| 7,8-dihydro | | |
| Paxilline: 4b- | 422.2695 | 3.4746 |
| Deoxy, 3β-alcohol | | |
| Antibiotic PM 94128 | 423.2495 | 3.2846 |
| Aconitane- | 424.2699 | 4.6631 |
| 1,4,8,9,14,16-hexol; | | |
| (1a,5β,14a,16β)- | | |
| form: O1,O14,O16- | | |
| Tri-Me, N-Et | | |
| 2,3-Epoxy- | 425.2539 | 1.3884 |
| 5,6,10,14,16- | | |
| grayanotoxanepentol; | | |
| (1a,2β,3β,5β,6β,10a, | | |
| 14β,16a)-form: 14- | | |
| Propanoyl | | |
| Bufogenin,, JAN: | 427.2484 | 1.7039 |
| Ac | | |
| 2-Amino-3,5- | 428.3012 | 4.2462 |
| dihydroxy-2- | | |
| (hydroxymethyl)- | | |
| 6,8-eicosadienoic | | |
| acid; (+)-(E,E)- | | |
| form: 5-Ac | | |
| Vismiaguianone D | 429.1702 | 0.2659 |
| Glycerol 1-alkyl | 429.358 | 1.9902 |
| ethers; Glycerol 1- | | |
| octadecyl ether: Di- | | |
| Ac | | |
| 8-Cevenene-3,6,20- | 430.3321 | 5.1789 |
| triol | | |
| Cholest-5-ene- | 431.3161 | 4.5908 |
| 3,16,22,26-tetrol; | | |
| (3β,25S)-form: | | |
| 16,22-Diketone | | |
| Proxiphomin | 432.2902 | 2.8552 |
| Aspochracin | 433.2815 | 2.5167 |
| 4-Methylaconitane- | 434.2906 | 3.3575 |
| 1,8,14,16-tetrol; | | |
| (1a,14a,16β)-form: | | |
| O1,O16-Di-Me,N- | | |
| Et,O14-Ac | | |
| Tetracosanedioic | 435.2796 | 2.8666 |
| acid: Dichloride | | |
| Gentamicin C; | 436.2771 | 3.2467 |
| Gentamicin C1a: | | |
| 3″-N-De-Me | | |
| 4,18:11,16:15,16- | 437.2539 | 1.3347 |
| Triepoxy-6,19- | | |
| clerodanediol; (ent- | | |
| 4β,6β,11R,13S,16R)- | | |
| form: Di-Ac | | |
| 4-Methylaconitane- | 438.2855 | 3.5344 |
| 1,7,8,14,16,18- | | |
| hexol; | | |
| (1a,5β,14a,16β)- | | |
| form: O14,O16,O18- | | |
| Tri-Me, N-Et | | |
| Isoleucylamiclenomycylglutamic | 439.2556 | 0.8386 |
| acid | | |
| Diploclidine | 440.2801 | 3.8411 |
| Mediterraneol A: 7- | 441.2641 | 1.38 |
| Epimer | | |
| Antibiotic NK | 442.2957 | 6.1632 |
| 170204B | | |
| 1-O-C | 443.1553 | 0.1402 |
| amoylglucose; β-D- | | |
| Pyranose-(E)-form: | | |
| 6-O-a-L- | | |
| Arabinofuranosyl | | |
| Ningnanmycin | 444.1843 | 0.2597 |
| Glucitol; D-form: | 447.3322 | 3.1233 |
| Mono-9- | | |
| octadecenoyl(Z-) | | |
| Gentamicin C; | 448.3135 | 3.4389 |
| Gentamicin C2b: 5- | | |
| Deoxy | | |
| 8,11′; 12,12′- | 449.3055 | 2.5872 |
| Bi[1(10),7- | | |
| eremophiladien-9- | | |
| one]: 11?-Hydroxy | | |
| Gentamicin C; | 450.2928 | 2.9733 |
| Gentamicin C1a | | |
| Seldomycin 5 | 451.288 | 2.2184 |
| Epoxycytochalasin | 452.2801 | 2.6228 |
| H: O-De-Ac | | |
| Withanolide N | 453.2641 | 0.9605 |
| 4-Methylaconitane- | 454.2805 | 2.7031 |
| 1,6,7,8,14,16,18- | | |
| heptol; | | |
| (1a,5β,6β,14a,16β)- | | |
| form: O6,O16,O18- | | |
| Tri-Me, N-Et | | |
| 9,11,15,18- | 455.2645 | 0.8156 |
| Tetrahydroxyprosta- | | |
| 5,13-dienoic acid; | | |
| (5Z,8R,9S,11R, | | |
| 12S,13E,15S, | | |
| 18?)-form: 11,18- | | |
| Di-Ac | | |
| 27(14?15)-Abeo-2,3- | 457.3318 | 1.2211 |
| dihydroxy-24-nor- | | |
| 4(23),12-ursadien- | | |
| 28-oic acid | | |
| Piericidin; Piericidin | 458.327 | 3.4809 |
| A4 | | |
| 2-Methoxy-4-(2- | 459.1866 | 0.0971 |
| propenyl)phenol: O- | | |
| [β-D- | | |
| Apiofuranosyl- | | |
| (1?6)-β-D- | | |
| glucopyranoside] | | |
| a-Kosin | 461.2175 | 0.5199 |
| 3-Hydroxy-6′-apo-β- | 461.3419 | 1.3665 |
| caroten-6′-al: 6′- | | |
| Alcohol | | |
| Echinulin | 462.312 | 3.8617 |
| Lysinomycin: 4′,5′- | 463.3244 | 3.2157 |
| Dihydro | | |
| Gentamicin C; | 464.3084 | 2.8117 |
| Gentamicin C1: 4″- | | |
| Demethyl | | |
| Psychotrine | 465.2753 | 0.9316 |
| Indanomycin: 16- | 466.2957 | 3.2076 |
| Deethyl | | |
| 3- | 467.2645 | 1.1724 |
| Hydroxyandrostan- | | |
| 17-one; (3a,5β)- | | |
| form: 3-(β-D- | | |
| Glucuronopyranoside) | | |
| 4-Methylaconitane- | 468.2961 | 1.7194 |
| 1,6,7,8,14,16,18- | | |
| heptol; | | |
| (1a,5β,6β,14a,16β)- | | |
| form: O1,O6,O14, | | |
| O16-Tetra-Me, | | |
| N-Et | | |
| Withanicandrin | 469.259 | 0.7612 |
| CytochalasinS | 470.2906 | 2.5544 |
| Malonganenone B | 471.3335 | 1.002 |
| Reticulataxanthin | 473.3419 | 0.7034 |
| Piericidin; Piericidin | 474.3219 | 3.3283 |
| A3: 10′-Me ether, | | |
| 11′,12′-epoxide | | |
| Etzionin | 475.3284 | 2.5489 |
| 1,2,15-Hetisantriol; | 476.2437 | 0.5128 |
| (1β,2a,15β)-form: 2- | | |
| Benzoyl, 1-Ac | | |
| Desoxophylloerythroetioporphyrin | 477.3018 | 1.5451 |
| Antibiotic L 697318: | 478.2957 | 2.4022 |
| Deoxy, 18β-hydroxy | | |
| Psychotrine: Me | 479.291 | 1.0318 |
| ether | | |
| Gentamicin C; | 480.3033 | 2.1988 |
| Gentamicin C2b: 2- | | |
| Hydroxy | | |
| Mitraspecine | 481.2702 | 0.7831 |
| Ochrolifuanines; (3S, | 482.3045 | 1.6104 |
| 17S,20R)-form: | | |
| 10-Hydroxy, 18,19- | | |
| didehydro, N2′,N4- | | |
| di-Me | | |
| Gentamicin A: 6′- | 483.2666 | 0.748 |
| Me | | |
| Jervine: N- | 484.3063 | 1.6257 |
| Methoxycarbonyl | | |
| Pseudomonic acid C | 485.3114 | 1.4903 |
| Alkaloid LO1 | 486.312 | 2.8076 |
| Eudesmagnolol | 489.3368 | 1.8612 |
| Borrelidin | 490.3168 | 3.097 |
| Pregn-5-ene- | 491.3009 | 2.4933 |
| 3,12,14,17,20- | | |
| pentol; | | |
| (3β,12β,14β,17βOH, | | |
| 20S)-form: 12- | | |
| Tigloyl, 20-Ac | | |
| Morusimic acid D: | 492.3172 | 2.749 |
| 3-O-β-D- | | |
| Glucopyranoside | | |
| Aphelandrine; (+)- | 493.2815 | 1.0111 |
| form: 17,18- | | |
| Diepimer | | |
| Sarcotragin A | 494.327 | 2.4172 |
| Thiobinupharidine | 495.3045 | 0.9783 |
| 4-Methylaconitane- | 496.3063 | 1.4938 |
| 1,8,14,16-tetrol; | | |
| (1a,14a,16β)-form: | | |
| O14,O16-Di-Me,N- | | |
| Et, O1-benzoyl | | |
| Pregn-5-ene- | 497.2903 | 0.7957 |
| 3,12,14,17,20- | | |
| pentol; | | |
| (3β,12β,14β,17βOH, | | |
| 20S)-form: 12-C | | |
| amoyl | | |
| Antibiotic WA 3854; | 498.2968 | 1.441 |
| Antibiotic WA | | |
| 3854A | | |
| Lateritiin II | 499.3383 | 1.1488 |
| Tetrahydro-4- | 501.3791 | 0.4719 |
| hydroxy-6-(2,4,6- | | |
| trihydroxyheneicosyl)- | | |
| 2H-pyran-2-one: | | |
| 2′-Ac | | |
| Oteromycin: 4″- | 506.327 | 1.8145 |
| Hydroxy, 16,17- | | |
| dihydro | | |
| 3,12-Dioxo- | 507.311 | 1.0634 |
| 13,15,17(20),22,24- | | |
| isomalabaricapentaen- | | |
| 26,22-olide; | | |
| (8a,9β,13E,15E, | | |
| 17(20)E)-form: 3β- | | |
| Alcohol, 3-Ac | | |
| 4-Methylaconitane- | 508.3274 | 2.5755 |
| 1,6,7,8,14,16-hexol; | | |
| (1a,5β,6β,14a,16β)- | | |
| form: O1,O6,O16- | | |
| Tri-Me, N-Et, 14-O- | | |
| (2- | | |
| methylpropanoyl) | | |
| 1(10),4- | 509.3114 | 0.8315 |
| Germacradien-6-ol; | | |
| (1(10)E,4E,6β)- | | |
| form: O-(2-O- | | |
| Angeloyl-6-O- | | |
| acetyl-β-O- | | |
| glucopyranoside) | | |
| Syringolin D: 2,3- | 510.3291 | 1.229 |
| Dihydro | | |
| Usambarensine: | 511.3073 | 0.6845 |
| 6′,10-Dimethoxy, | | |
| 1′S,2′,3′, 4′- | | |
| tetrahydro, N2′-Me | | |
| Tumonoic acid C | 512.3223 | 1.9689 |
| Hemiasterlin: N1- | 513.3441 | 1.2407 |
| De-Me | | |
| Molliorin D | 514.3685 | 2.1357 |
| CyclobuxoxazineC: | 515.3849 | 0.5882 |
| N,O-Di-Ac | | |
| 3-Aminospirostan-6- | 516.3689 | 1.7433 |
| ol; (3β,5a,6a,25R)- | | |
| form: O,N-Di-Ac | | |
| Cholest-5-en-3-ol; | 517.4045 | 0.6058 |
| 3β-form: C amoyl | | |
| 3,14-Dihydroxycard- | 518.3481 | 1.389 |
| 20(22)-enolide; | | |
| (3β,5a,14β,17a)- | | |
| form: 3-O-(4- | | |
| Amino-2,4,6- | | |
| trideoxy-3-O- | | |
| methyl-a-L-arabino- | | |
| hexopyranoside) | | |
| 3,25-Epoxy- | 519.3322 | 1.2589 |
| 1,2,3,11- | | |
| tetrahydroxy-12- | | |
| ursen-28-oic acid | | |
| Cadabicine: N16,O- | 520.2447 | 0.2303 |
| Di-Ac | | |
| Epolone A | 521.2903 | 0.6596 |
| AAL Toxin; AAL | 522.3278 | 1.198 |
| Toxin TA1 | | |
| Emetine,; (−)-form: | 523.3172 | 0.7721 |
| N—Ac | | |
| 4-Methylaconitane- | 524.3223 | 1.2484 |
| 1,6,7,8,14,16,18- | | |
| heptol; | | |
| (1a,5β,6β,14a,16β)- | | |
| form: O1,O6,O8,O16, | | |
| O18-Penta-Me, | | |
| N-Et, 14-Ac- | | |
| Nummularine E: | 525.3077 | 0.6785 |
| Dihydro | | |
| Picromycin | 526.338 | 1.4657 |
| Brasiliensic acid; | 527.3372 | 0.7689 |
| (2R*,3S*)-form | | |
| 3,16-Dihydroxy-24- | 529.3893 | 0.8696 |
| methyllanosta-8,25- | | |
| dien-21-oicacid; | | |
| (3a,16a,24?))-form: | | |
| 3-Ac | | |
| Beauverolide A | 530.3594 | 1.3329 |
| 2,3,5,11,14- | 533.2387 | 0.1227 |
| Pentahydroxy-12- | | |
| oxobufa-20,22- | | |
| dienolide; | | |
| (2β,3β,5β,11a,14β)- | | |
| form: 2,3-Di-Ac | | |
| 17,24:20,24- | 533.3842 | 0.6315 |
| Diepoxydammarane- | | |
| 3,12,25-triol; | | |
| (3a,12β,17a,20S, | | |
| 24S)-form: 3-Ac | | |
| Adouetine Y′ | 535.3284 | 0.8648 |
| Oxysporidinone: 4′- | 536.3587 | 1.6506 |
| Di-Me acetal | | |
| Vitixanthin: 4,5- | 537.3216 | 0.7628 |
| Dihydro | | |
| 3,4,12,14,16,17,20- | 538.338 | 0.9192 |
| Cevaneheptol; | | |
| (3β,4a,5a,16β)-form: | | |
| 3-Ac | | |
| Thiobinupharidine: | 539.3307 | 0.6243 |
| 6-or 6′-Ethoxy | | |
| Brasilicardin A: 16- | 540.3536 | 1.2827 |
| Demethoxy, 4′- | | |
| deacyl, 3′- | | |
| deglycosyl | | |
| 4-Methylaconitane- | 541.3277 | 1.0234 |
| 1,8,14,16,18-pentol; | | |
| (1a,14a,16β)-form: | | |
| O1,O14,O16-Tri- | | |
| Me, N-Et, 18-O-(2- | | |
| aminobenzoyl) | | |
| Picromycin: 14R- | 542.3329 | 1.0509 |
| Hydroxy | | |
| Daphniphylline: N- | 544.3638 | 1.3119 |
| Oxide | | |
| Geniposidic acid: | 545.2023 | 0.0299 |
| 10-(5-Phenyl-2E,4E- | | |
| pentadienoyl), Me | | |
| ester | | |
| Orthoesterol B | 545.4206 | 0.4139 |
| 3,14,19- | 549.27 | 0.5951 |
| Trihydroxycarda- | | |
| 5,20(22)-dienolide; | | |
| (3β,14β)-form: 19- | | |
| Aldehyde, 3-O-β-D- | | |
| glucopyranoside | | |
| 1-O-Alkylglycero- | 550.3872 | 1.2979 |
| 3-phosphocholines; | | |
| 1-(9Z- | | |
| Octadecenyl)glycero- | | |
| 3-phosphocholine: | | |
| 2-Ac | | |
| Manzamine B | 551.375 | 0.963 |
| O- | 552.3689 | 1.249 |
| Deethyltalaroconvolutin | | |
| C: 4′-Et ether | | |
| 1,3,16,20,24- | 553.374 | 0.7238 |
| Pentahydroxy-24- | | |
| (hydroxymethyl)cycloartan- | | |
| 28-oic acid | | |
| Antibiotic SH 50 | 554.3666 | 1.5081 |
| Helianol: Octanoyl | 555.5141 | 1.3649 |
| 8(17)-Labden-15-ol; | 557.5297 | 1.6652 |
| (ent-13R)-form: | | |
| Carboxylic acid, 9- | | |
| octadecenyl ester | | |
| Rhodopin: | 559.4879 | 0.6439 |
| 7′,8′,11′,12′- | | |
| Tetrahydro | | |
| 7,9(11)- | 560.4103 | 1.3141 |
| Multifloradiene- | | |
| 3,29-diol; 3a-form: | | |
| 3-O-(4- | | |
| Aminobenzoyl) | | |
| 8-Multiflorene- | 561.3944 | 0.4423 |
| 3,7,29-triol; (3a,7a)- | | |
| form: 7-Ketone, 3- | | |
| benzoyl | | |
| Thaimycin A | 563.3332 | 1.1155 |
| CytochalasinB: Di- | 564.2961 | 0.403 |
| Ac | | |
| Cholesta-7,22-diene- | 565.3376 | 0.5479 |
| 2,3,5,6,9,11,19- | | |
| heptol; | | |
| (2a,3β,5a,6β,11a)- | | |
| form: 11,19-Di-Ac | | |
| Strychnophylline | 566.3495 | 0.9624 |
| Cholest-5-ene- | 567.3897 | 0.6875 |
| 2,3,16,22-tetrol; | | |
| (2β,3β,16β,22S)- | | |
| form: 16-O-β-D- | | |
| Apiofuranoside | | |
| 3,22,24-Trihydroxy- | 569.3842 | 1.1517 |
| 12-oleanen-28-oic | | |
| acid; (3β,22β)-form: | | |
| 3-Ketone, 22- | | |
| angeloyl | | |
| PC-M4 | 572.374 | 1.0621 |
| 12-Oleanen-3-ol; 3β- | 573.4307 | 0.8154 |
| form: O-(4- | | |
| Hydroxy-E-c | | |
| amoyl) | | |
| Spirost-5-en-3-ol; | 577.374 | 0.5024 |
| (3β,25R)-form: 3-O- | | |
| β-D- | | |
| Glucopyranoside | | |
| Coumingaine | 578.3693 | 0.9153 |
| Herpestine | 579.3434 | 0.5602 |
| Destruxins; | 580.371 | 0.7041 |
| Destruxin B: N Val- | | |
| De-Me | | |
| Spirostane-2,3,11- | 581.3689 | 0.6593 |
| triol; | | |
| (2β,3a,5β,11a,25R)- | | |
| form: 11-O-a-L- | | |
| Arabinopyranoside | | |
| Toxiferine I: | 583.38 | 0.5944 |
| Bis(deoxy) | | |
| Rosamicin: 20- | 584.3798 | 0.8076 |
| Alcohol | | |
| Cholestane- | 585.4002 | 0.7676 |
| 3,6,8,15,24-pentol; | | |
| (3β,5a,6a,15β,24S)- | | |
| form: 24-O-a-L- | | |
| Arabinofuranoside | | |
| Geldanamycin: 17- | 586.3128 | 0.117 |
| Demethoxy, 17- | | |
| allylamino | | |
| 20(29)-Lupene-3,28- | 587.41 | 0.9138 |
| diol; 3β: 28- | | |
| Aldehyde, 3-O-(4- | | |
| hydroxy-E-c amoyl) | | |
| Cycloeucalenol: 3-O- | 589.4468 | 0.3706 |
| β-D- | | |
| Glucopyranoside | | |
| Korsevine: 3-O-β- | 590.4057 | 0.8977 |
| D-Glucopyranoside | | |
| Apicidin: N- | 594.3655 | 0.9689 |
| Demethoxy | | |
| 3,14,22,25- | 595.3635 | 0.5617 |
| Tetrahydroxycholest- | | |
| 7-en-6-one; | | |
| (3β,5a,22R)-form: | | |
| 25-Benzoyl, 22-Ac | | |
| Destruxin C; | 596.3659 | 0.8019 |
| Destruxin C2 | | |
| 2,3,14,20,22- | 597.3639 | 0.916 |
| Pentahydroxycholest- | | |
| 7-en-6-one; | | |
| (2β,3β,5β,14a,20R, | | |
| 22R)-form: 3-O-β- | | |
| D-Xylopyranoside | | |
| Mauritine D | 598.3968 | 0.7396 |
| Cholestane- | 599.4159 | 0.7401 |
| 3,6,8,15,24-pentol; | | |
| (3β,5a,6a,15a,24S)- | | |
| form: 24-O-(3-O- | | |
| Methyl-β-D- | | |
| xylopyranoside) | | |
| Cholestane- | 601.3952 | 0.3892 |
| 3,4,6,8,15,26-hexol; | | |
| (3β,4β,5a,6a,8β,15β, | | |
| 25S)-form: 26-O-β- | | |
| D-Xylopyranoside | | |
| Tetronasin,, | 602.3818 | 0.5437 |
| 12-Oleanene-3,28- | 603.4049 | 0.1905 |
| diol; 3β-form: 28- | | |
| Aldehyde, 3-(3,4- | | |
| dihydroxy-E-c | | |
| amoyl) | | |
| Lolicine A | 604.4002 | 0.3693 |
| Destruxin A5 | 606.3867 | 0.834 |
| 1-O-β-D- | 607.3693 | 0.6384 |
| Mannopyranosyl-L- | | |
| erythritol: 3- | | |
| Hexadecanoyl, 2,6- | | |
| di-Ac | | |
| Chymostatin; | 608.3196 | 0.8226 |
| Chymostatin A | | |
| Destruxin C; | 610.3816 | 0.6262 |
| Destruxin C | | |
| 3,14,22,25- | 611.3795 | 0.4012 |
| Tetrahydroxycholest- | | |
| 7-en-6-one; | | |
| (3β,5a,22R)-form: | | |
| 3-O-β-D- | | |
| Glucopyranoside | | |
| 5- | 612.4111 | 0.5926 |
| Hydroxydiscodermolicacid | | |
| DonnaieninB | 613.4679 | 0.9929 |
| 12-Oleanene- | 615.4261 | 0.6677 |
| 3,16,21,22,28- | | |
| pentol; | | |
| (3β,16a,21β,22a)- | | |
| form: 21-Tigloyl, | | |
| 28-Ac | | |
| 8,9,15-Trihydroxy- | 617.4053 | 0.1633 |
| 14-oxo- | | |
| 1(10),4,11(13)- | | |
| germacratrien-12,6- | | |
| olide; (1(10)E,4Z, | | |
| 6a,8β,9a)-form: 9- | | |
| Hexadecanoyl, 8-(2- | | |
| methylbutanoyl) | | |
| Fla-P5 | 621.3944 | 0.3965 |
| Ulongamide F | 622.3638 | 0.7641 |
| Ceanothic acid: 3- | 623.3584 | 0.4023 |
| (3,4- | | |
| Dihydroxybenzoyl) | | |
| Homodestruxin B: 4- | 624.3972 | 0.7646 |
| Hydroxy | | |
| 3,3′,4′,5,7- | 625.374 | 0.525 |
| Pentahydroxyflavone: | | |
| 3′,4′,5,7-Tetra-Me | | |
| ether, 3-O- | | |
| octadecanoyl | | |
| Enniatin B: N-De- | 626.4016 | 0.3996 |
| Me | | |
| Montanacin H | 627.4472 | 0.5349 |
| Daechuine S3 | 628.4074 | 0.5222 |
| Ferensimycin A | 629.4265 | 0.4947 |
| 12,12′- | 631.3998 | 0.3163 |
| Bifuranoeremophila- | | |
| 7,11-dien-3-ol; | | |
| (3β,3′β)-form: Di-O- | | |
| angeloyl | | |
| Taiwaniadduct F | 633.4155 | 0.2072 |
| Aurantinin A | 637.374 | 0.337 |
| 3,16,21,23- | 638.4057 | 0.5839 |
| Tetrahydroxy-12- | | |
| oxo-30-oleananal; | | |
| (3β,16β,21β)-form: | | |
| 21-(2- | | |
| Methylaminobenzoyl) | | |
| 1,1′-[(6- | 639.3897 | 0.3661 |
| Methylheptylidene)bis(3, | | |
| 4-dihydro-5,7- | | |
| dihydroxy-2,2- | | |
| dimethyl-2H-1- | | |
| benzopyran-6,6′- | | |
| diyl)]bis[2-methyl-1- | | |
| propanone] | | |
| AKYFL amide | 640.3822 | 0.5932 |
| 8,9,15-Trihydroxy- | 641.4053 | 0.3715 |
| 14-oxo- | | |
| 1(10),4,11(13)- | | |
| germacratrien-12,6- | | |
| olide; (1(10)E,4Z, | | |
| 6a,8β,9a)-form: 9- | | |
| (9,12- | | |
| Octadecadienoyl), 8- | | |
| (2-methylbutanoyl) | | |
| Ferensimycin B | 643.4421 | 0.5018 |
| DolastatinC | 644.4387 | 0.5269 |
| 9-Hydroxy-3- | 645.4764 | 0.425 |
| methyl-2-decenoic | | |
| acid; (2Z,9R)-form: | | |
| O-Tricosanoyl, 2- | | |
| sulfoethyl ester | | |
| Fla-P4 | 649.4257 | 0.2415 |
| 25-Amino-3,23- | 650.4268 | 0.7938 |
| dihydroxystigmast- | | |
| 5-ene-7,22-dione; | | |
| (3β,23R,24S)-form: | | |
| N-Me, 3-O-β-D- | | |
| glucopyranoside | | |
| Longisporin | 651.4108 | 0.3837 |
| 6(17),11- | 653.3901 | 0.1857 |
| Jatrophadiene- | | |
| 3,5,7,8,9,14,15- | | |
| heptol; | | |
| (3β,5a,7β,8a,9a,11E, | | |
| 14β,15β)-form: 5,7- | | |
| Bis(2- | | |
| methylpropanoyl), 8- | | |
| (2-methylbutanoyl), | | |
| 14-Ac | | |
| Globomycin | 656.4234 | 0.3265 |
| Monensin,,,; 2- | 657.4214 | 0.2964 |
| Demethylmonensin A | | |
| Acidissiminol: O- | 660.4992 | 0.5126 |
| Octadecanoyl | | |
| Nodulisporic acid A: | 666.4158 | 0.5412 |
| Deoxo | | |
| 2,3,21,23- | 667.4057 | 0.2455 |
| Tetrahydroxy-12- | | |
| oleanen-28-oic acid; | | |
| (2a,3β,21β)-form: | | |
| 21-O-β-D- | | |
| Glucopyranoside | | |
| Mycinamycin VI | 668.401 | 0.585 |
| Ficus Latex peptide 1 | 669.4048 | 0.3856 |
| Antibiotic SF | 670.4391 | 0.3536 |
| 1902A4a | | |
| Antibiotic VM | 671.3795 | 0.1409 |
| 44866: 23β-(2- | | |
| Methylpropanoyloxy) | | |
| Nephilatoxin 10 | 672.4309 | 0.3991 |
| 4,9,12,13,20- | 673.4679 | 0.3307 |
| Pentahydroxy-1,6- | | |
| tigliadien-3-one; | | |
| (4a,9a,12β,13a)- | | |
| form: 12,13- | | |
| Didecanoyl | | |
| Phosphatidylcholine; | 678.5074 | 0.5232 |
| Glycerol 1,2- | | |
| ditetradecanoate 3- | | |
| phosphocholine | | |
| Laidlomycin,,: 26- | 683.437 | 0.1988 |
| Deoxy | | |
| Pseudodestruxin A | 684.4336 | 0.3435 |
| Mytiloxanthin: 19- | 685.4468 | 0.072 |
| Butanoyloxy | | |
| Squalamine: N ?-(3- | 685.5301 | 0.1178 |
| Aminopropyl) | | |
| Pregna-5,20-dien-3- | 686.4268 | 0.1676 |
| ol; 3β: 3-O-(2- | | |
| Acetamido-3-O- | | |
| acetyl-2-deoxy-4,6- | | |
| di-O-butanoyl-β-D- | | |
| galactopyranoside) | | |
| N2-(17-Hydroxy- | 687.5312 | 0.3962 |
| 9,12,15- | | |
| octadecatrienoyl)glutamine; | | |
| (2S,9′Z, | | |
| 12′Z,15′Z,17′?): | | |
| 15′,16′-Dihydro, | | |
| 17′-(9Z,12Z- | | |
| octadecadienoyl) | | |
| Spirolide B | 694.4683 | 0.5068 |
| 2-Amino-11-(2- | 694.6349 | 0.173 |
| butylcyclopropyl)-6- | | |
| undecene-1,3,4-triol; | | |
| (2S,3S,4R,6E, | | |
| 12?,13?)-form: N- | | |
| (2R- | | |
| Hydroxypentacosanoyl) | | |
| Edeine; Edeine D: | 695.4568 | 0.5548 |
| Decarboxy | | |
| 20,24- | 697.4527 | 0.3181 |
| Epoxydammarane- | | |
| 3,12,17,25-tetrol; | | |
| (3a,12β,17aOH,20S, | | |
| 24R)-form: 12-Ac, | | |
| 3-O-β-D- | | |
| glucopyranoside | | |
| Mycinamycin VI: 9- | 698.4479 | 0.3999 |
| Alcohol, 2″,3″-di- | | |
| Me ether | | |
| Pepstatins; Pepstatin B | 700.486 | 0.266 |
| Pepstatins; Pepstatin | 702.4653 | 0.1412 |
| A: Hydroxy | | |
| Glycerol 1,2- | 702.552 | 0.3254 |
| dialkanoates; | | |
| !Glycerol 1,2- | | |
| dipentadecanoate: 3- | | |
| O-(6-Amino-6- | | |
| deoxy-β-D- | | |
| glucopyranoside) | | |
| Spirolide E | 710.4632 | 0.5456 |
| Tylosin,,: 23- | 712.4636 | 0.4511 |
| Deglucosyl, 23- | | |
| deoxy, 20-deoxo | | |
| DolabelideD | 713.484 | 0.5832 |
| Curromycin A | 714.3965 | 0.1848 |
| 2-Amino-4,8- | 714.552 | 0.537 |
| octadecadiene-1,3- | | |
| diol; (2S,3R,4E,8E)- | | |
| form: N-(2R- | | |
| Hydroxyhexadecanoyl), | | |
| 1-O-β-D- | | |
| glucopyranoside | | |
| Phosphatidylethanol | 718.5387 | 0.3752 |
| amine; Glycerol 1- | | |
| hexadecanoate 2-(9- | | |
| octadecenoate) 3- | | |
| phosphoethanolamine | | |
| Obtusine: 3-O-[6- | 726.4428 | 0.1112 |
| Deoxy-3-O-methyl- | | |
| β-D-glucopyranosyl- | | |
| (1?4)-2,6-dideoxy-3- | | |
| O-methyl-β-D-ribo- | | |
| hexopyranoside] | | |
| Pepstatins; Pepstatin I | 742.533 | 0.3659 |
| Erythromycin,,: | 750.464 | 0.164 |
| 16-Hydroxy | | |
| Pyrophaeophorbide | 813.5682 | 0.6006 |
| a: 3,7R,5,11R- | | |
| Tetramethyl-2E- | | |
| hexadecenyl ester | | |

TABLE 14
DART TOF-MS characterization of Nettle Extract 7.

| | | Rel. |
|---|---|---|
| | Calc. | Abund. |
| Compound Name | Mass | (%) |
| N,N- | 104.0711 | 6.3986 |
| Dimethylglycine | | |
| 1,4-Benzoquinone | 109.0289 | 1.8343 |
| 2-Aminophenol | 110.0606 | 0.3959 |
| Resorcinol | 111.0446 | 2.7184 |
| Cytosine; OH-form | 112.0511 | 0.8079 |
| 6-azacytosine | 113.0238 | 0.5267 |
| 3,4-Dihydroxy-2- | 115.0395 | 0.5289 |
| methylenebutanoicacid; | | |
| (S)-form: | | |
| Lactone | | |
| Levulinic acid | 117.0612 | 1.5184 |
| 2- | 124.0762 | 0.4848 |
| Hydroxybenzylamine | | |
| 3,4-Dihydroxy-2- | 126.0555 | 0.1449 |
| methylpyridine; Di- | | |
| OH-form | | |
| 5,6-Dihydro-5- | 129.0551 | 1.1271 |
| hydroxy-6-methyl- | | |
| 2H-pyran-2-one; | | |
| (5R,6S)-form | | |
| 3,4-Dihydro-4- | 130.0504 | 0.4182 |
| hydroxy-2H- | | |
| pyrrole-2-carboxylic | | |
| acid | | |
| Leucine | 132.066 | 0.9337 |
| Adenine | 136.0762 | 3.2497 |
| 5-Ethyl-2- | 138.0919 | 0.7776 |
| methylpyridine: N- | | |
| Oxide | | |
| 3,4- | 140.0711 | 0.5328 |
| Dihydroxybenzylamine | | |
| 2-Amino-3- | 146.0817 | 0.3304 |
| hydroxymethyl-3- | | |
| pentenoic acid | | |
| 3-Amino-3- | 150.0766 | 2.6219 |
| deoxyribose | | |
| 2-Aminobenzoic | 152.0824 | 1.4446 |
| acid: Hydrazide | | |
| Scopine: 3-Ketone | 154.0868 | 1.2112 |
| S-(1- | 162.0588 | 2.7067 |
| Propenyl)cysteine | | |
| Levoglucosan/Glyogen/ | 163.0615 | 2.7051 |
| Laminarin | | |
| N-[2-(4- | 164.0711 | 26.3477 |
| Hydroxyphenyl)ethenyl]formamide | | |
| Rhamnose; D-form | 165.0763 | 3.5042 |
| Synephrine | 168.0773 | 1.6241 |
| 1-(2,4- | 169.0864 | 1.9959 |
| Dihydroxyphenyl)- | | |
| 2-propanol | | |
| 2-Aminoethyl | 170.0582 | 0.217 |
| dihydrogen | | |
| phosphate: Mono-Et | | |
| ester | | |
| 2,4-Dihydroxy- | 173.1177 | 0.9509 |
| 2,6,6- | | |
| trimethylcyclohexanone | | |
| 2-Amino-4- | 174.0766 | 0.7041 |
| ethylidenepentanedioic | | |
| acid | | |
| Shikimic acid | 175.1572 | 0.4168 |
| 3- | 176.0923 | 7.2366 |
| Aminoheptanedioic | | |
| acid; (±)-form | | |
| N4-(2- | 177.0875 | 1.4599 |
| Hydroxyethyl)asparagine; | | |
| (S)-form | | |
| N-[2-(4- | 178.0868 | 34.1743 |
| Hydroxyphenyl)ethenyl]formamide: | | |
| Me | | |
| ether | | |
| N-Benzoylglycine: | 179.082 | 3.2202 |
| Amide | | |
| 1,2,3,4-Tetrahydro- | 180.1024 | 2.9961 |
| 4,8-dihydroxy-2- | | |
| methylisoquinoline | | |
| 2-(4- | 181.0864 | 1.2816 |
| Hydroxyphenyl)propanoic | | |
| acid; (R)- | | |
| form: Me ester | | |
| 2-Amino-3-(2- | 182.0817 | 2.0517 |
| hydroxyphenyl)propanoic | | |
| acid | | |
| 2-Hydroxybenzoic | 183.0657 | 1.4988 |
| acid: 2- | | |
| Hydroxyethyl ester | | |
| 4-Amino-2,5,6- | 186.0515 | 0.2526 |
| trihydroxypyrimidine: | | |
| 5-O—Ac | | |
| 2-(1-Aminopropyl)- | 187.0541 | 0.6965 |
| 4-thiazolecarboxylic | | |
| acid | | |
| 2-Amino-4- | 188.0923 | 1.053 |
| propylidenepentanedioic | | |
| acid | | |
| 4-Hydroxy-3-(3- | 189.0915 | 1.1094 |
| methyl-3-buten-1- | | |
| ynyl)benzoic acid: | | |
| 1′-Alcohol | | |
| Glycylglycylglycine | 190.0828 | 2.3493 |
| 4-Cyclohexylphenol: | 191.1436 | 5.9743 |
| Me ether | | |
| Hydrohydrastinine | 192.1024 | 44.8683 |
| Trichloroacetaldehyde: | 192.959 | 0.0273 |
| Di-Me acetal | | |
| (N- | 193.0977 | 7.8667 |
| Phenylacetyl)glycine: | | |
| Amide | | |
| 1,2,3,4-Tetrahydro- | 194.1181 | 7.3 |
| 7-hydroxy-6- | | |
| methoxy-1- | | |
| methylisoquinoline; | | |
| (S)-form | | |
| 3,4-Dihydro-3,8- | 195.0657 | 0.3782 |
| dihydroxy-3-methyl- | | |
| 1H-2-benzopyran-1- | | |
| one | | |
| DL-methyl-m- | 196.0973 | 1.7054 |
| tyrosine | | |
| 2-Amino-3-(2,3- | 198.0766 | 0.9621 |
| dihydroxyphenyl)propanoic | | |
| acid | | |
| Pedatisectine D | 199.1082 | 0.8777 |
| Anticapsin | 200.0923 | 0.923 |
| 3,6-Dihydro-4-(4- | 201.0771 | 0.2619 |
| methyl-3-pentenyl)- | | |
| 1,2-dithiin | | |
| 2,6- | 202.1079 | 1.5542 |
| Piperidinedicarboxylic | | |
| acid; (2RS,6RS)- | | |
| form: Di-Me ester | | |
| 4-Hydroxy-3-(3- | 203.1072 | 0.8964 |
| methyl-3-buten-1- | | |
| ynyl)benzoic acid: | | |
| 1′-Alcohol, Me ether | | |
| Glycylglycylglycine: | 204.0984 | 3.5584 |
| Me ester | | |
| 4-methyl-7- | 205.0937 | 3.0552 |
| ethoxycoumarin | | |
| N-(2- | 206.1181 | 8.3156 |
| Hydroxyethyl)camamide: | | |
| N-Me | | |
| 6-Deoxyglucose; β- | 207.1232 | 3.065 |
| L-Pyranose-form: 3- | | |
| Me, Et glycoside | | |
| Felinine | 208.1007 | 4.4301 |
| 6,10-Dimethyl-5,9- | 209.1541 | 10.3042 |
| undecadiene-2,8- | | |
| dione | | |
| Elaeokanine E | 210.1494 | 2.6934 |
| Linderazulene | 211.1123 | 1.2588 |
| 2-Amino-3-(2,4- | 212.0923 | 5.8423 |
| dihydroxy-6- | | |
| methylphenyl)propanoic | | |
| acid | | |
| 2-Amino-3,4- | 213.114 | 1.1772 |
| dimethylimidazo[4,5- | | |
| f]quinoline | | |
| 9H-Pyrido[3,4-b]indol- | 214.1106 | 3.1256 |
| 6-ol: Me | | |
| ether, N2-Me | | |
| 5,6-Dihydro-3- | 215.1184 | 1.7177 |
| phenyl-4H- | | |
| pyrrolo[1,2-b]pyrazole: | | |
| 4′- | | |
| Methoxy | | |
| 3-Hydroxy-5- | 216.1024 | 2.5619 |
| methyl-1- | | |
| naphthalenecarboxylic | | |
| acid: Me ether, | | |
| amide | | |
| 2,3,4,9-Tetrahydro- | 217.0977 | 5.2396 |
| 1H-pyrido[3,4-b]indol- | | |
| 1-one: 7- | | |
| Methoxy | | |
| Glycylglycylglycine: | 218.1141 | 5.6793 |
| Et ester | | |
| 5,7-Dimethoxy-2-(1- | 219.1021 | 2.462 |
| methylethenyl)benzofuran | | |
| Vitamin B5 | 220.1185 | 18.2713 |
| 2-Hydroxybenzoic | 221.1177 | 4.3266 |
| acid: 3-Hexenyl | | |
| ester(Z-) | | |
| Anhalonine; (±)- | 222.113 | 3.4978 |
| form | | |
| 4,5-Dihydro-2,5- | 224.1075 | 2.6889 |
| diphenyloxazole | | |
| Choline: O-(4- | 225.1365 | 2.1496 |
| Hydroxybenzoyl) | | |
| 2-Amino-4-hydroxy- | 226.1079 | 1.4287 |
| 4-(4- | | |
| hydroxyphenyl)-3- | | |
| methylbutanoic acid | | |
| 1-(2,5- | 227.1283 | 2.1278 |
| Dihydroxyphenyl)- | | |
| 1,2-propanediol; (1R*,2R*)- | | |
| form: 1-Et, | | |
| 5′-Me ether | | |
| 2′-Deoxycytidine | 228.0984 | 4.5874 |
| Faramol | 229.1228 | 2.3857 |
| Kinetin; 7H-form: | 230.1042 | 12.1439 |
| 7-Me | | |
| 9H-Pyrido[3,4-b]indol- | 231.1133 | 3.5842 |
| 7-ol: 1,2,3,4- | | |
| Tetrahydro, Me | | |
| ether, N9-formyl | | |
| Coryneine: Chloride | 232.1104 | 6.8349 |
| Sorbicillin | 233.1177 | 2.8656 |
| 4-(3-Indolyl)-2- | 234.1494 | 14.9677 |
| methyl-1-butanol; (R)- | | |
| form: N-Methoxy | | |
| 8-Hydroxy-13-nor- | 235.1334 | 7.7463 |
| 4(15),7(11)- | | |
| eudesmadien-12,8- | | |
| olide | | |
| Anhalonine; (S)- | 236.1286 | 6.8155 |
| form: N-Me | | |
| Theophylline,, JAN,: | 237.1351 | 3.3538 |
| N-(2- | | |
| Methylpropyl) | | |
| 2-Amino-3- | 238.1079 | 9.9489 |
| hydroxypentanoic | | |
| acid; (2RS,3RS)- | | |
| form: N-Benzoyl | | |
| isopropyl-B-D- | 239.1032 | 1.3728 |
| thiogalactopyranoside | | |
| 9H-Carbazole-3- | 240.1024 | 4.3322 |
| carboxylicacid: Et | | |
| ester | | |
| 3,5- | 241.1228 | 2.2607 |
| Dihydroxystilbene; | | |
| (E)-form: Di-Me | | |
| ether | | |
| Flindersine: N-Me | 242.1181 | 3.58 |
| phosphatidylcholine | 243.1232 | 3.7768 |
| Vertilecanine A; | 244.0973 | 27.289 |
| (R)-form: Me ester | | |
| osthole | 245.1177 | 7.713 |
| 3-Deoxy-3- | 246.1341 | 17.3286 |
| (methylamino)arabinose; | | |
| β-L-Furanose- | | |
| form: N—Ac, 1,2-O- | | |
| isopropylidene | | |
| 8-Hydroxy- | 247.1334 | 4.8968 |
| 1,4,7(11)-guaiatrien- | | |
| 12,8-olide | | |
| Evernitrose; L- | 248.1134 | 18.6051 |
| Pyranose-form: Ac | | |
| 4-shogaol | 249.1338 | 4.2894 |
| 2-Acetamido-2- | 250.129 | 7.9276 |
| deoxyglucose; D- | | |
| form: 3,4-Di-Me | | |
| Lachnumfuran A | 251.1283 | 2.4358 |
| N-2-Phenylethylcamamide | 252.1388 | 6.1621 |
| Sequirin D | 253.1228 | 2.2186 |
| Lincosamine; a- | 254.1062 | 6.0237 |
| Pyranose-form: 1- | | |
| Thio, Me glycoside | | |
| 5-Deoxy-3-C- | 255.1232 | 3.4487 |
| hydroxymethyllyxose; | | |
| a-L-Furanose- | | |
| form: Benzyl | | |
| glycoside | | |
| Kainic acid,, JAN: | 256.1185 | 5.4256 |
| N—Ac | | |
| Albonoursin; (E,Z)- | 257.129 | 5.2446 |
| form | | |
| Cytidine: 2′-Me | 258.109 | 28.307 |
| Arsenobetaine: | 258.9515 | 0.0403 |
| Hydrobromide | | |
| Elaeagin | 259.1334 | 8.3455 |
| Peganidine: Oxime | 260.1399 | 9.0417 |
| 1-(6-Hydroxy-2- | 261.149 | 4.1963 |
| methyl-2H-1- | | |
| benzopyran-2-yl)-4- | | |
| methyl-2-pentanone | | |
| 8-Methyl-8- | 262.1443 | 5.2507 |
| azabicyclo[3.2.1]octane- | | |
| 3,6-diol; (3R,6R)- | | |
| form: 3- | | |
| Benzoyl | | |
| Furodysinin; (−)- | 263.1469 | 3.0852 |
| form: 14- | | |
| (Methylthio) | | |
| 2-Acetamido-2- | 264.1447 | 6.2401 |
| deoxyglucose; D- | | |
| form: 3,4,6-Tri-Me | | |
| Eupomatenoid 6 | 265.1228 | 2.9389 |
| Anisomycin | 266.1392 | 7.9974 |
| Lachnumlactone A | 267.1232 | 2.2313 |
| 1,2- | 268.1337 | 7.6114 |
| Dihydroxyaporphine; | | |
| (±)-form | | |
| 8,8 a-Dihydro-8 a- | 269.1389 | 3.8778 |
| hydroxy-3,3,6,6,8,8- | | |
| hexamethyl-1,2- | | |
| benzodioxin-5,7(3H, 6H | | |
| Anantine‡; (R): 3′- | 270.1242 | 6.1647 |
| Hydroxy | | |
| 2-Hydroxybenzoic | 271.1334 | 5.3562 |
| acid: 4-tert- | | |
| Butylphenyl ester | | |
| 2,3,5-Triamino- | 272.1246 | 11.158 |
| 2,3,5- | | |
| trideoxyarabinonic | | |
| acid; D-form: 1,4- | | |
| Lactone, tri-N—Ac | | |
| Verimol F | 273.149 | 7.7608 |
| Furo[2,3-b]quinoline- | 274.1079 | 74.0257 |
| 4,7,8- | | |
| triol; NH-form: 7,8- | | |
| Di-Me ether, N-Et | | |
| 13,14-Dihydroxy- | 275.1647 | 26.5031 |
| 8,11,13- | | |
| podocarpatrien-7- | | |
| one | | |
| Isoplatydesmine; (?)- | 276.1236 | 26.3457 |
| form: 2′-Hydroxy | | |
| 9,11,13,15- | 277.2167 | 21.1205 |
| Octadecatetraenoic | | |
| acid; (9E,11E,13E,15E)- | | |
| form | | |
| Decanedioic acid: | 278.1756 | 7.5324 |
| Monoanilide | | |
| 6,9-Dihydroxy-15- | 279.1232 | 1.819 |
| oxo-1,3,11(13)- | | |
| elematrien-12,8- | | |
| olide; (ent- | | |
| 6a,7a,8β,9β)-form | | |
| Theophylline,, JAN,: | 280.1409 | 4.5715 |
| N7- | | |
| Morpholinomethyl | | |
| 1,7-Diphenyl-3,5- | 281.1541 | 2.9806 |
| heptanediol; (3S,5S)- | | |
| form: 1,2- | | |
| Didehydro(E-), 3- | | |
| ketone | | |
| Bharatamine; (±)- | 282.1494 | 5.7889 |
| form | | |
| Randainal: 9- | 283.1334 | 2.4483 |
| Alcohol | | |
| (N- | 284.1286 | 6.3391 |
| Phenylacetyl)glycine: | | |
| Benzyl ester | | |
| 2,3-Dihydro-2-(4- | 285.149 | 5.4475 |
| hydroxyphenyl)-5- | | |
| (2-hydroxypropyl)- | | |
| 3-methylbenzofuran | | |
| piperine/cocluarine/laurifoline | 286.1443 | 7.0707 |
| 4-(3,7-Dimethyl-2,6- | 287.1647 | 4.2456 |
| octadienyl)-5- | | |
| hydroxy-2(3H)- | | |
| benzofuranone | | |
| Cyclo(alanyltryptophyl); | 288.1348 | 14.0212 |
| (3?,6?)-form: | | |
| 4′-Methoxy | | |
| 13,14-Dihydroxy- | 289.1803 | 9.9809 |
| 8,11,13- | | |
| podocarpatrien-7- | | |
| one: 13-Me ether | | |
| Scopolamine; (−)- | 290.1392 | 17.0934 |
| form: N-De-Me | | |
| 8-Hydroxy-17- | 291.196 | 20.6817 |
| octadecene-9,11- | | |
| diynoic acid | | |
| (4-Aminobutyl)urea; | 292.1872 | 8.2635 |
| (2S,3′S)-form | | |
| Ecklonialactone A: | 293.2116 | 20.2864 |
| 6,7-Dihydro | | |
| 2-Amino-2- | 294.1916 | 6.6626 |
| deoxyglucose; D- | | |
| form: Di-Et acetal, | | |
| 5,6-O- | | |
| isopropylidene | | |
| 16-Hydroxy- | 295.2273 | 12.6467 |
| 9,12,14- | | |
| octadecatrienoic acid | | |
| Euchrestine A: O2- | 296.165 | 4.8536 |
| Me | | |
| Brefeldin A: 8- | 297.1702 | 2.5826 |
| Hydroxy | | |
| 4-[2-(3,5-Dimethyl- | 298.1654 | 4.6413 |
| 2-oxocyclohexyl)-2- | | |
| hydroxyethyl]-2,6- | | |
| piperidinedione9C,I; | | |
| (1S,3S,5S,aR)- | | |
| form: 4?-Hydroxy | | |
| Strychnochromine | 299.1759 | 5.2059 |
| Erythratine: 2-Deoxy | 300.1599 | 8.2349 |
| 12- | 301.1803 | 6.0817 |
| Hydroxyandrosta- | | |
| 1,4-diene-3,17- | | |
| dione; 12β-form | | |
| 3-Amino-3- | 302.1603 | 9.8273 |
| deoxyglucose; a-D- | | |
| Furanose-form: | | |
| 1,2:5,6-Di-O- | | |
| Isopropylidene, N—Ac | | |
| Trichostatin A | 303.1708 | 5.9877 |
| Schoberidine | 304.1813 | 13.0379 |
| 2,4,6-Trihydroxy-3- | 305.1753 | 5.8722 |
| prenylacetophenone; | | |
| 4-O-(3-Methyl-2- | | |
| butenyl) | | |
| Pyrrolidine: N-(3,6- | 306.2797 | 14.6468 |
| Hexadecadienoyl) | | |
| Eburnamenine; (−)- | 307.181 | 8.6079 |
| form: 11-Methoxy, | | |
| 14,15-didehydro | | |
| 2-Benzamidobenzoic | 308.165 | 8.3845 |
| acid | | |
| Taberpsychine | 309.1967 | 10.6802 |
| Atherosperminine | 310.1807 | 8.8878 |
| Uvarisesquiterpene B | 311.2375 | 4.4018 |
| Clausenamide; (±)- | 312.1599 | 5.8031 |
| form: 1′-Me ether | | |
| Conkurchine | 313.2643 | 7.162 |
| Dehydroisolongistrobine: | 314.1504 | 8.8326 |
| Dihydro | | |
| Flourensianol: | 315.1596 | 6.3336 |
| Tigloyl | | |
| Erythratine | 316.1549 | 7.3719 |
| 3,7-Dimethyl-2,6- | 317.1964 | 5.8108 |
| octadien-1-ol; (E)- | | |
| form: O-β-D- | | |
| Glucopyranoside | | |
| 8-Methyl-8- | 318.1705 | 6.1095 |
| azabicyclo[3.2.1]octane- | | |
| 3,6-diol; (3R,6R)- | | |
| form: 3-O- | | |
| Phenylacetyl, 6-Ac | | |
| 8- | 319.1909 | 5.7309 |
| dehydrogingerdione | | |
| Calabacine | 320.1974 | 11.907 |
| Diptocarpidine: S,S′- | 321.2034 | 5.3232 |
| Dideoxy | | |
| Garamine | 322.1978 | 8.4797 |
| Neomycin A | 323.193 | 5.139 |
| Naamine A | 324.1712 | 9.8224 |
| Hymeglusin | 325.2015 | 6.7473 |
| C-Fluorocurine | 326.1994 | 8.4315 |
| Azomultin | 327.1781 | 4.9597 |
| Valylprolylleucine | 328.2236 | 9.4995 |
| 12-Hydroxypregna- | 329.2116 | 7.3232 |
| 4,16-diene-3,20- | | |
| dione | | |
| sinomenin/deoxyharringtonine | 330.1705 | 6.1515 |
| picrocrocin/carnosol | 331.1909 | 4.116 |
| 4-Amino-4,6- | 332.1709 | 5.6237 |
| dideoxy-3-C- | | |
| methylmannose; β- | | |
| D-Pyranose-form: | | |
| Me glycoside, N,2,3- | | |
| tri-Ac | | |
| 6-Methyl-2-vinyl-5- | 333.1913 | 7.0032 |
| heptene-1,2-diol: 1- | | |
| O-β-D- | | |
| Glucopyranoside | | |
| 4-[2- | 334.2018 | 6.0682 |
| (Acetylmethylamino)ethyl]- | | |
| 4-(4- | | |
| hydroxy-3- | | |
| methoxyphenyl)-2,4- | | |
| cyclohexadien-1- | | |
| one: 2,3,5,6- | | |
| Tetrahydro, Me ether | | |
| Lysinomycin: De-N- | 335.2294 | 6.5792 |
| lysyl, 4′,5′-dihydro | | |
| Dendrine | 336.2175 | 7.2277 |
| Pregnane-2,3,6-triol | 337.2742 | 9.7832 |
| Daphnicadine | 338.212 | 10.3393 |
| 7,13-Labdadiene- | 339.2535 | 8.2665 |
| 2,15,16,17-tetrol | | |
| 4-[2-(3,5-Dimethyl- | 340.176 | 8.9287 |
| 2-oxocyclohexyl)-2- | | |
| hydroxyethyl]-2,6- | | |
| piperidinedione9C,I; | | |
| (1S,3S,5S,aR)- | | |
| form: 5R-Acetoxy | | |
| 1,3(15),6- | 341.2116 | 6.4919 |
| Humulatrien-9-ol: | | |
| O-(4- | | |
| Hydroxybenzoyl) | | |
| Sorbistin D | 342.1876 | 4.8587 |
| 12-Hydroxy-13- | 343.212 | 5.3742 |
| (hydroxymethyl)- | | |
| 3,5,7-trimethyl-2,4- | | |
| tetradecadiene-1,14- | | |
| dioic acid | | |
| Jamtine | 344.1862 | 6.5911 |
| Dihydrocorynantheol; | 345.2178 | 6.5901 |
| (−)-form: 10- | | |
| Methoxy, N4-β- | | |
| oxide | | |
| Athrocupressine: Me | 346.2018 | 6.3689 |
| ether, O-de-Me | | |
| Peripentadenine: N- | 347.2334 | 6.3924 |
| Dehexanoyl, N- | | |
| butanoyl | | |
| Bacchalejin 1 | 348.2175 | 6.9544 |
| Pregna-5,20-diene- | 349.2379 | 7.1535 |
| 3,7,11,19-tetrol | | |
| Holantogenin | 351.2535 | 11.8955 |
| Dendrobates | 352.2851 | 10.2329 |
| Alkaloid 351 | | |
| 3-(15- | 353.2692 | 10.6189 |
| Hydroxyhexadecyl)- | | |
| 4-methyl-2,5- | | |
| furandione | | |
| CyclostachineA; (±)- | 354.2069 | 10.4091 |
| form | | |
| Glycerol 1- | 355.2848 | 4.5467 |
| alkanoates; Glycerol | | |
| 1-(9Z,12Z- | | |
| octadecadienoate) | | |
| Martefragine A | 356.1974 | 8.5542 |
| Strictanine: 12- | 357.2178 | 5.6129 |
| Hydroxy, N- | | |
| deformyl, N—Ac | | |
| Antibiotic BAY q | 358.1726 | 5.9672 |
| 1313 | | |
| Siphonodictyoic acid | 359.2222 | 4.7361 |
| Alkaloid K7 | 360.2175 | 5.8911 |
| 3-Hydroxy-8- | 361.2379 | 5.6733 |
| kempen-6-one; | | |
| (3β,7β)-form: 2β- | | |
| Acetoxy | | |
| Gomandonine | 362.2331 | 7.6697 |
| 3-Methoxy-5-(1- | 363.2324 | 5.3411 |
| terpinen-4- | | |
| yloxy)stilbene | | |
| 4-Methylaconitane- | 364.2488 | 6.6657 |
| 1,7,8,14-tetrol; | | |
| (1a,5β,14a)-form: N- | | |
| Et | | |
| 13(16),14- | 365.2692 | 6.6838 |
| Labdadiene-3,6,8- | | |
| triol; (ent-3β,6a,8a)- | | |
| form: 6-Ac | | |
| 2-Pentadecyl-4(1H)- | 366.2797 | 8.5702 |
| quinolinone: | | |
| 6′,7′,9′,10′- | | |
| Tetradehydro, N-Me | | |
| (Z,Z-) | | |
| Homoplakotenin | 367.2637 | 7.0721 |
| Piperundecalidine | 368.2225 | 6.5392 |
| Aspidospermine: N- | 369.2542 | 6.1975 |
| De-Ac, N-propanoyl | | |
| Lomatin; (R)-form: | 371.1858 | 3.8331 |
| O-(Z)-4-Octenoyl | | |
| Smalogenin | 373.2379 | 4.5774 |
| Napelline: 12- | 374.2331 | 7.6679 |
| Ketone, N-oxide | | |
| Pregna-5,20-diene- | 375.2535 | 5.3352 |
| 3,7,11-triol; | | |
| (3a,7a,11a)-form: 3- | | |
| Ac | | |
| Cadiamine: O8-(2- | 376.2236 | 6.727 |
| Pyrrolecarbonyl) | | |
| 3,20- | 377.2692 | 5.7888 |
| Dihydroxypregnan- | | |
| 11-one; (3a,5β,20R)- | | |
| form: 3-Ac | | |
| Aconitane-1,8,14,16- | 378.2644 | 7.2101 |
| tetrol; | | |
| (1a,5β,14a,16β)- | | |
| form: O1,O16-Di- | | |
| Me, N-Et | | |
| Plakinic acid F | 379.2848 | 5.0455 |
| 3- | 380.2589 | 5.2751 |
| Greenwayodendrinol; | | |
| 3β-form: Ac | | |
| Phycomysterol B: | 381.3157 | 7.0892 |
| 24,28-Didehydro | | |
| Cyclomicrobuxeine | 382.2746 | 7.4488 |
| K: N-De-Me,N- | | |
| formyl | | |
| Ergosta-7,22-diene | 383.3678 | 22.3589 |
| CadlinolideB; | 385.3106 | 4.7686 |
| Human calcitonin | | |
| 23-Hydroxy-3- | 387.2535 | 4.6287 |
| oxochola-4,6-dien- | | |
| 24-oic acid | | |
| 7,16-Seco- | 389.2692 | 5.3933 |
| 7,11,15(17)- | | |
| trinervitatriene-3,10- | | |
| diol; (3a,10a)-form: | | |
| Di-Ac | | |
| Dammar-24-ene- | 390.2644 | 5.0009 |
| 3,20,26-triol; | | |
| Danomycin B | | |
| Vitamycin A | 392.2702 | 7.6406 |
| Ergosta-4,6,8,22- | 393.3157 | 8.0095 |
| tetraen-3-one | | |
| Norcassaidide | 394.2957 | 7.2568 |
| 24-Nor-4(23),9(11)- | 395.3678 | 20.4568 |
| fernadiene | | |
| Inandenin-10-one | 396.359 | 11.5622 |
| 24-Nor-12-ursene | 397.3834 | 100 |
| 1-Nonadecyl-1,3,5- | 399.3838 | 7.0375 |
| cyclohexanetriol | | |
| Mycalazoles; | 400.3215 | 7.9934 |
| Mycalazole 2 | | |
| Chalmicrin | 401.2903 | 6.8809 |
| Napelline: 12-Ac | 402.2644 | 4.813 |
| Istamycin C | 404.2873 | 5.6327 |
| Bacchalejin 1 | 405.3117 | 6.4222 |
| Aflavinine | 406.311 | 6.4004 |
| 4,7-Epoxy-11- | 407.3525 | 10.3555 |
| eremophilanol; | | |
| (4a,7a,10β)-form: | | |
| Undecanoyl | | |
| Antibiotic U 106305 | 408.3266 | 7.3383 |
| 11,13(18)- | 409.3834 | 38.7532 |
| Oleanadiene | | |
| 11-Hydroxy-21- | 411.3838 | 10.4432 |
| hexacosenoicacid; | | |
| (11?,21Z)-form | | |
| Veralobine | 412.3215 | 6.0934 |
| 14-Methyl-9,19- | 413.3783 | 12.4971 |
| cycloergost-24(28)- | | |
| en-3-ol | | |
| 3-Hydroxycholest-5- | 415.3576 | 5.4239 |
| en-7-one; 3β-form: | | |
| Me ether | | |
| 3-Hydroxycholest-5- | 416.3528 | 5.6997 |
| en-7-one; 3β-form: E- | | |
| Oxime | | |
| 3-Hydroxychol-5- | 417.3005 | 7.0119 |
| en-24-oicacid; 3β- | | |
| form: 3-Ac | | |
| 3-Amino-12- | 418.2957 | 4.8107 |
| hydroxypregnan-20- | | |
| one; (3β,5a,12β)- | | |
| form: N,O-Di-Ac | | |
| Axinellamine B‡ | 419.3426 | 5.6526 |
| 23,29-Imino-B (9a)- | 421.3582 | 7.6134 |
| homo-19- | | |
| norstigmasta- | | |
| 1(10),7,9(11),23(N)- | | |
| tetraen-3- | | |
| Paspaline | 422.3059 | 5.1109 |
| Cycloeucalenol: 3- | 425.3783 | 10.7022 |
| Ketone | | |
| Moenjodaramine | 427.3688 | 10.4102 |
| Korsevine | 428.3528 | 8.3791 |
| Cholest-22-en-3-ol; | 429.3732 | 15.8483 |
| (3β,5a,22Z)-form: | | |
| Ac | | |
| Cholest-4-ene-3,6- | 431.3889 | 15.1003 |
| diol; (3β,6a)-form: | | |
| Di-Me ether | | |
| Bacchalejin 1: N20,N20- | 433.343 | 4.8839 |
| Di-Me | | |
| 3-Hydroxycholan- | 434.327 | 5.3289 |
| 24-oicacid; (3a,5β)- | | |
| form: Glycine amide | | |
| 4,15,26- | 435.3263 | 6.1432 |
| Triacontatriene- | | |
| 1,12,18,29-tetrayne- | | |
| 3,28-diol; (3?,4E,15Z,26E,28?)- | | |
| form: 12,13- | | |
| Dihydro(Z-) | | |
| Cholestane- | 437.3631 | 5.8005 |
| 3,7,12,23-tetrol; | | |
| (3a,5β,7a,12a,23S)- | | |
| form | | |
| Teleocidin B1: N- | 438.312 | 5.9207 |
| De-Me | | |
| Amphiasterin B3 | 439.3787 | 9.8049 |
| Buxamine I: N3,N3,N20- | 441.3845 | 8.7644 |
| Tri-Me, N20- | | |
| Ac | | |
| CyclovirobuxeineI: | 443.3637 | 3.8983 |
| N3,N3,N20-Tri- | | |
| Me,N20-formyl | | |
| 2-(14-Hydroxy- | 444.3477 | 8.2616 |
| 14,15- | | |
| dimethylhexadecyl)- | | |
| 4(1H)-quinolinone; | | |
| (+)-form: 3-Methoxy | | |
| Stigmast-5-ene- | 445.3681 | 15.3813 |
| 3,7,22-triol; | | |
| (3β,7a,22R,24R)- | | |
| form: 7-Ketone | | |
| 3-Deamino-3- | 446.3634 | 8.214 |
| hydroxysolanocapsine: | | |
| 23-Me ether | | |
| Stigmast-5-ene- | 447.3838 | 10.8528 |
| 3,7,22-triol; | | |
| (3β,7a,22R,24R)- | | |
| form | | |
| Phloeodictyne A; | 448.4015 | 9.7286 |
| Phloeodictyne 4,9a | | |
| 2-Amino-1,3,4- | 450.3583 | 5.5 |
| eicosanetriol; | | |
| (2S,3S,4R)-form: N- | | |
| Benzoyl | | |
| Coscinoquinol | 451.3576 | 13.2703 |
| Mycalazoles; | 452.3528 | 6.8207 |
| Mycalazole 1: 7′,8′- | | |
| Dihydro | | |
| Stigmasta-5,7,22- | 453.3732 | 6.7116 |
| trien-3-ol; (3β,24R)- | | |
| form: Ac | | |
| Spirosol-4-en-3-one; | 454.3321 | 5.5549 |
| (22R,25R)-form: | | |
| N—Ac | | |
| 3-Hydroxy-12- | 455.3525 | 5.2995 |
| oleanen-27-oic acid; | | |
| 3a-form: 3-Ketone | | |
| 12-Hydroxy-3,7- | 457.3681 | 5.0764 |
| friedelanedione | | |
| Spirostan-3-amine; | 458.3634 | 6.771 |
| (3β,5a,25R)-form: | | |
| N—Ac | | |
| Tetrahydro-4- | 459.3685 | 8.326 |
| hydroxy-6-(2,4,6- | | |
| trihydroxyheneicosyl)- | | |
| 2H-pyran-2-one | | |
| 3,20- | 461.3743 | 6.3198 |
| Diaminopregnane- | | |
| 2,4-diol; | | |
| (2β,3β,4β,5a,20S)- | | |
| form: N20,N20-Di- | | |
| Me, N3-tigloyl | | |
| Phloeodictyne A; | 462.4172 | 9.0765 |
| Phloeodictyne 4,10a | | |
| Halicyclamine A | 463.4052 | 10.8438 |
| Lucidine B: | 464.3641 | 4.5082 |
| 14,15,16,17- | | |
| Tetradehydro | | |
| CallyspongyneA | 465.4096 | 5.7452 |
| Molliorin C | 466.3685 | 3.872 |
| 12,20(30)-Ursadien- | 467.3889 | 6.4527 |
| 3-ol; (3a,18aH)- | | |
| form: Ac | | |
| Panclicins; Panclicin E | 468.3689 | 4.3215 |
| Cholestane- | 469.3529 | 2.5238 |
| 2,3,7,12,26,27-hexol | | |
| Secodaphniphylline | 470.3634 | 3.3621 |
| 2-Methoxy-4-(2- | 473.2023 | 0.2818 |
| propenyl)phenol: O- | | |
| [a-L- | | |
| Rhamnopyranosyl- | | |
| (1?6)-β-D- | | |
| glucopyranoside] | | |
| 3,29-Dihydroxy-12- | 473.3631 | 4.2732 |
| oleanen-27-oic acid | | |
| Spirosolane-3,23- | 474.3583 | 4.283 |
| diol; (3β,5a,22R,23S,25R)- | | |
| form: | | |
| 23-Ac | | |
| 6-Tricosyl-1,2,4- | 477.4307 | 8.3053 |
| benzenetriol: 2,4-Di- | | |
| Me ether | | |
| 2,6,10,15,19,23- | 479.41 | 5.0977 |
| Hexamethyl- | | |
| 2,6,18,22- | | |
| tetracosatetraene- | | |
| 10,11,14,15-tetrol | | |
| Molliorin A | 480.3841 | 3.916 |
| 25,28- | 481.4045 | 6.3683 |
| Dimethylstigmasta- | | |
| 5,22,28-trien-3-ol; | | |
| (3β,22E,24?)-form: | | |
| Ac | | |
| Rhodopeptin; | 482.3706 | 3.6661 |
| Rhodopeptin C1 | | |
| Plakinamine A: 3- | 483.395 | 3.6658 |
| Epimer, 4β-hydroxy, | | |
| N3, N3-di-Me, N30- | | |
| oxide | | |
| Verazine: 22S, N- | 484.379 | 3.491 |
| Dihydro, N, O-di-Ac | | |
| 3,20-Diaminopregn- | 485.3743 | 2.7421 |
| 16-en-4-ol; | | |
| (3β,4β,5a,20S)- | | |
| form: N20,N20-Di- | | |
| Me, N3-(3-methyl- | | |
| 2-butenoyl), 4-Ac | | |
| 17,21-Epoxy-3,16- | 487.4151 | 1.3546 |
| hopanediol; | | |
| (3β,16a,17β,21β)- | | |
| form: 16-Et ether | | |
| 3,5-Acarnidine: N- | 488.3964 | 2.5596 |
| Dedodecanoyl, N- | | |
| (5Z,8Z,11Z- | | |
| tetradecatrienoyl) | | |
| 2,29-Diamino- | 489.4056 | 5.086 |
| 5,9,11,14,17,20- | | |
| triacontahexaene- | | |
| 3,8,28-triol | | |
| Furospinulosin 3 | 491.4253 | 7.7091 |
| 25-(3,5- | 493.4257 | 5.936 |
| Dihydroxyphenyl)- | | |
| 9,10-pentacosanediol | | |
| Lycocaroline: | 494.411 | 4.1167 |
| Didehydro | | |
| Quassiol A | 495.4049 | 5.0879 |
| Rhodopeptin; | 496.3863 | 4.6383 |
| Rhodopeptin C2 | | |
| CyclovirobuxeineI: | 497.4107 | 3.6858 |
| N3,N3,N20-Tri- | | |
| Me,O-tigloyl | | |
| Rubijervine: Di-Ac | 498.3583 | 2.4673 |
| 20-Taraxastene- | 499.4151 | 3.0862 |
| 3,19-diol; (3β,19a)- | | |
| form: 19-Me ether, | | |
| 3-Ac | | |
| 3,20- | 501.4056 | 2.7213 |
| Diaminopregnan-4- | | |
| ol; (3a,4β,5a,20S)- | | |
| form: N3,N20,N20- | | |
| Tri-Me, N3-(3- | | |
| methyl-2-butenoyl), | | |
| O—Ac | | |
| CyclomicrophyllineI: | 503.3485 | 4.8057 |
| 6,7-Dihydro, N3,O16- | | |
| di-Ac,N20- | | |
| formyl | | |
| 16,17,19- | 505.4257 | 3.4984 |
| Kauranetriol; (ent- | | |
| 16βOH)-form: 19- | | |
| Dodecanoyl | | |
| Ergostane- | 509.3842 | 3.5957 |
| 1,3,5,6,25-pentol; | | |
| (1β,3β,5a,6β,24S)- | | |
| form: 25-Ac | | |
| Estra-1,3,5(10)- | 511.4151 | 2.7094 |
| triene-3,17-diol; | | |
| 17β-form: 17- | | |
| Hexadecanoyl | | |
| 2,6,10,15,19,23- | 513.4155 | 3.0449 |
| Hexamethyl- | | |
| 14,18,22- | | |
| tetracosatriene- | | |
| 2,3,6,7,10,11-hexol | | |
| 5,14:7,8-Diepoxy-5- | 515.41 | 1.8275 |
| marasmanol; | | |
| (5β,7β,8β)-form: 9Z- | | |
| Octadecenoyl | | |
| 5,14:7,8-Diepoxy-5- | 517.4257 | 3.7185 |
| marasmanol; | | |
| (5β,7β,8β)-form: | | |
| Octadecanoyl | | |
| Cyclostellettamines; | 519.4678 | 4.7142 |
| !Cyclostellettamine | | |
| D: 7′,8′Z- | | |
| Didehydro | | |
| Kotolactone A | 521.457 | 3.8888 |
| Hoprominol | 523.4587 | 4.632 |
| Rhodopeptin; | 524.4176 | 3.4896 |
| Rhodopeptin B5 | | |
| Niphatoxin B | 525.4083 | 3.2666 |
| Sarain C | 526.4134 | 2.7243 |
| 12-Oleanene-3,28- | 527.41 | 1.7876 |
| diol; 3β-form: Di-Ac | | |
| Buxidienine I: 16- | 529.4369 | 3.3343 |
| Deoxy, N3,N20,N20- | | |
| tri-Me, N3-(2R- | | |
| hydroxy-3?- | | |
| methylpentanoyl) | | |
| Pentacosyl glucoside | 531.4624 | 3.5029 |
| 16,17,19- | 533.457 | 6.4929 |
| Kauranetriol; (ent- | | |
| 16βOH)-form: 19- | | |
| Tetradecanoyl | | |
| Cyclostellettamines; | 535.4991 | 7.9792 |
| CyclostellettamineE | | |
| ?,?-Carotene | 537.446 | 15.631 |
| 7,7′-Dihydro-β- | 539.4617 | 3.5786 |
| carotene | | |
| Phloeodictyne C; | 540.4298 | 2.5824 |
| Phloeodictyne C2 | | |
| 1,2,7,8,11,12- | 543.493 | 5.2046 |
| Hexahydrolycopene | | |
| 2-(4- | 545.4933 | 7.613 |
| Hydroxyphenyl)ethanol: | | |
| 1-O- | | |
| Octacosanoyl | | |
| 3-Methyl-2-[6-(3- | 550.426 | 21.1897 |
| methyl-2-butenyl)- | | |
| 1H-indol-3-yl]-1,3- | | |
| butanediol; (R)- | | |
| form: O2′-(9Z,12Z- | | |
| Octadecadienoyl) | | |
| 3-Methyl-2-[6-(3- | 552.4416 | 11.6698 |
| methyl-2-butenyl)- | | |
| 1H-indol-3-yl]-1,3- | | |
| butanediol; (R)- | | |
| form: O2′-(9Z- | | |
| Octadecenoyl) | | |
| β,β-Carotene-2,2′- | 553.4409 | 8.6729 |
| diol; (2R,2′R)- | | |
| form: Monodeoxy | | |
| Helianol: Octanoyl | 555.5141 | 12.2786 |
| Salvianolic acid K | 557.1295 | 0.0589 |
| 8(17)-Labden-15-ol; | 557.5297 | 16.5978 |
| (ent-13R)-form: | | |
| Carboxylic acid, 9- | | |
| octadecenyl ester | | |
| Pillaromycin A | 559.1815 | 0.1234 |
| 35-Amino- | 562.4835 | 5.6967 |
| 30,31,32,33,34- | | |
| bacteriohopanepentol; | | |
| (28?,30R,31R,32R,33S,34S)- | | |
| form: 30-Deoxy | | |
| Isoclathriaxanthin | 563.3889 | 8.9868 |
| Secamine: | 565.427 | 5.3085 |
| Bis(demethoxycarbonyl), | | |
| 15,15′,20,20′- | | |
| tetrahydro | | |
| 5-(2,3-Epoxy-3- | 568.4366 | 5.9584 |
| methylbutyl)-3-[2- | | |
| hydroxy-1- | | |
| (hydroxymethyl)-2- | | |
| methylpropyl]-1H- | | |
| indole; (1′S,2″S)- | | |
| form, 5′-(9- | | |
| Octadecenoyl)(Z-) | | |
| Griseochelin | 569.4417 | 7.0012 |
| Rhodopin: 7′,8′- | 571.4879 | 4.8642 |
| Dihydro, Me ether | | |
| Coriacyclodienin | 573.4883 | 7.2915 |
| Stephasubine; (R)- | 575.2182 | 0.2817 |
| form: 1,2- | | |
| Didehydro, N-de- | | |
| Me | | |
| Montecristin | 575.5039 | 14.568 |
| Fritillebinide C: 3- | 577.4984 | 9.6724 |
| Deacetoxy | | |
| 6-Benzoyl-5,7- | 578.1002 | 0.0311 |
| dihydroxy-2-methyl- | | |
| 2-(4-methyl-3- | | |
| pentenyl)chroman; | | |
| β-D-form: Tetra-Ac | | |
| 35-Amino- | 578.4784 | 6.6778 |
| 30,31,32,33,34- | | |
| bacteriohopanepento | | |
| 1; (28?,30R,31R,32R,33S,34S)- | | |
| form | | |
| Bengamide Z: 6- | 583.4322 | 4.4873 |
| Deoxy, 5′-O- | | |
| tetradecanoyl | | |
| 16-Kaurene-7,18- | 585.4883 | 3.261 |
| diol; (ent-7a)-form: | | |
| 18-Hexadecanoyl, 7- | | |
| Ac | | |
| 3-(3,4- | 587.5039 | 3.5948 |
| Dihydroxyphenyl)- | | |
| 2-propenoicacid; (E)- | | |
| form: 4′-Me ether, | | |
| octacosyl ester | | |
| 1,1′,2,2′,7,8- | 589.4984 | 5.6427 |
| Hexahydro-Ψ,Ψ- | | |
| carotene-1,1′-diol; | | |
| 1′-Me ether | | |
| 2-Amino-4,8,10- | 590.5148 | 5.154 |
| octadecatriene-1,3- | | |
| diol; (2S,3R,4E,8E,10E)- | | |
| form: N-(2R- | | |
| Hydroxy-15R- | | |
| methyl-3E- | | |
| octadecenoyl) | | |
| 2-[14-[3-(1,5- | 591.5141 | 6.558 |
| Dimethylhexyl)cyclopentyl]- | | |
| 3,7,11- | | |
| trimethyltetradecyl]- | | |
| 3-methyl-1,4- | | |
| naphthoquinone | | |
| Fritillebin C | 593.4933 | 4.2244 |
| Human ocular | 594.4675 | 3.92 |
| retinalpigment | | |
| Minalemines; | 597.4928 | 3.7552 |
| Minalemine A | | |
| 5,7-Dihydroxy-6- | 599.5039 | 8.0648 |
| methyl-2-nonacosyl- | | |
| 4H-1-benzopyran-4- | | |
| one | | |
| 3-(3,4- | 601.5196 | 7.8676 |
| Dihydroxyphenyl)- | | |
| 2-propenoicacid; (E)- | | |
| form: Triacontyl | | |
| ester | | |
| Bombiprenone | 603.5505 | 5.6071 |
| 19(10?9)-Abeo-3,4- | 605.5145 | 2.3915 |
| secotirucall-4-ene- | | |
| 3,24,25-triol; (24R)- | | |
| form: 4,5a-Epoxide, | | |
| 3-octanoyl | | |
| 2,6,10,15,19,23- | 607.4574 | 2.4525 |
| Hexamethyl- | | |
| 2,6,14,18,22- | | |
| tetracosapentaene- | | |
| 10,11-diol: 11-O-β- | | |
| D-Mannopyranoside | | |
| 4-Aminotetrahydro- | 608.5618 | 3.6813 |
| 2-(4-tetradecenyl)-3- | | |
| furanol; | | |
| (2R*,3R*,4R*,4′Z)- | | |
| form, N-(2R- | | |
| Hydroxyeicosanoyl) | | |
| Glycerol | 611.525 | 5.2797 |
| trialkanoates (diacid, | | |
| unsymmetrical); | | |
| Glycerol 1,2- | | |
| didecanoate 3- | | |
| tetradecanoate | | |
| Iristectorene A: 16- | 615.5352 | 6.267 |
| Deoxy, 22,23- | | |
| dihydro, deacyl, 3-O- | | |
| decanoyl | | |
| 3-(3,4- | 617.5509 | 7.4899 |
| Dihydroxyphenyl)- | | |
| 1-propanol: 1-O- | | |
| Hentriacontanoyl | | |
| 1,3,25,27- | 621.4941 | 2.2023 |
| Octacosanetetrol; | | |
| (3R,25S,27R)- | | |
| form: 1-O-a-D- | | |
| Mannopyranoside | | |
| Myxovirescin N1: 4- | 622.4319 | 3.5286 |
| Epimer | | |
| Homodestruxin B: 4- | 624.3972 | 0.7946 |
| Hydroxy | | |
| Batzelladine F | 625.5169 | 3.4315 |
| 2-Hentriacontyl-5,7- | 627.5352 | 3.8316 |
| dihydroxy-6-methyl- | | |
| 4H-1-benzopyran-4- | | |
| one | | |
| Ormosinine | 631.5427 | 2.7096 |
| Calebassine: 18- | 633.3804 | 0.4598 |
| Hydroxy | | |
| Hypsiziprenols; | 635.5614 | 3.2048 |
| Hypsiziprenol B8 | | |
| Annoglaucin | 639.4836 | 2.1391 |
| Enniatin B | 640.4173 | 1.2942 |
| 22,26-Epoxyergost- | 641.3901 | 0.2087 |
| 5-ene-1,3,24,25,26- | | |
| pentol; | | |
| (1a,3β,22R,24R,25S,26R)- | | |
| form: 26-O-β-D- | | |
| Glucopyranoside | | |
| Antibiotic WB | 641.5105 | 1.4814 |
| 3559C | | |
| 2-Tetradecyl-2- | 643.5513 | 2.0589 |
| octadecene-1,4-diol; | | |
| (R,Z)-form: 1-O-β- | | |
| D-Glucopyranoside | | |
| 1-O-(14,16- | 649.5771 | 2.5485 |
| Dimethyloctadecanoyl)- | | |
| 3-O-(9,12- | | |
| octadecadienoyl)glycerol | | |
| 4-Aminotetrahydro- | 650.6087 | 4.7662 |
| 2-(4-tetradecenyl)-3- | | |
| furanol; | | |
| (2R*,3R*,4R*,4′Z)- | | |
| form, N-(2R- | | |
| Hydroxytricosanoyl) | | |
| Spirost-5-en-3-ol; | 653.5509 | 2.0234 |
| (3β,25R)-form: | | |
| Hexadecanoyl | | |
| 4,21,27,43- | 655.5454 | 1.7762 |
| Hexatetracontatetraene- | | |
| 1,12,15,45- | | |
| tetrayne-3,14-diol; | | |
| (3R,4E,14R,21Z,27Z,43Z)- | | |
| form | | |
| 4-Methylaconitane- | 658.3591 | 0.214 |
| 1,6,8,14,16,18- | | |
| hexol; | | |
| (1a,5β,6a,14a,16β)- | | |
| form: O1,O6,O16,O18- | | |
| Tetra-Me, | | |
| N-Et, 14-(3,4- | | |
| dimethoxybenzoyl), | | |
| 8-Ac | | |
| 2-Amino-6,9- | 664.6244 | 3.3156 |
| heptacosadiene- | | |
| 1,3,5-triol; | | |
| (2?,3?,5?,6E,9E)- | | |
| form: N- | | |
| Pentadecanoyl | | |
| Cholest-5-ene- | 666.4581 | 2.6681 |
| 3,15,26-triol; | | |
| (3a,15a,25R)-form: | | |
| 15-O-[2- | | |
| (Acetylamino)-2- | | |
| deoxy-β-D- | | |
| glucopyranosyl], 26- | | |
| Ac, 5a,6-dihydro | | |
| Petroformyne 1: | 667.509 | 2.0345 |
| 8,9,14,15- | | |
| Tetradehydro(Z,Z-), | | |
| 12,13-dihydro | | |
| Batzelladine G | 669.5431 | 1.6082 |
| Tabersonine: Dimer | 671.3597 | 0.1696 |
| Bacchalejin 1 | 673.4468 | 0.328 |
| Cholest-5-en-3-ol; | 673.5923 | 0.6732 |
| 3β-form: 3-O- | | |
| (5Z,8Z,11Z,14Z- | | |
| Eicosatetraenoyl) | | |
| 4,8-Diethyl-10- | 675.608 | 1.9843 |
| phenyldecanoic acid: | | |
| 3β-Cholestanyl ester | | |
| Stevastelin C3: 5R- | 676.3843 | 0.37 |
| Hydroxy, 13-deoxy, | | |
| 12,13-didehydro, 14- | | |
| O-sulfate | | |
| Pycnocomolide: 4″- | 681.3427 | 0.1321 |
| Methoxy | | |
| 12-Oleanene-3,28- | 681.6185 | 4.9073 |
| diol; 3β-form: 3- | | |
| Hexadecanoyl | | |
| 4,8-Diethyl-10- | 689.5872 | 1.8197 |
| phenyldecanoic acid: | | |
| 3-Hydroxy, | | |
| cholesteryl ester | | |
| Ergosta-7,22-diene- | 693.5822 | 1.9447 |
| 3,5,6-triol; | | |
| (3β,5a,6β,22E,24R)- | | |
| form: 6-O-(9Z,12Z- | | |
| Octadecadienoyl) | | |
| 5,8-Epidioxyergosta- | 695.5978 | 2.1886 |
| 6,22-dien-3-ol; | | |
| (3β,5a,8a,22E,24R)- | | |
| form: | | |
| Octadecanoyl | | |
| 20-Taraxastene- | 697.6135 | 2.5187 |
| 3,16,22-triol; | | |
| (3β,16β,22a)-form: | | |
| 3-Hexadecanoyl | | |
| Glycerol 1,2- | 702.552 | 1.6724 |
| dialkanoates; | | |
| !Glycerol 1,2- | | |
| dipentadecanoate: 3- | | |
| O-(6-Amino-6- | | |
| deoxy-β-D- | | |
| glucopyranoside) | | |
| Cordiaquinone A: | 705.5822 | 0.9003 |
| Hexacosanoyl | | |
| 29-(2,3,4,5- | 706.5258 | 1.3065 |
| Tetrahydroxypentyl)hopane; | | |
| (21βH,22?,29?,30?,33?,34?)- | | |
| form: 32-Ketone, | | |
| 35-O-(2-amino-2- | | |
| deoxy-β-D- | | |
| glucopyranoside) | | |
| 29-(2,3,4,5- | 708.5414 | 1.789 |
| Tetrahydroxypentyl)hopane; | | |
| (21βH,32R,33R,34S)- | | |
| form: | | |
| 35-O-(6-Amino-6- | | |
| deoxy-β-D- | | |
| glucopyranoside) | | |
| 2-Amino-4,8- | 714.552 | 1.1009 |
| octadecadiene-1,3- | | |
| diol; (2S,3R,4E,8E)- | | |
| form: N-(2R- | | |
| Hydroxyhexadecanoyl), | | |
| 1-O-β-D- | | |
| glucopyranoside | | |
| Vitamin K2; | 717.561 | 1.6744 |
| !Vitamin K2(40) | | |
| 23-Methyl-1,23- | 719.5673 | 0.9975 |
| nonacosadiene- | | |
| 4,6,8,10,12,14,16,18,20,22- | | |
| decol; | | |
| (4S,6S,8S,10S,12S,14S,- | | |
| 16S,18S,20S,22S,- | | |
| 23E)-form: Deca- | | |
| Me ether | | |
| 2-[3,7,11,15,19,23- | 721.5923 | 1.6154 |
| Hexamethyl-25- | | |
| (2,6,6-trimethyl-2- | | |
| cyclohexenyl)pentacosa- | | |
| Bacterioruberin: 3″- | 725.5872 | 0.8345 |
| Deoxy, 2″,3″- | | |
| didehydro, 3′,4′- | | |
| dihydro | | |
| 2-Amino-9-methyl- | 726.552 | 1.0706 |
| 4,8,10- | | |
| octadecatriene-1,3- | | |
| diol; (2S,3R,4E,8E,10E)- | | |
| form: N-(2R- | | |
| Hydroxyhexadecanoyl), | | |
| 1-O-β-D- | | |
| glucopyranoside | | |
| Mycinamycin II | 728.4221 | 0.2193 |
| 2-Amino-9-methyl- | 728.5676 | 0.7456 |
| 4,8-octadecadiene- | | |
| 1,3-diol; (2S,3R,4E,8E)- | | |
| form: N-(2R- | | |
| Hydroxyhexadecanoyl), | | |
| 1-O-β-D- | | |
| glucopyranoside | | |
| Glycerol 1,2- | 731.5673 | 0.7901 |
| dialkanoates; | | |
| Glycerol 1,2- | | |
| dihexadecanoate: 3- | | |
| O-β-D- | | |
| Glucopyranoside | | |
| Pseudotheonamide C | 732.3469 | 0.1623 |
| Tabernaelegantinine C | 732.4125 | 0.1495 |
| 20(29)-Lupene-3,28- | 733.4679 | 0.6184 |
| diol; 3β-form: 3-O- | | |
| (4-Hydroxy-E-camoyl), | | |
| 28-O-(3- | | |
| ethoxycarbonyl-2- | | |
| hydroxypropanoyl) | | |
| Cabufiline: Deepoxy | 735.4121 | 0.2471 |
| Lepidostatin 1 | 738.5295 | 1.6259 |
| Bacterioruberin: 3″- | 739.5665 | 1.3474 |
| Deoxy, 2″,3″- | | |
| didehydro, 3′,4′- | | |
| epoxide | | |
| Manzamenone A | 743.5826 | 1.1845 |
| 2-Amino-1,3,4- | 746.6146 | 1.7296 |
| octadecanetriol; | | |
| (2S,3S,4R)-form: N- | | |
| Octadecanoyl, 1-O- | | |
| β-D- | | |
| galactopyranoside | | |
| Glycerol | 751.6815 | 2.4666 |
| trialkanoates (diacid, | | |
| unsymmetrical); | | |
| Glycerol 1,2- | | |
| ditetradecanoate 3- | | |
| hexadecanoate | | |
| Pepstatins; Pepstatin J | 756.5486 | 1.0563 |
| Glycerol 1,2- | 757.583 | 0.8365 |
| dialkanoates; | | |
| !Glycerol 2-(9Z- | | |
| octadecenoate) 1- | | |
| hexadecanoate: 3- | | |
| O-β-D- | | |
| Galactopyranoside | | |
| Phosphatidylcholine; | 758.57 | 0.6886 |
| Glycerol 2-(9,12- | | |
| octadecadienoate) 1- | | |
| hexadecanoate 3- | | |
| phosphocholine | | |
| 2-Amino-8- | 760.5938 | 1.1366 |
| octadecene-1,3,4- | | |
| triol; (2S,3S,4R,8Z)- | | |
| form: N-(2R- | | |
| Hydroxyoctadecanoyl), | | |
| 1-O-β-D- | | |
| glucopyranoside | | |
| Phosphatidylethanol | 768.5543 | 1.4242 |
| amine; Glycerol 2- | | |
| (5Z,8Z,11Z,14Z- | | |
| eicosatetraenoate) 1- | | |
| octadecanoate 3- | | |
| phosphoethanolamine | | |
| 2- | 771.608 | 0.8525 |
| (3,7,11,15,19,23,27,31,35- | | |
| Nonamethyl- | | |
| 2,6,10,14,18,22,26,30,34- | | |
| Oligomycin A: 12- | 775.536 | 0.2671 |
| Deoxy | | |
| 25-Methyl-1,25- | 777.6092 | 1.0464 |
| hentriacontadiene- | | |
| 4,6,8,10,12,14,16,18,20,22,24- | | |
| undecol; | | |
| (4S,6S,8S,10S, | | |
| 12S,14S,16S,18S, | | |
| 20S,22S,24S,25E)- | | |
| form: Undeca-Me | | |
| ether | | |
| Rhodoquinone 9 | 780.6294 | 1.3846 |
| Erythromycin,,: | 792.5109 | 0.4367 |
| 14-(1- | | |
| Hydroxypropyl) | | |
| 1-Hentriacontene- | 795.6197 | 0.8965 |
| 4,6,8,10,12,14,16,18, | | |
| 20,22,24,26-dodecol; | | |
| (4S,6S,8S,10S,12S, | | |
| 14S,16R,18R,20R, | | |
| 22R,24R,26R)- | | |
| form: Dodeca-Me | | |
| ether | | |
| Crambidine | 800.6139 | 0.942 |
| 2-Polyprenyl-1,4- | 803.5648 | 0.8709 |
| benzenediol; !2- | | |
| Nonaprenyl-1,4- | | |
| benzenediol: 1- | | |
| Sulfate | | |
| 2-Amino-4,8,10- | 808.6302 | 1.0116 |
| octadecatriene-1,3- | | |
| diol; (2S,3R,4E,8E,10E)- | | |
| form: N-(2R- | | |
| Hydroxy-14Z- | | |
| tricosenoyl), 1-O-β- | | |
| D-glucopyranoside | | |
| Siphonaxanthin: 19- | 809.6084 | 1.0763 |
| O-(2E- | | |
| Tetradecenoyl) | | |
| Diacylphosphatidylcarnitine; | 810.586 | 0.958 |
| Dihexadecanoylphosphatidylcarnitine | | |
| 3,3′,4′,5,7- | 814.1804 | 0.1324 |
| Pentahydroxy-5′- | | |
| methoxyflavylium(1+); | | |
| 3,7-Bis-O-(6-O- | | |
| malonyl-β-D- | | |
| glucopyranoside) | | |
| 2-Amino-8- | 816.6564 | 1.0722 |
| octadecene-1,3,4- | | |
| triol; (2S,3S,4R, | | |
| 8Z)-form: N-(2R- | | |
| Hydroxydocosanoyl), | | |
| 1-O-β-D- | | |
| glucopyranoside | | |
| Phosphatidylcholine; | 818.6639 | 1.7078 |
| Glycerol 1,2- | | |
| dinonadecanoate 3- | | |
| phosphocholine | | |
| Halovir; Halovir D: | 822.6068 | 1.0074 |
| 4′-Deoxy | | |
| 4-Methylaconitane- | 824.5676 | 0.614 |
| 1,6,8,14,16,18- | | |
| hexol; | | |
| (1a,5β,6a,14a,16β)- | | |
| form: O1,O6,O16,O18- | | |
| Tetra-Me, | | |
| N-Et, 14-(4- | | |
| methoxybenzoyl), 8- | | |
| hexadecanoyl | | |
| Crambescidin826 | 827.6374 | 1.0674 |
| Crambescidin830 | 831.6323 | 0.9229 |

TABLE 15
DART TOF-MS characterization of Nettle Extract 8.

| | | Rel. |
|---|---|---|
| | Calc. | Abund. |
| Compound Name | Mass | (%) |
| 1,5-Dihydro-5- | 100.0398 | 0.8591 |
| hydroxy-2H-pyrrol- | | |
| 2-one | | |
| 4-Hydroxy-2- | 103.0395 | 13.9001 |
| oxobutanal | | |
| N,N- | 104.0711 | 35.8039 |
| Dimethylglycine | | |
| Butanoic acid: | 107.0263 | 2.6431 |
| Chloride | | |
| 1,4-Benzoquinone | 109.0289 | 14.1503 |
| 1H-Pyrrole-3- | 110.0718 | 3.6859 |
| carboximidamide | | |
| Resorcinol | 111.0446 | 6.9863 |
| 2,3-Dihydro-1H- | 112.0762 | 5.2948 |
| pyrrole: N—Ac | | |
| 6-azacytosine | 113.0238 | 5.8461 |
| 3-Azetidinone: N—Ac | 114.0555 | 4.4375 |
| 3,4-Dihydroxy-2- | 115.0395 | 5.5046 |
| methylenebutanoicacid; | | |
| (S)-form: | | |
| Lactone | | |
| Proline | 116.0534 | 2.6483 |
| Levulinic acid | 117.0612 | 29.1802 |
| 2-Amino-3,4- | 118.0504 | 2.2536 |
| dihydroxybutanoic | | |
| (1a,5β,6a,14a,16β)- | | |
| form: O1,O6,O | | |
| Succinic acid | 119.0344 | 14.1227 |
| Threonine | 120.066 | 2.2499 |
| Nitrosofungin | 121.0613 | 2.8986 |
| 3- | 122.0639 | 1.2275 |
| (Methylthio)propylamine: | | |
| S-Oxide | | |
| Vitamin B3 | 123.0446 | 0.8337 |
| (niacinamide) | | |
| 2- | 124.0762 | 6.8706 |
| Hydroxybenzylamine | | |
| Hydroxy-1,4- | 125.0238 | 0.7304 |
| benzoquinone | | |
| 1,4-Benzenediol: | 125.0602 | 1.526 |
| Mono-Me ether | | |
| 3,4-Dihydroxy-2- | 126.0555 | 5.56 |
| methylpyridine; Di- | | |
| OH-form | | |
| 5-Hydroxy-3-vinyl- | 127.0395 | 51.7266 |
| 2(5H)-furanone | | |
| 2-Ethyl-4- | 128.0534 | 8.0464 |
| methylthiazole | | |
| 5,6-Dihydro-5- | 129.0551 | 6.7549 |
| hydroxy-6-methyl- | | |
| 2H-pyran-2-one; | | |
| (5R,6S)-form | | |
| 3,4-Dihydro-4- | 130.0504 | 12.2547 |
| hydroxy-2H- | | |
| pyrrole-2-carboxylic | | |
| acid | | |
| 2-Nonene-4,6,8- | 131.0497 | 0.8694 |
| triyn-1-ol | | |
| Leucine | 132.0773 | 4.5202 |
| 3,4-Dihydroxy-2- | 133.0501 | 6.9241 |
| methylenebutanoicacid | | |
| 1H-Indol-3-ol | 134.0606 | 1.3676 |
| Adenine | 136.061 | 17.6354 |
| 4-Methylbenzoic | 137.0602 | 33.5404 |
| acid | | |
| 5-Ethyl-2- | 138.0919 | 13.6092 |
| methylpyridine: N- | | |
| Oxide | | |
| 2-Hydroxybenzoic | 139.0395 | 3.6799 |
| acid | | |
| Choline: Chloride | 140.0842 | 6.3247 |
| a-Amino-2- | 142.0868 | 2.3487 |
| cyclopentene-1- | | |
| acetic acid | | |
| 2- | 144.066 | 10.4184 |
| Hydroxymethylclavam | | |
| 2,3-Dihydro-3,6- | 145.0501 | 52.9997 |
| dihydroxy-2-methyl- | | |
| 4H-pyran-4-one | | |
| 1H-Indole-3- | 146.0606 | 8.4713 |
| carboxaldehyde | | |
| 2,5-Furandiacetic | 147.0558 | 8.1801 |
| acid: Dinitrile | | |
| 2-Hydroxybenzoic | 148.0762 | 2.8839 |
| acid: Et ether, nitrile | | |
| O-Carbamoylserine | 149.0562 | 10.0849 |
| 4-Methylbenzoic | 150.0919 | 7.4692 |
| acid: Methylamide | | |
| Diisopropyldisulfide | 151.0615 | 2.5271 |
| 2-Hydroxybenzoic | 153.0664 | 3.2776 |
| acid: Hydrazide | | |
| Scopine: 3-Ketone | 154.0868 | 9.916 |
| 3,4- | 155.0708 | 3.7103 |
| Dihydroxybenzyl | | |
| alcohol: 4-Me ether | | |
| 4-Amino-2-hydoxy- | 156.0773 | 6.7595 |
| 5- | | |
| (hydroxymethyl)pyrimidine; | | |
| OH-form: | | |
| 2-Me ether | | |
| 3-(2-Hydroxyethyl)- | 158.0817 | 3.7067 |
| 4-oxa-1- | | |
| azabicyclo[3.2.0]heptan- | | |
| 7-one | | |
| 4-Hydroxy-1,1- | 160.0973 | 3.1421 |
| dimethylpyrrolidinium- | | |
| 2-carboxylate; (2S,4R)- | | |
| form | | |
| 2,8-Decadiene-4,6- | 161.0602 | 1.9697 |
| diynoic acid; (2E,8Z)- | | |
| form | | |
| 3-Aminotetrahydro- | 162.0766 | 16.0397 |
| 5-(hydroxymethyl)- | | |
| 3-furancarboxylic | | |
| acid | | |
| Levoglucosan/Glyogen/ | 163.0615 | 85.6267 |
| Laminarin | | |
| N-[2-(4- | 164.0711 | 95.9778 |
| Hydroxyphenyl)ethenyl]formamide | | |
| Rhamnose; D-form | 165.0763 | 16.337 |
| 2-Hydroxybenzoic | 167.0708 | 2.9161 |
| acid: Et ester | | |
| Synephrine | 168.1024 | 11.9063 |
| Tetramethylpyrazine: | 169.0977 | 8.8411 |
| 1,4-Dioxide | | |
| 2-Amino-3-(3- | 170.0817 | 5.2077 |
| furanyl)propanoic | | |
| acid; (S)-form: N- | | |
| Me | | |
| 2-Amino-4-hexenoic | 172.0973 | 3.9625 |
| acid; (±)-(Z)-form: | | |
| N—Ac | | |
| Octanedioic acid: | 173.129 | 5.5743 |
| Diamide | | |
| 8-Methyl-8- | 174.113 | 6.445 |
| azabicyclo[3.2.1]octane- | | |
| 3,6,7-triol | | |
| Shikimic acid | 175.0606 | 17.2509 |
| Muscarine‡ | 175.1572 | 46.5283 |
| 4-Amino-1-phenyl- | 176.1075 | 22.1547 |
| 1-penten-3-one | | |
| 2,4(1H,3H)- | 177.0664 | 7.8624 |
| Quinazolinedione; | | |
| Di-NH-form: 1-Me | | |
| N-[2-(4- | 178.0868 | 72.6328 |
| Hydroxyphenyl)ethenyl]formamide: | | |
| Me | | |
| ether | | |
| N-Benzoylglycine: | 179.082 | 19.2852 |
| Amide | | |
| 6-Deoxymannonic | 180.0872 | 53.545 |
| acid; L-form: | | |
| Amide | | |
| 2-(4- | 181.0864 | 21.209 |
| Hydroxyphenyl)propanoic | | |
| acid; (R)- | | |
| form: Me ester | | |
| (2- | 182.0946 | 17.1342 |
| Aminoethyl)phosphonic | | |
| acid: Di-Et ester | | |
| Dihydro-5-(5- | 183.1021 | 11.0558 |
| hydroxy-1,3- | | |
| hexadienyl)-2(3H)- | | |
| furanone | | |
| Pyridoxine,,: 5-Me | 184.0973 | 4.3014 |
| ether | | |
| 4-(1H-Indol-3-yl)-3- | 186.0919 | 6.9941 |
| buten-2-one | | |
| 4-Hydroxy-3-(3- | 187.0759 | 8.0705 |
| methyl-3-buten-1- | | |
| ynyl)benzoic acid: | | |
| 1′-Aldehyde | | |
| 2-Amino-4- | 188.0923 | 9.0792 |
| propylidenepentanedioic | | |
| acid | | |
| Glutamine,,; (S)- | 189.1239 | 2.8399 |
| form: N 5-Isopropyl | | |
| 2-Ethyl-4- | 190.098 | 8.4746 |
| hydroxyquinazoline; | | |
| 3H-form: N-Amino | | |
| Carnitine,; (R)- | 191.1521 | 52.594 |
| form: Et ester | | |
| Hydrohydrastinine | 192.1024 | 100 |
| 2,6,8,10- | 193.1228 | 45.7334 |
| Dodecatetraenoicacid | | |
| 2-Amino-2- | 194.1028 | 22.8225 |
| deoxygulose; D- | | |
| form: N-Me | | |
| 3,4-Dihydro-3,8- | 195.0657 | 3.7066 |
| dihydroxy-3-methyl- | | |
| 1H-2-benzopyran-1- | | |
| one | | |
| DL-methyl-m- | 196.0973 | 12.7012 |
| tyrosine | | |
| 7-Deoxy-D-glycero- | 197.1025 | 7.8065 |
| D-manno-heptitol | | |
| Stizolamine | 198.0991 | 23.1145 |
| 5-(1-Hydroxybutyl)- | 199.097 | 7.3395 |
| 6-hydroxymethyl-2H- | | |
| pyran-2-one | | |
| N-(2- | 200.1035 | 9.28 |
| Cyanoethyl)glutamine; | | |
| (±)-form | | |
| 4-Hydroxy-3-(3- | 201.0915 | 1.1854 |
| methyl-3-buten-1- | | |
| ynyl)benzoic acid: | | |
| 1′-Aldehyde, Me | | |
| ether | | |
| 2,6- | 202.1079 | 7.2597 |
| Piperidinedicarboxylic | | |
| acid; (2RS,6RS)- | | |
| form: Di-Me ester | | |
| 3,10(14)- | 203.18 | 7.7042 |
| Aromadendradiene | | |
| Glycylglycylglycine: | 204.0984 | 14.3461 |
| Me ester | | |
| 4-methyl-7- | 205.0765 | 27.5251 |
| ethoxycoumarin | | |
| N-(2- | 206.1181 | 28.3543 |
| Hydroxyethyl)camamide: | | |
| N-Me | | |
| Eremopetasinorol: 3- | 207.1385 | 18.4943 |
| Ketone | | |
| (N- | 208.1086 | 23.5276 |
| Phenylacetyl)glycine: | | |
| Hydrazide | | |
| 6,10-Dimethyl-5,9- | 209.1541 | 84.8735 |
| undecadiene-2,8- | | |
| dione | | |
| Elaeokanine E | 210.1494 | 21.642 |
| Antibiotic A 41-89; | 211.1334 | 10.4957 |
| Antibiotic A 41-89I | | |
| 4-Methylbenzoic | 212.1075 | 13.2325 |
| acid: Anilide | | |
| Linderazulene: 2,3- | 213.1279 | 3.3579 |
| Dihydro | | |
| 9H-Pyrido[3,4-b]indol- | 214.1106 | 10.204 |
| 6-ol: Me | | |
| ether, N2-Me | | |
| Cyclo(aspartylleucyl) | 215.1032 | 11.271 |
| 3-Hydroxy-5- | 216.1024 | 14.5614 |
| methyl-1- | | |
| naphthalenecarboxylic | | |
| acid: Me ether, | | |
| amide | | |
| 2,3,4,9-Tetrahydro- | 217.0977 | 12.0336 |
| 1H-pyrido[3,4-b]indol- | | |
| 1-one: 7- | | |
| Methoxy | | |
| Glycylglycylglycine: | 218.1141 | 17.6006 |
| Et ester | | |
| 2-Amino-3-(3- | 219.1133 | 11.9787 |
| indolyl)butanoic acid | | |
| Vitamin B5 | 220.1185 | 75.2096 |
| 5-Aminopentanoic | 221.129 | 25.8775 |
| acid: N-Benzoyl, | | |
| amide | | |
| Anhalonine; (±)- | 222.113 | 22.4152 |
| form | | |
| Cystathionine | 223.0752 | 3.1157 |
| Cerulenin | 224.1286 | 12.0524 |
| Murexine | 225.1477 | 9.1648 |
| 2-Amino-4-hydroxy- | 226.1079 | 8.7859 |
| 4-(4- | | |
| hydroxyphenyl)-3- | | |
| methylbutanoic acid | | |
| 1-(2,5- | 227.1283 | 7.3846 |
| Dihydroxyphenyl)- | | |
| 1,2-propanediol; (1R*,2R*)- | | |
| form: 1-Et, | | |
| 5′-Me ether | | |
| 2-Amino-4-hexynoic | 228.1236 | 7.6809 |
| acid; (R)-form: N- | | |
| tert- | | |
| Butyloxycarbonyl | | |
| Faramol | 229.1228 | 11.0973 |
| Kinetin; 7H-form: | 230.1042 | 22.6678 |
| 7-Me | | |
| 9H-Pyrido[3,4-b]indol- | 231.1133 | 9.2241 |
| 7-ol: 1,2,3,4- | | |
| Tetrahydro, Me | | |
| ether, N9-formyl | | |
| 2-Oxohexanedioic | 232.1185 | 16.0795 |
| acid: Di-Et ester, | | |
| oxime | | |
| Cytisine; (−)-form: | 233.129 | 14.2379 |
| N—Ac | | |
| 4-(3-Indolyl)-2- | 234.1494 | 51.0539 |
| methyl-1-butanol; (R)- | | |
| form: N-Methoxy | | |
| 8-Hydroxy-13-nor- | 235.1334 | 22.8104 |
| 4(15),7(11)- | | |
| eudesmadien-12,8- | | |
| olide | | |
| Anhalonine; (S)- | 236.1286 | 22.0952 |
| form: N-Me | | |
| 7-Hydroxy-13-nor- | 237.149 | 12.4241 |
| 3,9-bisaboladiene- | | |
| 2,11-dione | | |
| 2-Amino-3- | 238.1079 | 17.4859 |
| hydroxypentanoic | | |
| acid; (2RS,3RS)- | | |
| form: N-Benzoyl | | |
| isopropyl-B-D- | 239.1032 | 2.4369 |
| thiogalactopyranoside | | |
| Mescaline: N- | 240.1236 | 9.1217 |
| Formyl | | |
| 3,5- | 241.1228 | 6.7131 |
| Dihydroxystilbene; | | |
| (E)-form: Di-Me | | |
| ether | | |
| 2-Hydroxybenzoic | 242.1392 | 10.6948 |
| acid: Choline ester | | |
| phosphatidylcholine | 243.1232 | 5.4219 |
| 4-Oxo-2- | 244.1185 | 22.9826 |
| piperidinecarboxylic | | |
| acid; (S)-form: N- | | |
| tert- | | |
| Butyloxycarbonyl | | |
| osthole | 245.1137 | 14.6491 |
| 3-Deoxy-3- | 246.1341 | 26.2198 |
| (methylamino)arabinose; | | |
| β-L-Furanose- | | |
| form: N—Ac, 1,2-O- | | |
| isopropylidene | | |
| 8-Hydroxy- | 247.1334 | 16.7389 |
| 1,4,7(11)-guaiatrien- | | |
| 12,8-olide | | |
| 3-(3,4- | 248.1286 | 24.4732 |
| Methylenedioxyphenyl)- | | |
| 2-propenoic acid; | | |
| (E)-form: 2- | | |
| Methylpropylamide | | |
| 4-shogaol | 249.1338 | 9.409 |
| 2-Acetamido-2- | 250.129 | 20.077 |
| deoxyglucose; D- | | |
| form: 3,4-Di-Me | | |
| Lachnumfuran A | 251.1283 | 7.7813 |
| N-2-Phenylethylcamamide | 252.1388 | 16.8327 |
| Undecanedioic acid: | 253.0762 | 3.5253 |
| Dichloride | | |
| 2,4-Dihydroxy- | 253.144 | 1.6651 |
| 3,5,6- | | |
| trimethylbenzoicacid: | | |
| Di-Me ether, Et | | |
| ester | | |
| Anantine‡; (R)- | 254.1293 | 12.0259 |
| form | | |
| 5-Deoxy-3-C- | 255.1232 | 8.9449 |
| hydroxymethyllyxose; | | |
| a-L-Furanose- | | |
| form: Benzyl | | |
| glycoside | | |
| 2-Amino-3-phenyl- | 256.1337 | 13.013 |
| 1-propanol; (S)- | | |
| form: N-Benzoyl | | |
| Cordiachrome G | 257.1177 | 11.2864 |
| Cyclo(alanyltryptophyl); | 258.1242 | 15.7373 |
| (3?,6?)-form | | |
| Elaeagin | 259.1334 | 14.1544 |
| 3-(Dimethylamino)- | 260.1498 | 17.4424 |
| 2,3,6-trideoxy-lyxo- | | |
| hexose; a-L- | | |
| Pyranose-form: Di- | | |
| Ac | | |
| Cyclo(leucylphenylalanyl) | 261.1603 | 18.0034 |
| 8-Methyl-8- | 262.1443 | 18.5124 |
| azabicyclo[3.2.1]octane- | | |
| 3,6-diol; (3R,6R)- | | |
| form: 3- | | |
| Benzoyl | | |
| 6,10,14-Trimethyl- | 263.2375 | 22.774 |
| 5,9,13- | | |
| pentadecatrien-2-one | | |
| 2-Pyrrolidineacetic | 264.1236 | 17.622 |
| acid; (S)-form: N- | | |
| Benzyloxycarbonyl | | |
| Eupomatenoid 6 | 265.1228 | 7.4198 |
| Anisomycin | 266.1392 | 13.6251 |
| 1,5-Bis(4- | 267.1021 | 1.196 |
| hydroxyphenyl)-1,4- | | |
| pentadien-3-one; | | |
| (E,E)-form | | |
| Peyoruvic acid | 268.1185 | 17.3753 |
| 8,8a-Dihydro-8a- | 269.1389 | 8.2623 |
| hydroxy-3,3,6,6,8,8- | | |
| hexamethyl-1,2- | | |
| benzodioxin-5,7(3H, | | |
| 6H | | |
| 1,2,3,4-Tetrahydro- | 270.1341 | 9.9341 |
| 5-hydroxy-1- | | |
| hydroxymethyl-6,7- | | |
| dimethoxy-2- | | |
| methylisoquinoline; | | |
| (?)-form: N-Oxide | | |
| 2-Hydroxybenzoic | 271.1334 | 15.6454 |
| acid: 4-tert- | | |
| Butylphenyl ester | | |
| 2,3,5-Triamino- | 272.1246 | 16.1735 |
| 2,3,5- | | |
| trideoxyarabinonic | | |
| acid; D-form: 1,4- | | |
| Lactone, tri-N—Ac | | |
| Verimol F | 273.149 | 13.4584 |
| 3-Amino-3- | 274.129 | 34.0065 |
| deoxyribose; a-D- | | |
| Furanose-form: 1,2- | | |
| O-Isopropylidene, N,5- | | |
| di-Ac | | |
| 8,11,13- | 275.2011 | 34.2404 |
| Podocarpatriene- | | |
| 12,13-diol: 12-Me | | |
| ether | | |
| 8-Methyl-8- | 276.1599 | 28.1913 |
| azabicyclo[3.2.1]octane- | | |
| 3,6-diol; (3R,6R)- | | |
| form: 3-O- | | |
| Phenylacetyl | | |
| 9,11,13,15- | 277.2167 | 86.7457 |
| Octadecatetraenoic | | |
| acid; (9E,11E,13E,15E)- | | |
| form | | |
| Stenine | 278.212 | 29.6347 |
| 6,9-Dihydroxy-15- | 279.1232 | 1.5275 |
| oxo-1,3,11(13)- | | |
| elematrien-12,8- | | |
| olide; (ent- | | |
| 6a,7a,8β,9β)-form | | |
| 7-Hydroxy-14,15- | 279.2324 | 76.3732 |
| dinor-8(17)-labden- | | |
| 13-one | | |
| Poranthericine: Ac | 280.2276 | 30.671 |
| 1,13-Dihydroxy-4- | 281.1389 | 3.9093 |
| oxo-2-pseudoguaien- | | |
| 12,6-olide | | |
| 9,12- | 281.248 | 11.9552 |
| Octadecadienoic | | |
| acid; (9E,12Z)- | | |
| form | | |
| Bharatamine; (±)- | 282.1494 | 16.4697 |
| form | | |
| Randainal: 9- | 283.1334 | 4.0636 |
| Alcohol | | |
| (N- | 284.1286 | 7.1766 |
| Phenylacetyl)glycine: | | |
| Benzyl ester | | |
| 2,3-Dihydro-2-(4- | 285.149 | 7.6093 |
| hydroxyphenyl)-5- | | |
| (2-hydroxypropyl)- | | |
| 3-methylbenzofuran | | |
| piperine/cocluarine/laurifoline | 286.1443 | 10.499 |
| Komaroine | 287.1548 | 9.1777 |
| Trichostatic acid | 288.1599 | 17.7116 |
| chiro-Inositol; D- | 289.1651 | 30.0224 |
| form: 1,4-Di-Me, | | |
| 2,3:5,6-di-O- | | |
| isopropylidene | | |
| 4-Amino-4,6- | 290.1603 | 24.7326 |
| dideoxy-3-C- | | |
| methylmannose; β- | | |
| D-Pyranose-form: | | |
| Me glycoside, N- | | |
| Me, N,2-di-Ac | | |
| 8-Hydroxy-17- | 291.196 | 35.1867 |
| octadecene-9,11- | | |
| diynoic acid | | |
| 1,2,3,5- | 292.1661 | 17.3192 |
| Benzenetetrol: | | |
| Tris(ethylamide) | | |
| Ecklonialactone A: | 293.2116 | 36.459 |
| 6,7-Dihydro | | |
| 2-Amino-2- | 294.1916 | 15.0713 |
| deoxyglucose; D- | | |
| form: Di-Et acetal, | | |
| 5,6-O- | | |
| isopropylidene | | |
| 16-Hydroxy- | 295.2273 | 28.3671 |
| 9,12,14- | | |
| octadecatrienoic acid | | |
| 2-(3,7-Dimethyl-2,6- | 296.2014 | 16.4224 |
| octadienyl)-3- | | |
| methyl-4(1H)- | | |
| quinolinone | | |
| Tanshindiol A: 18- | 297.1127 | 0.9215 |
| Deoxy | | |
| 4-[2-(3,5-Dimethyl- | 298.1654 | 8.2921 |
| 2-oxocyclohexyl)-2- | | |
| hydroxyethyl]-2,6- | | |
| piperidinedione9C,I; | | |
| (1S,3S,5S,aR)- | | |
| form: 4?-Hydroxy | | |
| 1,10:4,5-Diepoxy- | 299.1494 | 4.5513 |
| 3,6,8-trihydroxy-11- | | |
| germacren-9-one; | | |
| (1β,3a,4a,5a,6a,8a,10β)- | | |
| form | | |
| Erythratine: 2-Deoxy | 300.1599 | 9.567 |
| Febrifugine‡; (+)- | 301.1664 | 8.4802 |
| form: Oxime | | |
| Pulchelline‡ | 302.1756 | 14.7237 |
| Trichostatin A | 303.1708 | 10.7598 |
| 2-Amino-6-(1H- | 304.1661 | 14.407 |
| indol-3- | | |
| ylacetyl)hexanoic | | |
| acid | | |
| Malindine: 19- | 305.1892 | 13.8275 |
| Epimer | | |
| 1,4-Epoxy-3-aza-A- | 306.2433 | 23.3699 |
| homoandrostan-17- | | |
| ol | | |
| Eburnamenine; (−)- | 307.181 | 15.7696 |
| form: 11-Methoxy, | | |
| 14,15-didehydro | | |
| C-Alkaloid B | 308.1888 | 17.7973 |
| Taberpsychine | 309.1967 | 20.7503 |
| Brevicarine: N—Ac | 310.1919 | 14.9683 |
| 1-O—C | 311.1131 | 0.8509 |
| amoylglucose; β-D- | | |
| Pyranose-(E)-form | | |
| Clausenamide; (±)- | 312.1599 | 6.7357 |
| form: 1′-Me ether | | |
| 5-[(4- | 313.144 | 3.8762 |
| Hydroxyphenyl)ethenyl]- | | |
| 2-(3-methyl-1- | | |
| butenyl)-1,3- | | |
| benzenediol: 3′- | | |
| Hydroxy | | |
| Dehydroisolongistrobine: | 314.1504 | 11.1748 |
| Dihydro | | |
| Flourensianol: | 315.1596 | 6.6907 |
| Tigloyl | | |
| Ipanguline D3: 3″- | 316.176 | 11.0249 |
| Ac | | |
| Franklinol | 317.1753 | 11.1576 |
| 8-Methyl-8- | 318.1705 | 10.2281 |
| azabicyclo[3.2.1]octane- | | |
| 3,6-diol; (3R,6R)- | | |
| form: 3-O- | | |
| Phenylacetyl, 6-Ac | | |
| 8- | 319.1909 | 11.1821 |
| dehydrogingerdione | | |
| Calabacine | 320.1974 | 11.3796 |
| Arphamenine A | 321.1926 | 9.1521 |
| 8-Methyl-8- | 322.2018 | 13.2466 |
| azabicyclo[3.2.1]octane- | | |
| 3,6-diol; (3R,6R)- | | |
| form: Ditigloyl | | |
| Schizogaline | 323.1759 | 6.814 |
| Stravidin S3 | 324.2287 | 17.1545 |
| 1,7-Bis(4- | 325.1803 | 5.9258 |
| hydroxyphenyl)- | | |
| 1,4,6-heptatrien-3- | | |
| one: 1,2,6,7- | | |
| Tetrahydro, di-Me | | |
| ether | | |
| C-Fluorocurine | 326.1994 | 12.5192 |
| Plicane | 327.1345 | 2.2076 |
| Cryptostyline I; (R)- | 328.1549 | 4.6368 |
| form | | |
| 2-Aminoethanol: N- | 328.3215 | 1.5932 |
| Octadecanoyl | | |
| 5-Isopropyl-2- | 329.16 | 4.4525 |
| methyl-1,3- | | |
| benzenediol: Mono- | | |
| O-β-D- | | |
| glucopyranoside | | |
| sinomenin/deoxyharringtonine | 330.1705 | 6.8304 |
| picrocrocin/carnosol | 331.1757 | 7.7432 |
| 4-Amino-4,6- | 332.1709 | 8.0369 |
| dideoxy-3-C- | | |
| methylmannose; β- | | |
| D-Pyranose-form: | | |
| Me glycoside, N,2,3- | | |
| tri-Ac | | |
| 4-(5-Phenyl-2,4- | 333.1854 | 10.7614 |
| pentadien-1- | | |
| yl)tetracyclo[5.4.0.02,5.03,9]undec- | | |
| 10-ene-8-carboxylic | | |
| acid | | |
| 4-[2- | 334.2018 | 9.297 |
| (Acetylmethylamino)ethyl]- | | |
| 4-(4- | | |
| hydroxy-3- | | |
| methoxyphenyl)-2,4- | | |
| cyclohexadien-1- | | |
| one: 2,3,5,6- | | |
| Tetrahydro, Me ether | | |
| 3-Hydroxy-17-oxo- | 335.2222 | 10.5256 |
| 13-labden-15-oic | | |
| acid; (ent-3β,8aH, | | |
| 13Z)-form: 3- | | |
| Ketone | | |
| Dendrine | 336.2175 | 11.8435 |
| Pregnane-2,3,6-triol | 337.2742 | 9.3809 |
| 1-Tetracosene | 337.3834 | 0.8027 |
| Gramodendrine | 338.2232 | 13.6109 |
| ribo-Hexos-3-ulose; | 339.1807 | 4.6442 |
| a-D-Furanose- | | |
| form: 1,2:5,6-Di-O- | | |
| cyclohexylidene | | |
| 4-[2-(3,5-Dimethyl- | 340.176 | 8.4396 |
| 2-oxocyclohexyl)-2- | | |
| hydroxyethyl]-2,6- | | |
| piperidinedione9C,I; | | |
| (1S,3S,5S,aR)- | | |
| form: 5R-Acetoxy | | |
| 3,4-Dihydro-3,4,5,6- | 341.1389 | 4.5294 |
| tetramethoxy-2- | | |
| phenyl-2H-furo[2,3- | | |
| h]-1-benzopyran; | | |
| (2R*,3S*,4R*)- | | |
| form: 3-Demethoxy | | |
| Isocorypalmine | 342.1705 | 6.757 |
| Sorbistin C | 343.1716 | 5.0762 |
| Reticuline; (S)- | 345.194 | 6.9221 |
| form: N-Me | | |
| Suaveoline‡: N—Ac | 346.1919 | 10.3909 |
| 5-Hydroxy-19- | 347.2222 | 9.2508 |
| vouacapanoic acid; | | |
| 5a-form: Me ester | | |
| Mahanine; (?)-form: | 348.1963 | 7.8688 |
| Deoxy, ? | | |
| Amorphastilbol | 349.2167 | 9.5235 |
| 2-Pentadecyl-4(1H)- | 350.2484 | 13.5694 |
| quinolinone: | | |
| 6′,7′,9′,10′,12′,13′- | | |
| Hexadehydro (all-E) | | |
| Holantogenin | 351.2535 | 13.4435 |
| 2-Amino-3-hydroxy- | 352.2521 | 13.877 |
| 15-methyl-1- | | |
| hexadecanesulfonic | | |
| acid | | |
| 3-(15- | 353.2692 | 11.3765 |
| Hydroxyhexadecyl)- | | |
| 4-methyl-2,5- | | |
| furandione | | |
| Isostemonidine | 354.228 | 9.6722 |
| Colletochlorin A; (E)- | 355.1312 | 0.802 |
| form: 6′-Ketone | | |
| Halfordinol: O-(2,3- | 356.1736 | 3.9331 |
| Dihydroxy-3- | | |
| methylbutyl), N-Me | | |
| Xanthoascin | 357.1603 | 2.8829 |
| Belactosin C | 358.1978 | 4.9012 |
| Kouminidine | 359.1971 | 6.2393 |
| Lincosamine; a- | 360.2022 | 8.4368 |
| Pyranose-form: 7- | | |
| Me, 1,2:3,4-di-O- | | |
| isopropylidene, N—Ac | | |
| Cypholophine: O—Ac | 361.2127 | 9.7039 |
| Gomandonine | 362.2331 | 9.9227 |
| 12-Hydroxy- | 363.2171 | 6.8766 |
| 8(17),13-labdadien- | | |
| 15,12-olid-19-oic | | |
| acid; (12?)-form: 12- | | |
| Me ether | | |
| Nonylprodigiosin | 364.2389 | 9.165 |
| 3,14,17,21- | 365.2328 | 7.1234 |
| Tetrahydroxypregn- | | |
| 5-en-20-one; | | |
| (3β,14β,17βOH)- | | |
| form | | |
| Bufotenine: O-β-D- | 367.1869 | 2.4899 |
| Glucopyranoside | | |
| Ajmalicine; (−)- | 368.21 | 6.1786 |
| form: 20-Epimer, Nb- | | |
| Me | | |
| Madindoline A | 370.2018 | 5.086 |
| Sucrose: 1′,6′-Di- | 371.1553 | 3.1522 |
| Me | | |
| Schelhammerine: Ac | 372.1811 | 2.6491 |
| Strictanine: 11,12- | 373.2127 | 4.7864 |
| Dihydroxy, N- | | |
| deformyl, N—Ac | | |
| Dendrowardine: | 374.2098 | 6.2225 |
| Chloride | | |
| Ochropposinine | 375.2284 | 9.3513 |
| oxindole | | |
| Acsine | 376.2124 | 5.9189 |
| 3,9- | 377.2539 | 7.3745 |
| Megastigmanediol; | | |
| (3S,5R,6S,9R)- | | |
| form: 9-O-β-D- | | |
| Glucopyranoside | | |
| Aurachin F | 378.2433 | 7.8026 |
| 6′-Oxo-6,5′-diapo-6- | 379.2273 | 7.2933 |
| carotenoic acid | | |
| 2-Phenyl-1,5,9- | 380.2338 | 8.6877 |
| triazacyclotridecan- | | |
| 4-one; (S)-form: N9- | | |
| Benzoyl | | |
| 8,17-Epoxy- | 381.1913 | 2.4779 |
| 2,3,9,12- | | |
| tetrahydroxy-5,13- | | |
| briaradien-18,7-olide | | |
| Echitamidine; (±)- | 382.2229 | 7.2613 |
| form | | |
| Vismiaguianone B | 383.1858 | 1.9662 |
| Sorbistin B | 384.1982 | 4.2519 |
| 1-O- | 385.1498 | 1.3512 |
| Coumaroylglycerol; | | |
| (S,E)-form: 4′- | | |
| Deoxy, 2-O-β-D- | | |
| glucopyranoside | | |
| Antibiotic BE 48021 | 386.1967 | 2.6218 |
| Anomaline‡ | 387.2284 | 7.4364 |
| Delorine | 388.2124 | 5.4791 |
| Norcaperatic acid | 389.2539 | 7.8435 |
| Protoemetine; (−)- | 390.228 | 6.0436 |
| form: Alcohol, O9- | | |
| de-Me, di-O—Ac | | |
| 5,11-Epoxy-7- | 391.2332 | 6.8418 |
| megastigmene-3,9- | | |
| diol; (1S,3S,5R,6R,7E,9R)- | | |
| form: 7,8- | | |
| Dihydro, 3-O-β-D- | | |
| glucopyranoside | | |
| 4-Methylaconitane- | 392.2437 | 5.2205 |
| 1,8,14,16-tetrol; | | |
| (1a,14a,16β)-form: | | |
| O16-Me, O14-Ac | | |
| 3,9,13- | 393.2488 | 6.0505 |
| Megastigmanetriol: | | |
| 3-O-β-D- | | |
| Glucopyranoside | | |
| Macrocentrine | 394.2593 | 7.8383 |
| Protostrychnine: | 395.1971 | 3.8948 |
| 10,11-Dimethoxy, | | |
| 17-deoxy, 17,18- | | |
| didehydro | | |
| 24-Nor-4(23),9(11)- | 395.3678 | 7.8963 |
| fernadiene | | |
| Karatavic acid | 397.2015 | 1.9521 |
| 24-Nor-12-ursene | 397.3834 | 82.7805 |
| Sorbistin A; | 398.2138 | 3.7639 |
| Sorbistin A1 | | |
| Taraxacoside | 399.1291 | 0.5387 |
| Stigmast-8-ene | 399.3991 | 1.4507 |
| Glaucamine: Me | 400.176 | 1.3183 |
| ether | | |
| 2-Amino-19- | 400.379 | 1.4222 |
| tetracosene-1,3,4- | | |
| triol; (2S,3S,4R,19E)- | | |
| form | | |
| Chalmicrin | 401.2903 | 8.6982 |
| Napelline: 12-Ac | 402.2644 | 5.5147 |
| Cholestan-3-one; 5a- | 402.3736 | 0.2191 |
| form: Oxime | | |
| 19,25-Dihydroxy- | 403.2848 | 7.012 |
| 16-scalaren-24,25- | | |
| olide | | |
| Fortimicin KG: N1- | 404.2509 | 5.3874 |
| Aminoacetyl | | |
| Iguesterin: ?20(29)- | 405.2793 | 9.6503 |
| Isomer | | |
| Fortimicin A | 406.2665 | 6.276 |
| 13,17,19- | 407.3161 | 9.9904 |
| Villanovanetriol; | | |
| (ent-13β)-form: 19- | | |
| O-(3- | | |
| Methylbutanoyl) | | |
| Antibiotic BE | 408.2902 | 8.7704 |
| 14106: 8-Deoxy | | |
| 7,20-Epoxy-16- | 409.2226 | 2.3632 |
| kaurene-1,6,7,15,19- | | |
| pentol; (ent- | | |
| 1β,6a,7aOH,15a)- | | |
| form: 19-Ac | | |
| 11,13(18)- | 409.3834 | 15.7433 |
| Oleanadiene | | |
| Fructigenine B | 410.2443 | 3.5481 |
| Cudratricusxanthone | 411.1807 | 0.7713 |
| A: 2-Me ether | | |
| Sorbistin A; | 412.2295 | 3.5054 |
| Sorbistin A2 | | |
| 5-(2,3-Dihydroxy-3- | 413.2175 | 7.0982 |
| methylbutyl)-4-(3,4- | | |
| epoxy-4- | | |
| methylpentanoyl)- | | |
| 3,4-dihydroxy-2- | | |
| isopentanoyl-2- | | |
| cyclopenten-1-one | | |
| Alkaloid TC-D | 414.2393 | 10.5309 |
| Imbricaric acid | 417.1913 | 1.8379 |
| 3,20-Diamino-18- | 417.3117 | 2.5983 |
| hydroxypregn-5-ene- | | |
| 11,16-dione; (3β,20S)- | | |
| form: N-Tetra-Me | | |
| Vilmoritine | 418.2593 | 5.0583 |
| 26,27-Dinorergosta- | 419.3161 | 10.1765 |
| 8(14),22-diene- | | |
| 3,6,15,25-tetrol | | |
| Norerythrostachamide: | 420.275 | 6.8995 |
| 3-Deoxy, 7-ketone | | |
| 8(17),13-Labdadien- | 421.2954 | 7.4809 |
| 15-ol; (ent-13E)- | | |
| form: 15-Carboxylic | | |
| acid, (3-acetoxy-2S- | | |
| hydroxypropyl) ester | | |
| Graciline: 6β,16β- | 422.2542 | 5.833 |
| Dimethoxy | | |
| N—(N-L-Arginyl-D- | 423.2356 | 3.3492 |
| allothreonyl)-L- | | |
| phenylalanine | | |
| Aconitane- | 426.2492 | 2.3959 |
| 1,4,6,7,8,14,16- | | |
| heptol; | | |
| (1a,5β,6β,14a,16β)- | | |
| form: O6,O16-Di- | | |
| Me, N-Et | | |
| Daphnezomine K: | 428.2437 | 2.7233 |
| 1,4-Dideoxy, 14,15- | | |
| didehydro, 21-Ac, N- | | |
| oxide | | |
| Stigmast-4-en-3-one: | 428.3892 | 3.1827 |
| Oxime | | |
| Vismiaguianone D | 429.1702 | 0.911 |
| Cholest-22-en-3-ol; | 429.3732 | 21.8434 |
| (3β,5a,22Z)-form: | | |
| Ac | | |
| Mycalazoles; | 430.3685 | 14.0059 |
| Mycalazole 3: | | |
| 11′,12′,20′,21′- | | |
| Tetrahydro | | |
| Cholest-4-ene-3,6- | 431.3889 | 16.5216 |
| diol; (3β,6a)-form: | | |
| Di-Me ether | | |
| Antibiotic SF 1739 | 432.2134 | 2.4276 |
| Radiosumin | 433.2451 | 2.6382 |
| Solacapine | 433.3794 | 1.7208 |
| 4-Methylaconitane- | 434.2906 | 7.2077 |
| 1,8,14,16-tetrol; | | |
| (1a,14a,16β)-form: | | |
| O1,O16-Di-Me, N- | | |
| Et, O14-Ac | | |
| Grandone | 435.2535 | 3.5971 |
| Acophine | 436.2618 | 4.0659 |
| 4,18:11,16:15,16- | 437.2539 | 2.6223 |
| Triepoxy-6,19- | | |
| clerodanediol; (ent- | | |
| 4β,6β,11R,13S,16R)- | | |
| form: Di-Ac | | |
| Lythrancine I: 4- | 438.2644 | 3.919 |
| Deoxy | | |
| 4-Methylaconitane- | 440.2648 | 3.5984 |
| 1,6,7,8,14,16,18- | | |
| heptol; | | |
| (1a,5β,6β,14a,16β)- | | |
| form: O6,O16-Di- | | |
| Me, N-Et | | |
| Dehydronapelline: | 442.2593 | 2.3892 |
| Di-Ac | | |
| Stigmast-5-ene- | 445.3681 | 5.9033 |
| 3,7,22-triol; | | |
| (3β,7a,22R,24R)- | | |
| form: 7-Ketone | | |
| Piericidin; Piericidin | 446.2906 | 1.3596 |
| A1: 10′-Me ether, | | |
| 11′,12′-epoxide | | |
| 3-Deamino-3- | 446.3634 | 2.181 |
| hydroxysolanocapsine: | | |
| 23-Me ether | | |
| Stigmast-5-ene- | 447.3838 | 11.0232 |
| 3,7,22-triol; | | |
| (3β,7a,22R,24R)- | | |
| form | | |
| 20-Aminopregnane- | 448.3427 | 8.4776 |
| 3,4-diol; | | |
| (3β,4a,5a,20S)- | | |
| form: N,N-Di-Me, | | |
| Di-O—Ac | | |
| Spirostane-1,3,5- | 449.3267 | 6.26 |
| triol | | |
| Norerythrostachamine: | 450.2855 | 5.0866 |
| 3-Deoxy, 6a- | | |
| hydroxy, 7-ketone, | | |
| N-Me | | |
| Encecanescin | 451.2484 | 2.3543 |
| Epoxycytochalasin | 452.2801 | 6.3876 |
| H: O-De-Ac | | |
| Ajmalinimine: Ac | 453.2389 | 1.3902 |
| Phosphatidylcholine; | 454.257 | 3.3313 |
| Glycerol 1,2- | | |
| dihexanoate 3- | | |
| phosphocholine | | |
| Palinurine A‡ | 458.2906 | 1.3138 |
| Spirostan-3-amine; | 458.3634 | 2.1359 |
| (3β,5a,25R)-form: | | |
| N—Ac | | |
| Phloeodictyne A; | 462.4172 | 2.0079 |
| Phloeodictyne 4,10a | | |
| Xestospongin A: 7 | 463.3899 | 8.2043 |
| S-Hydroxy | | |
| 3,6,14,16,20- | 464.3376 | 4.3328 |
| Cevanepentol; | | |
| (3β,5a,6a,16?,20?)- | | |
| form | | |
| Erinacine G | 465.2488 | 0.6971 |
| 4-Methylaconitane- | 466.2805 | 2.8951 |
| 1,6,7,8,14,16,18- | | |
| heptol; | | |
| (1a,5β,6β,14a,16β)- | | |
| form: 14-Ketone, | | |
| O1,O6,O16,O18- | | |
| tetra-Me, N-Et | | |
| Fissistin | 467.2433 | 2.6469 |
| 4-Methylaconitane- | 468.2597 | 2.158 |
| 1,6,7,8,14,16,18- | | |
| heptol; | | |
| (1a,5β,6β,14a,16β)- | | |
| form: O1,O6,O14,O16- | | |
| Tetra-Me, N- | | |
| formyl | | |
| 4-Methylaconitane- | 470.2754 | 1.7333 |
| 1,6,7,8,10,14,16,18- | | |
| octol; | | |
| (1a,6β,14a,16β)- | | |
| form: O6,O16,O18- | | |
| Tri-Me, N-Et | | |
| 4,6′- | 472.3063 | 6.5798 |
| Anhydrooxysporidinone | | |
| 3,5,12,14- | 475.2332 | 0.6375 |
| Tetrahydroxy-11- | | |
| oxobufa-20,22- | | |
| dienolide; | | |
| (3β,5β,12β,14β)- | | |
| form: 3-Ac | | |
| Austalide H | 477.2488 | 0.8383 |
| 4-Methylaconitane- | 478.2805 | 2.2442 |
| 1,6,7,8,14,16,18- | | |
| heptol; | | |
| (1a,5β,6β,14a,16β)- | | |
| form: 6-Ketone,7,8- | | |
| methylene ether, | | |
| O1,O14,O16,O18- | | |
| tetra-Me, N-Et | | |
| Ethyl glucoside; a- | 479.2433 | 1.8358 |
| D-Pyranose-form: | | |
| 3,4,6-Tribenzyl | | |
| CytochalasinB | 480.275 | 2.9096 |
| 2,6,9,10- | 481.2437 | 1.8005 |
| Tetrahydroxy-5-oxo- | | |
| 11(13)-germacren- | | |
| 12,8-olide; | | |
| (2a,4β,6a,8a,9β,10β)- | | |
| form: 6-Angeloyl, | | |
| 9-(2- | | |
| methylbutanoyl) | | |
| Malyngamide F: O—Ac | 482.2673 | 2.1894 |
| Schizanthine E | 491.2757 | 1.745 |
| Morusimic acid D: | 492.3172 | 3.2351 |
| 3-O-β-D- | | |
| Glucopyranoside | | |
| Aphelandrine; (+)- | 493.2815 | 1.2011 |
| form: 17,18- | | |
| Diepimer | | |
| Epoxycytochalasin H | 494.2906 | 1.6725 |
| 4,18-Epoxy- | 495.2594 | 1.21 |
| 6,8,11,19- | | |
| tetrahydroxy-13- | | |
| cleroden-15,16- | | |
| olide; (ent- | | |
| 4β,6β,8a,11R)-form: | | |
| 6- | | |
| (Methylpropanoyl), | | |
| 11-Ac | | |
| 4-Methylaconitane- | 496.291 | 1.8854 |
| 1,6,7,8,14,16,18- | | |
| heptol; | | |
| (1a,5β,6β,14a,16β)- | | |
| form: O6,O16,O18- | | |
| Tri-Me, N-Et, | | |
| 14-Ac | | |
| 14,15-Seco-16- | 497.275 | 0.9088 |
| kauren-15,14-olid- | | |
| 19-oic acid; (ent- | | |
| 14a)-form: 16β,17- | | |
| Dihydro, β-D- | | |
| glucopyranosyl ester | | |
| Spermidine: N,N″- | 498.2604 | 1.4532 |
| Bis(4-hydroxy-3- | | |
| methoxyc amoyl) | | |
| 3,7- | 500.3046 | 1.1323 |
| Dihydroxycholan- | | |
| 24-oicacid; | | |
| (3a,5β,7a)-form: N- | | |
| (2-Sulfoethyl)amide | | |
| Edgeworthin: 7-O- | 501.1033 | 0.4844 |
| β-D- | | |
| Glucopyranoside | | |
| Lactucin 8-O- | 505.2226 | 1.0796 |
| hypoglabrate | | |
| Cassaidine: 7- | 506.3481 | 4.1045 |
| Ketone, 3-O-(3- | | |
| hydroxy-3- | | |
| methylbutanoyl) | | |
| Viridomycin A | 507.0001 | 0.5364 |
| CassiaminC | 507.108 | 0.742 |
| 4,18:8,13-Diepoxy- | 507.2594 | 2.2541 |
| 6,11,19-trihydroxy- | | |
| 15,16-clerodanolide; | | |
| (ent- | | |
| 4β,6β,8a,11β,13R)- | | |
| form: 6-Tigloyl,19- | | |
| Ac | | |
| Broussonetinine A: | 508.3121 | 2.6611 |
| 3-O-β-D- | | |
| Glucopyranoside | | |
| Anhydrobonellin | 509.2552 | 0.8466 |
| Trideacetylpyripyropene | 510.2492 | 0.1093 |
| A: 7,11- | | |
| Dideoxy, 3,19-di-Ac | | |
| 4-Methylaconitane- | 510.3067 | 0.4267 |
| 1,6,7,8,14,16,18- | | |
| heptol; | | |
| (1a,5β,6β,14a,16β)- | | |
| form: O1,O6,O14,O16- | | |
| Tetra-Me, | | |
| N-Et, 18-Ac | | |
| Farnesin: 15-O-β-D- | 511.2543 | 0.7718 |
| Glucopyranoside | | |
| Cyclozoanthamine | 512.3012 | 1.9116 |
| 3,6,16-Cevanetriol; | 514.3532 | 1.6336 |
| (3β,5a,6β,25β)-form: | | |
| 6-Ketone, di-Ac | | |
| Ciliatocholicacid | 516.309 | 1.9638 |
| 3,14-Dihydroxycard- | 518.3481 | 1.8198 |
| 20(22)-enolide; | | |
| (3β,5a,14β,17a)- | | |
| form: 3-O-(4- | | |
| Amino-2,4,6- | | |
| trideoxy-3-O- | | |
| methyl-a-L-arabino- | | |
| hexopyranoside) | | |
| 3,25-Epoxy- | 519.3322 | 1.8311 |
| 1,2,3,11- | | |
| tetrahydroxy-12- | | |
| ursen-28-oic acid | | |
| 4-Methylaconitane- | 522.3067 | 2.223 |
| 1,6,8,14,16,18- | | |
| hexol; | | |
| (1a,5β,6a,14a,16β)- | | |
| form: O6,O16,O18- | | |
| Tri-Me, N-Et, | | |
| 8,14-di-Ac | | |
| Anhydrobonellin: | 523.2709 | 1.2379 |
| Me ester | | |
| Isodesmosine | 526.2877 | 1.5732 |
| Destomycin A | 528.2404 | 0.5476 |
| 16,17,19- | 533.457 | 0.9777 |
| Kauranetriol; (ent- | | |
| 16βOH)-form: 19- | | |
| Tetradecanoyl | | |
| Neocochliodinol: | 535.2597 | 0.7368 |
| Di-Me ether | | |
| Vitixanthin | 535.3059 | 1.0244 |
| 4-Methylaconitane- | 536.2859 | 0.991 |
| 1,6,7,8,10,14,16- | | |
| heptol; | | |
| (1a,6β,14a,16β)- | | |
| form: 7,8- | | |
| Methylene, 1,16-di- | | |
| Me ether, N-Et, | | |
| 6,14-di-Ac | | |
| 14,20:18,20- | 537.2852 | 1.4613 |
| Diepoxypregnane- | | |
| 3,11,12-triol; | | |
| (3β,5a,11a,12β,14β,20S)- | | |
| form: 12-C | | |
| amoyl, 11-Ac | | |
| 1,2,7,8,11,12- | 543.493 | 1.987 |
| Hexahydrolycopene | | |
| Theopederin A | 544.3121 | 1.5168 |
| 1,2,7,7′,8,8′,11,12- | 545.5086 | 1.9164 |
| Octahydrolycopene | | |
| 3,14,19- | 549.27 | 1.1738 |
| Trihydroxycarda- | | |
| 5,20(22)-dienolide; | | |
| (3β,14β)-form: 19- | | |
| Aldehyde, 3-O-β-D- | | |
| glucopyranoside | | |
| Haliclotriol B | 551.41 | 4.6015 |
| 1-O-Alkylglycero- | 552.4029 | 3.2532 |
| 3-phosphocholines; | | |
| 1-Octadecylglycero- | | |
| 3-phosphocholine: 2- | | |
| Ac | | |
| Helianol: Octanoyl | 555.5141 | 7.9534 |
| 4-Methylaconitane- | 556.3274 | 3.1885 |
| 1,8,14,16,18-pentol; | | |
| (1a,14a,16β)-form: | | |
| O8,O14,O16-Tri- | | |
| Me, N-Et, 18-O-(2- | | |
| methoxybenzoyl) | | |
| 8(17)-Labden-15-ol; | 557.5297 | 10.6 |
| (ent-13R)-form: | | |
| Carboxylic acid, 9- | | |
| octadecenyl ester | | |
| Nummularine P | 558.3291 | 1.9092 |
| Edpetilidinine: 3-O- | 562.4107 | 5.9366 |
| β-D-Xylopyranoside | | |
| Thaimycin A | 563.3332 | 3.1821 |
| 3-Hydroxycholan- | 564.2665 | 1.0722 |
| 24-oicacid; (3a,5β)- | | |
| form: N-(2- | | |
| Sulfoethyl)amide,3- | | |
| O-sulfate | | |
| Manzamine A; (+)- | 567.4063 | 1.8895 |
| form: 1S,2,3,4- | | |
| Tetrahydro, N2-Me | | |
| β,β-Carotene-2,2′- | 569.4358 | 3.8154 |
| diol | | |
| Rhodopin: 7′,8′- | 571.4879 | 5.316 |
| Dihydro, Me ether | | |
| 4-Methylaconitane- | 572.3223 | 1.299 |
| 1,6,7,8,14,16,18- | | |
| heptol; | | |
| (1a,5β,6β,14a,16β)- | | |
| form: O1,O6,O16,O18- | | |
| Tetra-Me, | | |
| N-Et, 14-benzoyl | | |
| 2-Acetamido-2- | 572.4162 | 0.5111 |
| deoxyglucose; a-D- | | |
| Pyranose-form: 3- | | |
| Octadecanoyl, 6-(3- | | |
| methylbutanoyl) | | |
| Coriacyclodienin | 573.4883 | 18.8653 |
| Purpuramine C | 575.0819 | 1.9082 |
| Montecristin | 575.5039 | 27.7655 |
| 4-Methylaconitane- | 578.3329 | 3.9297 |
| 1,6,7,8,10,14,16- | | |
| heptol; | | |
| (1a,6β,14a,16β)- | | |
| form: 7,8- | | |
| Methylene, 1,16-di- | | |
| Me ether, N-Et, 14- | | |
| O-(2R- | | |
| methylbutanoyl), 6- | | |
| Ac | | |
| Hermonionic acid | 579.3322 | 2.0507 |
| Rosamicin | 582.3642 | 1.6958 |
| 11,13(18)- | 583.3998 | 1.6478 |
| Oleanadiene- | | |
| 3,16,28-triol; | | |
| (3β,16β)-form: Tri- | | |
| Ac | | |
| Cholestane- | 585.4002 | 1.8081 |
| 3,6,8,15,24-pentol; | | |
| (3β,5a,6a,15β,24S)- | | |
| form: 24-O-a-L- | | |
| Arabinofuranoside | | |
| 1,2-Dihydro-1- | 587.4828 | 1.8024 |
| methoxy-?,?- | | |
| caroten-4-one: | | |
| 7′,8′,11′,12′- | | |
| Tetrahydro | | |
| Annodienin | 589.4832 | 4.0554 |
| 2,2′,3,4,4′- | 590.7441 | 0.2456 |
| Pentabromo-6- | | |
| hydroxydiphenyl | | |
| ether: Me ether | | |
| Rhizoxin: 2,3:11,12- | 594.3431 | 1.843 |
| Bis(deepoxy), | | |
| 2,3,11,12- | | |
| tetradehydro | | |
| Vimose | 595.3329 | 1.9588 |
| Kahalalide D | 596.356 | 2.0571 |
| Didemniserinolipid | 598.4319 | 3.0108 |
| C: O10-Ac | | |
| 5,7-Dihydroxy-6- | 599.5039 | 5.9939 |
| methyl-2-nonacosyl- | | |
| 4H-1-benzopyran-4- | | |
| one | | |
| Destruxin A5 | 606.3867 | 4.4309 |
| 1-O-β-D- | 607.3693 | 1.6949 |
| Mannopyranosyl-L- | | |
| erythritol: 3- | | |
| Hexadecanoyl, 2,6- | | |
| di-Ac | | |
| Irciniastatin A: 11- | 608.3071 | 0.9978 |
| Ketone | | |
| 4-Aminotetrahydro- | 608.5618 | 1.8226 |
| 2-(4-tetradecenyl)-3- | | |
| furanol; | | |
| (2R*,3R*,4R*,4′Z)- | | |
| form, N-(2R- | | |
| Hydroxyeicosanoyl) | | |
| Destruxin C; | 610.3816 | 0.8303 |
| Destruxin C | | |
| 1,2- | 611.406 | 1.132 |
| Dicaffeoylglucose; | | |
| (E,E)-form | | |
| Ergost-5-en-3-ol; | 611.5767 | 0.9812 |
| (3β,24R)-form: | | |
| Tetradecanoyl | | |
| Amphilactam B | 612.39 | 1.3718 |
| 2-Alkyl-5,7- | 613.5196 | 8.6488 |
| dihydroxy-4H-1- | | |
| benzopyran-4-ones; | | |
| 2-Hentriacontyl-5,7- | | |
| dihydroxy-4H-1- | | |
| benzopyran-4-one | | |
| 4-Methylaconitane- | 615.3281 | 0.3659 |
| 1,6,7,8,14,16,18- | | |
| heptol; | | |
| (1a,5β,6β,14a,16β)- | | |
| form: O1,O6,O16- | | |
| Tri-Me, N-Et, 18-O- | | |
| (2- | | |
| acetamidobenzoyl) | | |
| 2-Amino-4- | 620.5981 | 1.3499 |
| octadecene-1,3-diol; | | |
| (2S,3R,4E)-form: | | |
| N-(13Z- | | |
| Docosenoyl) | | |
| 2-Amino-4- | 622.6138 | 2.1151 |
| octadecene-1,3-diol; | | |
| (2S,3R,4E)-form: | | |
| N-Docosanoyl | | |
| Cotylenin A | 623.3431 | 2.2171 |
| DestruxinD | 624.3608 | 2.0109 |
| 3,3′,4′,5,7- | 625.374 | 1.0326 |
| Pentahydroxyflavone: | | |
| 3′,4′,5,7-Tetra-Me | | |
| ether, 3-O- | | |
| octadecanoyl | | |
| Cholest-5-en-3-ol; | 625.5923 | 1.2527 |
| 3β-form: | | |
| Hexadecanoyl | | |
| Apicidin: 8′S- | 626.3917 | 1.2619 |
| Alcohol | | |
| 2-Amino-9- | 626.5723 | 0.8005 |
| docosene-1,3,4-triol; | | |
| (2S,3S,4R,9Z)- | | |
| form: N-(2R- | | |
| Hydroxyhexadecanoyl) | | |
| 4-Methylaconitane- | 628.3485 | 0.6086 |
| 1,6,8,14,16,18- | | |
| hexol; | | |
| (1a,5β,6a,14a,16β)- | | |
| form: O1,O6,O16,O18- | | |
| Tetra-Me, | | |
| N-Et, 14-(4- | | |
| methoxybenzoyl), 8- | | |
| Ac | | |
| 4-Aminotetrahydro- | 636.5931 | 5.6741 |
| 2-(4-tetradecenyl)-3- | | |
| furanol; | | |
| (2R*,3R*,4R*,4′Z)- | | |
| form, N-(2R- | | |
| Hydroxydocosanoyl) | | |
| Fomentaric acid | 637.6135 | 1.823 |
| Rosamicin: 2′- | 638.3904 | 2.6361 |
| Propanoyl | | |
| Variceramides; | 638.6087 | 1.4588 |
| !Variceramide 2 | | |
| Apicidin: 9′- | 640.371 | 1.6849 |
| Hydroxy | | |
| 4-Aminotetrahydro- | 650.6087 | 7.1632 |
| 2-(4-tetradecenyl)-3- | | |
| furanol; | | |
| (2R*,3R*,4R*,4′Z)- | | |
| form, N-(2R- | | |
| Hydroxytricosanoyl) | | |
| Stigmasta-5,22-dien- | 651.608 | 1.1899 |
| 3-ol; (3β,22E,24S)- | | |
| form: Hexadecanoyl | | |
| 2-Amino-6,9- | 664.6244 | 5.6768 |
| heptacosadiene- | | |
| 1,3,5-triol; | | |
| (2?,3?,5?,6E,9E)- | | |
| form: N- | | |
| Pentadecanoyl | | |
| 9(11)-Fernen-20-ol; | 665.6236 | 2.7579 |
| 20β-form: 20- | | |
| Hexadecanoyl | | |
| Rosamicin: 2′,3-Di- | 666.3853 | 4.0515 |
| Ac | | |
| Rodiasine: Me | 667.3747 | 1.5467 |
| ether, N2,N2′-di- | | |
| Me | | |
| Mycinamycin VI | 668.401 | 1.3502 |
| 4-Aminotetrahydro- | 678.64 | 2.659 |
| 2-(4-tetradecenyl)-3- | | |
| furanol; | | |
| (2R*,3R*,4R*,4′Z)- | | |
| form, N-(2R- | | |
| Hydroxypentacosanoyl) | | |
| 2-Amino-6- | 682.6349 | 2.6268 |
| octadecene-1,3,4- | | |
| triol; (2S,3S,4R,6E)- | | |
| form: N-(2R- | | |
| Hydroxytetracosanoyl) | | |
| 1,34- | 687.5927 | 0.9548 |
| Tetratriacontanediol: | | |
| O-(4-Hydroxy-3- | | |
| methoxyc amoyl)(E-) | | |
| Fasciculic acid C: | 694.453 | 2.7078 |
| 21-Deoxy, 2-acyl | | |
| isomer | | |
| 5,8-Epidioxyergosta- | 695.5978 | 1.4912 |
| 6,22-dien-3-ol; | | |
| (3β,5a,8a,22E,24R)- | | |
| form: | | |
| Octadecanoyl | | |
| CirramycinA1: 4′-O- | 710.4115 | 2.6253 |
| (Tetrahydro-(6R)- | | |
| methyl-5-oxo-2H- | | |
| pyran-2-yl) | | |
| Pyrophaeophorbide | 813.5682 | 5.4023 |
| a: 3,7R,5,11R- | | |
| Tetramethyl-2E- | | |
| hexadecenyl ester | | |

We claim:

1. A nettle extract comprising at least one compound selected from the group consisting of 6-azacytosine, levulinic acid, threonine, niacinamide, DL-methyl-m-tyrosine, 4-methyl-7-ethoxy coumarin, vitamin B5, isopropyl-B-D-thiogalactopyranoside, osthole, phosphatidylcholine, 4-shogaol, piperine/cocluarine/laurifoline, 8-dehydrogingerdione, sinomenin/deoxyharringtonine, and picrocrocin/carnosol.

2. The nettle extract of claim 1, comprising at least one compound selected from: about 1 to 3000 μg 6-azacytosine, about 5 to 5000 μg levulinic acid, about 5 to 1000 μg threonine, about 5 to 1000 μg niacinamide, about 10 to 1000 μg DL-methyl-m-tyrosine, about 10 to 2500 μg 4-methyl-7-ethoxy coumarin, about 50 to 3000 μg vitamin B5, about 5 to 250 μg isopropyl-B-D-thiogalactopyranoside, about 10 to 1000 μg osthole, about 10 to 500 μg phosphatidylcholine, about 10 to 1000 μg 4-shogaol, about 10 to 1000 μg piperine/cocluarine/laurifoline, about 10 to 750 μg 8-dehydrogingerdione, about 10 to 500 μg sinomenin/deoxyharringtonine, and about 10 to 500 μg picrocrocin/carnosol, per 100 mg of the extract.

3. The nettle extract of claim 1, comprising at least one compound selected from: about 1 to 1750 μg 6-azacytosine, about 10 to 2000 μg levulinic acid, about 10 to 500 μg threonine, about 10 to 300 μg niacinamide, about 30 to 300 μg DL-methyl-m-tyrosine, about 50 to 1500 μg 4-methyl-7-ethoxy coumarin, about 100 to 2000 μg vitamin B5, 20 to 100 μg isopropyl-B-D-thiogalactopyranoside, about 50 to 500 μg osthole, about 50 to 200 μg μg phosphatidylcholine, about 50 to 500 μg 4-shogaol, about 50 to 400 μg piperine/cocluarine/laurifoline, about 50 to 400 μg 8-dehydrogingerdione, about 30 to 250 μg sinomenin/deoxyharringtonine, and about 50 to 250 μg picrocrocin/carnosol, per 100 mg of the extract.

4. The nettle extract of claim 1, comprising about 1 to 1750 μg 6-azacytosine per 100 mg of the extract.

5. The nettle extract of claim 1, comprising about 20 to 100 μg isopropyl-B-D-thiogalactopyranoside per 100 mg of the extract.

6. The nettle extract of claim 1, comprising about 30 to 250 μg sinomenin/deoxyharringtonine per 100 mg of the extract.

7. The nettle extract of claim 1, comprising about 10 to 2000 μg levulinic acid per 100 mg of the extract per 100 mg of the extract.

8. The nettle extract of claim 1, comprising about 10 to 500 μg threonine per 100 mg of the extract per 100 mg of the extract.

9. The nettle extract of claim 1, comprising about 10 to 300 μg niacinamide per 100 mg of the extract.

10. The nettle extract of claim 1, comprising about 30 to 300 μg DL-methyl-m-tyrosine per 100 mg of the extract.

11. The nettle extract of claim 1, comprising about 50 to 1500 μg 4-methyl-7-ethoxy coumarin per 100 mg of the extract.

12. The nettle extract of claim 1, comprising about 100 to 2000 μg vitamin B5 per 100 mg of the extract.

13. The nettle extract of claim 1, comprising about 50 to 500 μg osthole per 100 mg of the extract.

14. The nettle extract of claim 1, comprising about 50 to 200 μg phosphatidylcholine per 100 mg of the extract.

15. The nettle extract of claim 1, comprising about 50 to 500 μg 4-shogaol per 100 mg of the extract.

16. The nettle extract of claim 1, comprising about 50 to 400 μg piperine/cocluarine/laurifoline per 100 mg of the extract.

17. The nettle extract of claim 1, comprising about 50 to 400 μg 8-dehydrogingerdione per 100 mg of the extract.

18. The nettle extract of claim 1, comprising about 50 to 250 μg picrocrocin/carnosol, per 100 mg of the extract.

19. The extract of claim 3, comprising about 1 to 1750 μg 6-azacytosine, about 10 to 2000 μg levulinic acid, about 10 to 500 μg threonine, about 10 to 300 μg niacinamide, about 30 to 300 μg DL-methyl-m-tyrosine, about 50 to 1500 μg 4-methyl-7-ethoxy coumarin, about 100 to 2000 μg vitamin B5, 20 to 100 μg isopropyl-B-D-thiogalactopyranoside, about 50 to 500 μg osthole, about 50 to 200 μg phosphatidylcholine, about 50 to 500 μg 4-shogaol, about 50 to 400 μg piperine/cocluarine/laurifoline, about 50 to 400 μg 8-dehydrogingerdione, about 30 to 250 μg sinomenin/deoxyharringtonine, and about 50 to 250 μg picrocrocin/carnosol, per 100 mg of the extract.

20. The extract of claim 19, comprising about 1500 to 1750 μg 6-azacytosine, about 1800 to 2000 μg levulinic acid, about 50 to 75 μg threonine, about 150 to 250 μg niacinamide, about 200 to 300 μg DL-methyl-m-tyrosine, about 1000 to 1300 μg 4-methyl-7-ethoxy coumarin, about 100 to 200 μg vitamin B5, 30 to 60 μg isopropyl-B-D-thiogalactopyranoside, about 250 to 400 μg osthole, about 100 to 200 μg phosphatidylcholine, about 50 to 100 μg 4-shogaol, about 75 to 150 μg piperine/cocluarine/laurifoline, about 50 to 150 μg 8-dehydrogingerdione, about 30 to 100 μg sinomenin/deoxyharringtonine, and about 75 to 150 μg picrocrocin/camosol, per 100 mg of the extract.

21. The extract of claim 1, further comprising at least one of resorcinol, proline, leucine, adenine, levoglucosan/glycogen/laminarin, synephrine, or shikimic acid.

22. The extract of claim 21, further comprising at least one of about 10 to 1500 μg of resorcinol, about 50 to 1500 μg of proline, about 5 to 1500 μg of leucine, about 10 to 5000 μg of adenine, about 300 to 10,000 μg of levoglucosan/glycogen/laminarin, about 100 to 3,000 μg of synephrine, or about 50 to 1000 μg of shikimic acid, per 100 mg of the extract.

23. The extract of claim 1, further comprising 3,4-dimethoxy chalcone.

24. The extract of claim 23, comprising about 25 to 200 μg of 3,4-dimethoxychalcone per 100 mg of the extract.

25. A nettle extract comprising about 1500 to 1750 μg 6-azacytosine, about 1800 to 2000 μg levulinic acid, about 50 to 75 μg threonine, about 150 to 250 μg niacinamide, about 200 to 300 μg DL-methyl-m-tyrosine, about 1000 to 1300 μg 4-methyl-7-ethoxy coumarin, about 100 to 200 μg vitamin B5, 30 to 60 μg isopropyl-B-D-thiogalactopyranoside, about 250 to 400 μg osthole, about 100 to 200 μg phosphatidylcholine, about 50 to 100 μg 4-shogaol, about 75 to 150 μg piperine/cocluarine/laurifoline, about 50 to 150 μg 8-dehydrogingerdione, about 30 to 100 μg sinomenin/deoxyharringtonine, about 75 to 150 μg μg picrocrocin/carnosol, 300 to 600 μg of resorcinol, about 100 to 250 μg of proline, about 150 to 350 μg of leucine, about 2000 to 3000 μg of adenine, about 4000 to 6000 μg of levoglucosan/glycogen/laminarin, about 1500 to 2,000 μg of synephrine, about 250 to 700 μg of shikimic acid, and about 50 to 150 μg of 3,4-dimethoxychalcone per 100 mg of the extract.

26. A nettle extract comprising a fraction having a Direct Analysis in Real Time (DART) TOF mass spectrometry chromatogram of any of FIGS. 3 to 10.

27. The nettle extract of claim 1, wherein the extract has an IC₅₀ value for COX-1 inhibition of less than 1000 μg/mL.

28. The nettle extract of claim 27, wherein the IC₅₀ value for COX-1 inhibition is about 1 μg/mL to 500 μg/mL.

29. The nettle extract of claim 27, wherein the IC₅₀ value for COX-1 inhibition is about 5 μg/mL to 400 μg/mL.

30. The nettle extract of claim 27, wherein the IC₅₀ value for COX-1 inhibition is about 50 μg/mL to 350 μg/mL.

31. The nettle extract of claim 1, wherein the extract has an IC₅₀ value for COX-2 inhibition is less than 1000 μg/mL.

32. The nettle extract of claim 31, wherein the IC₅₀ value for COX-2 inhibition is about 1 μg/mL to 500 μg/mL.

33. The nettle extract of claim 31, wherein the IC₅₀ value for COX-2 inhibition is about 5 μg/mL to 400 μg/mL.

34. The nettle extract of claim 31, wherein the IC₅₀ value for COX-2 inhibition is about 50 μg/mL to 300 μg/mL.

35. The nettle extract of claim 1, wherein the extract has an IC₅₀ value for 5-LOX inhibition of less than 1000 μg/mL

36. The nettle extract of claim 35, wherein the IC₅₀ for 5-LOX inhibition about 1 μg/mL to 1000 μg/mL.

37. The nettle extract of claim 35, wherein the IC₅₀ for 5-LOX inhibition about 50 μg/mL to 750 μg/mL.

38. The nettle extract of claim 35, wherein the IC₅₀ for 5-LOX inhibition about 100 μg/mL to 500 μg/mL.

39. The nettle extract of claim 1, wherein the IC₅₀ for HPGDS is less than 1000 μg/mL.

40. The nettle extract of claim 39, wherein the extract has an IC₅₀ for HPGDS is about 1 to 1000 μg/mL.

41. The nettle extract of claim 39, wherein the IC₅₀ for HPGDS is about 1 to 500 μg/mL.

42. The nettle extract of claim 39, wherein the IC₅₀ for HPGDS is about 10 to 300 μg/mL.

43. The nettle extract of claim 1, wherein the extract has an IC₅₀ for H₁ antagonism is less than 1000 μg/mL.

44. The nettle extract of claim 43, wherein the IC₅₀ for H₁ antagonism is about 1 to 900 μg/mL.

45. The nettle extract of claim 43, wherein the IC₅₀ for H₁ antagonism is about 1 to 750 μg/mL.

46. The nettle extract of claim 43, wherein the IC₅₀ for H₁ antagonism is about 50 to 500 μg/mL.

47. The nettle extract of claim 43, wherein the IC₅₀ for H₁ antagonism is about 50 to 250 μg/mL.

48. The nettle extract of claim 1, wherein the extract has an IC₅₀ for H₁ negative agonism is less than 1000 μg/mL.

49. The nettle extract of claim 48, wherein the IC₅₀ for H₁ negative agonism is about 1 to 900 μg/mL.

50. The nettle extract of claim 48, wherein the IC₅₀ for H₁ negative agonism is about 1 to 750 μg/mL.

51. The nettle extract of claim 48, wherein the IC₅₀ for H₁ negative agonism is about 50 to 500 μg/mL.

52. The nettle extract of claim 48, wherein the IC₅₀ for H₁ negative agonism is about 50 to 250 μg/mL.

53. The nettle extract of claim 1, wherein the extract has an IC₅₀ for tryptase inhibition is less than 1000 μg/mL.

54. The nettle extract of claim 53, wherein the wherein the IC₅₀ for tryptase is about 1 to 500 μg/mL.

55. The nettle extract of claim 53, wherein the wherein the IC₅₀ for tryptase is about 1 to 250 μg/mL.

56. The nettle extract of claim 53, wherein the wherein the IC₅₀ for tryptase is about 10 to 200 μg/mL.

57. The nettle extract of claim 53, wherein the wherein the IC₅₀ for tryptase is about 20 to 150 μg/mL.

58. A pharmaceutical composition comprising a nettle extract of claim 1 and a pharmaceutically acceptable carrier.

59. The pharmaceutical composition of claim 58, which is formulated as an oral dosage form.

60. The pharmaceutical composition of claim 58, which is formulated as a functional food, dietary supplement, powder or beverage.

61. A method of treating or preventing an inflammatory disorder in a subject comprising administering to a subject in need thereof a therapeutically effective amount of the extract of claim 1.

62. A method of treating or preventing symptoms of an inflammatory disorder in a subject comprising administering to a subject in need thereof a therapeutically effective amount of the extract of claim 1.

63. The method of claim 61, wherein the inflammatory disorder is acute.

64. The method of claim 61, wherein the inflammatory disorder is chronic.

65. The method of claim 61, wherein the inflammatory disorder is allergic rhinitis.

66. The method of claim 61, wherein the inflammatory disorder is selected from rheumatism, osteoarthritis, eczema, arthritis, gout, anemia, enlarged prostate, joint pain, tendonitis, sprains, insect bites, asthma, hay fever, and itchy skin conditions.
